(19) United States
(12) Patent Application Publication
(10) Pub. No.: US 2026/0325565 A1
(43) Pub. Date: Oct. 22, 2026

(54) COMPOSITIONS AND METHODS FOR ACTIVATION AND MATURATION OF IMMUNE CELLS

(71) Applicants: The Broad Institute, Inc., Cambridge, MA (US); Massachusetts Institute of Technology, Cambridge, MA (US)

(72) Inventors: Feng Zhang, Cambridge, MA (US); Mirco Julian Friedrich, Cambridge, MA (US)

(21) Appl. No.: 19/464,793

(22) Filed: Jan. 30, 2026

Related U.S. Application Data
(63) Continuation of application No. PCT/US2024/040682, filed on Aug. 2, 2024.
(60) Provisional application No. 63/530,465, filed on Aug. 2, 2023.
(60) Provisional application No. 63/631,183, filed on Apr. 8, 2024.

(51) Int. Cl. A61K 31/711; A61K 9/51; A61K 38/17; A61K 38/20; A61K 47/18; G01N 15/14; G01N 33/50; G01N 33/68

(52) U.S. Cl. CPC A61K 31/711; A61K 9/5123; A61K 38/1774; A61K 38/20; A61K 47/18; G01N 15/14; G01N 33/505; G01N 33/68; G01N 2500/10

(57) ABSTRACT

Mammals rely on adaptive immunity to protect against infections and tumors, with the thymus playing a crucial role by producing T cells that target infected or cancerous cells. The thymus, however, undergoes functional decline with age, significantly impairing the immune system in the elderly. To investigate reversing this decline, compositions and methods for activation and differentiation of T cell progenitors in extrathymic tissues are disclosed that stimulate production of differentiated T cells. For example, LNP-mediated delivery of mRNAs encoding DLL-1, IL-7, and FLT3L to the liver is shown to create a temporary site for T cell maturation that enhances T cell-mediated immunity in aged mice without causing autoimmunity. This approach facilitates ectopic T cell development from hematopoietic precursors and activates dendritic cells, providing evidence that engineering adaptive immunity in vivo can effectively mitigate immune aging.

### CROSS-REFERENCE TO RELATED APPLICATIONS

This application is a continuation application of International Patent Application No. PCT/US2024/040682, filed Aug. 2, 2024, which claims the benefit of U.S. Provisional Application No. 63/530,465, filed Aug. 2, 2023, and U.S. Provisional Application No. 63/631,183, filed Apr. 8, 2024. The entire contents of the above-identified applications are hereby fully incorporated herein by reference in their entireties.

### STATEMENT REGARDING FEDERALLY SPONSORED RESEARCH

This disclosure was made with government support from Grant No.(s) 5F31CA275339-02 and 1DP2GM146245-01 awarded by the National Institutes of Health. The government has certain rights in the invention.

### REFERENCE TO AN ELECTRONIC SEQUENCE LISTING

The contents of the electronic sequence listing (BROD-5835US_ST26.xml”; Size is 1,137,586 bytes and it was created on Jan. 8, 2026) is herein incorporated by reference in its entirety.

### TECHNICAL FIELD

The subject matter disclosed herein is generally directed to compositions and methods for activating and differentiating T cell progenitors in targeted tissues to stimulate the production of differentiated T cells.

### BACKGROUND

The thymus is a vital organ for establishing and maintaining the adaptive immune system, serving as the site for the selection, proliferation, development, and differentiation of T cells. Over time, the thymus undergoes age-related atrophy (i.e., thymus involution or thymic involution), which results in less efficient T cell development and decreased output of naïve T cells. This can increase susceptibility to opportunistic infections, autoimmunity, and cancer. The inability to establish adaptive immunity can also result in poor vaccine response and increased morbidity and mortality. Thus, there is a need for compositions and methods for the stimulation and differentiation of T-cell progenitors in non-thymic tissues to rejuvenate the adaptive immune system.

Citation or identification of any document in this application is not an admission that such a document is available as prior art to the present disclosure.

### SUMMARY

The present disclosure provides compositions and methods for the activation and differentiation of T cell progenitors in targeted tissues to stimulate the production of differentiated T cells. In one embodiment, the present disclosure provides a composition comprising (a) one or more polynucleotides encoding one or more activating proteins that induce T cell differentiation of T cell progenitor cells and (b) a delivery vehicle configured to deliver the one or more polynucleotides to a target tissue where T cell progenitors differentiate.

In one embodiment, one or more activating proteins is an agonist of a Notch signaling pathway or a T cell receptor (TCR) signaling pathway. In another embodiment, the agonist of the Notch signaling pathway comprises DLL1, DLL4, Jagged1, Jagged2, or a combination thereof. In another embodiment, an agonist of the TCR signaling pathway is a major histocompatibility complex (MHC) class I or MHC class II ligand. In another embodiment, the MHC class I ligand comprises HLA-A, HLA-B, HLA-C, or a homolog thereof. In another embodiment, the MHC class II ligand comprises HLA-DP, HLA-DQ, HLA-DR, or a homolog thereof.

In one embodiment, the agonist of the Notch signaling pathway does not comprise DDL-1 or DLL4. In one embodiment, the agonist of the Notch signaling pathway does not comprise IL-7. In one embodiment, the agonist of the Notch signaling pathway does not comprise FLT3L.

In one embodiment, the composition further comprises one or more cytokines or polynucleotides encoding said cytokines. In another embodiment, the one or more cytokines comprise IL-7, FLT3L, KIT ligand, IL-2, IL-4, IL-15, CCL25, or a combination thereof. In another embodiment, one or more cytokines induce the expression of the transcription factor RUNX3.

In one embodiment, the one or more polynucleotides of any of the compositions described herein comprise one or more plasmid DNAs encoding the one or more activating proteins and/or cytokines; one or more mRNA molecules encoding the one or more activating proteins and/or cytokines; or a combination thereof. In another embodiment, the one or more mRNAs comprise one or more modifications that increase mRNA stability, reduce immunogenicity, and/or improve translation efficiency.

In one embodiment, the delivery vehicle comprises a lipid nanoparticle, an exosome, a microvesicle, a viral vector, an engineered retroelement vector, a polynucleotide-based nanostructure, or an extracellular contractile injection system. In another embodiment, the viral vector comprises a retroviral, lentiviral, adenoviral, adeno-associated, or herpes simplex viral vector.

In one embodiment, the target tissue is the liver, spleen, small intestine, thymus, or a combination thereof.

In one embodiment, the present disclosure provides a method for inducing T cell progenitor differentiation, said method comprising delivering, to a target tissue where T cell progenitor differentiation occurs, one or more polynucleotides encoding one or more activating proteins that induce differentiation of T cell progenitor cells.

In one embodiment, the one or more polynucleotides are formulated so that they are delivered to endothelial cells in the target tissue and that the activating proteins are displayed on the surface of the endothelial cells.

In one embodiment, the target tissue is the liver, spleen, small intestine, or thymus, or a combination thereof.

In one embodiment, the one or more activating proteins induce differentiation of T cell progenitors to double-positive (DP) T cells.

In one embodiment, the one or more activating proteins do not induce autoimmunity.

In one embodiment, the one or more activating proteins is an agonist of a Notch signaling pathway or a T cell receptor (TCR) signaling pathway. In another embodiment, the agonist of the Notch signaling pathway comprises DLL1, DLL4, Jagged1, Jagged2, or a combination thereof. In another embodiment, the agonist of the TCR signaling pathway is an MHC class I or MHC class II ligand. In another embodiment, the MHC class I ligand comprises HLA-A, HLA-B, HLA-C, or a homolog thereof. In another embodiment, the MHC class II ligand comprises HLA-DP, HLA-DQ, HLA-DR, or a homolog thereof.

In one embodiment, the one or more polynucleotides of any methods described herein comprise one or more plasmid DNAs encoding the one or more activating proteins, one or more mRNAs encoding the one or more activating proteins, or a combination thereof. In another embodiment, the one or more mRNAs comprise one or more modifications that increase mRNA stability, reduce immunogenicity, and/or improve translation efficiency.

In one embodiment, the one or more polynucleotides are delivered via a lipid nanoparticle, an exosome, a microvesicle, a viral vector, an engineered retroelement vector, a polynucleotide-based nanostructure, or an extracellular contractile injection system.

In one embodiment, the method further comprises delivering one or more cytokines or polynucleotides encoding one or more cytokines. In another embodiment, the one or more cytokines comprise IL-7, FLT3L, KIT ligand, IL-2, IL-4, IL-15, or CCL25, or a combination thereof. In another embodiment, the one or more cytokines induce expression of the transcription factor RUNX3. In another embodiment, the one or more cytokines are co-delivered with the one or more polynucleotides encoding the one or more activating proteins. In another embodiment, the cytokines are delivered separately, temporally or spatially, from the one or more polynucleotides encoding the activating proteins.

In one embodiment, the one or more cytokines induce differentiation of double-positive (DP) T cells into single-positive (SP) T cells.

In one embodiment, the present disclosure provides a method for identifying candidate activating proteins, or combinations of activating proteins, that induce T cell proliferation and differentiation, said method comprising (a) delivering one or more polynucleotides encoding one or more candidate activating proteins that induce T cell differentiation of T cell progenitor cells to a population of cells derived from a tissue of interest, (b) selecting a sample of single clone cells expressing the candidate activating proteins from the population of cells derived from the tissue of interest, (c) treating a population of T cell progenitors with the sample of single clone cells expressing the candidate activating proteins, (d) quantifying the percentage of T cells differentiated from the population of T cell progenitors, and (e) selecting the polynucleotides encoding the candidate activating proteins that resulted in T cell differentiation for evaluation in vivo, wherein the percentage of differentiated T cells is at or above 50% of the total cell population consisting of the differentiated T cells and T cell progenitors.

In one embodiment, the tissue of interest is the liver, spleen, small intestine, or thymus, or a combination thereof.

In one embodiment, the sample of single clone cells is selected using flow cytometry.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1A-1I depicts the dysfunction of cortical thymic epithelial cells in aging mice.

FIG. 1A depicts a simplified model of T cell development starting from bone marrow (BM)-derived common lymphoid progenitors (CLP) and multipotent progenitors (MPP) homing into the thymus (T), where they receive differentiation signals followed by positive selection by cortical thymic epithelial cells (cTEC) and negative selection by stromal antigen-presenting cells, including medullary thymic epithelial cells (mTECs). Mature CD8⁺ or CD4⁺ single-positive (SP) T cells then circulate systemically, including in the secondary lymphoid organs spleen (S) and lymph nodes (LN). DN, double-negative; DP, double-positive.

FIG. 1B shows the quantification of circulating CLPs (% Sca-1⁺c-Kit⁺ of Lin⁻ IL7ra⁺) in n=35 animals of various ages. Linear regression shown.

FIG. 1C shows quantification of circulating MPPs (% CD34⁺Flt3L⁺ of Lin⁻ Sca-1⁺c-Kit*) in n=35 animals of various ages. Linear regression shown.

FIGS. 1D-1E shows the relative fraction of circulating CLPs within live blood mononuclear cells across age. Each dot is the value for one mouse (n=36). The arrowhead annotates the average start of macroscopic thymic involution in C57BL/6J mice.

FIGS. 1F-1G shows the changes to selected cell-type interactions: cTEC with DN T cells (left, FIG. 1F) and mTEC with DP T cells (right, FIG. 1G). Linear regression plotted and Pearson's R indicated. P-value denotes significance of Pearson's correlation with age. The arrowhead annotates the average start of macroscopic thymic involution in C57BL/6J mice. Violin plots demonstrate the average interaction z-scores pre- and post-thymic involution. P values were calculated using two-tailed unpaired t-tests.

FIGS. 1H-1I shows the decreasing receptor-ligand interactions (normalized interaction strength) with age. Each row is a unique cell-type-cell-type receptor-ligand pair and is annotated by common receptors on TEC (FIG. 1H) or ligands on DN cells (FIG. 1I). Bar charts demonstrate the average interaction z-scores for select interactions pre- and post-thymic involution. P values were calculated using Mann-Whitney tests. For FIGS. 1H-1I, n=47 spatial arrays over 21 time points.

Receptor-ligand analysis: Squidpy integration of CellPhoneDB and Omnipath were used to identify shifts in receptor-ligand interactions at each time point. Subsequently, the mean values of each receptor ligand cell-cell pair across time were employed to calculate Spearman's R correlation and a pvalue. After correcting the p-values through Bonferroni correction, unique interactions were categorized into those decreasing and increasing with age for visualization.

FIG. 2A-2I depicts the formulation and characteristics of lipid nanoparticles (LNPs) containing Dll1, Flt3, and Il7 mRNA (DFI).

FIG. 2A shows maps of linearized plasmids used for in vitro transcription (IVT) of DFI mRNAs.

FIG. 2B show a gel electrophoresis of in vitro transcribed and capped DFI mRNAs.

FIG. 2C shows TapeStation RNA ScreenTape results of in-vitro transcribed and capped DFI mRNAs.

FIG. 2D shows encapsulation efficiencies of DFI-containing SM-102 lipid nanoparticles (LNPs). Three measurements each of two independent batches shown. Data are mean±s.e.m. P value was calculated using a two-tailed unpaired t-test.

FIG. 2E shows LNP diameter p and polydispersity indices (% PD) as measured by dynamic light scattering. Individual LNP batches shown in grey, average calculated and shown in green.

FIGS. 2F-2I Validation of DLL1 mRNA expression in immortalized liver cells.

FIG. 2F shows primary hepatocytes transfected with 2.5 μg EGFP mRNA in 7.5 μL Lipofectamine™ 3000 and subsequently stained with anti-DLL1 monoclonal antibody (HMD1-5) PE for surface expression of DLL1.

FIG. 2G shows primary hepatocytes transfected with 2.5 μg DLL1 mRNA in 7.5 μL Lipofectamine™ 3000 and subsequently stained with anti-DLL1 monoclonal antibody (HMD1-5) PE for surface expression of DLL1.

FIG. 2H show percentage of AML12 primary hepatocytes positive for expression of DLL1 or EGFP.

FIG. 2I shows percentage of HEK 293T cells positive for expression of DLL1 or EGFP.

FIG. 3A-3H: Intravenously injected Dll1-Il7-Flt31 LNPs improve immune responses in aged animals.

FIG. 3A shows a schematic of triple mRNA delivery.

FIGS. 3B-3C shows fluorescence images (FIG. 3B) and quantification (FIG. 3C) of surface DLL1 protein expression in immortalized mouse hepatocytes transfected with 0.25 μg DLL1 mRNA in 7.5 μL Lipofectamine™ 3000 and subsequently stained with anti-DLL1 antibody (AB10554) or mock transfected. β-Catenin staining was used to delineate cell membranes. Scale bar, 100 μm.

FIG. 3D shows the extracellular concentrations of biologically active IL-7 (left) and FLT3L (right) secreted by immortalized hepatocytes transfected with 2.5 μg FLT3L mRNA or 2.5 μg IL-7 mRNA in 7.5 μL Lipofectamine™ 3000 or 7.5 μL empty Lipofectamine™ 3000 as determined by IL-7 and FLT3L ELISA, respectively. Data are mean±s.e.m. P values were calculated using two-tailed unpaired t-tests. n=3 independent transfection experiments.

FIGS. 3E-3F shows mRNA-pulsed hepatocytes secrete processed FTL3L and IL-7. (FIG. 3E) Primary hepatocytes were treated with 2.5 μg FLT3L mRNA in 7.5 μL Lipofectamine™ 3000 or 7.5 μL empty Lipofectamine™ 3000. (FIG. 3F) Primary hepatocytes were treated with 2.5 μg IL-7 mRNA in 7.5 μL Lipofectamine™ 3000 or 7.5 μL empty Lipofectamine™ 3000. ELISA was performed using anti-FLT3L and anti-IL-7 monoclonal antibodies.

FIG. 3G shows serum Flt3L and IL-7 levels in mice treated with either Flt3l/Il7 mRNA LNPs or Luc LNPs. Serum levels measured by Flt3L and IL-7 cytokine ELISA, respectively. Data are mean s.e.m. P values were calculated using a two-tailed unpaired t-test.

FIG. 3H shows IVIS luciferase imaging of Foxn1−/− mice that have received either mock SM-102 LNPs or Luc SM-102 LNPs intravenously 8 hours before imaging. N=4 mice.

FIGS. 4A-4B—In vitro T cell maturation by AML12-DLL1 is not inferior to OP9-DLL1.

FIG. 4A shows schematic of procedure used to show DLL1-expressing hepatocytes induce the differentiation of T cells from hematopoietic stem cells in vitro.

FIG. 4B shows percentage of lymphocytes that are CD4⁻ CD8⁻ double negative, T cell receptor negative (DN TCR−), CD4⁻ CD8⁻ double negative, T cell receptor-positive (DN TCR+), CD4⁺ CD8⁺ double positive (DP), CD4 single positive (SP CD4), or CD8 single positive (SP CD8), following co-incubation of hematopoietic stem cells with AML12 hepatocytes expressing GFP (AML12-GFP) or DLL1 (AML12-DLL1), or with OP9 stromal cells expressing GFP (OP9-GFP) or DLL1 (OP9-GFP).

FIGS. 5A-5B—Screening for in vitro differentiation cocktail combinations.

FIG. 5A shows the procedure for screening for combinations of activating proteins that induce T cell differentiation.

FIG. 5B shows the relative percentages of differentiated T cells for each of the combinations tested, wherein the combinations yielding the highest percentage of differentiated T cells were used for in vivo testing.

FIGS. 6A-6F—In vivo testing in immunocompetent mice.

FIG. 6A Vaccination schedule used to evaluate the efficacy of SYNTHYMUS (DLL1 mRNA in combination with IL-7/FLT3L mRNAs) in athymic mice.

FIGS. 6B-6F shows the treatment of athymic mice with SYNTHYMUS preferentially induces mature CD8+ SP T cell development.

FIG. 7 shows a trajectory of artificially produced T cells. DLL1-mediated T cell production occurs in the liver, followed by cytokine-mediated maturation in the blood and spleen. A total number of T cells harvested from the liver (left), peripheral blood (middle), and spleen (right) of athymic mice treated with empty lipid nanoparticles, cytokines only, DLL1 mRNA, or DLL1 mRNA in combination with cytokines. The relative proportion of T cells that are double negative (DN), double positive (DP), CD4 single positive (SP CD4), or CD8 single positive (SP CD8) is shown.

FIGS. 8A-8F show artificial thymus-produced T cells can be activated ex vivo.

FIGS. 9A-9C show artificial thymus-produced T cells can lyse target cells.

FIG. 9A shows a diagram of the experimental sequence for one-sided mixed lymphocyte reaction (MLR) of T cells derived from spleens of DFI mRNA-treated of Foxn1−/− animals as effector cells and NSG splenocytes as target cells. NSG-mice are T cell-deficient and DFI-induced T cells were additionally labeled with CFSE to allow unambiguous identification and proliferation assessment of effector cells

FIG. 9B shows histograms indicating the abundance and proliferation of CFSE+ T cells after MLR (left) or without target cells (right). Each histogram represents one biological replicate.

FIG. 9C shows the absolute counts of target cells (CFSE−) 24 h after mixed lymphocyte reaction (MLR). Each dot is the value for one mouse. n=5 for all conditions. Effector-to-target (E:T) ratios indicated.

FIGS. 10A-10K show an immune aging model for cancer immunotherapy.

FIG. 10A shows an overview of the establishment of age-induced decline in anti-tumor response due to thymic involution in mice and subsequent evaluation of treatment with the compositions described herein. One group of aged mice was treated with SYNTHYMUS (n=10), and a separate control group was treated with empty LNP (n=10). Following treatment, each group was inoculated with a B16-OVA melanoma cell line on day 0 and treated with an anti-PD-L1 antibody every three days. Tumor growth in response to immune checkpoint blockade (ICB) and overall survival (OS) were evaluated.

FIGS. 10B-10C show a comparison of tumor size in aged mice following tumor inoculation with B16-OVA melanoma cells and subsequent treatment with immune checkpoint blockade (ICB).

FIG. 10D shows the overall probability of survival for aged mice following tumor inoculation with B16-OVA melanoma cells and following treatment with anti-PD-L1 antibody.

FIG. 10E shows a diagram of the experimental sequence for subcutaneous tumor implantation with 50.000 B16-F10-OVA cells into C57BL/6J mice aged 5 weeks (young) or 72 weeks (aged) followed by two treatments with 100 μg anti-PD-L1 in NaCl subcutaneous (d3, d6) and measurement of tumor size and survival analysis.

FIG. 10F shows a quantification of tumor sizes at d14, when the first animal reached the experimental endpoint. Data are mean±s.e.m. P value was calculated using a two-tailed unpaired t-test.

FIG. 10G shows a Kaplan-Meier survival analysis of young and aged animals treated as outlined in d. P value was calculated using a Log-rank (Mantel-Cox) test. Young+ICI (n=5), young+NaCl (n=5), aged+ICI (n=5), aged+NaCl (n=5).

FIG. 10H shows tumor immunogenicity experimental workflow (2^{nd} experiment).

FIG. 10I shows tumor size diagrams (2^{nd} experiment). Each line represents the consecutively measured tumor size values of one mouse.

FIG. 10J shows a quantification of tumor sizes at d12, when the first animal reached the experimental endpoint (2^{nd} experiment). Data are mean±s.e.m. P value was calculated using a two-tailed unpaired t-test.

FIG. 10K shows a Kaplan-Meier survival analysis of aged animals treated as outlined in FIG. 10H (2^{nd} experiment). P value was calculated using a Log-rank (Mantel-Cox) test. n==8 for both conditions.

FIGS. 11A-11H shows an immune aging model for vaccine response.

FIG. 11A shows an overview of establishment of age-induced decline in vaccination response due to thymic involution in mice and subsequent evaluation of treatment with the compositions described herein. One group of aged mice was treated with SYNTHYMUS (n=5), and a separate group of aged mice was treated with empty LNP (n=5). Following treatment, each group was immunized with ovalbumin with complete Freund's adjuvant (OVA/CFA) on day 0 and subsequently provided a booster immunization with ovalbumin with incomplete Freund's adjuvant (OVA/IFA) on day 14. Following immunization, mice were treated with ovalbumin-derived SIINFEKL (SEQ ID NO: 753) and ISQAVHAAHAEINEAGR (SEQ ID NO: 754) (ISQ) tetramer peptides, and T cell adaptive response was evaluated.

FIG. 11B shows an analysis of peripheral blood drawn at day 7 revealed that SYNTHYMUS led to a greater percentage of double positive (DP) T cells.

FIG. 11C shows the percentage of SIINFEKL (SEQ ID NO: 753) tetramer-specific CD8+ T cells isolated from peripheral blood, spleen, liver, and thymus.

FIG. 11D shows epitope-specific secretion of granzyme B (GzmB), IFN-γ, and IL-2.

FIGS. 11E-11F show a comparison of SIINFEKL (SEQ ID NO: 753) tetramer-specific T cells in young mice (Young), aged mice treated with DLL1 mRNA in combination with IL-7 mRNA and FLT3L mRNA (Aged+SYNTHYMUS), and aged mice treated with empty lipid nanoparticle (Aged+LNP).

FIG. 11G shows the vaccination response as an immune aging function, where the percentage of SIINFEKL (SEQ ID NO: 753) tetramer-specific T cells decreases with age.

FIG. 11H shows the treatment with SYNTHYMUS can augment SIINFEKL (SEQ ID NO: 753) tetramer-specific immune response by 20 weeks.

FIG. 11I shows the fold change in intracellular cytokine expression of IL-2 (left) and IFN-γ (MFI) (right) after 6 h recall of splenocytes with SIINFEKL (SEQ ID NO: 753) relative to DMSO vehicle. n=4 animals for both conditions. Data are mean±s.e.m. P values were calculated using two-tailed unpaired t-tests.

FIG. 11J shows the weights of explanted spleens at d21 post vaccination. Each dot is the value for one mouse. n=4 animals for all conditions. Data are mean±s.e.m. P values were calculated using one-way ANOVA followed by two-tailed Dunnett's T3 multiple-comparison post hoc test. Scale bar for representative spleen images, 1 cm.

FIG. 11K shows the relative frequencies of SIINFEKL-H-2K(b) (SEQ ID NO: 753) Tetramer-binding T cells in spleens (left) and peripheral blood (right). Each dot is the value for one mouse. n 4 for all conditions. Data are mean±s.e.m. P values were calculated using one-way ANOVA followed by two-tailed Dunnett's T3 multiple-comparison post hoc test.

FIG. 11L shows the relative frequencies of naïve (CD44⁻ CD62L⁺) T cells in spleens. Each dot is the value for one mouse. n=4 for all conditions. Data are mean±s.e.m. P values were calculated using one-way ANOVA followed by two-tailed Dunnett's T3 multiple-comparison post hoc test.

FIG. 11M shows the longitudinal vaccination experiment of mice with increasing age. Second order polynomial (quadratic) interpolation of relative frequencies of SIINFEKL-H-2K(b) (SEQ ID NO: 753) Tetramer-binding T cells in spleens with 95% confidence bands shown. df=29, R²=0.9116. This standard curve was used to project the “vaccination response age” of numerically 52-week-old animals that have been pre-treated with DFI or Luc mRNA prior to vaccination. Each dot is the value for one mouse. Vaccinated animals (N=32; n=4 per age group), aged+vaccination+Luc mRNA (n=4), aged+vaccination+DFI mRNA (n=4). P value to compare the estimated vaccination response ages of the two treatment groups was calculated using a two-tailed unpaired t-test.

FIGS. 11N-11T: Single-factor delivery does not fully recapitulate the effects of DFI combination treatment.

FIG. 11N. Diagram of the experimental sequence for subcutaneous peptide vaccination with Ovalbumin into mRNA-LNP-treated C57BL/6J followed by measurement of vaccine-induced T cells by flow cytometry. Sham vaccinated mice were used as negative controls.

FIG. 11O. Weights of explanted spleens at d21 post vaccination. Each dot is the value for one mouse. n=4 animals for all conditions. Data are mean±s.e.m. P values were calculated using oneway ANOVA followed by two-tailed Dunnett's T3 multiple-comparison post hoc test. Y, young; O, aged.

FIG. 11P. Relative frequencies of SIINFEKL-H-2K(b) (SEQ ID NO: 753) Tetramer-binding T cells in peripheral blood. Each dot is the value for one mouse. n=4 for all conditions. Data are mean±s.e.m. P values were calculated using one-way ANOVA followed by two-tailed Dunnett's T3 multiple comparison post hoc test. Y, young; O, aged.

FIG. 11Q. Relative frequencies of SIINFEKL-H-2K(b) (SEQ ID NO: 753) Tetramer-binding T cells in the spleen. Each dot is the value for one mouse. n=4 for all conditions. Data are mean±s.e.m. P values were calculated using one-way ANOVA followed by two-tailed Dunnett's T3 multiple-comparison post hoc test. Y, young; O, aged.

FIG. 11R. Relative frequencies of naïve (CD44− CD62L+) T cells in spleens. Each dot is the value for one mouse. n=4 for all conditions. Data are mean±s.e.m. P values were calculated using oneway ANOVA followed by two-tailed Dunnett's T3 multiple-comparison post hoc test. Y, young; O, aged.

FIG. 11S. Relative frequencies of effector (CD44+ CD62L−) T cells in spleens. Each dot is the value for one mouse. n=4 for all conditions. Data are mean±s.e.m. P values were calculated using oneway ANOVA followed by two-tailed Dunnett's T3 multiple-comparison post hoc test. Y, young; O, aged.

FIG. 11T. Relative frequencies of memory (CD44+ CD62L+) T cells in spleens. Each dot is the value for one mouse. n=4 for all conditions. Data are mean±s.e.m. P values were calculated using one way ANOVA followed by two-tailed Dunnett's T3 multiple-comparison post hoc test. Y, young; O, aged.

FIGS. 12A-12B show mRNA delivery results in a slight increase of FLT3L, but not IL-7 serum levels.

FIG. 12A shows serum levels of IL-7 in athymic mice following treatment with DLL1 mRNA.

FIG. 12B shows serum levels of FLT3L in athymic mice following treatment with DLL1 mRNA.

FIG. 13A-13H: DLL1 mRNA does not induce liver toxicity

FIG. 13A shows a diagram of the experimental sequence for safety analysis experiments.

FIG. 13B shows the weights of aged animals (72 weeks) treated for 35 days with Luc mRNA (n=5) or LIFT mRNA (n=5).

FIG. 13C shows liver function parameters measured in serum of aged animals (72 weeks) treated for 35 days with NaCl (n=10), LIFT mRNA (n=9) or recombinant IL-7/FLT3L (n=10) shown. Data are mean±s.e.m.

FIG. 13D shows multiplexed cytokine measurements in peripheral blood of aged animals (72 weeks) treated for 35 days with NaCl (n=10), LIFT mRNA (n=9) or recombinant IL-7/FLT3L (n=10). Average concentration of each analyte is shown.

FIG. 13E shows a representative flow cytometry gating strategy to quantify frequencies of KYNKANAFL-H-2Kd (SEQ ID NO: 781) specific T cells in peripheral blood of NOD mice by tetramer staining.

FIG. 13F shows a diagram of the experimental sequence for i.v. adoptive cell transfer (ACT) of thymus derived CD4+/CD8+ double-positive (DP) T cells (n=5 CD45.1 mice, pooled in NaCl) or NaCl into aged recipients (n=5 CD45.2 mice aged 72 weeks) followed by verification of successful ACT in blood and detection and phenotyping of CD45.1 cells in thymi and spleens.

FIG. 13G shows relative frequencies of CD45.1⁺ in peripheral blood (top, d0), thymus (middle, d7), spleen (bottom, d7). Each dot is the value for one mouse. Aged CD45.2+ACT (n=5), aged CD45.2+NaCl (n=5). Data are mean±s.e.m. P values were calculated using two-tailed unpaired t-tests.

FIG. 13H shows relative frequencies of double-negative (DN), double positive (DP) and single-positive (SP) CD45.1 cells in thymi and spleens of recipients at d7 after ACT. Aged CD45.2+ACT (n=5). Data are mean±s.e.m. P values were calculated using repeated measures one-way ANOVA followed by Sidak multiple-comparison post hoc test.

FIGS. 14A-14L: DFI promotes ectopic T cell development.

FIG. 14A shows the experimental workflow for treatment of athymic B6.Cg-Foxn1−/−/J (Foxn1^{-/-}) mice with DFI or Luc LNPs

FIG. 14B shows fluorescence images of CD3⁺ cells in explanted livers following 7 days of treatment. Scale bar, 5 μm. Representative of three independent experiments

FIG. 14C shows a flow cytometry quantification of absolute counts of CD45⁺ cells (left), CD3⁺ TCRβ⁺ DN T cells (middle) and relative fraction of CD3⁺ TCRβ⁺ DN T cells of CD45⁺ cells in the parenchyma of explanted livers following 7 days of treatment. n=3 animals for both conditions. Data are mean±s.e.m. P values were calculated using two-tailed unpaired t-tests.

FIGS. 14D-14H show single-cell RNA/V(D)J sequencing of CD3⁺ cells isolated from liver parenchyma of DFI-treated Foxn1−/− mice at day 7 by density centrifugation followed by FACS according to the gating shown in FIG. 14C.

FIG. 14D show a Uniform Manifold Approximation and Projection (UMAP) of liver-parenchymal T cell progenitor cells with annotations of three transcriptional clusters I-III (left), density plots of representative marker genes CD44, GATA3, TRBC (right). N=4 DFI mRNA-treated animals; n=706 single cells post QC.

FIG. 14E shows a heatmap of the averaged relative expressions of hematopoietic and T cell progenitor lineage markers, T cell development phase I-specific transcription factors (TFs), TFs overlapping phases I and II, phase II-specific TFs, and TCR genes Trbc and Trac in clusters I-III defined in FIG. 14D.

FIG. 14F shows a UMAP from FIG. 14D annotated by TCR status of each cell.

FIG. 14G shows stacked bar charts split by experiment replication (mice M1/2 or M3/4) and TCR chain indicating the proportion and absolute number of rearrangements of unproductive and productive TCR chains (top) and the proportion of genetic events leading to unproductive TCRs (bottom).

FIG. 14H shows a heatmap of differentially expressed genes in cells split by TCR recombination status.

FIG. 14I shows a flow cytometry analysis of relative fractions of CD3+ TCRβ+ DN T cells of CD45+ cells in peripheral blood following 35 days of treatment. Each dot is the value for one mouse. n=5 animals for both conditions. Data are mean±s.e.m. P value was calculated using a two-tailed unpaired t-test.

FIG. 14J shows the relative fraction of CD4+ and CD8+ SP T cells of CD45+ cells in peripheral blood following 35 days of treatment. Each dot is the value for one mouse. Luc (n=5), DFI (n=4). Data are mean±s.e.m. P values were calculated using two-way ANOVA followed by two-tailed Sidak's post hoc test.

FIGS. 14K-14L show the V(D)J sequencing of peripheral blood samples following 35 days of treatment.

FIG. 14K shows a violin plot of overall Simpson clonality shown.

FIG. 14L shows representative tree maps that denote the up to top 100 TCR clones and their relative frequencies within the V(D)J repertoire of a Luc and DFI-treated animal, respectively. (left). Bar charts depicting the number of unique V(D)J rearrangements (right). Each dot is the value for one mouse. Luc (n=8), DFI (n=6). Data are mean±s.e.m. P values were calculated using two-tailed unpaired t-tests for the number of unique rearrangements

FIGS. 15A-15I: Ectopically developed T cells accumulate in the spleens of athymic animals.

FIGS. 15A-15B show representative images (A) and weights (B) of explanted spleens following 35 days of treatment. Each dot is the value for one mouse. Foxn1^{wt} (n=4), Foxn1^{-/-}+Luc mRNA (n=4), Foxn1^{-/-}+DFI mRNA (n 4) animals. Data are mean±s.e.m. P values were calculated using one-way ANOVA followed by two-tailed Dunnett's T3 multiple-comparison post hoc test. Scale bar for representative spleen images, 1 cm.

FIG. 15C shows the relative fraction of CD3⁺ TCRβ⁺ DN T cells of CD45⁺ cells in spleens following 35 days of treatment. Each dot is the value for one mouse. Luc mRNA (n==4), DFI mRNA (n==4). Data are mean±s.e.m. P value was calculated using a two-tailed unpaired t-test.

FIG. 15D shows the relative fraction of CD4⁺ and CD8⁺ SP T cells of CD45⁺ cells in spleens following 35 days of treatment. Each dot is the value for one mouse. n=4 for both conditions. Data are mean±s.e.m. P values were calculated using two-way ANOVA followed by two-tailed Sidak's post hoc test.

FIG. 15E shows the STARmap workflow for in situ splenocyte profiling.

FIG. 15F shows a representative 2D-projection of major cell types and T cell/dendritic cell subtypes in white pulps from wild-type (WT)/DFI/Luc mice.

FIG. 15G shows: Top: Relative immature T cell abundance per field of view (FOV) in WT/DFI/Luc mice normalized to the WT condition. n=386(WT)/387(DFI)/361(Luc). Middle: Relative naïve CD4⁺/CD8⁺ T cell abundance per field of view (FOV) in WT/DFI/Luc mice normalized to the WT condition. n=249(WT)/247(DFI)/176(Luc). Bottom: Relative naïve cDC1/cDC2 abundance per field of view (FOV) in WT/DFI/Luc mice normalized to the WT condition. n=404(WT)/408(DFI)/386(Luc). P values are calculated by unpaired two-sided t-test.

FIG. 15H shows the differential gene expression analysis of DCs across conditions indicate activation of all DC subtypes.

FIG. 15I shows a neighborhood enrichment quantification by cell-cell adjacency analysis of major cell types.

FIG. 15J-15O: Characterization of ectopically generated T cells in the liver parenchyma.

FIG. 15J shows a representative flow cytometry gating strategy to quantify frequencies of intraparenchymal CD45+ CD3+ T cell (progenitors) in the livers of DFI or Luc mRNA-treated Foxn1−/− animals.

FIG. 15K shows histograms indicating the abundance of CD45+CD3+ T cell (progenitors) in the livers of DFI or Luc mRNA-treated Foxn1−/− animals. Each histogram represents one biological replicate.

FIG. 15L shows a Uniform Manifold Approximation and Projection (UMAP) of liver-parenchymal T cell progenitor cells with annotations of the two biological replicates from 4 DFI mRNA-treated animals (left), density plots of representative marker genes Gata3, Trbc1 (right). N=4 DFI mRNA-treated animals; n=706 single cells post QC.

FIG. 15M shows a heatmap showing averaged relative expressions of transcription factors (TFs) Gata3, Id2, Zbtb16 as well as transcripts of the CD3 complex Cd3g, Cd3d, Cd3e split by V(D)J recombination status and TCR expression in T cell progenitors.

FIG. 15N shows a representative flow cytometry gating strategy to quantify frequencies of ex vivo matured liver-derived T cell progenitors from DFI mRNA-treated Foxn1−/− animals co-cultured with MHC I/II WT or knockout (KO) splenocytes as antigen-presenting cells (APCs). APC fractions were T cell-depleted and liver-derived T cells were additionally labeled with CFSE to allow unambiguous identification.

FIG. 15O shows a quantification of flow cytometry readout outlined in (E). Liver parenchymal T cell progenitors were co-cultured with splenic APCs for 8 days prior to analysis. Proportions of all transferred and live cells, double-negative (DN), CD4/CD8 double-positive (DP) and CD4 or CD8 single positive (SP) T cells shown. n=6 biological replicates for all conditions. Data are mean±s.e.m. P values were calculated using one-way ANOVA followed by two-tailed Dunnett's T3.

FIGS. 16A-16G: DFI demonstrates favorable toxicity and immunological safety.

FIG. 16A shows the experimental workflow to test for the development of type 1 diabetes (T1D). NOD.Cg-Tg(TcraTcrbNY8.3)1Pesa/DvsJ (NY8.3) mice transgenic for the autoreactive TCR recognizing NRP-V7 (KYNKANAFL) (SEQ ID NO: 781) included as positive control group. Experimental endpoint (Onset of T1D) was reached in animals with blood glucose levels >200 mg/dL in two independent measurements or in animals developing glucosuria.

FIG. 16B shows a longitudinal blood glucose levels (top) and frequency of NRP-V7-specific TCRs in the peripheral blood (bottom) of NOD animals treated as outlined in (D).

FIG. 16C shows a Kaplan-Meier analysis of cumulative diabetes incidence analysis of NOD animals treated as outlined in (D). Onset of T1D was defined as in (D). P values were calculated using Log-rank (Mantel-Cox) tests. NY8.3 (n=5), NOD+Luc mRNA (n=9), NOD+DFI mRNA (n=9).

FIG. 16D shows an experimental workflow to monitor for the development of OVA-targeted T cell immunity. C57BL/6-Tg(TcraTcrb)1100Mjb/J (OT-I) mice transgenic for the TCR recognizing OVA₂₅₇₋₂₆₄ included as positive control group for CD8-driven OVA-targeted responses, B6.Cg-Tg(TcraTcrb)425Cbn/J (OT-II) mice transgenic for the TCR recognizing OVA₃₂₉₋₃₃₇ included as positive control group for CD8-driven OVA-targeted responses.

FIG. 16E shows the relative frequencies of SIINFEKL-H-2K(b) (SEQ ID NO: 753) Tetramer-binding T cells in peripheral blood. Each dot is the value for one mouse (n 5 for all conditions). Data are mean s.e.m. P values were calculated using one-way ANOVA followed by two-tailed Dunnett's T3 multiple-comparison post hoc test. P value comparing Act-mOVA groups indicated.

FIG. 16F shows the relative frequencies of AAHAEINEA-I-A(b) (SEQ ID NO: 782) Tetramer-binding T cells in peripheral blood. Each dot is the value for one mouse (n=5 for all conditions). Data are mean s.e.m. P values were calculated using one-way ANOVA followed by two-tailed Dunnett's T3 multiple-comparison post hoc test. P value comparing Act-mOVA groups indicated.

FIG. 16G shows an anti-OVA IgG ELISA with sera of Act-mOVA mice treated with Luc (n 5) or DFI (n=5) mRNA for 35 days and challenged by OVA/CFA vaccination and OVA/IFA boost (O) or sham vaccination (S). Vaccinated C57BL/6J wild-type (WT, n=5) mice used as positive controls. OD_{450 nm-570 nm} shown.

FIG. 17A-17F: STARmap PLUS identifies ectopically generated T cells and new cell-cell interactions in the spleens of DFI-treated mice.

FIG. 17A. Computational workflow for cell typing of splenocytes by STARmap. After quality control cells were clustered into regions by SPIN and cells in the identified white pulp regions were further clustered by K-means clustering using listed marker genes.

FIG. 17B. Gene expression dot plots for level 1 clustering of major cell types.

FIG. 17C. Gene expression dot plots for level 2 clustering of major cell types.

FIG. 17D. Gene expression dot plots for level 3 clustering of major cell types.

FIG. 17E. Heat map showing the relative gene expression (normalized by gene) for ectopically generated T cell subtypes.

FIG. 17F. Neighborhood enrichment quantification by CCA analysis showing all T cell subtypes.

FIG. 18A-18J: Ectopically generated T cells are functional.

FIG. 18A. Diagram of the experimental sequence for DFI mRNA treatment of Foxn1−/− animals for 35 days followed by sort of splenic T cells and the outlined functional assessments. C57BL/6J mice were used as positive controls.

FIG. 18B. Relative frequencies of IFN-γ-positive T cells after activation with the indicated reagents. Each dot is the value for one mouse. n=5 for all conditions. Data are mean s.e.m. P values were calculated using one-way ANOVA followed by two-tailed Dunnett's T3 multiple-comparison post hoc test. nu, Foxn1−/−; wt, C57BL/6J.

FIG. 18C. Representative flow cytometry gating strategy to quantify frequencies of cytokine-producing T cells after ex vivo stimulation with the indicated reagents.

FIG. 18D. Relative frequencies of IL-2-positive T cells after activation with the indicated reagents. Each dot is the value for one mouse. n=5 for all conditions. Data are mean s.e.m. P values were calculated using one-way ANOVA followed by two-tailed Dunnett's T3 multiple-comparison post hoc test. nu, Foxn1−/−; wt, C57BL/6J.

FIG. 18E. Absolute T cell counts 24 h after activation with the indicated reagents. Each dot is the value for one mouse. n=5 for all conditions. Baseline (0 h) value indicated by dotted line. Data are mean±s.e.m. P values were calculated using one-way ANOVA followed by two-tailed Dunnett's T3 multiple-comparison post hoc test. nu, Foxn1−/−; wt, C57BL/6J.

FIG. 18F. Diagram of the experimental sequence for one-sided mixed lymphocyte reaction (MLR) of T cells derived from spleens of DFI mRNA-treated of Foxn1−/− animals as effector cells and NSG splenocytes as target cells. NSG-mice are T cell-deficient and DFI-induced T cells were additionally labeled with CFSE to allow unambiguous identification and proliferation assessment of effector cells.

FIG. 18G. Representative flow cytometry gating strategy to identify CFSE-labeled effector cells and unlabeled target cells after MLR.

FIG. 18H. Histograms indicating the abundance and proliferation of CFSE+ T cells after MLR (left) or without target cells (right). Each histogram represents one biological replicate.

FIG. 18I. CFSE median fluorescence intensity in effector cells 24 h after MLR. Each dot is the value for one mouse. n=5 for all conditions. J. Absolute counts of target cells (CFSE−) 24 h after MLR. Each dot is the value for one mouse. n=5 for all conditions. Effector-to-target (E:T) ratios indicated.

FIG. 18J shows CFSE median fluorescence intensity in effector cells 24 h after MLR. Each dot is the value for one mouse. n=5 for all conditions.

FIG. 19 shows a graphical overview of the experimental approach and the proposed DFI action mechanism. LNPs, lipid nanoparticles; TCR, T cell receptor; DN, double-negative T cell; DP, double-positive T cell; SP, single-positive T cell.

The figures herein are for illustrative purposes only and are not necessarily drawn to scale.

### DETAILED DESCRIPTION OF THE EXAMPLE EMBODIMENTS

### I. General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); Marten H. Hofker and Jan van Deursen, Transgenic Mouse Methods and Protocols, 2^{nd} edition (2011) and CRISPR Gene Editing: Methods and Protocols, Editor: Yonglun Luo, Publisher: Springer New York, 2019.

As used herein, the singular forms “a,” “an,” and “the” include both singular and plural referents unless the context dictates otherwise.

The term “optional” or “optionally” means that the subsequently described event, circumstance, or substituent may or may not occur and that the description includes instances where the event or circumstance occurs and instances where it does not.

The recitation of numerical ranges by endpoints includes all numbers and fractions subsumed within the respective ranges and the recited endpoints.

The terms “about” or “approximately,” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed disclosure. It is understood that the value to which the modifier “about” or “approximately” refers is also specifically, and preferably, disclosed.

As used herein, a “biological sample” may contain whole cells and live cells and/or cell debris. The biological sample may contain (or be derived from) a “bodily fluid.” The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example, by puncture or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and the progeny of a biological entity obtained in vivo or cultured in vitro are also encompassed.

In an embodiment, the terms SYNTHYMUS or DFI are used interchangeably herein to refer to DLL1 mRNA in combination with IL-7/FLT3L mRNAs or Lipid nanoparticles (LNPs) containing DLL1, FLT3L and IL-7 mRNAs.

In an embodiment, an immune checkpoint inhibitor may be any molecule that inhibits an immune checkpoint. Immune checkpoints are well known in the art and include, without limitation, PD-1, PD-L1, PD-L2, CTLA4, B7-H3, B7-H4, BTLA, IDO, KIR, LAG3, A2AR, TIM-3, and VISTA. In some embodiments, the inhibitor is an antibody against the immune checkpoint protein. In certain embodiments, the immune checkpoint inhibitor is an inhibitor of PD-1 or PD-L1, e.g., an antibody that specifically binds PD-1 or PD-L1. In some embodiments, the immune checkpoint inhibitor is nivolumab, pembrolizumab, ipilimumab, durvalumab, or atezolizumab. In one embodiment, the immune checkpoint inhibitor is nivolumab.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment,” “an embodiment,” and “an example embodiment” means that a particular feature, structure, or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment but may.

Furthermore, the particular features, structures, or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

Titles or subtitles may be used in the specification for the sole convenience of the reader but are not intended to influence the scope of the present disclosure or to limit any aspect of the disclosure to any subsection, subtitle, or paragraph.

All publications, published patent documents, and patent applications cited herein are hereby incorporated by reference to the same extent as though each publication, published patent document, or patent application was specifically and individually indicated as being incorporated by reference.

### II. Overview

The present disclosure provides compositions and methods for the activation and differentiation of T cell progenitors in non-thymic tissues to stimulate the production of differentiated T cells. The compositions and methods described herein can restore an adaptive immune response where T cell production is diminished due to thymic atrophy or involution. In general, the compositions comprise one or more polynucleotides encoding one or more activating proteins that induce or promote T cell differentiation of T cell progenitor cells, and a delivery vehicle configured to deliver said polynucleotides to a target tissue where T cell progenitors differentiate. The compositions described herein may be delivered to a subject in need (e.g., a subject experiencing thymic atrophy or involution), wherein the compositions are directed to one or more target tissues. Once taken up by the cells comprising the target tissues, the one or more polynucleotides are transcribed and/or translated, yielding the one or more activating proteins. The polynucleotide may encode activating proteins expressed on the surface of the target tissue cells. Circulating T cell progenitors that may pass through or otherwise make contact with the target tissue, may interact with the activating proteins now expressed by the target tissue cells via Notch receptors on the surface of the T cell progenitors. For example, a circulating T cell progenitor expressing Notch1 may interact with a hepatic cell expressing transgenic Delta-like ligand 4 (DLL4) via a Notch1-DLL4 interaction. Binding of the activating protein on the surface of target tissue cells to the Notch receptor on the CD4⁻ CD8⁻ double negative (DN) T cell progenitors induces a series of transcriptional events that lead to the differentiation of the T cell progenitors into CD4⁺ CD8⁺ double positive (DP) T cells. Subsequent exposure of the DP T cells to cytokines delivered to the subject in need thereof induces the DP T cells to further differentiate and mature into CD4⁺ or CD8⁺ single positive (SP) T cells.

In another aspect, the present disclosure provides methods for inducing T cell progenitor differentiation comprising delivering, to a target tissue where T cell progenitor differentiation occurs, one or more polynucleotides encoding one or more activating proteins that induce or promote differentiation of T cell progenitor cells.

In another aspect, the present disclosure provides methods for identifying candidate activating proteins, or combinations of activating proteins, that induce T cell proliferation and differentiation.

The compositions and methods described herein address the need to restore adaptive immune response where T cell production is diminished. Delivery of the compositions to target non-thymic tissues and presentation of the activating proteins and cytokines therein provide an alternative environment for in vivo differentiation of T cell progenitors and maturation of T cells. The increased production of naïve and mature T cells can improve adaptive immune response and prove particularly useful for improving vaccine efficacy and clinical outcomes in cancer immunotherapy.

### III. Immune Rejuvenation Compositions

The present disclosure provides compositions for the activation and differentiation of T cell progenitors in non-thymic tissues to stimulate the production of differentiated T cells. In one embodiment, the present disclosure provides a composition comprising (a) one or more polynucleotides encoding one or more activating proteins that induce T cell differentiation of T cell progenitor cells, and (b) a delivery vehicle configured to deliver the one or more polynucleotides to a target tissue where T cell progenitors differentiate. In one embodiment, the composition may further comprise one or more cytokines or one or more polynucleotides encoding said cytokines.

As used herein, the term “T cell progenitor” (used interchangeably with “progenitor T cell(s)”, “T cell precursor(s)”, “precursor T cell(s)”, “thymic progenitor(s)”, “thymic progenitor cell(s)”, “early thymic progenitor cell(s)”, “thymic seeding progenitor cell(s)”, or with any other applicable term or terms known by one skilled in the art) refers to any cell or cell type that may be induced to differentiate and develop into a T cell. The term may encompass any undifferentiated cell subject to any stage of T cell development up to, but not including differentiation into CD4+CD8+ double positive (DP) cells. Examples of T cell progenitors include but are not limited to, hematopoietic stem cells, hematopoietic stem precursor cells, pluripotent stem cells, induced pluripotent stem cells, multipotent stem cells, multipotent progenitors, oligopotent stem cells.

As used herein, the term “differentiated T cell” refers to any cell or cell type subject to any stage of T cell development, wherein said cell or cell type is a CD4+ CD8+ DP cell, a CD4+ CD8− single positive (SP) cell, or a CD4− CD8+ SP cell.

1) Activating Proteins that Induce T-Cell Differentiation

The compositions described herein may comprise one or more polynucleotides encoding one or more activating proteins that induce T cell differentiation of T cell progenitor cells. As used herein, the term “activating protein” refers to any protein that induces a T cell progenitor to differentiate into a CD4+ CD8+ DP T cell, a CD4+ CD8− SP cell, or a CD4− CD8+ T cell; or mediates positive and/or negative selection of T cells. In one embodiment, the one or more activating proteins is an agonist of a Notch signaling pathway. In one embodiment, the one or more activating proteins is an agonist of a T cell receptor (TCR) signaling pathway. The compositions described herein may further comprise one or more cytokines or one or more polynucleotides encoding said cytokines.

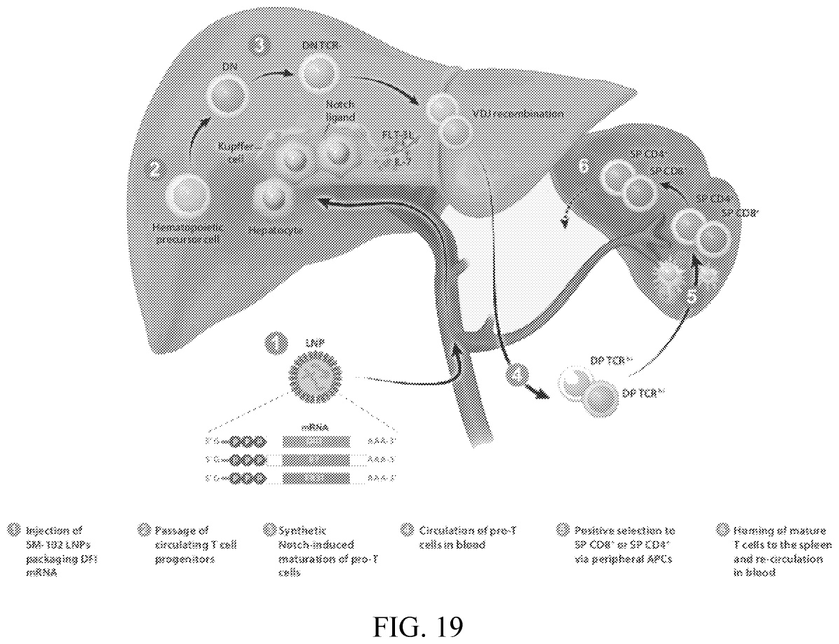

As contemplated in the present disclosure, the compositions described herein, comprising one or more polynucleotides encoding one or more activating proteins, can be delivered to a non-thymic target tissue wherein T cell differentiation can take place (FIG. 19). In a non-limiting example, the compositions described herein can be delivered intravenously and migrate to the liver or spleen via the circulatory system. Once taken up by hepatic or splenic cells, the one or more polynucleotides are transcribed and/or translated, yielding the one or more activating proteins (e.g., an agonist of the Notch signaling pathway or TCR signaling pathway) to be expressed on the surface of the cells. Circulating T cell progenitors that may pass through or otherwise make contact with the liver or spleen, may interact with the activating proteins now expressed by the hepatic or splenic cells via Notch receptors on the surface of the T cell progenitors or TCRs in the case of differentiated T cells.

### 2) Notch Signaling Pathway Agonists

In one embodiment, the one or more activating proteins that induce T cell differentiation of T cell progenitors may comprise an agonist of a Notch signaling pathway. Notch is a transmembrane receptor that regulates various processes in the development and differentiation of most cells, including T cells. The Notch family includes four cell surface receptors and five cell surface Notch ligands in mammals. The four receptors consist of Notch1, Notch2, Notch3, and Notch4. The Notch ligands fall into two families known as Delta-like ligands (DLL), which consist of DLL1, DLL3, and DLL4, and Serrate-like Jagged ligands, which consist of Jagged1 and Jagged2. DLL3 functions as an antagonist of these ligands, while the remaining four ligands (DLL1, DLL4, Jagged1, and Jagged2) function as agonists. Kopan et al., The canonical signaling pathway: unfolding the activation mechanism, Cell, 2009, 137: 216-233.

The canonical Notch receptor is a transmembrane protein expressed as a heterodimer, having an extracellular domain and an intracellular domain. The extracellular domain contains epidermal growth factor (EGF)-like repeats (i.e., repeats of six cysteine residues that form three disulfide bonds) that bind either DLL or Jagged ligands. The binding of an agonist ligand (e.g., DLL1, DLL4, Jagged1, Jagged2) induces proteolytic cleavage that releases the intracellular domain of Notch into the cytoplasm. The intracellular domain then migrates to the nucleus, where it interacts and forms a complex with the transcription factor RBP-J and a Mastermind-like transcriptional co-activator (e.g., MAML1, MAML2, MAML3). This complex recruits additional co-activators and induces transcription of the genes involved in T-cell development. Once the transcription complex has formed, the MAML transcriptional co-activator recruits kinases to phosphorylate the Notch intracellular domain, leading to E3 ligase-dependent degradation.

Notch signaling plays an essential role in the development of mature T cells from lymphocyte and T cell progenitors. In the cortical region of the thymus, the Notch receptors on the surface of CD4⁻ CD8⁻ double negative (DN) T cell progenitors interact with DLL expressed on thymic stromal cells, leading to the development of CD4⁺ CD8⁺ double positive (DP) T cells. The primary Notch-DLL interaction necessary for T cell differentiation and development is that between Notch1 and DLL4. Although DN T cell progenitors interact primarily with DLL4, they can also interact with DLL1, Jagged1, and Jagged2, among other Notch signaling agonists.

In one embodiment, the agonist of the Notch signaling pathway may comprise DLL1, DLL4, Jagged1, Jagged2, or a combination thereof. In another embodiment, the agonist of the Notch signaling pathway may comprise one or more fragments of DLL1, DLL4, Jagged1, Jagged2, or a combination thereof. In another embodiment, the agonist of the Notch signaling pathway may comprise a recombinant or fusion protein comprising any one of DLL1, DLL4, Jagged1, Jagged2, or a combination thereof. In another embodiment, the recombinant or fusion protein may comprise any one of DLL1, DLL4, Jagged1, or Jagged2, and a fragment of an immunoglobulin protein. Non-limiting examples of such recombinant or fusion proteins include Notch signaling agonists comprising the extracellular domain of DLL1 or DLL4 fused to the Fc fragment of IgG as described in U.S. Pub. No. 2023/0073449 and U.S. Pat. No. 11,525,119.

In an embodiment, DLL1 has a protein accession of NP_005609.3. In an embodiment, DLL1 has a genomic accession of NG_027940.1. In an embodiment, DLL1 has a mRNA accession of NM_005618.4. In an embodiment, DLL1 has a secondary protein accession of XP_005266991.1, XP_054184659.1, or XP_054211199.1. In an embodiment, DLL1 has a secondary mRNA accession of XM_005266934.5, XM_054328684.1, or XM_054355224.1. In an embodiment, DLL1 has a secondary genomic accession of NC_000006.12, NT_187553.1, or NC_060930.1. In an embodiment, DLL1 has a secondary protein accession of AAG09716.1, EAW47425.1, AAF05834.1, BAG36904.1, AAQ89251.1, ACH57449.1, or AD022155.1. In an embodiment, DLL1 has a secondary mRNA accession of AF196571.1, AK311474.1, AK314234.1, AY358892.1, BC028096.1, DA243552.1, DR000029.1, EU927387.1, or GQ891293.1. In an embodiment, DLL1 has a secondary genomic accession of AF222310.1, AL078605.30, CH471051.2, CP068272.2, or KT583886.1. In an embodiment, DLL1 has a protein accession of 000548.2. In an embodiment, DLL1 has a secondary protein accession of B2RAK7, B5M0B3, Q9NU41, or Q9UJV2.

### 3) T Cell Receptor (TCR) Signaling Pathway Agonists

In one embodiment, the one or more activating proteins that induce T cell differentiation of T cell progenitors may comprise an agonist of a T-cell receptor (TCR) signaling pathway. In another embodiment, the agonist of the TCR signaling pathway may comprise a ligand of the major histocompatibility complex (MHC). Ligands encoded by the MHC are divided into main classes, class I molecules or ligands and class II molecules or ligands. Class I loci in humans are denoted as human leukocyte antigen (HLA)-A, HLA-B, and HLA-C, and are among the most polymorphic loci in the entire human genome. Class I ligands bind intracellular peptides derived from the cytosol for presentation to CD8+ T cells. Class II loci in humans are denoted as HLA-DP, HLA-DQ, and HLA-DR. These molecules, which are found on the surface of dendritic cells and other specialized antigen-presenting cells (APCs), present peptides to CD4+ T cells for the initiation of cytokine production or antibody production via T helper functions.

For developing T cells to progress to mature SP T cells, they must exhibit a degree of self-reactivity to distinguish between self and non-self. In the thymus, MHC ligands on the surface of thymic epithelial cells present self-peptides (forming a complex termed “peptide-MHC” or “pMHC” complex) to immature DP T cells. T cells that react weakly with these self-pMHC complexes are selected for further differentiation to CD4+ or CD8+ SP T cells in a process called positive selection. T cells that do not react with self-pMHC complexes die by neglect, and those that react too strongly undergo clonal deletion by apoptosis via negative selection.

As contemplated in the present disclosure, the compositions described herein, comprising one or more polynucleotides encoding one or more agonists of the TCR signaling pathway (e.g., an MHC class I or MHC class II ligand), can be delivered to a non-thymic target tissue wherein T cell differentiation can take place (FIG. 19). Once taken up by the cells comprising the non-thymic target tissue, the one or more polynucleotides are transcribed and/or translated, leading to the expression of the one or more TCR signaling pathway agonists on the surface of the target tissue cells. Circulating T cells that may pass through or otherwise make contact with the target tissue, may interact with the TCR signaling agonists expressed on the surface of the target tissue cells via, for example, TCR-pMHC interactions.

In one embodiment, the agonist of the TCR signaling pathway may comprise a major histocompatibility complex (MHC) class I ligand. In another embodiment, the MHC class I ligand comprises HLA-A, HLA-B, HLA-C, or a homolog thereof. In one embodiment, the agonist of the TCR signaling pathway may comprise an MHC class II ligand. In another embodiment, the MHC class II ligand may comprise HLA-DP, HLA-DQ, HLA-DR, or a homolog thereof.

In one embodiment, the agonist of the TCR signaling pathway may be a synthetic peptide-MHC complex or an altered peptide ligand.

### 4) Cytokines

In one embodiment, the compositions described herein may further comprise one or more cytokines or polynucleotides encoding the one or more cytokines. Cytokines play a crucial role throughout the differentiation of DN T cell progenitors to DP T cells. During early T cell development, IL-7 and KIT ligands in the thymic microenvironment promote rapid proliferation of T cell progenitors to maintain the pool of such cells for further differentiation and development. Buono et al., A dynamic niche provides Kit ligand in a stage-specific manner to the earliest thymocyte progenitors, Nat Cell Biol, 2016, 18: 157-167. FLT3L has also been observed to play a role in the proliferation of hematopoietic progenitor cells. Gabbianelli et al., Multi-level effects of FLT3 ligand on human hematopoiesis: expansion of putative stem cells and proliferation of granulomonocytic progenitors monocytic precursors, Blood, 1995, 86 (5): 1661-1670. IL-7 is required for expression of the transcription factor RUNX3, which is necessary for CD8 lineage commitment. Park et al., Signaling by intrathymic cytokines, not T cell antigen receptors, specifies CD8 lineage choice and promotes the differentiation of cytotoxic-lineage T cells, Nat. Immunol., 2010, 11: 257-264. In combination with IL-15, IL-7 is also required for development of CD8+ T cells.

In one embodiment, the one or more cytokines may comprise IL-7, FLT3L, KIT ligand, IL-2, IL-4, IL-15, CCL25, or a combination thereof. In another embodiment, the one or more cytokines induce the expression of RUNX3.

In an embodiment, IL-7 has a protein accession of NP_000871.1, NP_001186815.1, NP_001186816.1, or NP_001186817.1. In an embodiment, FLT3L has a mRNA accession of NM 000880.4, NM_001199886.2, NM_001199887.2, or NM_001199888.2. In an embodiment, IL-7 has a secondary protein accession of XP_011515825.1, XP_011515824.1, XP_047277724.1, XP 047277723.1, XP_047277721.1, XP 047277722.1, XP_054216413.1, XP_054216412.1, or XP_054216414.1. In an embodiment, IL-7 has a secondary mRNA accession of XM_011517523.4, XM 011517522.4, XM 047421768.1, XM 047421767.1, XM_047421765.1, XM_047421766.1, XM_054360438.1, XM_054360437.1, or XM_054360439.1. In an embodiment, IL-7 has a secondary genomic accession of NC_000008.11 or NC_060932.1. In an embodiment, IL-7 has a secondary protein accession of AAC63047.1, EAW87060.1, ABK41904.1, BAD89408.1, BAD89409.1, BAD89411.1, BAD89412.1, BAD89414.1, BAD89422.1, BAF84227.1, AAH47698.1, ACX53627.1, AAA59156.1, or ANQ68335.1. In an embodiment, IL-7 has a secondary mRNA accession of AB102879.1, AB102880.1, AB102882.1, AB102883.1, AB102885.1, AB102893.1, AK226000.1, AK291538.1, AU136355.1, BC032487.1, BC047698.1, CD013923.1, GQ919059.1, J04156.1, or KX160446.1. In an embodiment, IL-7 has a secondary genomic accession of AC083837.4, AC100854.2, AH006906.2, CH471068.1, EAW87061.1, EAW87062.1, CP068270.2, EF064721.1, or LC461681.1. In an embodiment, IL-7 has a protein accession of P13232. In an embodiment, IL-7 has a secondary protein accession of A0N0L3, Q5FBY5, or Q5FBY9.

In an embodiment, FLT3L has a protein accession of NP_001389760.1, NP_001389761.1, NP 001389762.1, NP_001389763.1, NP_001389764.1, NP_001389765.1, NP_001389766.1, or NP_038548.3. In an embodiment, FLT3L has a mRNA accession of NM_001402831.1, NM 001402832.1, NM_001402833.1, NM_001402834.1, NM_001402835.1, NM_001402836.1, NM_001402837.1, or NM_013520.4. In an embodiment, FLT3L has a RNA accession of NR_102725.2, NR_175301.1, NR_175302.1, NR_175303.1, NR_175304.1, NR_175305.1, NR_175306.1, or NR_175307.1. In an embodiment, FLT3L has a secondary protein accession of XP_036008569.1, XP_036008568.1, XP_036008567.1, XP_006540675.1, XP 036008570.1, XP 036008566.1, XP_036008564.1, XP_036008565.1, or XP_036008563.1. In an embodiment, FLT3L has a secondary mRNA accession of XM_036152676.1, XM_036152675.1, XM_036152674.1, XM 006540612.4, XM_036152677.1, XM_036152673.1, XM_036152671.1, XM_036152672.1, or XM_036152670.1. In an embodiment, FLT3L has a secondary RNA accession of XR_882018.2, XR_004934016.1, XR 882016.2, XR_004934014.1, XR_882017.3, XR_004934015.1, XR_882012.3, XR_882010.3, XR_004934013.1, XR 004934012.1, XR_004934011.1, XR_004934010.1, XR_004934009.1, or XR_004934008.1. In an embodiment, FLT3L has a secondary genomic accession of NC_000073.7. In an embodiment, FLT3L has a secondary protein accession of EDL22807.1, EDL22808.1, EDL22809.1, EDL22810.1, EDL22811.1, EDL22812.1, EDL22813.1, AAA90951.1, AAA90952.1, AAA93305.1, AAA93306.1, AAA93307.1, BAB31997.1, AAH19801.1, ABX76044.1, AAA39436.1, AAB33069.1, AAB33070.1, AAB33071.1, or AAA18000.1. In an embodiment, FLT3L has a secondary mRNA accession of AK020105.1, BB224229.2, BC019801.1, CK622267.1, EU274583.1, L23636.1, S76459.1, S76461.1, S76464.1, or U04807.2. In an embodiment, FLT3L has a secondary genomic accession of AC126256.4, CH466603.1, U29875.2, or U44024.1. In an embodiment, FLT3L has a protein accession of P49772.1.

### 5) Polynucleotides

The compositions and methods described herein may comprise one or more polynucleotides encoding one or more activating proteins that induce T cell differentiation of T cell progenitor cells (e.g., Notch signaling pathway agonists, TCR signaling agonists, cytokines). As used herein with reference to the relationship between DNA, cDNA, RNA, mRNA, protein/peptides, and the like, “corresponding to” or “encoding” (used interchangeably herein) refers to the underlying biological relationship between these different molecules. As such, one skilled in the art would understand that operatively “corresponding to” can direct them to determine the possible underlying and/or resulting sequences of other molecules given the sequence of any other molecule that has a similar biological relationship with these molecules. For example, from a DNA sequence, an RNA sequence can be determined, and from an RNA sequence, a cDNA sequence can be determined.

As used herein, “polynucleotide” (used interchangeably herein with “nucleic acid” and “nucleotide sequence”) can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region, often an oligonucleotide. “Polynucleotide” and “nucleic acids” also encompasses such chemically, enzymatically, or metabolically modified forms of polynucleotides, as well as the chemical forms of DNA and RNA characteristic of viruses and cells, including simple and complex cells, inter alia. For instance, the term polynucleotide, as used herein, can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide,” “nucleotide sequences,” and “nucleic acids” also include PNAs (peptide nucleic acids), phosphorothioates, and other variants of the phosphate backbone of native nucleic acids. Natural nucleic acids have a phosphate backbone; artificial nucleic acids can contain other types of backbones but contain the same bases. Thus, DNAs or RNAs with backbones modified for stability or for other reasons are “nucleic acids” or “polynucleotides” as that term is intended. herein. As used herein, “nucleic acid sequence” and “oligonucleotide” also encompasses a nucleic acid and polynucleotide as defined elsewhere herein.

6) Messenger RNA (mRNA)

In certain example embodiments of the compositions described herein, the one or more polynucleotides encoding the one or more activating proteins and/or cytokines may comprise one or more messenger RNA (mRNA) molecules.

In an embodiment, one or more mRNA molecules include one or more mRNA molecules that encode one or more Notch signaling pathways, one or more cytokines, or a combination thereof. In an embodiment, one or more mRNA molecules include one or more mRNA molecules that encode one or more Notch signaling pathways and one or more cytokines. In an embodiment, the one or more mRNA molecules include one or more mRNA molecules that encode DLL1, IL-7, FLT3L, or a combination thereof. In an embodiment, the one or more mRNA molecules include one or more mRNA molecules that encode DLL1, IL-7, and FLT3L. In an embodiment, the one or more mRNA molecules include one or more mRNA molecules that encode DLL1 and IL-7. In an embodiment, the one or more mRNA molecules include one or more mRNA molecules that encode DLL1 and FLT3L. In an embodiment, the one or more mRNA molecules include one or more mRNA molecules that encode IL-7 and FLT3L.

a) mRNA Modifications

In certain example embodiments of the compositions described herein, the one or more mRNA molecules may comprise one or more modifications that increase mRNA stability, reduce immunogenicity, and/or improve translation efficiency. Modifications may be made to any of the various portions of an mRNA molecule, including the 5′-cap, the coding sequence, and the 3′-poly-A tail.

### b) 5′-Cap Modifications

In one embodiment, the one or more mRNA molecules may comprise a 5′-cap. In one embodiment, the 5′-cap may comprise one or more modifications that increase stability, reduce immunogenicity, and/or improve the translation efficiency of the mRNA molecule. The 5′-end of the mRNA (also known as the 5′-cap) is essential for modulating mRNA stability and translational efficiency. In eukaryotes, the 5′-cap consists of a modified 7-methyl-guanosine (m⁷G) cap, which is formed co-transcriptionally and is able to protect the mRNA from 5′-end degradation by cellular nucleases. The m⁷G is ligated to the first transcribed nucleotide via a 5′-5′ trisphosphate bond to form the minimal 5′-cap structure (m⁷GpppN, wherein ‘N’ represents the first transcribed nucleotide of the mRNA). The 5′-cap plays a role in the initiation of translation, serving as the site to which the eukaryotic initiation factor eIF4E binds and recruits the translational machinery to initiate translation of the encoded protein. Given the role that 5′-cap plays in mRNA stability and translation, optimizing the 5′-cap is crucial for designing effective mRNA vectors.

In one embodiment, the one or more modifications comprise a m⁷GpppG or m⁷GpppG analog cap. Synthetic mRNAs are generally capped using one of two strategies: post-transcriptional in vitro capping using commercially available Vaccinia virus capping enzymes, or co-transcriptional capping with the dinucleotide m⁷GpppG or its analogs. Analogs of m⁷GpppG that may be used in capping include, but are not limited to, anti-reverse cap analogs (ARCA), two-m⁷G-headed caps, phosphorothiolate caps, phosphoroselenoate caps, boranophosphate caps, imidodiphosphate caps, and locked nucleic acid (LNA) caps. ARCAs are modified dinucleotide caps containing 3′-O,7′-dimethylguanosine or 3′-deoxy-7-methylguanosine, which prevent incorrect incorporation into synthetic mRNA transcripts via the 3′-OH in m⁷G and have been shown to more than double translation efficiencies relative to unmodified cap dinucleotides. Stepinski et al., Synthesis and Properties of mRNAs Containing the Novel “Anti-reverse” Cap Analogs 7-Methyl(3′-O-methyl)GpppG and 7-methyl (3′-deoxy)GpppG, RNA, 2001, 7 (10), 1486-1495; Jemielity et al., Novel “anti-reverse” cap analogs with superior translational properties, RNA, 2003, 9 (9), 1108-1122. Modified ARCAs that further improve mRNA stability and translational efficiency include, but are not limited to, tetraphosphate analogs (m⁷GppppG) that have a higher binding affinity for eIF4E, Muttach et al., Synthetic mRNA Capping, Beilstein J. Org. Chem., 2017, 13, 2819-2832, as well as phosphorotioate modified analogs that reduce susceptibility of the mRNA to decapping by DCP1/DCP2. Grudzien-Nogalska, et al., Phosphorothioate cap analogs stabilize mRNA and increase translational efficiency in mammalian cells, RNA, 2007, 13 (1), 1745-1755; Kowalska, et al., Synthesis and characterization of mRNA cap analogs containing phosphorothioate substitutions that bind tightly to eIF4E and are resistant to the decapping pyrophosphatase DcpS, RNA, 2008, 14 (6), 1119-1131; Kuhn et al., Phosphorothioate cap analogs increase stability and translational efficiency of RNA vaccines in immature dendritic cells and induce superior immune responses in vivo, Gene Therapy, 2010, 17 (8), 961-971. Additional modifications to the 5′-cap, including those not described herein but appreciated by a person skilled in the art, are also contemplated within the context and scope of the present disclosure.

### c) Coding Sequence Modifications

In one embodiment, the one or more mRNA molecules comprise a coding sequence that encodes the one or more activating proteins and/or cytokines. The coding sequence may comprise one or more modifications that increase stability, reduce immunogenicity, and/or improve the translation efficiency of the mRNA molecule. Modifications to the coding sequence of the mRNA molecule may take the form of nucleoside substitutions and/or phosphodiester modifications.

In one embodiment, the one or more modifications may comprise one or more nucleoside substitutions. Nucleoside substitutions comprise modifying or replacing natural nucleosides in the mRNA molecules with nucleosides that include, but are not limited to, pseudouridine (T), N¹-methyl pseudouridine (m¹Ψ), 2-thiouridine (s²U), 5-methylcytidine (m⁵C), N6-methyladenosine (m⁶A), 2′-O-methyluridine (Um), 2′-O-methylcytidine (Cm), 2′-O-methyladenosine (Am), and 2′-O-methylguanosine (Gm). Nucleoside substitutions with T and m¹Ψ are the most widely used for reducing the immunogenicity of mRNA molecules, as well as increasing translational efficiency. Ψ and m¹Ψ can be used to replace uracil-rich regions in mRNA, which activate the innate immune system through interaction with toll-like receptors (TLRs). Kautffian et al., Efficacy and immunogenicity of unmodified and pseudouridine-modified mRNA delivered systematically with lipid nanoparticles in vivo, Biomaterials, 2016, 109, 78-87. Substitutions with Ψ and m¹Ψ have also been shown to increase translation efficiency. Kariko et al., Incorporation of Pseudouridine into mRNA Yields Superior Nonimmunogenic Vector with Increased Translational Capacity and Biological Stability, Molecular Therapeutics, 2008, 16 (11), 1833-1840; Svitkin et al., N1-methyl-pseudouridine in mRNA Enhances Translation through eIF2a-dependent and Independent Mechanisms by Increasing Ribosome Density, Nucleic Acids Research, 2017, 45 (10), 6023-6036.

In one embodiment, the one or more modifications may comprise one or more phosphodiester modifications. Common phosphodiester modifications include, but are not limited to, phosphorothioate, boranophosphate, and 2′-O-methylation modifications. Additional modifications to the coding sequence, including those not described herein but appreciated by a person skilled in the art, are also contemplated within the context and scope of the present disclosure.

### d) Poly-A Tail Modifications

In one embodiment, the one or more mRNA molecules comprise a poly-A tail. In one embodiment, the poly-A tail may comprise one or more modifications that increase stability, reduce immunogenicity, and/or improve the translation efficiency of the mRNA molecule. The poly-A tail is a chain of adenosine residues located at the 3′-end of the mRNA molecule, which contributes to mRNA stability and translational efficiency. Modifications to the poly-A tail include but are not limited to, the addition of 3′-deoxyadenosine (also known as cordycepin) or 8-aza-adenosine, phosphorothioate modification (Strzelecka et al., Phosphodiester Modifications in mRNA Poly(A) Tail Prevent Deadenylation without Compromising Protein Expression, RNA, 2020, 26 (12), 1815-1837), and addition of sulforhodamine B (Anhauser et al., Multiple Covalent Fluorescence Labeling of Eukarotic mRNA at the Poly(A) Tail Enhances Translation and Can Be Performed in Living Cells, Nucleic Acids Research, 2019, 47 (7), e42). Additional modifications to the poly-A tail, including those not described herein but appreciated by a person skilled in the art, are also contemplated within the context and scope of the present disclosure.

In addition to structural modifications, the poly-A tail may comprise various lengths of adenosine residues. In one embodiment, the poly-A tail may comprise from 1 to 500, from 50 to 400, from 50 to 350, from 50 to 300, from 100 to 250, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350 adenosine residues.

### e) Codon Optimization

In one embodiment, the one or more polynucleotides can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit a particular bias for certain codons of a particular amino acid. Codon bias (differences in codon usage between organisms) often correlates with the efficiency of translation of messenger RNA (mRNA), which is, in turn, believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y. et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In one embodiment, one or more codons (e.g., 1, 2, 3, 4, 5, 10, 15, 20, 25, 50, or more, or all codons) in a polynucleotide sequence encoding one or more activating proteins and/or cytokines corresponds to the most frequently used codon for a particular amino acid.

The polynucleotide can be codon optimized for expression in a specific cell type, tissue type, organ type, and/or subject type. In one embodiment, a codon-optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon-optimized sequences are within the ambit of the ordinary skilled artisan in view of the description herein. In one embodiment, the polynucleotide is a codon-optimized for a specific cell type. Such cell types can include but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon-optimized sequences are within the ambit of the ordinary skilled artisan in view of the description herein. In one embodiment, the polynucleotide is codon optimized for a specific tissue type. Such tissue types can include but are not limited to, muscle tissue, connective tissue, connective tissue, nervous tissue, and epithelial tissue. Such codon-optimized sequences are within the ambit of the ordinary skilled artisan in view of the description herein. In one embodiment, the polynucleotide is codon optimized for a specific organ. Such organs include but are not limited to, muscles, skin, intestines, liver, spleen, brain, lungs, stomach, heart, kidneys, gallbladder, pancreas, bladder, thyroid, bone, blood vessels, blood, and combinations thereof. Such codon-optimized sequences are within the ambit of the ordinary skilled artisan in view of the description herein.

In one embodiment, a polynucleotide coding sequence encoding one or more activating proteins and/or cytokines of the compositions and methods described herein is codon. optimized for expression in particular cells, such as prokaryotic or eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate.

### IV. Delivery Vehicles

In one embodiment, the compositions and methods described herein may comprise one or more delivery vehicles for the delivery of the activating proteins and/or cytokines. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles, in accordance with the present disclosure, may comprise the greatest dimension (e.g., diameter) of less than 100 microns (μm). In one embodiment, the delivery vehicles may comprise the greatest dimension of less than 10 m. In one embodiment, the delivery vehicles may comprise a greatest dimension of less than 2000 nanometers (nm). In one embodiment, the delivery vehicles may comprise a greatest dimension of less than 1000 nanometers (nm). In one embodiment, the delivery vehicles may comprise the greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In one embodiment, the delivery vehicles may comprise the greatest dimension ranging between 25 nm and 200 nm.

In one embodiment, the delivery vehicles may comprise particles. For example, the delivery vehicle may be or comprise nanoparticles (e.g., particles with the greatest dimension (e.g., diameter) no greater than 1000 nm. The particles may be provided in different forms, including but not limited to, solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

In one embodiment, nanoparticles may be used to deliver the compositions described herein. In general, a “nanoparticle” refers to any particle having a diameter of less than 1000 nm. In certain preferred embodiments, nanoparticles of the disclosure have a greatest dimension (e.g., diameter) of 500 nm or less. In other preferred embodiments, nanoparticles of the disclosure have a greatest dimension ranging between 25 nm and 200 nm. In other preferred embodiments, nanoparticles of the disclosure have a greatest dimension of 100 nm or less. In other preferred embodiments, nanoparticles of the disclosure have the greatest dimension ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present disclosure. Semi-solid and soft nanoparticles have been manufactured and are within the scope of the present disclosure. Nanoparticles with one-half hydrophilic and the other half hydrophobic are termed Janus particles and are particularly effective for stabilizing emulsions. They can self-assemble at water/oil interfaces and act as solid surfactants.

Particle characterization (including, e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to e.g., one or more polynucleotides encoding one or more activating proteins and/or cytokines, one or more cytokines, or any combination thereof, and may include additional carriers and/or excipients) to provide particles of an optimal size for delivery for any in vitro, ex vivo, and/or in vivo application of the present disclosure. In certain preferred embodiments, particle dimension (e.g., diameter) characterization is based on measurements using dynamic laser scattering (DLS). Mention is made of U.S. Pat. Nos. 6,007,845; 5,855,913; 5,985,309; 5,543,158; and the Dhalman et al., In vivo endothelial siRNA delivery using polymeric nanoparticles with low molecular weight, Nature Nanotechnology, 2014, 9, 648-655, doi:10.1038/nnano.2014.84, describing particles, methods of making and using them, and measurements thereof.

### 1) Vectors and Vector Systems

Also provided herein are vectors that can be used to deliver any of the polynucleotides and/or cytokines described herein. The vectors can be useful in producing human cells, animal cells, and transgenic animals that can express one or more components of the compositions described herein. Other uses for the vectors and vector systems described herein are also within the scope of this disclosure. In general, and as described elsewhere herein, the term “vector” refers to a tool that allows or facilitates the transfer of an entity from one environment to another. In some contexts, which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends or no free ends (e.g., circular); nucleic acid molecules that comprise DNA, RNA, or both; and other varieties of polynucleotides known in the art. One type of vector is a “plasmid,” which refers to a circular double-stranded DNA molecule into which additional DNA segments can be inserted, such as by standard molecular cloning techniques. Another type of vector is a viral vector, wherein virally derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids.

Recombinant expression vectors can comprise a nucleic acid (e.g., a polynucleotide) of the disclosure in a form suitable for expression in a host cell. This means that the recombinant expression vectors include one or more regulatory elements, which can be selected on the basis of the host cells to be used for expression, operatively linked to the nucleic acid sequence to be expressed. Within a recombinant expression vector, “operably linked” and “operatively linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In one embodiment, the vector can be a bicistronic vector. In one embodiment, a bicistronic vector can be used for one or more elements of the compositions described herein. In one embodiment, expression of elements of the compositions described herein can be driven by the CBh promoter or other ubiquitous promoter. Where the element of any of the compositions described herein is an mRNA, its expression can be driven by a Pol III promoter, such as a U6 promoter. In one embodiment, the two are combined.

In one embodiment, a vector capable of delivering to a cell one or more polynucleotides and/or cytokines of the compositions described herein, can be composed of or contain a minimal promoter operably linked to a polynucleotide encoding a first activating protein or cytokine and a second minimal promoter operably linked to a polynucleotide encoding a second activating protein or cytokine, wherein the length of the vector sequence comprising the minimal promoters and polynucleotide sequences is less than 4.4 Kb. In an embodiment, the vector can be a viral vector. In certain embodiments, the viral vector is an is an adeno-associated virus (AAV) or an adenovirus vector. In one embodiment, the first regulatory element is a polymerase III promoter. In one embodiment, the second regulatory element is a polymerase II promoter.

These and other vectors are further described in detail in this section and elsewhere herein.

### 2) Vector Features

The vectors can include additional features that can confer one or more functionalities to the vector, the polynucleotide to be delivered, a virus particle produced therefrom, or a polypeptide expressed thereof. Such features include but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### a) Regulatory Elements

In certain embodiments, the polynucleotides and/or vectors thereof described herein can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences), and cellular localization signals (e.g., nuclear localization signals). Such regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cells and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle-dependent or developmental stage-dependent manner, which may or may not also be tissue or cell-type specific. In one embodiment, a vector comprises one or more pol III promoters (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In one embodiment, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In one embodiment, the vector can contain a minimal promoter. In one embodiment, the minimal promoter is the Mecp2 promoter, tRNA promoter, or U6. In a further embodiment, the minimal promoter is tissue specific. In one embodiment, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In one embodiment, a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In one embodiment, the regulatory element can be a regulated promoter. “Regulated promoter” refers to promoters that direct gene expression not constitutively, but in a temporally and/or spatially regulated manner, and includes tissue-specific, tissue-preferred and inducible promoters. Regulated promoters include conditional promoters and inducible promoters. In one embodiment, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Fer1l4), endothelial cell specific promoters (e.g. ENG), pluripotent and embryonic germ layer cell specific promoters (e.g., Oct4, NANOG, Synthetic Oct4, T brachyury, NES, SOX17, FOXA2, MIR122), and muscle cell specific promoter (e.g., Desmin). Other tissue and/or cell specific promoters are generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome)., such as a Light Inducible Transcriptional Effector (LITE) that direct changes in transcriptional activity in a sequence-specific manner. In one embodiment, the vector can include one or more of the inducible DNA binding proteins provided in International Patent Publication No. WO 2014/018423 and US Patent Publication Nos. 2015/0291966, 2017/0166903, 2019/0203212, which describe e.g., embodiments of inducible DNA binding proteins and methods of use and can be adapted for use with the present disclosure.

In one embodiment, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

### b) Selectable Markers and Tags

One or more of the polynucleotides encoding the one or more activating proteins and/or cytokines of the compositions described herein can be operably linked, fused to, or otherwise modified to include a polynucleotide that encodes or is a selectable marker or tag, which can be a polynucleotide or polypeptide. In one embodiment, the polynucleotide encoding a polypeptide selectable marker can be incorporated in a polynucleotide encoding an activating protein and/or cytokine such that the selectable marker polypeptide, when translated, is inserted between two amino acids between the N- and C-terminus of the activating protein or cytokine polypeptide or at the N- and/or C-terminus of the activating protein or cytokine polypeptide. In one embodiment, the selectable marker or tag is a polynucleotide barcode or unique molecular identifier (UMI).

It will be appreciated that the polynucleotide encoding such selectable markers or tags can be incorporated into a polynucleotide encoding one or more components of the compositions described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the compositions described herein via suitable linker, such as a glycine or glycine serine linkers as short as GS or GG up to (GGGGG)₃ (SEQ ID NO: 755) or (GGGGS)₃ (SEQ ID NO: 756). Other suitable linkers are described elsewhere herein.

The vector or vector system can include one or more polynucleotides encoding one or more targeting moieties. In one embodiment, the targeting moiety encoding polynucleotides can be included in the vector or vector system, such as a viral vector system, such that they are expressed within and/or on the virus particle(s) produced such that the virus particles can be targeted to specific cells, tissues, organs, etc. In one embodiment, the targeting moiety encoding polynucleotides can be included in the vector or vector system such that the polynucleotides encoding the one or more activating proteins and/or cytokines of the compositions described herein include the targeting moiety and can be targeted to specific cells, tissues, organs, etc. In one embodiment, such as non-viral carriers, the targeting moiety can be attached to the carrier (e.g., polymer, lipid, inorganic molecule etc.) and can be capable of targeting the carrier and any attached or polynucleotides encoding the one or more activating proteins and/or cytokines to specific cells, tissues, organs, etc.

### c) Codon Optimization of Vector Polynucleotides

As described elsewhere herein, the polynucleotides encoding the one or more activating proteins and/or cytokines of the compositions described herein can be codon optimized. In one embodiment, one or more polynucleotides contained in a vector (“vector polynucleotides”) described herein that are in addition to an optionally codon optimized polynucleotides encoding the one or more activating proteins and/or cytokines of the compositions described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In one embodiment, one or more codons (e.g., 1, 2, 3, 4, 5, 10, 15, 20, 25, 50, or more, or all codons) in a sequence encoding one or more of the activating proteins or cytokines corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gown, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type. In one embodiment, a codon optimized sequence is a sequence optimized for expression in a eukaryote, e.g., humans (i.e., being optimized for expression in a human or human cell), or for another eukaryote, such as another animal (e.g. a mammal or avian) as is described elsewhere herein. Such codon optimized sequences are within the ambit of the ordinary skilled artisan in view of the description herein. In one embodiment, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells etc.), muscle cells (e.g. cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In one embodiment, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In one embodiment, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In one embodiment, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### 3) Vector Construction

The vectors described herein can be constructed using any suitable process or technique. In one embodiment, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

In one embodiment, a vector comprises one or more insertion sites, such as a restriction endonuclease recognition sequence (also referred to as a “cloning site”). In one embodiment, one or more insertion sites (e.g., about or more than about 1, 2, 3, 4, 5, 6, 7, 8, 9, 10, or more insertion sites) are located upstream and/or downstream of one or more sequence elements of one or more vectors.

### 4) Viral Vectors

The cargos, e.g., the polynucleotides and/or cytokines described herein, can be introduced to cells by transduction by a viral or pseudoviral particle. Methods of packaging the cargos in viral particles can be accomplished using any suitable viral vector or vector systems.

As used in this context herein “transduction” refers to the process by which foreign nucleic acids and/or proteins are introduced to a cell (prokaryote or eukaryote) by a viral or pseudoviral particle. After packaging in a viral particle or pseudoviral particle, the viral particles can be exposed to cells (e.g., in vitro, ex vivo, or in vivo) where the viral or pseudoviral particle infects the cell and delivers the cargo to the cell via transduction. Viral and pseudoviral particles can be optionally concentrated prior to exposure to target cells. In one embodiment, the virus titer of a composition containing viral and/or pseudoviral particles can be obtained and a specific titer be used to transduce cells.

The term of art “viral vector” and as used herein in this context refers to polynucleotide based vectors that contain one or more elements from or based upon one or more elements of a virus that can be capable of expressing and packaging a polynucleotide, such as the polynucleotides encoding the one or more activating proteins and/or cytokines of the compositions described herein, into a virus particle and producing said virus particle when used alone or with one or more other viral vectors (such as in a viral vector system). Viral vectors and systems thereof can be used for producing viral particles for delivery of and/or expression of one or more components of the compositions described herein. The viral vector can be part of a viral vector system involving multiple vectors. In one embodiment, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produce therefrom are described elsewhere herein. In one embodiment, the viral vectors are configured to produce replication incompetent viral particles for improved safety of these systems.

In certain embodiments, the virus structural component, which can be encoded by one or more polynucleotides in a viral vector or vector system, comprises one or more capsid proteins including an entire capsid. In certain embodiments, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the disclosure can comprise one or more of VP1, and/or one or more of VP2, and/or one or more of VP3. Accordingly, the present disclosure is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Thus, a virus of within the family Adenoviridae is contemplated as within the disclosure with discussion herein as to adenovirus applicable to other family members. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cell, glioblastoma cells, coronary artery endothelial cells and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8(1): 3-11 (2011)), as well as regarding modifications of AAV, the skilled person can readily obtain a modified adenovirus that has a large payload protein, despite that heretofore it was not expected that such a large protein could be provided on an adenovirus. And as to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

### a) Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the gene of interest into the target cell to provide permanent transgene expression. Suitable retroviral vectors for use with the polynucleotide and/or cytokine components of the compositions described herein can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). Selection of a retroviral gene transfer system may therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In one embodiment, an HIV-based lentiviral vector system can be used. In one embodiment, a FIV-based lentiviral vector system can be used.

In one embodiment, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8: 275-285). In another embodiment, RetinoStat®, (see, e.g., Binley et al., HUMAN GENE THERAPY 23:980-991 (September 2012)), which describes RetinoStat®, an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for use with the polynucleotides encoding the one or more activating proteins and/or cytokines of the compositions described herein.

In one embodiment, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In one embodiment, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In one embodiment, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In one embodiment, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In one embodiment, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included up-stream of the LTRs), and they can include one or more deletions in the 3′LTR to create self-inactivating (SIN) vectors having disrupted promoter/enhancer activity of the LTR. In one embodiment, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g. gag, pol, and rev) and upstream regulatory sequences (e.g. promoter(s)) to drive expression of the features present on the packaging vector, and (iii) an “envelope vector” that contains one or more envelope protein genes and upstream promoters. In certain embodiments, the third-generation lentiviral vector system can include at least two packaging vectors, with the gag-pol being present on a different vector than the rev gene.

In one embodiment, the pseudotype and infectivity or tropisim of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In one embodiment, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD 114) (see e.g., Hanawa et al. Molec. Ther. 2002 5(3) 242-251), modified Sindbis virus envelope proteins (see e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124: 1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In one embodiment, the tropism of the resulting lentiviral particle can be tuned by incorporating cell targeting peptides into a lentiviral vector such that the cell targeting peptides are expressed on the surface of the resulting lentiviral particle. In one embodiment, a lentiviral vector can contain an envelope protein that is fused to a cell targeting protein (see e.g., Buchholz et al. 2015. Trends Biotechnol. 33:777-790; Bender et al. 2016. PLoS Pathog. 12(e1005461); and Friedrich et al. 2013. Mol. Ther. 2013. 21: 849-859.

In one embodiment, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233. In these embodiments, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In one embodiment, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In one embodiment, a lentiviral vector can include an N-terminal PDZ domain of InaD protein (PDZ1) and its pentapeptide ligand (TEFCA) (SEQ ID NO: 757) from NorpA, which can conjugate the cell targeting peptide to the virus particle via a covalent bond (e.g., a disulfide bond). In one embodiment, the PDZ1 protein can be fused to an envelope protein, which can optionally be binding deficient and/or fusion competent virus envelope protein and included in a lentiviral vector. In one embodiment, the TEFCA (SEQ ID NO: 757) can be fused to a cell targeting peptide and the TEFCA-CPT (SEQ ID NO:757) fusion construct can be incorporated into the same or a different lentiviral vector as the PDZ1-envenlope protein construct. During virus production, specific interaction between the PDZ1 and TEFCA (SEQ ID NO: 757) facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015. Any of these systems or a variant thereof can be used to deliver the polynucleotides encoding the one or more activating proteins and/or cytokines of the compositions described herein to a cell.

In one embodiment, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (Ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

In another embodiment, Cocal vesiculovirus envelope pseudotyped retroviral or lentiviral vector particles are contemplated (see, e.g., US Patent Publication No. 20120164118 assigned to the Fred Hutchinson Cancer Research Center). Cocal virus is in the Vesiculovirus genus and is a causative agent of vesicular stomatitis in mammals. Cocal virus was originally isolated from mites in Trinidad (Jonkers et al., Am. J. Vet. Res. 25:236-242 (1964)), and infections have been identified in Trinidad, Brazil, and Argentina from insects, cattle, and horses. Many of the vesiculoviruses that infect mammals have been isolated from naturally infected arthropods, suggesting that they are vector-borne. Antibodies to vesiculoviruses are common among people living in rural areas where the viruses are endemic and laboratory-acquired; infections in humans usually result in influenza-like symptoms. The Cocal virus envelope glycoprotein shares 71.5% identity at the amino acid level with VSV-G Indiana, and phylogenetic comparison of the envelope gene of vesiculoviruses shows that Cocal virus is serologically distinct from, but most closely related to, VSV-G Indiana strains among the vesiculoviruses. Jonkers et al., Am. J. Vet. Res. 25:236-242 (1964) and Travassos da Rosa et al., Am. J. Tropical Med. & Hygiene 33:999-1006 (1984). The Cocal vesiculovirus envelope pseudotyped retroviral vector particles may include for example, lentiviral, alpharetroviral, betaretroviral, gammaretroviral, deltaretroviral, and epsilonretroviral vector particles that may comprise retroviral Gag, Pol, and/or one or more accessory protein(s) and a Cocal vesiculovirus envelope protein. In certain embodiments of these embodiments, the Gag, Pol, and accessory proteins are lentiviral and/or gammaretroviral. In one embodiment, a retroviral vector can contain encoding polypeptides for one or more Cocal vesiculovirus envelope proteins such that the resulting viral or pseudoviral particles are Cocal vesiculovirus envelope pseudotyped.

### b) Adenoviral Vectors, Helper-Dependent Adenoviral Vectors, and Hybrid Adenoviral Vectors

In one embodiment, the vector can be an adenoviral vector. In one embodiment, the adenoviral vector can include elements such that the virus particle produced using the vector or system thereof can be serotype 2 or serotype 5. In one embodiment, the polynucleotide to be delivered via the adenoviral particle can be up to about 8 kb. Thus, in one embodiment, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In one embodiment, the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In certain embodiments of the helper-dependent adenoviral vector system one vector (the helper) can contain all the viral genes required for replication but contains a conditional gene defect in the packaging domain. The second vector of the system can contain only the ends of the viral genome, one or more polynucleotides encoding the one or more activating proteins and/or cytokines of the compositions described herein, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the polynucleotides encoding the one or more activating proteins and/or cytokines of the compositions described herein. In one embodiment, the polynucleotide to be delivered via the viral particle produced from a helper-dependent adenoviral vector or system thereof can be up to about 37 kb. Thus, in one embodiment an adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In one embodiment, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In one embodiment, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use with the polynucleotides encoding the one or more activating proteins and/or cytokines of the compositions described herein. In one embodiment, a hybrid-adenoviral vector can include one or more features of a retrovirus and/or an adeno-associated virus. In one embodiment, the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use with the polynucleotides encoding the one or more activating proteins and/or cytokines of the compositions described herein. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use with the polynucleotides encoding the one or more activating proteins and/or cytokines of the compositions described herein.

### c) Adeno-Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer than adenoviral vectors. In one embodiment, the AAV can integrate into a specific site on chromosome 19 of a human cell with no observable side effects. In one embodiment, the capacity of the AAV vector, system thereof, and/or AAV particles can be up to about 4.7 kb.

The AAV vector or system thereof can include one or more regulatory molecules. In one embodiment, the regulatory molecules can be promoters, enhancers, repressors and the like, which are described in greater detail elsewhere herein. In one embodiment, the AAV vector or system thereof can include one or more polynucleotides that can encode one or more regulatory proteins. In one embodiment, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In one embodiment, the AAV capsid can contain 60 capsid proteins. In one embodiment, the ratio of VP1:VP2:VP3 in a capsid can be about 1:1:10.

In one embodiment, the AAV vector or system thereof can include one or more adenovirus helper factors or polynucleotides that can encode one or more adenovirus helper factors. Such adenovirus helper factors can include, but are not limited, E1A, EiB, E2A, E4ORF6, and VA RNAs. In one embodiment, a producing host cell line expresses one or more of the adenovirus helper factors.

The AAV vector or system thereof can be configured to produce AAV particles having a specific serotype. In one embodiment, the serotype can be AAV-1, AAV-2, AAV-3, AAV-4, AAV-5, AAV-6, AAV-8, AAV-9 or any combinations thereof. In one embodiment, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in one embodiment, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In one embodiment, an AAV vector or system thereof capable of producing AAV particles capable of targeting cardiac tissue can be configured to generate an AAV particle having an AAV-4 serotype. In one embodiment, an AAV vector or system thereof capable of producing AAV particles capable of targeting the liver can be configured to generate an AAV having an AAV-8 serotype. In one embodiment, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the second plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et al, J. Virol. 82: 5887-5911 (2008), which is recapitulated in Table 1 below.

TABLE 1

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

In one embodiment, the AAV vector or system thereof is configured as a “gutless” vector, similar to that described in connection with a retroviral vector. In one embodiment, the “gutless” AAV vector or system thereof can have the cis-acting viral DNA elements involved in genome amplification and packaging in linkage with the heterologous sequences of interest (e.g., the polynucleotides encoding the one or more activating proteins and/or cytokines of the compositions described herein).

In one embodiment, the AAV vectors are produced in in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. nAAV vectors are discussed elsewhere herein.

### d) Herpes Simplex Viral Vectors

In one embodiment, the vector can be a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use with the components of the compositions described herein. In one embodiment, where an HSV vector or system thereof is utilized, the host cell can be a complementing cell. In one embodiment, HSV vector or system thereof can be capable of producing virus particles capable of delivering a polynucleotide cargo of up to 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use with the components of the compositions described herein.

### e) Poxvirus Vectors

In one embodiment, the vector can be a poxvirus vector or system thereof. In one embodiment, the poxvirus vector can result in cytoplasmic expression of one or more polynucleotides encoding the one or more activating proteins and/or cytokines of the compositions described herein. In one embodiment, the capacity of a poxvirus vector or system thereof can be about 25 kb or more.

Virus Particle Production from Viral Vectors

### f) Retroviral Production

In one embodiment, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In one embodiment, the suitable host cell for virus production from viral vectors and systems thereof described herein can stably express one or more genes involved in packaging (e.g., pol, gag, and/or VSV-G) and/or other supporting genes.

In one embodiment, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., the polynucleotides encoding the one or more activating proteins and/or cytokines of the compositions described herein), and virus particle assembly, and secretion of mature virus particles into the culture media. Various other methods and techniques are generally known to those of ordinary skill in the art.

Mature virus particles can be collected from the culture media by a suitable method. In one embodiment, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In one embodiment, the resulting composition containing virus particles can contain 1×10¹-1×10²⁰ particles/mL.

Lentiviruses may be prepared from any lentiviral vector or vector system described herein. In one example embodiment, after cloning pCasES10 (which contains a lentiviral transfer plasmid backbone), HEK293FT at low passage (p=5) can be seeded in a T-75 flask to 50% confluence the day before transfection in DMEM with 10% fetal bovine serum and without antibiotics. After 20 hours, the media can be changed to OptiMEM (serum-free) media and transfection of the lentiviral vectors can done 4 hours later. Cells can be transfected with 10 μg of lentiviral transfer plasmid (pCasES10) and the appropriate packaging plasmids (e.g., 5 μg of pMD2.G (VSV-g pseudotype), and 7.5 ug of psPAX2 (gag/pol/rev/tat)). Transfection can be carried out in 4 mL OptiMEM with a cationic lipid delivery agent (50 uL Lipofectamine 2000 and 100 ul Plus reagent). After 6 hours, the media can be changed to antibiotic-free DMEM with 10% fetal bovine serum. These methods can use serum during cell culture, but serum-free methods are preferred.

Following transfection and allowing the producing cells (also referred to as packaging cells) to package and produce virus particles with packaged cargo, the lentiviral particles can be purified. In an exemplary embodiment, virus-containing supernatants can be harvested after 48 hours. Collected virus-containing supernatants can first be cleared of debris and filtered through a 0.45 um low protein binding (PVDF) filter. They can then be spun in an ultracentrifuge for 2 hours at 24,000 rpm. The resulting virus-containing pellets can be resuspended in 50 ul of DMEM overnight at 4 degrees C. They can be then aliquoted and used immediately or immediately frozen at −80 degrees C. for storage.

### g) AAV Particle Production

There are two main strategies for producing AAV particles from AAV vectors and systems thereof, such as those described herein, which depend on how the adenovirus helper factors are provided (helper v. helper free). In one embodiment, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the polynucleotides encoding the one or more activating proteins and/or cytokines of the compositions described herein). In one embodiment, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., the polynucleotides encoding the one or more activating proteins and/or cytokines of the compositions described herein) between 2 ITRs; (2) a vector that carries the AAV Rep-Cap encoding polynucleotides; and (helper polynucleotides. One of skill in the art will appreciate various methods and variations thereof that are both helper and -helper free and as well as the different advantages of each system.

### Non-Viral Vectors

In one embodiment, the vector is a non-viral vector or vector system. The term of art “non-viral vector” and as used herein in this context refers to molecules and/or compositions that are vectors but that are not based on one or more components of a virus or virus genome (excluding any nucleotide to be delivered and/or expressed by the non-viral vector) that can be capable of incorporating one or more polynucleotides of the compositions described herein and delivering said polynucleotides to a cell and/or expressing the polynucleotides in the cell. Non-viral vectors can include, without limitation, naked polynucleotides and polynucleotide (non-viral) based vector and vector systems.

### 5) Naked Polynucleotides

In one embodiment, one or more polynucleotides of the compositions described herein can be included as a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In one embodiment, the naked polynucleotide contains only the one or more polynucleotides encoding the one or more activating proteins and/or cytokines. In one embodiment, the naked polynucleotide can contain other polynucleotides in addition to the polynucleotides of the compositions described herein.

### 6) Non-Viral Polynucleotide Vectors

In one embodiment, one or more of polynucleotides of the compositions described herein can be included in a non-viral polynucleotide vector. Suitable non-viral polynucleotide vectors include, but are not limited to, transposon vectors and vector systems, plasmids, bacterial artificial chromosomes, yeast artificial chromosomes, AR(antibiotic resistance)-free plasmids and miniplasmids, circular covalently closed vectors (e.g. minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In one embodiment, the non-viral polynucleotide vector can have a conditional origin of replication. In one embodiment, the non-viral polynucleotide vector can be an ORT plasmid. In one embodiment, the non-viral polynucleotide vector can have a minimalistic immunologically defined gene expression. In one embodiment, the non-viral polynucleotide vector can have one or more post-segregationally killing system genes. In one embodiment, the non-viral polynucleotide vector is AR-free. In one embodiment, the non-viral polynucleotide vector is a minivector. In one embodiment, the non-viral polynucleotide vector includes a nuclear localization signal. In one embodiment, the non-viral polynucleotide vector can include one or more CpG motifs. In one embodiment, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present disclosure. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In certain embodiments, the S/MAR sequence is located downstream of an actively transcribed polynucleotide (e.g., one or more of the polynucleotides encoding the activating proteins and/or cytokines of the compositions described herein) included in the non-viral polynucleotide vector. In one embodiment, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present disclosure.

In one embodiment, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In one embodiment, the non-viral polynucleotide vector can be a retrotransposon vector. In one embodiment, the retrotransposon vector includes long terminal repeats. In one embodiment, the retrotransposon vector does not include long terminal repeats. In one embodiment, the non-viral polynucleotide vector can be a DNA transposon vector. DNA transposon vectors can include a polynucleotide sequence encoding a transposase. In one embodiment, the transposon vector is configured as a non-autonomous transposon vector, meaning that the transposition does not occur spontaneously on its own. In one embodiment, the transposon vector lacks one or more polynucleotide sequences encoding proteins required for transposition. In one embodiment, the non-autonomous transposon vectors lack one or more Ac elements.

In one embodiment, a non-viral polynucleotide transposon vector system can include a first polynucleotide vector that contains the one or more polynucleotides encoding the activating proteins and/or cytokines described herein flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector and integrate it into one or more positions in the host cell's genome. In one embodiment, the transposon vector or system thereof can be configured as a gene trap. In one embodiment, the TIRs can be configured to flank a strong splice acceptor site followed by a reporter and/or other gene and a strong poly A tail. When transposition occurs while using this vector or system thereof, the transposon can insert into an intron of a gene and the inserted reporter or other gene can provoke a mis-splicing process and, as a result, it inactivates the trapped gene.

Any suitable transposon system can be used. Suitable transposon and systems thereof can include, but are not limited to, Sleeping Beauty transposon system (Tcl/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tcl/mariner superfamily) (see e.g., Miskey et al. 2003 Nucleic Acid Res. 31(23):6873-6881) and variants thereof.

### 7) Non-Viral Delivery Vehicles

The delivery vehicles may comprise non-viral vehicles. In general, methods and vehicles capable of delivering nucleic acids and/or proteins may be used for delivering the polynucleotides and/or cytokines of the compositions described herein. Examples of non-viral vehicles include lipid nanoparticles, cell-penetrating peptides (CPPs), DNA nanoclews, gold nanoparticles, streptolysin O, multifunctional envelope-type nanodevices (MENDs), lipid-coated mesoporous silica particles, and other inorganic nanoparticles.

### a) Lipid Particles

The delivery vehicles may comprise lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### b) Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations. In some examples. LNPs may be used for delivering the polynucleotides encoding the one or more activating proteins and/or cytokines of the compositions described herein.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG), (heptatriaconta-6,9,28,31-tetraen-19-yl-4-(dimethylamino)butanoate) (DLin-MC3-DMA), (4-hydroxybutyl) azanediyl)bis(hexane-6,1-diyl)bis(2-hexyldecanoate) (ALC-0315), heptadecan-9-yl 8-((2-hydroxyethyl) (6-oxo-6-(undecyloxy) hexyl) amino) octanoate (SM-102), and any combination thereof. Preparation of LNPs and encapsulation may be adapted from Rosin et al, Molecular Therapy, vol. 19, no. 12, pages 1286-2200, Dec. 2011).

In example embodiments, the LNP is SM-102, see e.g., K. J. Hassett, et al., Molecular Therapy—Nucleic Acids 2019, 15, 1-11 and F. Ferraresso, et al., Mol. Pharmaceutics 2022, 19, 2175-2182.

In one embodiment, the LNP contains a nucleic acid, wherein the charge ratio of nucleic acid backbone phosphates to cationic lipid nitrogen atoms is about 1:1.5-7 or about 1:4.

In one embodiment, the LNP also includes a shielding compound, which is removable from the lipid composition under in vivo conditions. In one embodiment, the shielding compound is a biologically inert compound. In one embodiment, the shielding compound does not carry any charge on its surface or on the molecule as such. In one embodiment, the shielding compounds are polyethylenglycoles (PEGs), hydroxyethylglucose (HEG) based polymers, polyhydroxyethyl starch (polyHES) and polypropylene. In one embodiment, the PEG, HEG, polyHES, and a polypropylene weight between about 500 to 10,000 Da or between about 2000 to 5000 Da. In one embodiment, the shielding compound is PEG2000 or PEG5000.

In one embodiment, the LNP can include one or more helper lipids. In one embodiment, the helper lipid can be a phosphor lipid or a steroid. In one embodiment, the helper lipid is between about 20 mol % to 80 mol % of the total lipid content of the composition. In one embodiment, the helper lipid component is between about 35 mol % to 65 mol % of the total lipid content of the LNP. In one embodiment, the LNP includes lipids at 50 mol % and the helper lipid at 50 mol % of the total lipid content of the LNP.

Other non-limiting, exemplary LNP delivery vehicles are described in U.S. Patent Publication Nos. US 20160174546, US 20140301951, US 20150105538, US 20150250725, Wang et al., J. Control Release, 2017 Volume 263, p. 39-45; Altmoglu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthcare Mater., 3(9):1398-403, Sep. 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi:10.1038/nnano.2014.84; Coelho et al., N Engl J Med 2013; 369:819-29; Aleku et al., Cancer Res., 68(23): 9788-98 (Dec. 1, 2008), Strumberg et al., Int. J. Clin. Pharmacol. Ther., 50(1): 76-8 (Jan. 2012), Schultheis et al., J. Clin. Oncol., 32(36): 4141-48 (Dec. 20, 2014), and Fehring et al., Mol. Ther., 22(4): 811-20 (Apr. 22, 2014); Novobrantseva, Molecular Therapy—Nucleic Acids (2012) 1, e4; doi:10.1038/mtna.2011.3; WO2012135025; US 20140348900; US 20140328759; US 20140308304; WO 2005/105152; WO 2006/069782; WO 2007/121947; US 2015/082080; US 20120251618; 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316.

### c) Liposomes

In one embodiment, a lipid particle may be liposome. Liposomes are spherical vesicle structures composed of a uni- or multilamellar lipid bilayer surrounding internal aqueous compartments and a relatively impermeable outer lipophilic phospholipid bilayer. In one embodiment, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In one embodiment, the liposome can be a Trojan Horse liposome (also known in the art as Molecular Trojan Horses), see e.g., cshprotocols.cshlp.org/content/2010/4/pdb.prot5407.long, the teachings of which can be applied and/or adapted to generated and/or deliver the polynucleotide and/or cytokine components of the compositions described herein.

Other non-limiting, exemplary liposomes can be those as set forth in Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi:10.1155/2011/469679; WO 2008/042973; U.S. Pat. No. 8,071,082; WO 2014/186366; 20160257951; US20160129120; US 20160244761; 20120251618; WO2013/093648; Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif), and Eufectins (JBL, San Luis Obispo, Calif.).

### d) Stable Nucleic Acid Lipid Particles (SNALPs)

In one embodiment, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMAo).

Other non-limiting, exemplary SNALPs that can be used to deliver the polynucleotide and/or cytokine components of the compositions described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375: 1896-905; Judge, J. Clin. Invest. 119:661-673 (2009); and Semple et al., Nature Niotechnology, Volume 28 Number 2 Feb. 2010, pp. 172-177.

### e) Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG. In one embodiment, the delivery vehicle can be or include a lipidoid, such as any of those set forth in, for example, U.S. Pub. No. 2011/0293703. In one embodiment, the delivery vehicle can be or include an amino lipid, such as any of those set forth in, for example, Jayaraman, Angew. Chem. Int. Ed. 2012, 51, 8529-8533. In one embodiment, the delivery vehicle can be or include a lipid envelope, such as any of those set forth in, for example, Korman et al., 2011. Nat. Biotech. 29:154-157.

### f) Lipoplexes Polyplexes

In one embodiment, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), [symbol] (e.g., forming DNA/Ca²⁺ microcomplexes), polyethenimine (PEI) (e.g., branched PEI), and poly(L-lysine) (PLL).

### g) Cell Penetrating Peptides

In one embodiment, the delivery vehicles comprise cell penetrating peptides (CPPs). CPPs are short peptides that facilitate cellular uptake of various molecular cargo (e.g., from nanosized particles to small chemical molecules and large fragments of DNA). CPPs may comprise different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only non-polar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl). Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached directly (i.e., functionalized) to the polynucleotides (e.g., plasmid DNA, mRNA) and/or cytokines described herein and delivered to cells. In some examples, the CPP-functionalized polynucleotides encoding the one or more activating proteins may be delivered in combination with the CPP-functionalized polynucleotides encoding the one or more cytokines. In some examples, the CPP-functionalized polynucleotides encoding the one or more activating proteins may be delivered in combination with the CPP-functionalized cytokines. In some examples, the CPP-functionalized polynucleotides encoding the one or more activating proteins may be delivered separately from the CPP-functionalized cytokines.

### h) DNA Nanoclews

In one embodiment, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. A DNA nanoclew may be coated, e.g., coated with PEI to induce endosomal escape.

### i) Gold Nanoparticles

In one embodiment, the delivery vehicles comprise gold nanoparticles (also referred to AuNPs or colloidal gold). Gold nanoparticles may form complex with cargos, e.g., the polynucleotides encoding the one or more activating proteins and/or cytokines of the compositions described herein. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901.

j) iTOP

In one embodiment, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015) Cell 161:674-690.

### k) Polymer-Based Particles

In one embodiment, the delivery vehicles may comprise polymer-based particles (e.g., nanoparticles). In one embodiment, the polymer-based particles may mimic a viral mechanism of membrane fusion. The polymer-based particles may comprise components that mimic Influenza virus machinery and form transfection complexes with various types of nucleic acids (e.g., plasmid DNA, mRNA, siRNA, miRNA, shRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In one embodiment, the polymer-based particles may comprise alkylated and carboxyalkylated branched polyethylenimine. In some examples, the polymer-based particles include, but are not limited to, VIROMER, e.g., VIROMER RNAi, VIROMER RED, VIROMER mRNA. Example methods of delivering the compositions or components of the compositions described herein include those described in Viromer® RED, a powerful tool for transfection of keratinocytes. doi: 10.13140/RG.2.2.16993.61281, Viromer® Transfection—Factbook 2018: technology, product overview, users' data., doi:10.13140/RG.2.2.23912.16642.

Other exemplary and non-limiting polymeric particles are described in US 20170079916, US 20160367686, US 20110212179, US 20130302401, 6,007,845, 5,855,913, 5,985,309, 5,543,158, WO2012135025, US 20130252281, US 20130245107, US 20130244279; US 20050019923, and 20080267903.

### l) Streptolysin O (SLO)

The delivery vehicles may comprise streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### m) Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### n) Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In one embodiment, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### o) Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### p) Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr 28. doi: 10.1039/d0bm00427h.

Other non-limiting, exemplary exosomes include any of those set forth in Alvarez-Erviti et al. 2011, Nat Biotechnol 29: 341; [1401] El-Andaloussi et al. (Nature Protocols 7:2112-2126(2012); and Wahlgren et al. (Nucleic Acids Research, 2012, Vol. 40, No. 17 e130).

### q) Self-Assembling Nanoparticles

In one embodiment, the delivery vehicle is a self-assembling nanoparticle. The self-assembling nanoparticles can contain one or more polymers. The self-assembling nanoparticles can be PEGylated. Self-assembling nanoparticles are known in the art. Examples of self-assembling nanoparticles include, but are not limited to, those described in Schiffelers et al., Nucleic Acids Research, 2004, Vol. 32, No. 19, Bartlett et al. (PNAS, Sep. 25, 2007, vol. 104, no. 39; Davis et al., Nature, Vol 464, 15 Apr. 2010.

### r) Supercharged Proteins

In one embodiment, the delivery vehicle can be a supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### s) Nucleic Acid Assemblies

In one embodiment, the delivery vehicle can be a nucleic acid assembly. As described herein, a nucleic acid assembly is a structure primarily assembled from and composed of nucleic acid molecules wherein the assemblies are structured and held together by nucleic acid hybridization. The nucleic acid molecules can be any form of nucleic acid, including but not limited to, DNA, RNA, mixtures of DNA and RNA, and peptide nucleic acids. Nucleic acid assemblies may be designed and fabricated in any number of geometric shapes including, but not limited to, two-dimensional crystalline arrays or three-dimensional polyhedrons. In one embodiment, the nucleic acid assembly may comprise DNA origami. As used herein, the term “DNA origami” describes nucleic acid assemblies comprising a long single-stranded polynucleotide (i.e., a scaffold strand) folded into a desired shape or structure and held together using numerous short single strands of nucleic acids (i.e., staple strands). Exemplary DNA origami structures include three-dimensional solid figures, in which each side is a flat surface. The flat surfaces are typically polygons and are joined at their edges and held together by staple strands. Additional nucleotides can be added to the 5′ or 3′ end of the staple strands to generate staple overhangs. These staple overhangs can be functionalized with specific nucleic acid sequences to hybridize to a target nucleic acid or form a complex with a desired cargo. For example, a staple overhang may hybridize with one or more of the mRNA molecules described herein for delivery of the mRNA molecules into cells. Staple overhangs may be outwardly facing, such as, for example, when functionalized with a DNA aptamer for targeting to the HER2 receptor. Staple overhangs may also be inwardly facing, such as for example, when hybridized with one or more of the mRNA molecules described herein such that the nucleic acid assembly encloses and shields the mRNA molecules. Examples of nucleic acid assemblies are described in Wamhoff et al., ACS Appl. Bio Mater. 2023, DOI: 10.1021/acsabm.3c00155; Parsons et al., Nat Commun 14, 382 (2023), DOI: 10.1038/s41467-02336156-1; Knappe et al., Nat Rev Mater 8, 123-138 (2023), DOI: 10.1038/s41578-022-00517; Pan et al., Nucleic Acids Research, 2017, Vol. 45, No. 11, DOI: 10.1093/nar/gkx378; as well as in International Publication Nos. WO 2017/189870 and WO 2020/051507.

### t) Endogenous Retroelement Derived Delivery Systems

Eukaryotic genomes contain domesticated genes from integrating viruses and mobile genetic elements. Some of these domesticated genes have been retooled by mammalian cells to perform different biological functions. As described in Segel et al., Science. 2021 Aug. 20; 373(6557): 882-889. DOI: 10.1126/science.abg6155, as well as in International Publications Nos. WO 2021/055855 and WO 2022/165262, which are incorporated by reference, such systems have been engineered to function as delivery systems for a wide range of cargo. Accordingly in one embodiment, the delivery system is an endogenous retroelement derived delivery system. An endogenous retroelement derived delivery system typically comprises a retroelement polypeptide capable of self-assembly into a delivery particle and one or more packaging elements that can be associated with the cargo to be packaged and which direct encapsulation of the cargo into the delivery particle. The endogenous retroelement system may be a retroviral gag protein or homolog thereof, such as Arc1, Asprvl, PNMA1, PNMA3, PNMA4, PNMA5, PNMA6, PNMA7, PEG10, RTL1, MOAP1, or ZCCHC12. In one embodiment, the packaging elements may be a 5′ UTR and/or 3′ UTR derived from an mRNA encoding a retroelement polypeptide.

u) Extracellular Contractile Injection Systems (eCIS)

In one embodiment, the delivery vehicle may comprise an extracellular contractile injection system (eCIS). Contractile injection systems (CIS) are a class of syringe-like macromolecular complexes used by endosymbiotic bacteria to inject protein payloads into eukaryotic host cells by driving a spike through the cellular membrane. See Kreitz et al., Nature 616, 357-364 (2023), DOI: 10.1038/s41586-023-05870-7. These macromolecular complexes contain a rigid tube structure housed in a contractile sheath anchored to a baseplate and sharpened by a spike protein. Payload proteins loaded into the lumen of the inner tube form associations with the spike, which upon target cell recognition, is forced through the cellular membrane via sheath contraction. CISs can be anchored to the bacterial membrane or the thylakoid membrane in cyanobacteria, or can be produced as free eCISs. Bacterial payloads used in eCISs have been shown to exhibit a variety of functions including host cytoskeleton modulation, DNA cleavage, induction of metamorphosis, and host toxicity. An exemplary eCIS is the Photorhabdus virulence cassette (PVC) derived from the entomopathogenic bacterium Photorhabdus asymbiotica. See Kreitz et al. (2023). The P. asymbiotica PVC locus contains 16 core structural and accessory genes necessary for the assembly of a functional eCIS (denoted as pvc1-16). Downstream of these genes are the payload genes Pdp1 and Pnf, as well as a series of putative regulatory genes.

eCISs may be engineered to alter the payload and reprogrammed to modulate tropism towards a desired cell type. As has been demonstrated using PVCs, the endogenous payload proteins (i.e., Pdp1 and Pnf) contain highly disordered N-terminal regions that are involved in loading the payload proteins into the tube structure of the eCIS. These N-terminal “packaging domains” can be fused to novel payload proteins not naturally loaded onto eCISs. Thus, in one embodiment, the delivery vehicle may comprise an engineered eCIS wherein the one or more activating proteins and/or cytokines is fused to an N-terminal packaging domain or one or more polynucleotides encoding the fusion protein thereof. Tropism or homing of the eCIS may be modulated by engineering the Pvc13 tail fiber protein, which is involved in eukaryotic cell recognition. The C-terminus of the Pvc13 tail fiber protein comprises a helical structure with a globular binding domain that interacts with eukaryotic cell receptors. Kreitz et al. demonstrated that PVCs can be reprogrammed to target human cells by replacing the C-terminal binding domain with either the trimeric human adenovirus 5 knob domain (Ad5-knob) or epidermal growth factor receptor (EGFR)-specific designed ankyrin repeat protein (DARPin) E01 (E01-DARPin). See Kreitz et al. (2023). Accordingly, in one embodiment, the delivery vehicle may be an engineered eCIS further comprising one or more tail fiber proteins engineered to bind to a cell of interest.

The compositions and delivery vehicles described herein (i.e., vectors, viral vectors, lipid particles, etc.) may be delivered through a number of mechanisms such as, for example, those described below.

### 8) Physical Non-Viral Delivery Methods

### a) Microinjection

Microinjection of the cargo directly to cells can achieve high efficiency, e.g., above 90% or about 100%. In one embodiment, microinjection may be performed using a microscope and a needle (e.g., with 0.5-5.0 m in diameter) to pierce a cell membrane and deliver the cargo directly to a target site within the cell. Microinjection may be used for in vitro and ex vivo delivery.

Plasmids or mRNAs encoding the one or more activating proteins and/or cytokines, may be microinjected. In some cases, microinjection may be used to i) deliver DNA directly to a cell nucleus, and/or ii) deliver mRNA (e.g., in vitro transcribed) to a cell nucleus or cytoplasm. In certain examples, microinjection may be used to deliver plasmids directly to the nucleus or mRNA encoding the activating proteins and/or cytokines to the cytoplasm.

### b) Electroporation

In one embodiment, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

c) Hydrodynamic delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids, mRNA molecules, and proteins. The delivered cargos may be enriched in liver, kidney, lung, muscle, and/or heart.

### d) Transfection

The cargos, e.g., the polynucleotides described herein, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### e) Biolistics

The cargos, e.g., the polynucleotides and/or cytokines described herein, can be introduced to cells using a biolistic method or technique. The term of art “biolistic”, as used herein refers to the delivery of nucleic acids to cells by high-speed particle bombardment. In one embodiment, the cargo(s) can be attached, associated with, or otherwise coupled to particles, which than can be delivered to the cell via a gene gun (see e.g., Liang et al. 2018. Nat. Protocol. 13:413-430; Svitashev et al. 2016. Nat. Comm. 7:13274; Ortega-Escalante et al., 2019. Plant. J. 97:661-672). In one embodiment, the particles can be gold, tungsten, palladium, rhodium, platinum, or iridium particles.

### f) Implantable Devices

In one embodiment, the delivery system can include an implantable device that incorporates or is coated with the compositions or components of the compositions described herein. Various implantable devices are described in the art, and include any device, graft, or other composition that can be implanted into a subject.

### V. Methods

### 1) Methods for Inducing T Cell Progenitor Differentiation

In one embodiment, the present disclosure provides for methods for inducing T cell progenitor differentiation into mature T cells, which comprises introducing into a target tissue one or more of the compositions or polynucleotides described herein. In one embodiment, the methods comprise delivering to a target tissue where T cell progenitor differentiation occurs, one or more polynucleotides encoding one or more activating proteins that induce differentiation of T cell progenitors. In one embodiment, the methods comprise delivering to a target tissue one or more compositions comprising one or more polynucleotides encoding one or more activating proteins that induce differentiation of T cell progenitors. In one embodiment, the one or more compositions may be formulated in one or more delivery vehicles as described herein and configured to deliver the one or more polynucleotides to the target tissue. In one embodiment, the one or more polynucleotides may be formulated such that the one or more polynucleotides are delivered to endothelial cells in the target tissue wherein the activating proteins are displayed on a surface of the endothelial cells. In one embodiment, the one or more activating proteins induce differentiation of T cell progenitors to double positive (DP) T cells. In some examples, the one or more activating proteins comprise an agonist of a Notch signaling pathway or a T cell receptor (TCR) signaling pathway, or both. In some examples, the one or more activating proteins comprise DLL1, DLL4, Jagged1, Jagged2, or a combination thereof. In one embodiment, the target tissue can be an extrathymic tissue.

In one embodiment, the methods described herein may further comprise delivering one or more cytokines or polynucleotides encoding the one or more cytokines. In one embodiment, the one or more cytokines may be selected to induce further differentiation of T cell progenitors such as from DP T cells to single positive (SP) T cells. In one embodiment, the cytokines are chosen from IL-7, FLT3L, KIT ligand, IL-2, IL-4, IL-15, or a combination thereof. In one embodiment, the one or more cytokines comprise IL-7, FLT3L, or a combination thereof. In one embodiment, the one or more cytokines may be co-delivered with the one or more polynucleotides encoding the one or more activating proteins or delivered separately from the one or more activating proteins. In one embodiment, the cytokines may be delivered using any of the delivery systems disclosed herein. When delivered in a separate delivery vehicle from the one or more activating proteins, the one or more cytokines may be delivered prior to, concurrently with, or subsequently to delivery of the one or more activating proteins. In one embodiment, separate delivery of the one or more cytokines or polynucleotides encoding the cytokines takes place temporally with respect to the delivery of the one or more polynucleotides encoding the activating proteins.

As used herein, the term “temporally” or “temporal” with respect to cytokine delivery refers to the administration or delivery of cytokines or polynucleotides encoding the cytokines at a specific point in time that differs from the point in time during which the one or more polynucleotides encoding the one or more activating proteins are administered or delivered.

In one embodiment, the one or more cytokines or polynucleotides encoding the cytokines are delivered at a point in time after the delivery of the polynucleotides encoding the one or more activating proteins. In one embodiment, the one or more cytokines or polynucleotides encoding the cytokines are delivered at a point in time before the delivery of the polynucleotides encoding the one or more activating proteins. In one embodiment, the separate delivery of the one or more cytokines or polynucleotides encoding the cytokines takes place spatially with respect to the delivery of the one or more polynucleotides encoding the activating proteins.

As used herein, the term “spatially” or “spatial” with respect to cytokine delivery refers to the administration or delivery of cytokines or polynucleotides encoding the cytokines to a cell, tissue, or organ that differs from the cell, tissue, or organ to which the one or more polynucleotides encoding the one or more activating proteins are administered or delivered.

For example, after the one or more polynucleotides encoding the one or more activating proteins are delivered to the liver, the one or more cytokines or polynucleotides encoding the cytokines may be delivered intravenously into the blood circulation or may be delivered to a different tissue such as, for example, the spleen, small intestine, etc.

In one embodiment, one or more activating protein compositions or polynucleotides, encoding the activating proteins capable of inducing the differentiation of T cell progenitors as disclosed herein, may be delivered to a target cell population by ex vivo transduction prior to transplantation of the transduced target cell population into the subject. In one embodiment, the transduced target cell population may be cultured in vitro, and optionally cell sorted, prior to transplantation into the subject.

In one embodiment, the target cell population may be isolated from the subject, for example, from a biopsy, and the cells cultured in vitro prior to ex vivo transduction. In one embodiment, the target cell population may comprise stem cells resident in the target tissue. In one embodiment, the target cell population may comprise liver stem cells, e.g., oval stem cells. In one embodiment, the target cell population may comprise hematopoietic stem cells (HSCs) or lymphocyte progenitor cells. In one embodiment, the target cell population may comprise thymocytes or thymocyte progenitors. In one embodiment, the target cell population may comprise T cell progenitor cells isolated from a subject's bone marrow. In one embodiment, the target cell population may comprise induced pluripotent stem cells (IPSCs), embryonic stem cells (ESCs) or HSCs that, after ex vivo transduction, are cultured in vitro under conditions that induce differentiation along the T cell lineage prior to transplantation into the subject (see, for example, Gras-Pena et al. (2022) Human stem cell-derived thymic epithelial cells enhance human T-cell development in a xenogeneic thymus. J Allergy Clin Immunol. 2022 May; 149(5):1755-1771). In one embodiment, the transduced target cell population may be cell-sorted to select for CD2⁺, CD5+ and CD7+ and/or CD1+, CD34− cells (see, for example, Tjønnfjord et al. (1995) Haemopoietic progenitor cell differentiation: flow cytometric assessment in bone marrow and thymus. Br J Haematol. 91(4):1006-16). In one embodiment, the target cell population may comprise pleiotropic multipotent stem cells (SCs) (see, for example, Ragazzini et al. (2023) Defining the identity and the niches of epithelial stem cells with highly pleiotropic multilineage potency in the human thymus. Dev Cell. 20; 58(22):2428-2446.e9). In one embodiment, the target cell population may be isolated from a thymus organoid (see, for example, the published U.S. Patent Application No. 2021/0187164, the content of which is incorporated by reference herein in its entirety).

In one embodiment, the transduced target cell population may be transplanted directly into a target tissue, for example, by injection, e.g., into the subject's liver, thymus, and/or spleen. In another embodiment, the transduced target cell population may comprise transduced bone marrow T cell progenitors or thymocytes that can be administered to the subject by intravenous injection. The transduced bone marrow T cell progenitors may then migrate to and colonize a target tissue, e.g., the subject's liver, thymus, and/or spleen. For example, colonization of the thymus ensures thymocytes having a high affinity for self-peptides or the subject's major histocompatibility antigens (MHC) undergo negative selection, whereas those bone marrow T cell progenitors or thymocytes having a weak affinity for MHC class I or class II molecules may be positively selected. In one embodiment, the transduced bone marrow T cell progenitors may comprise Early Lymphoid Progenitors (ELPs). In one embodiment, the ELPs may comprise CD44+, CD25−, CD117+ cells (see, for example, Schwarz et al. (2007). Selective thymus settling regulated by cytokine and chemokine receptors. The Journal of Immunology, 178(4), 2008-2017). In one embodiment, the transduced bone marrow T cell progenitors may express ephrin type B receptor 2 (EphB2) or EphB3 (see, for example, Alfaro et al. (2015) EphB2 and EphB3 play an important role in the lymphoid seeding of murine adult thymus. J Leukoc Biol. 2015; 98(6):883-96).

In one embodiment, the one or more polynucleotides comprise one or more plasmid DNAs encoding the one or more activating proteins. In one embodiment, the one or more polynucleotides comprise one or more mRNAs encoding the one or more activating proteins. In one embodiment, the one or more polynucleotides comprise a combination of one or more plasmid DNAs and mRNAs. In one embodiment, the one or more mRNAs comprise one or more modifications that increase mRNA stability, reduce immunogenicity, and/or improve translation efficiency. In one embodiment, the mRNAs comprise self-replicating mRNA. In one embodiment, the one or more polynucleotides are delivered via a lipid nanoparticle, an exosome, a microvesicle, a viral vector, an engineered retroelement vector, a polynucleotide-based nanostructure, or an extracellular contractile injection system or as described elsewhere herein.

### 2) Methods for Identifying Candidate Activating Proteins

The present disclosure also provides a method for identifying candidate activating proteins, or combinations of activating proteins, that induce T cell proliferation and differentiation in a target tissue. In one embodiment, the method comprises (a) delivering one or more polynucleotides encoding one or more candidate activating proteins that induce T cell differentiation of T cell progenitor cells to a population of cells derived from a tissue of interest, (b) selecting a sample of single clone cells expressing the candidate activating proteins from the population of cells derived from the tissue of interest, (c) treating a population of T cell progenitors with the sample of single clone cells expressing the candidate activating proteins, (d) quantifying the percentage of T cells differentiated from the population of T cell progenitors, and (e) selecting the polynucleotides encoding the candidate activating proteins that resulted in T cell differentiation for evaluation in vivo, wherein the percentage of differentiated T cells is at or above 50% of the total cell population consisting of the differentiated T cells and T cell progenitors. In one embodiment, the tissue of interest can be the liver, spleen, small intestine, thymus, or a combination thereof. In one embodiment, the sample of single clone cells can be selected using flow cytometry.

### VI. Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In one embodiment, the pharmaceutical formulation can include, such as an active ingredient, any of the compositions or components thereof described in greater detail elsewhere herein.

In one embodiment, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In one embodiment, the subject in need thereof has or is suspected of having a hematopoietic disease or a symptom thereof. Exemplary diseases are described in greater detail elsewhere herein, such as in connection with therapeutic methods. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### 1) Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In one embodiment, the pharmaceutical formulation can also include an effective amount of secondary active agents, including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, imagining agents, sensitizers, and combinations thereof.

### 2) Effective Amounts

In one embodiment, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In one embodiment, the one or more therapeutic effects are to modify a nucleic acid in vitro, ex vivo, in situ, or in vivo.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges.

In one embodiment, the effective amount, least effective amount, and/or therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 pM, nM, μM, mM, or M or be any numerical value with any of these ranges.

In other embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In one embodiment, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In one embodiment, where a cell population is present in the pharmaceutical formulation (e.g., as a primary and/or or secondary active agent), the effective amount of cells can range from about 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL.

In one embodiment, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In one embodiment, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In one embodiment, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L. In one embodiment, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In one embodiment, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In one embodiment, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### 3) Dosage Forms

In one embodiment, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In one embodiment, the given site is proximal to the administration site. In one embodiment, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In one embodiment, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In one embodiment, compounds, molecules, compositions, vectors, vector systems, cells, or a combination thereof described herein can be the ingredient whose release is delayed. In one embodiment, the primary active agent is the ingredient whose release is delayed. In one embodiment, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wlkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In one embodiment for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In one embodiment, a primary active ingredient, optional secondary active ingredient, and/or pharmaceutically acceptable salt thereof where appropriate can be in a dosage form adapted for inhalation is in a particle-size-reduced form that is obtained or obtainable by micronization. In one embodiment, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In one embodiment, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable formulation. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In one embodiment, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in one embodiment, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### 4) Co-Therapies and Combination Therapies

In one embodiment, the pharmaceutical formulation(s) described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In one embodiment, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, imaging agents, sensitizers, and combinations thereof.

### 5) Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In one embodiment, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In one embodiment, the first one or a few initial amount(s) administered can be a higher dose than subsequent doses. This is typically referred to in the art as a loading dose or doses and a maintenance dose, respectively. In one embodiment, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### VI. Kits

Also described herein are kits containing any one or more of the elements disclosed in the above compositions and methods. In one embodiment, the kit comprises a vector system as taught herein or one or more of the components of the composition as taught herein, such as the one or more polynucleotides encoding the one or more activating proteins that induce T cell differentiation of T cell progenitor cells and/or one or more cytokines or polynucleotides encoding said polynucleotides, and/or a delivery vehicle configured to deliver said polynucleotides and/or cytokines, and instructions for using the kit. Elements may be provided individually or in combinations, and may be provided in any suitable container, such as a vial, a bottle, or a tube. In one embodiment, the kit includes instructions in one or more languages. The instructions may be specific to the applications and methods described herein. In one embodiment, a kit comprises one or more reagents for use in a process utilizing one or more of the elements described herein. Reagents may be provided in any suitable container, such as a vial, a bottle, or a tube. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In one embodiment, the buffer is alkaline. In one embodiment, the buffer has a pH from about 7 to about 10. In one embodiment, the kit comprises one or more of the vectors and/or one or more of the polynucleotides described herein. The kit may advantageously allow to provide all elements of the systems of the present disclosure.

Further embodiments are illustrated in the following Examples which are given for illustrative purposes only and are not intended to limit the scope of the disclosure.

### EXAMPLES

### Example 1: Evaluation of Candidate Activating Proteins for Expression in Hepatocytes

### 1) Materials and Methods

### a) Plasmid Construction

In vitro transcription vectors were cloned by inserting UTRs into a pET45 vector via Gibson assembly using Gibson Assembly Master Mix (NEB E2611L) and transformation into chemically competent Stbl3 cells. G-blocks encoding the human alpha globin 5′ UTR and 3′ FI element were synthesized de novo by IDT. A hard-coded A30LA70 polyA tail was added by PCR and ligation using KLD enzyme mix (New England Biolabs). Subsequent coding sequences were inserted by digestion with NcoI and XhoI and Gibson assembly. Plasmid sequences were verified via next generation sequencing, long read sequencing (Primordium Labs), and PCR to verify the length of polyA tails.

b) In Vitro Transcription and LiCl Purification of mRNA

Plasmids were linearized, and a T7-driven in vitro transcription reaction (Life Technologies, Carlsbad, CA) was performed to generate mRNA with 101 nucleotide long poly(A) tails. The 5′ UTR and the 3′ FI elements contain sequences from the human alpha globin gene. Capping of mRNA was performed in concert with transcription through addition of a trinucleotide cap1 analog CleanCap, and m1Ψ-5′-triphosphate (TriLink, San Diego, CA) was incorporated into the reaction instead of uridine-5′-triphosphate (UTP). LiCl-based purification of mRNA was performed.

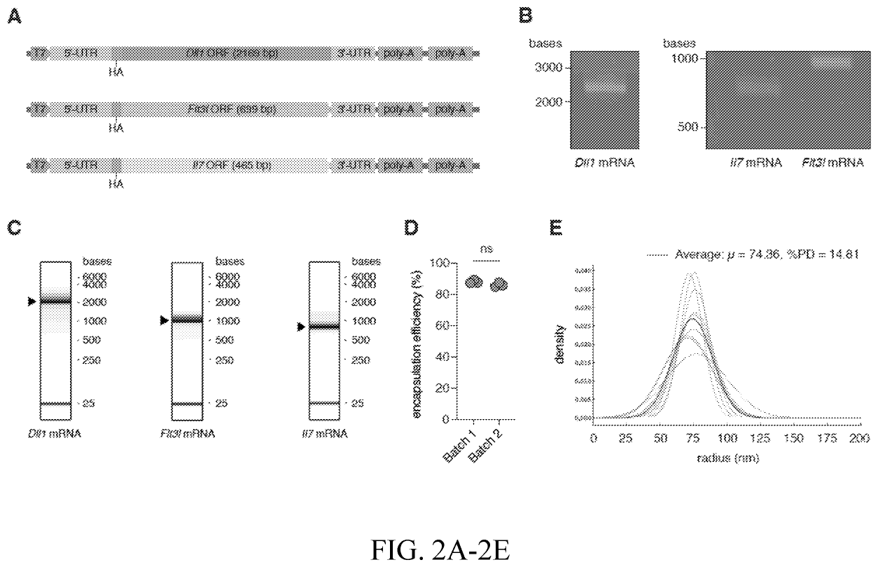

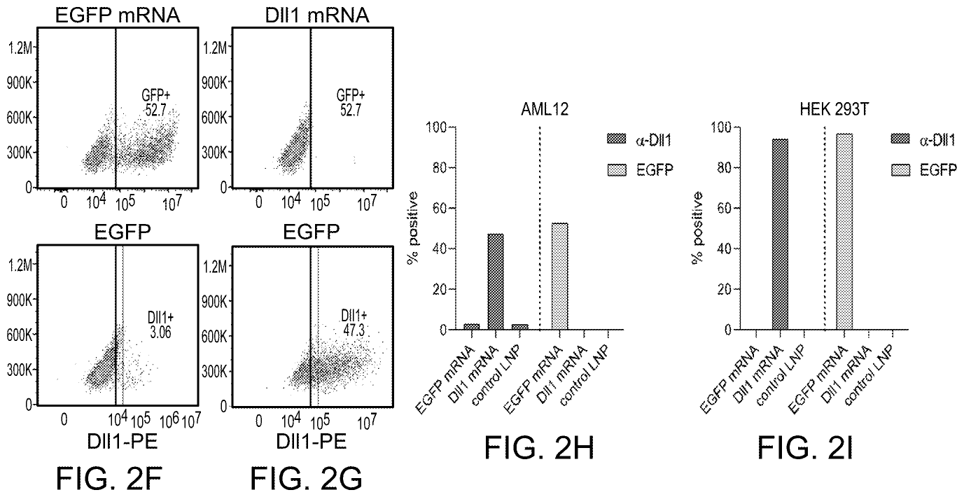

Maps of linearized plasmids used for in vitro transcription (IVT) of the mRNAs encoding candidate activating proteins DLL1 (D), FLT-3L (F), and IL-7 (I) mRNAs (DFI mRNAs) are depicted in FIG. 2A. The integrity of the in vitro transcribed and capped DFI mRNAs was then verified by agarose gel electrophoresis (FIG. 2B) and TapeStation RNA ScreenTape Analysis (Agilent) (FIG. 2C) before aliquoting at 1 μg/μl and storing at −80° C.

### c) DFI LNP Production

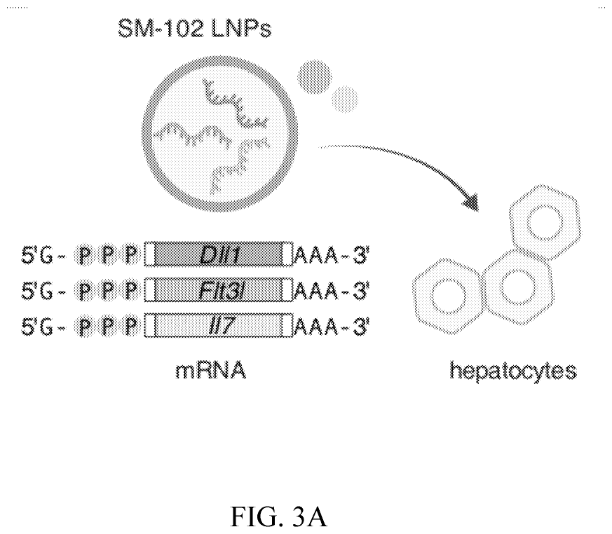

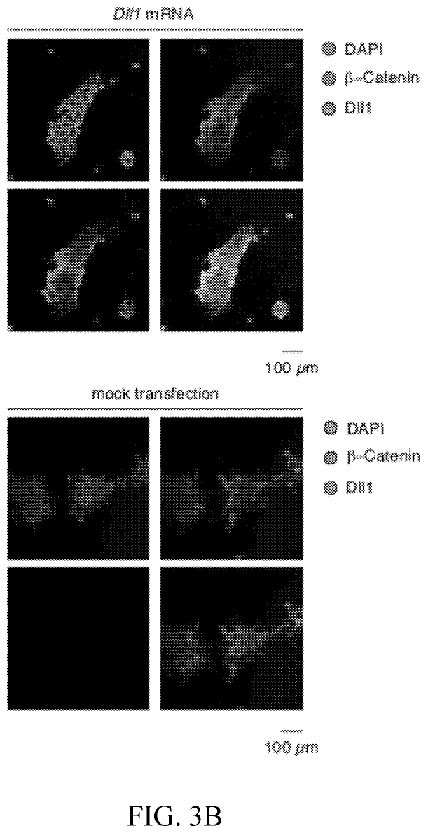

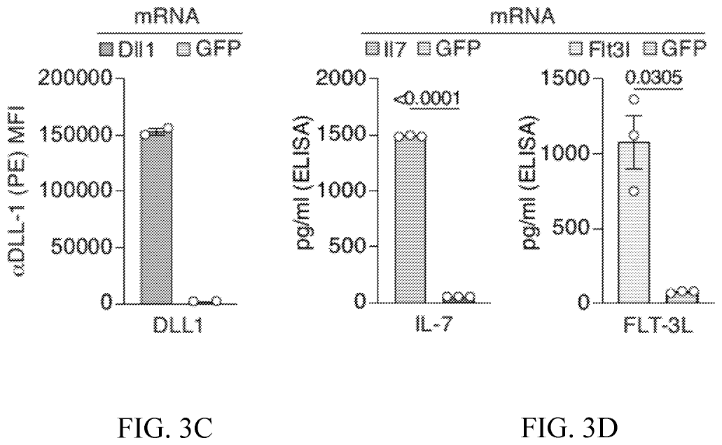

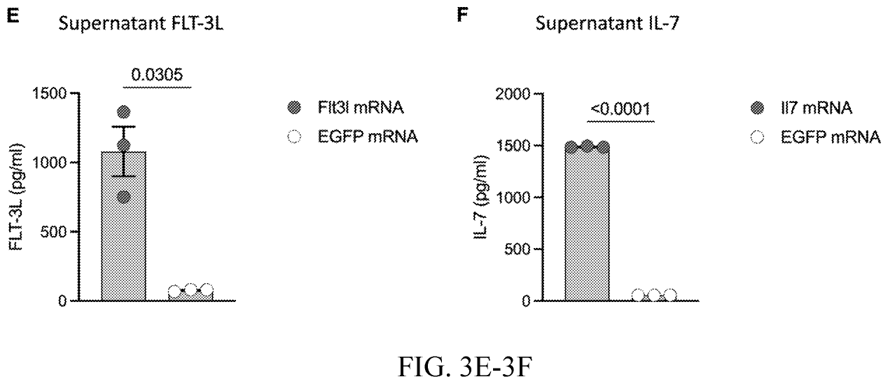

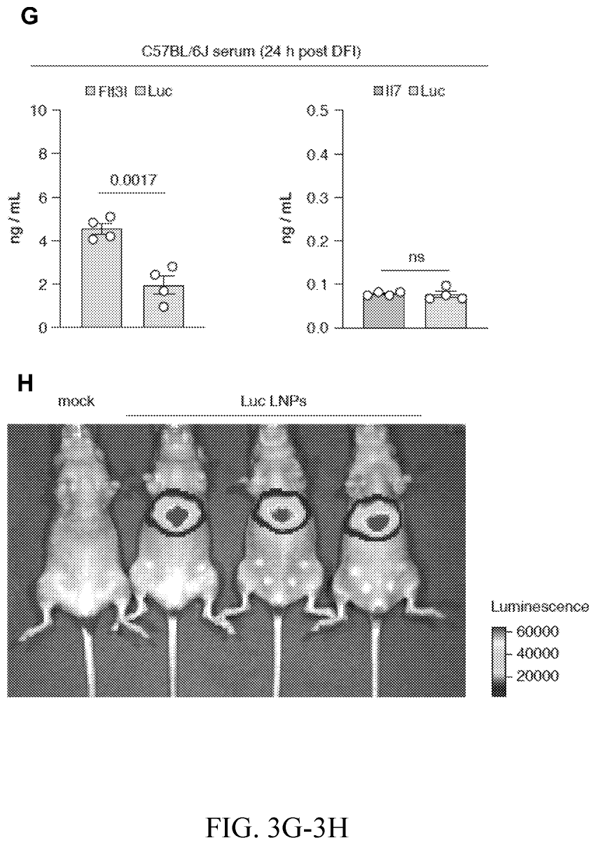

Lipid nanoparticles (LNPs) were formulated by combining SM-102 as ionizable lipid, 1,2-distearoyl-sn-glycero-3-phosphocholine (DSPC), cholesterol, and 1,2-dimyristoyl-racglycero-3-methoxypolyethylene glycol-2000 (DMG-PEG-2000) in a molar ratio of 50:10:38.5:1.5. These were formulated into LNPs along with mRNA using microfluidic mixing. Briefly, an ethanol phase containing lipidoid, phospholipid, cholesterol and DMG-PEG at a designated molar ratio was mixed with an aqueous phase (10 mM citrate buffer, pH 3) containing mRNA at a flow rate ratio of 1:3 and at a lipidoid/RNA weight ratio of 10:1 in a microfluidic chip device. LNPs were dialyzed against 1×PBS in a 20 kDa molecular weight cut-off cassette for 2 h, sterilized through a 0.22 m filter and stored at 4° C. The efficiency of mRNA encapsulation within the LNPs was determined to be ~85%, indicating that the formulation is capable of effectively complexing with and encapsulating microgram quantities of DFI mRNA. These SM-102 LNPs encapsulating W-modified and m7GpppNm-capped DFI, firefly luciferase (Luc) or GFP mRNA, respectively were used for all subsequent experiments. Consistent with prior findings, when delivered intravenously, these lipid nanoparticles (LNPs) primarily target the liver, and mRNA expression was observed specifically in liver cells. Primary hepatocytes produced DLL1 protein, which was displayed on the cell surface of transfected hepatocytes as well as biologically active IL-7 and FLT-3L, which are released from the liver cells into the bloodstream (FIGS. 3B-3D).

### d) LNP Characterization

The hydrodynamic size, PDI and zeta potential of LNPs were measured using a Zetasizer Nano ZS90 (Malvern Instruments). The mRNA encapsulation efficiency of LNPs were determined using a modified Quant-iT RiboGreen RNA assay (Invitrogen) and found to be >85% on average (FIG. 2D). LNP endotoxin levels were consistently found to be <1 endotoxin unit per ml. The average hydrodynamic LNP diameter was ~75 nm with a polydispersity index of 0.02-0.03 as measured by dynamic light scattering (FIG. 2E).

### e) Cell Culture and Transfection

Unless otherwise stated, mammalian cells were maintained in T75 flasks (ThermoFisher 156499) at 37° C. with 5% CO2 in either DMEM-GlutaMAX (ThermoFisher 10569044) or RPMIGlutaMAX (ThermoFisher 61870127). All media was supplemented with 10% FBS (VWR 97068-085) and 1× penicillin-streptomycin (ThermoFisher 15140122). For growth of primary T cells, media was also supplemented with 50 μM 2-mercaptoethanol (ThermoFisher 21985023). Images of transfected cells were acquired on a Leica DMI8 Confocal Microscope running Leica Application Suite X (1.4.3), equipped with a Lecia Stellaris 5 camera using an HC PL APO CS2 20×/0.75 DRY objective and a pinhole setting of 1 Airy Unit. Images were processed using Fiji (www.imagej.net/software/fiji/downloads).

### f) Flow Cytometry

Cells were prepared and stained according to the staining protocol of each experiment outlined below, pelleted at 500 g for 5 min, and resuspended in 200 μL of flow cytometry buffer (PBS supplemented with 2% EDTA (Life Technologies 15575020) and 5% FBS (VWR 97068-085)). Samples were run on a Beckman Coulter Cytoflex LX flow cytometer, and analysis was performed using the FlowJo v10 software. All antibodies used can be found in the table below.

### g) Antigen-Specific Tetramer Staining

All peptide-MHC tetramers were provided by the NIH Tetramer Core Facility. Before staining of cells with viability dye and surface antibody cocktails, samples were stained with titrated tetramer combinations. For OVA vaccination experiments, SIINFEKL-H-2Kb-PE (SEQ ID NO: 753) (1:100 dilution) and SIINFEKL-H-2Kb-APC (SEQ ID NO: 753) (1:100 dilution) were used. For central tolerance experiments, SIINFEKLH-2Kb-PE (SEQ ID NO: 753) (1:100 dilution) and SIINFEKL-H-2Kb-APC (SEQ ID NO: 753) (1:100 dilution) and AAHAEINEA-I-Ab-PE (SEQ ID NO: 782) (1:20 dilution) and AAHAEINEA-I-Ab-PE (SEQ ID NO: 782) (1:20 dilution) were used. T cells isolated from OTI or OT-II mice were used as staining controls, respectively. For NOD diabetes experiments, KYNKANAFL-H-2Kd-PE (SEQ ID NO: 781) (1:50 dilution) and KYNKANAFL-H-2Kd-APC (SEQ ID NO: 781) (1:50 dilution) were used. T cells isolated from NY8.3 mice were used as staining controls. In all experiments, tetramers were diluted in PBS and cells were stained for 20 min on ice before addition of surface antibody cocktails and/or fixation.

### h) Animal Experiments

All animal experiments were approved by the Institutional Animal Care and Use Committee (IACUC) of the Broad Institute (Protocol ID 0017-09-14-2). Animal maintenance complied with all relevant ethical regulations and were consistent with local, state and federal regulations as applicable, including the National Institutes of Health Guide for the Care and Use of Laboratory Animals. Animals were kept on a 12-h light/dark cycle between 68° F. and 79° F. and 30-70% humidity. Mice were acclimated at the animal facility for at least 7 days before performing any experiments.

### i) Thymus Tissue Processing for Slide-Seq

Thymus samples for spatial Slide-TCR-seq were derived from C57BL/6J male mice (Jackson Laboratories). The thymus lobes were isolated, weighed, and embedded in optimal cutting temperature (OCT) compound. Following this, the specimens were promptly frozen on dry ice and stored at −80° C. The right lobes from 21 distinct time points were selectively chosen for spatial Slide-TCR-seq.

### j) Isolation of Liver Parenchymal Immune Cells

Harvesting of livers from Foxn1−/− mice treated with DFI was performed under terminal anesthesia to minimize the risk of thrombosis. The abdomen was sterilized using 70% ethanol. An incision was initiated at the pelvis with scissors, lifting the skin with forceps, and continued to the sternum to expose the peritoneal cavity membrane. The peritoneum was carefully opened from the pelvis to the sternum, and the abdomen was further opened by incising along the flanks. The abdominal organs were moved aside using cotton tip applicators to expose the liver. The liver was perfused by locating and incompletely severing the inferior vena cava at the top of the liver, followed by the insertion of a 23 G needle attached to a 10 mL syringe containing cold PBS. Correct perfusion was indicated by the liver blanching and appearing pale. The gallbladder was removed and discarded, and the liver was carefully resected, taking care to sever any adhesions to surrounding tissues, then placed in DMEM and kept on ice. It was critical to ensure the entire liver was well perfused to prevent the contamination of the tissue-resident leukocyte populations of interest with blood-borne cells.

Single-cell suspension from the liver were prepared by placing the liver lobes into individual 70 m cell strainers atop 50 mL tubes. The liver tissues were mashed through the strainer with the rubber end of a 5 mL syringe plunger, rinsing it with FACS buffer. The cell suspension was then centrifuged at 300×g with swinging buckets for 3 minutes at 21° C.-23° C., and the supernatant was carefully removed and discarded. The pellet was resuspended in a freshly prepared 42% isotonic Percoll solution (2.1 mL 10×PBS, 29 mL 1×PBS, 18.9 mL Percoll), mixed well, and transferred to a 15 mL tube. The original tube was washed with more Percoll solution, which was then combined in the 15 mL tube. The suspension was centrifuged at 800×g for 20 minutes at 21° C.-23° C. with the brake off to separate hepatocytes (top cellular layer) and leukocytes (pellet). The top cellular layer and Percoll were carefully removed and discarded, and the pellet was resuspended in ACK Lysis buffer and transferred to a new 15 mL tube containing additional ACK Lysis buffer. After a 5-minute incubation at 21° C.-23° C. with occasional shaking, the suspension was centrifuged again at 300×g for 3 minutes at 21° C.-23° C. to repellet the leukocytes. Finally, the pellet was resuspended in FACS buffer for further analysis and the cell count was determined using a small aliquot of the cell suspension.

### k) Isolation and Staining of Blood-Circulating T Cells

For all vaccination experiments, at the indicated time points post-vaccination, approximately 100 μL of blood was collected with EDTA-coated capillary tubes from each mouse and then transferred to an EDTA-coated tube. The collected blood samples were centrifuged at 2000 g for 10 min, followed by transferring the resulting plasma into another tube and antibody staining was performed using the eBioscience™ 1-step Fix/Lyse Solution (10×) (ThermoFisher, 00-5333-54). Briefly, 50 or 100 μl of blood samples were incubated with 50 or 100 μl 2-fold concentrated antibody cocktails (1:100 final dilution) as well as TruStain FCX (anti-mouse CD16/32, clone 93; BioLegend) (1:50 final dilution) for 20 min at 4° C. in the dark, followed by the addition of 2 mL of 1-step Fix/Lyse Solution and 15 min incubation at room temperature in the dark. Samples were then washed twice with 10 mL of PBS followed by centrifugation at 500×g for 5 min and finally resuspended in 200 μl FACS buffer. In the case of tetramer staining, samples were pre-incubated with the respective 2-fold concentrated tetramers (1:20-1:100 final dilution), followed by incubation with 2-fold concentrated antibody cocktails (1:100 final dilution) as well as TruStain FCX (anti-mouse CD16/32, clone 93; BioLegend) (1:50 final dilution).

### l) Antibodies and Dyes Used for Flow Cytometry and Immunofluorescence Staining

| | | | | | Catalog |
|---|---|---|---|---|---|
| Name | Fluorophore | Clone | Lot # | Mannfactorer | number |
| PE anti-mouse IL-2 | PE | JES6- | B351622, | Biolegend | 503808 |
| | | 5H4 | B377599 | | |
| anti-m/rDLL1 Affinity Purified | | | YXZ0422031 | R&D | AF3970 |
| Sheep IgG | | | | systems | |
| Brilliant Violet 510 anti-mouse | BV510 | 30-F11 | B386738, | Biolegend | 103138 |
| CD45 | | | B360620, | | |
| | | | B384034 | | |
| APC anti-mouse IFN-γ | APC | XMG1.2 | B354911, | Biolegend | 505810 |
| | | | B370994, | | |
| | | | B396246 | | |
| anti-sheep IgG | | | AAFQ0623011 | R&D | NL011 |
| | | | | systems | |
| PE anti-mouse/human CD44 | PE | IM7 | B343363 | Biolegend | 103024 |
| Anti-Mo CD8a, eBioscience | eFluor 450 | 53-6.7 | 2527379 | Invitrogen | 48-0081-82 |
| BD GolgiPlug Protein Transport | | | 1277727 | BD | 51-2301KZ |
| Inhibitor | | | | bioscience | |
| PerCP Rat Anti-Mouse CD4 | PerCP | RM4-5 | 2279727, | BD | 553052 |
| | | | 3201956, | bioscience | |
| | | | 1334056 | | |
| APC anti-mouse CD62L | APC | MEL-14 | B371017 | Biolegend | 104412 |
| FITC anti-mouse CD3 | FITC | 17A2 | B388315, | Biolegend | 100204 |
| | | | B388061, | | |
| | | | B406287 | | |
| APC Hamster Anti-Mouse | APC | H57- | 3030398, | BD | 553174 |
| TCR β | | 597 | 2076848 | bioscience | |
| TruStain FcX (anti-mouse | | 93 | B398113, | Biolegend | 101320 |
| CD16/32) | | | B380119, | | |
| | | | B368516, | | |
| | | | B372578 | | |
| Anti-Mo CD45.1, eBioscience | APC | A20 | 2536046 | Invitrogen | 17-0453-82 |
| Anti-Mo CD45.2, eBioscience | PE | 104 | 2534115 | Invitrogen | 12-0454-82 |
| Anti-Mo CD135 (Flt3), eBioscience | PerCP-eFluor | A2F10 | 2408298 | Invitrogen | 46-1351-82 |
| | 710 | | | | |
| FITC anti-mouse Ly-6A/E (Sca-1) | FITC | D7 | B359231 | Biolegend | 108106 |
| APC anti-mouse CD34 | APC | HM34 | B383558 | Biolegend | 128612 |
| PE anti-mouse CD117 (c-kit) | PE | 2B8 | B343465 | Biolegend | 105808 |
| PE/Cyanine7 anti-mouse CD127 | PE/Cyanine7 | A7R34 | B375472 | Biolegend | 135014 |
| (IL-7Rα) | | | | | |
| eBioscience Fixable Viability Dye | eFluor 780 | | 2752774 | Invitrogen | 65-0865-14 |
| eFluor 780 | | | | | |

### m) Chemicals and Enzymes

Chemicals and enzymes are listed as name (vendor, catalog number): Tissue-Tek O.C.T. Compound (SAKURA, 4583). Glass bottom 24-well plates (Cellvis, P24-1.5HN). 3-(Trimethoxysilyl) propyl methacrylate (Sigma-Aldrich, M6514). poly-D-lysine (Sigma-Aldrich, A-003-M). 16% PFA (Electron Microscopy Sciences, 15710-S). UltraPure™ DNase/RNase-Free Distilled Water (Invitrogen, 10977023). Methanol (Sigma-Aldrich, 34860-1LR). PBS (Gibco, 10010-023). Tween-20, 10% solution (Calbiochem, 655206). Yeast tRNA (ThermoFisher Scientific, AM7119). SUPERase In RNase Inhibitor (ThermoFisher Scientific, AM2696). UltraPure™ SSC, 20× (Invitrogen, 15557044). Formamide (Calbiochem, 655206). Ribonucleoside vanadyl complex (New England Biolabs, S1402S). T4 DNA ligase, 5 Weiss U/μL (ThermoFisher Scientific, EL0012). Phi29 DNA polymerase (ThermoFisher Scientific, EP0094). Deoxynucleotide (dNTP) Solution Mix (New England Biolabs, N0447L). UltraPure BSA (ThermoFisher Scientific, AM2618). 5-(3-aminoallyl)-dUTP (ThermoFisher Scientific, AM8439). Methacrylic acid NHS ester, 98% (Sigma-Aldrich, 730300). DMSO, anhydrous (Invitrogen, D12345). Acrylamide solution, 40% (Bio-Rad, 161-0140). Bis Solution, 2% (Bio-Rad, 161-0142). Ammonium persulfate (Sigma-Aldrich, A3678). N,N,N′,N′-Tetramethylethylenediamine (Sigma-Aldrich, T9281). Gel Slick® Solution (Lonza Bioscience, 50640). OminiPur SDS, 20% (Calbiochem, 7991). Proteinase K Solution, RNA grade (ThermoFisher Scientific, 25530049). Antarctic Phosphatase (New England Biolabs, M0289L). DAPI (Molecular Probes, D1306). 10% Triton X-100 (Sigma-Aldrich, 93443). Flamingo Fluorescent Protein Gel Stain (Bio-Rad, 1610490).

2) In Vitro Delivery of mRNAs Encoding Candidate Activating Proteins

### a) Expression of Candidate Activating Proteins in Primary Hepatocytes

The expression vectors encoding candidate activating proteins were screened and evaluated for successful expression in hepatocytes and subsequent differentiation of T cell progenitors (FIG. 2F-2K). DLL1 expression was validated by FACS analysis of primary hepatocytes transfected with DLL1 mRNA (FIG. 2F-2G). Primary hepatocytes were stained with anti-DLL1 monoclonal antibody (HMD1-5) PE for surface expression of DLL1 following treatment with either 2.5 μg EGFP mRNA in 7.5 μL Lipofectamine™ 3000 (FIG. 2F) or 2.5 μg DLL1 mRNA in 7.5 μL Lipofectamine™ 3000 (FIG. 2G). Percentage of primary hepatocytes positive for expression of DLL1 or EGFP was then quantified (FIG. 2H) and compared to the percentage of HEK 293T cells positive for expression of DLL1 or EGFP (FIG. 2I).

In vitro expression of candidate activating proteins (e.g., DLL1 mRNA) was further validated in hepatocytes (FIG. 3B). In the example provided herein, immortalized hepatocytes were stained with anti-DLL1 monoclonal antibody (AB10554) for surface expression of DLL1 following treatment with either 0.25 μg DLL1 mRNA in 7.5 μL Lipofectamine™ 3000 or 7.5 μL empty Lipofectamine™ 3000.

Cytokine expression in primary hepatocytes following transfection with FLT3L, and IL-7 mRNA was also evaluated (FIG. 3D). ELISA was performed using anti-FLT3L and anti-IL-7 monoclonal antibodies in primary hepatocyte samples treated with either 2.5 μg FLT3L mRNA in 7.5 μL Lipofectamine™ 3000 or 7.5 μL empty Lipofectamine™ 3000 or 2.5 μg IL-7 mRNA in 7.5 μL Lipofectamine™ 3000 or 7.5 μL empty Lipofectamine™ 3000. FIGS. 3E-3F show that transfection of FTL-1 and IL-7 LNPs into primary hepatocytes induces protein expression, processing and secretion of biologically active activating proteins into the culture supernatant. Taken together, these data indicate that hepatocytes are able to detectably express activating proteins and cytokines following transfection in vitro.

### b) Expression of a Candidate Activating Protein Induces Differentiation of T Cell Progenitors In Vitro

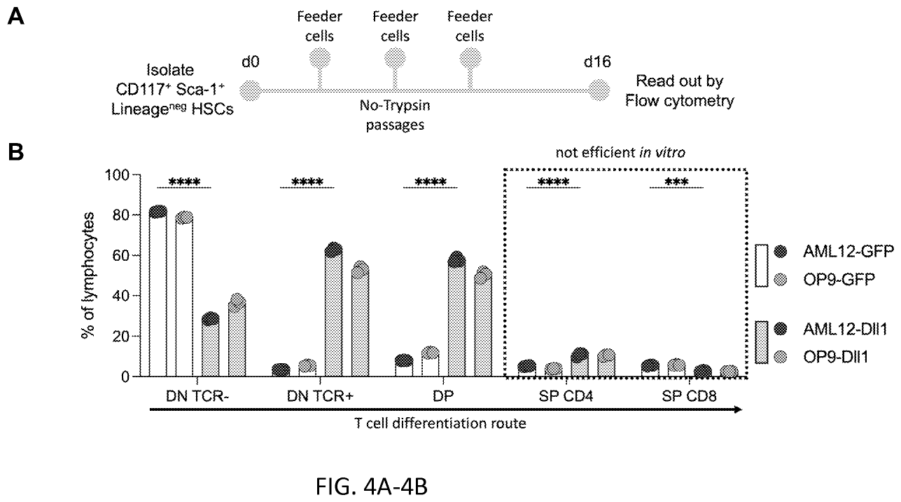

To determine whether hepatocytes expressing DLL1 were capable of inducing differentiation of T cell progenitors, hematopoietic stem cells were co-incubated with AML12 hepatocytes expressing GFP (AML12-GFP) or DLL1 (AML12-DLL1), or with OP9 stromal cells expressing GFP (OP9-GFP) or DLL1 (OP9-GFP), and the percentage of differentiated lymphocytes was evaluated (FIG. 4A-4B). Data are presented as the percentage of lymphocytes that are CD4⁻ CD8⁻ double negative, T cell receptor negative (DN TCR−), CD4⁻ CD8⁻ double negative, T cell receptor positive (DN TCR+), CD4⁺ CD8⁺ double positive (DP), CD4 single positive (SP CD4), or CD8 single positive (SP CD8), following co-incubation. Results indicate that DLL1-expressing hepatocytes and stromal cells are able to induce differentiation of hematopoietic stem cells into TCR+ and DP T cells.

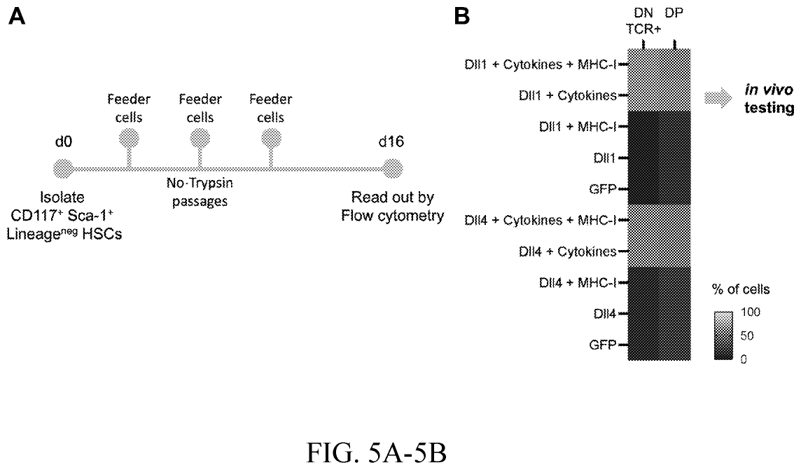

FIG. 5A shows the procedure for screening for combinations of activating proteins that induce T cell differentiation. FIG. 5B shows the relative percentages of differentiated T cells for each of the combinations tested, wherein the combinations yielding the highest percentage of differentiated T cells were used for in vivo testing.

3) In Vivo Delivery of mRNAs Encoding Candidate Activating Proteins

Upon formulation, concentrations and encapsulation efficiency of mRNA were determined using Quant-it RiboGreen RNA Assay Kit (ThermoFisher, R11490). mRNA-LNPs (equal molar, normalized to the control RNA) in a total volume of 100 μL were injected through retro-orbital injection into each mouse. For Luciferase imaging, PBS injection was used as negative control. For all other experiments investigating DFI mRNA-LNPs, Luc LNPs was used as a negative control.

### Serum Cytokine Analyses

Heart blood was aspirated by cardiac puncture using EDTA-precoated syringes and collected in BD Microtainer® Tubes with Potassium EDTA additive (BD, 365974). Serum was isolated by centrifugation at 2000 rpm for 10 mins at room temperature and snap-frozen on dry ice. All samples were received by IDEXX BioAnalytics and stored securely at −80° C. prior to analysis. Samples were tested on the Milliplex MAP Mouse Cytokine/Chemokine Magnetic Bead Panel (MilliporeMCYTOMAG-70K-PMX) according to the kit protocol as qualified. Data were collected by xPONENT® 4.3 (Luminex) and data analysis was completed using BELYSA® 1.1.0 software. The data collected by the instrument software are expressed as Median Fluorescence Intensity (MFI). MFI values for each analyte are collected per each individual sample well. Analyte standards, quality controls, and sample MFI values were adjusted for background. Calibrator data were fit to either a five-parameter logistic (5PL) or four parameter logistic (4PL) model depending on best fit to produce accurate standard curves for each analyte. Quality control and sample data were interpolated from the standard curves and then adjusted according to dilution factor to provide calculated final concentrations of each analyte present in the sample. Samples demonstrating hemolysis were excluded from the analysis.

Serum Flt3L and IL-7 levels in mice treated with either Flt3l/Il7 mRNA LNPs or Luc LNPs were detected by Flt3L and IL-7 cytokine ELISA, respectively (FIG. 3G).

### c) In Vivo Luciferase Imaging

In vivo activity of Luciferase following delivery of Luc mRNA-LNPs was measured using a Competent IVIS-Perkin Elmer IVIS Spectrum CT System (Perkin Elmer). Mice were injected through retro-orbital injection with Luc mRNA-LNPs at a dose of 5 μg mRNA per mouse., and bioluminescence imaging was performed on an IVIS imaging system (PerkinElmer). At 6 h postinjection, mice were anesthetized with isoflurane and intraperitoneally (i.p.) injected with Dluciferin potassium salt (150 mg per kg (body weight)). Mice were imaged 5 mins post injection using auto exposure settings. The luminescent activity was quantified using Aura 4.0 imaging software. The background was determined by including mice injected with PBS control and subtracted from each measurement.

Consistent with prior findings, when delivered intravenously, the LNPs target primarily the liver. FIG. 3H shows IVIS luciferase imaging of Foxn1−/− mice that have received either mock SM-102 LNPs or Luc SM-102 LNPs intravenously 8 hours before imaging. N=4 mice.

### Example 2: Evaluation of T Cell Production in Mice Following Treatment with Activating Protein Expression Vectors

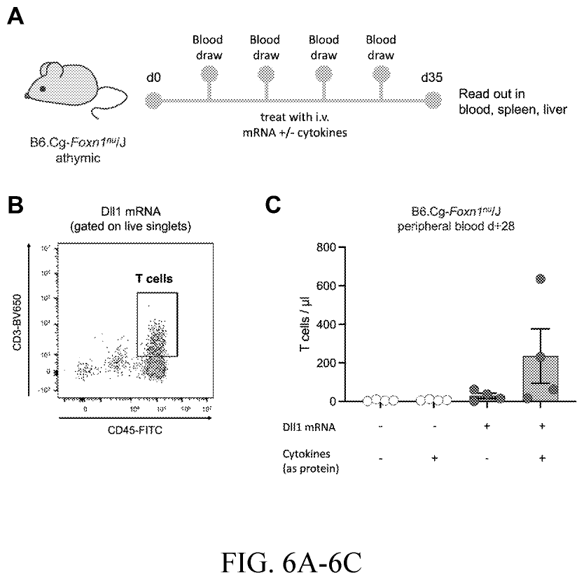

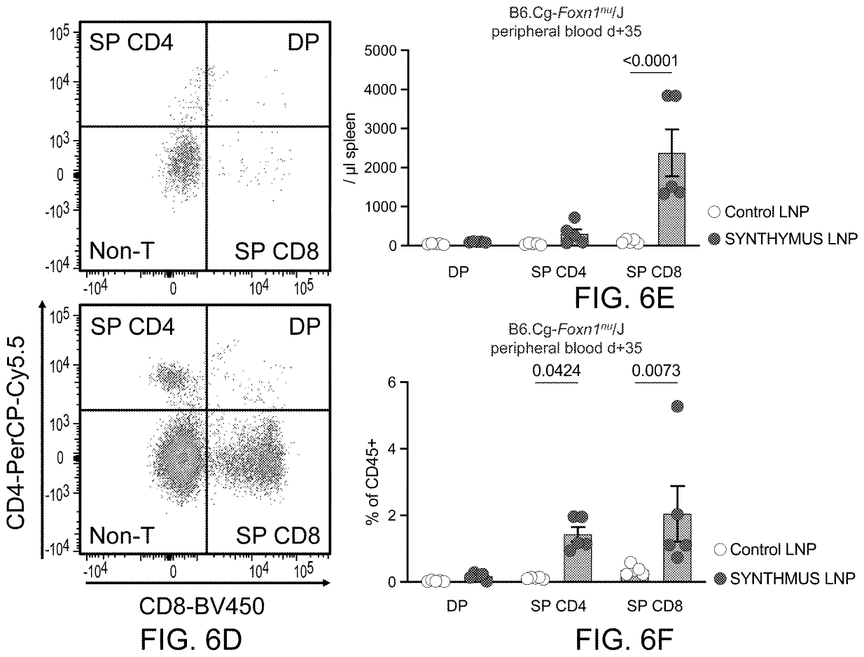

To evaluate whether the above in vitro results could be validated in vivo, athymic B6.Cg-Foxn1^{nu}/J mice were treated with DLL1 mRNA or DLL1 mRNA in combination with recombinant IL-7/FLT3L for 35 days, and peripheral blood was drawn at various intervals and analyzed using FACS to detect differentiated T cells (FIG. 6A-6C). Treatment of athymic mice with SYNTHYMUS was shown to preferentially induce development of mature CD8⁺ SP T cells (FIG. 6B-6F). Based on these observations, Applicants next sought to identify the non-thymic tissues or organs where these DP and SP T cells were produced.

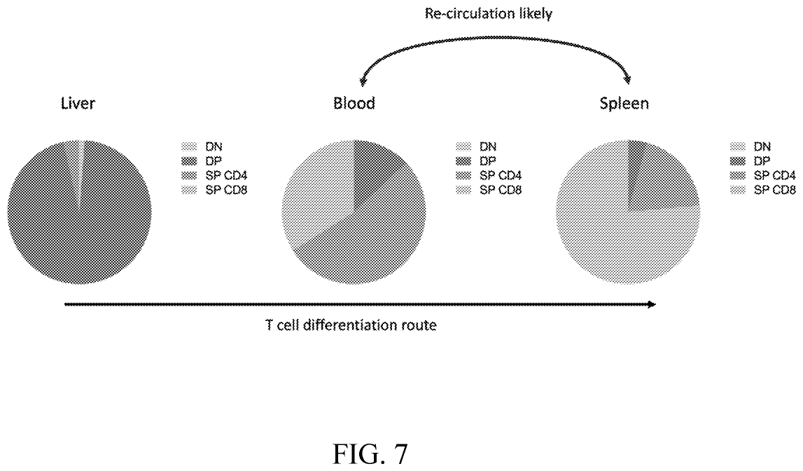

Analysis of lymphocytes isolated from peripheral blood and spleen of athymic B6.Cg-Foxn1^{nu}/J mice demonstrated that co-receptor expression of the T cell population varies between T cells in the liver, in circulation, and in the spleen (FIG. 7). Although treatment with DLL1 mRNA with or without cytokines leads to the production of DP T cells in the liver, only treatment with DLL1 in combination with cytokines seemed to induce T cell differentiation in peripheral blood, with most T cells being either DN or CD4+ SP (FIG. 7). In the spleen, T cell differentiation was largely induced by treatment with DLL1 mRNA in combination with cytokines, with most T cells being CD8+ SP T cells (FIG. 7).

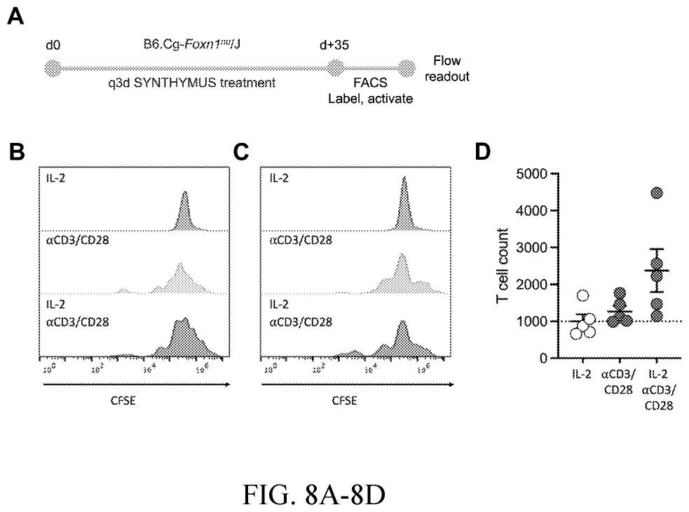

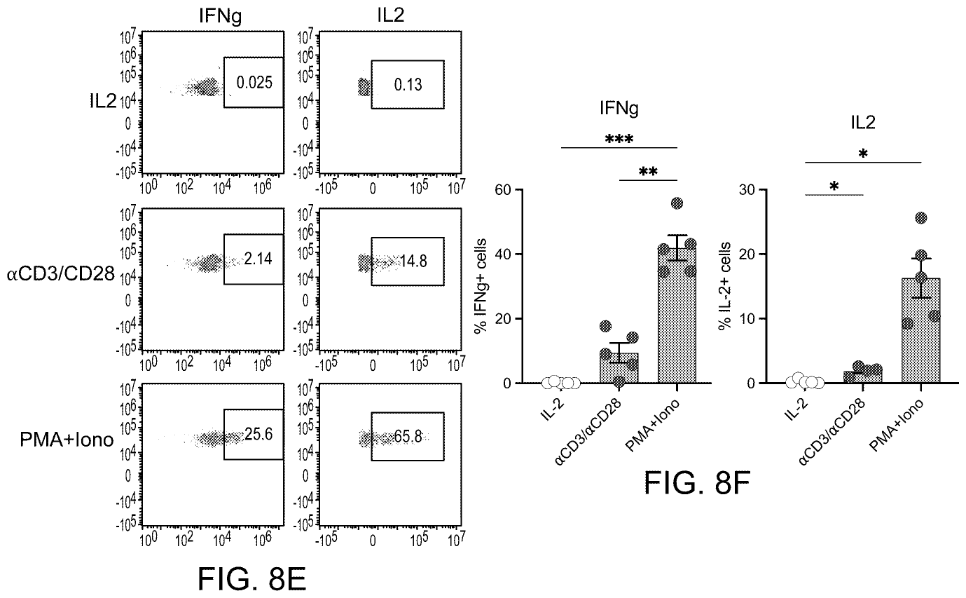

FIGS. 8A-8F further show artificial thymus-produced T cells can be activated ex vivo.

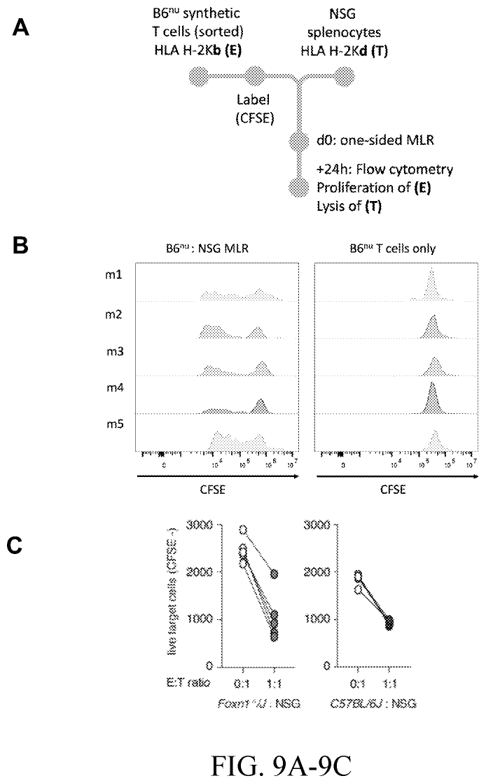

FIGS. 9A-9C show the artificial thymus-produced T cells can also lyse target cells.

### Serum Toxicity Analyses

Heart blood was aspirated by cardiac puncture using EDTA-precoated syringes and collected in BD Microtainer® Tubes with Potassium EDTA additive (BD, 365974). Serum was isolated by centrifugation at 2000 rpm for 10 mins at room temperature and snap-frozen on dry ice. All samples were received by IDEXX BioAnalytics and stored securely at −80° C. prior to analysis. Serum AST, ALT, CK, albumin, triglycerides and GGT were measured by an Olympus AU5400 (IDEXX BioAnalytics). Samples demonstrating hemolysis were excluded from the analysis.

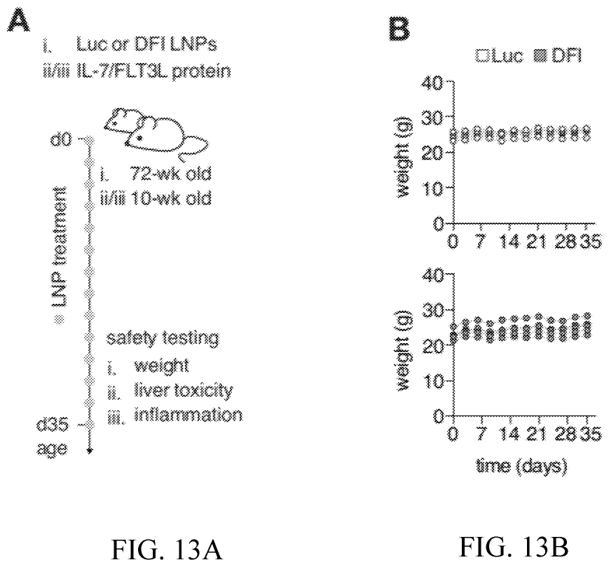

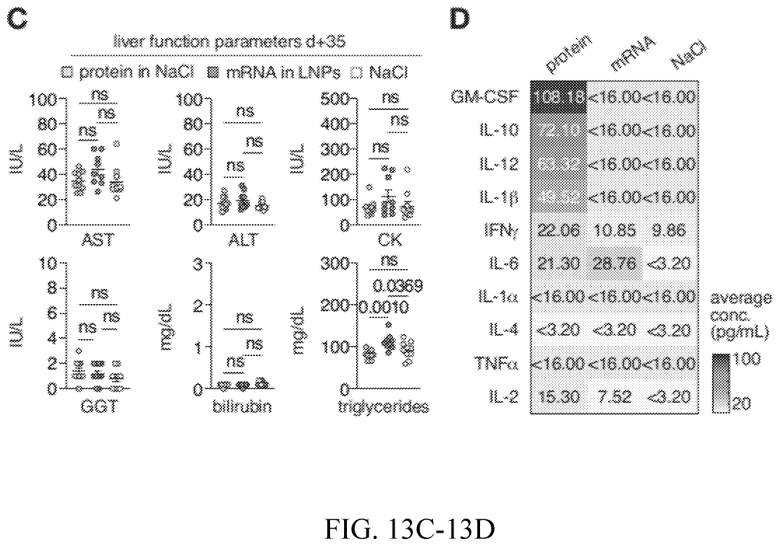

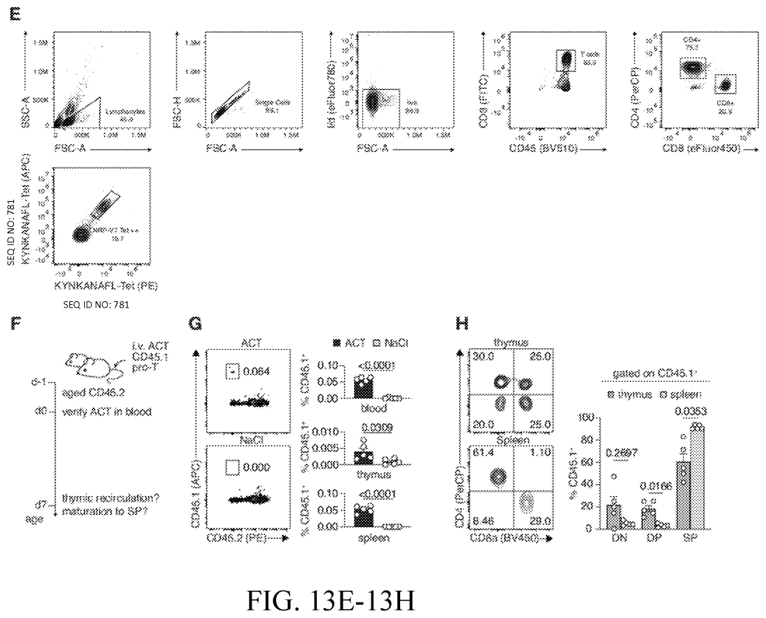

Treatment of athymic mice with DLL1 mRNA does not result in weight loss or liver toxicity (FIG. 13A-13B). Safety of the compositions described herein was confirmed using liver function and toxicity tests for aspartate transaminase (AST), alanine transaminase (ALT), gamma-glutamyl transferase (GGT), and albumin (FIG. 13C).

### Example 3: Reprogramming T Cell Aging with a Transient mRNA Intervention

Mammals have evolved adaptive immunity to safeguard against infections and tumor development. The thymus is central to this system, producing T cells that identify infected and cancerous cells. However, as the thymus ages, its functional decline contributes significantly to immune system defects in the elderly. To explore the feasibility of reversing this decline, Applicants delivered mRNAs encoding DLL-1, IL-7, and FLT3L, key factors for T cell development, which are depleted with thymic aging, to the liver to generate a transient ectopic site of T cell maturation. Applicants demonstrate that LNP-mediated delivery of these factors improves T cellular immunity in aged mice without generating autoimmunity. Mechanistically, Applicants find it induces ectopic development of T cells from hematopoietic precursors and activates dendritic cells, providing mechanistic evidence that in vivo engineering of adaptive immunity can effectively counteract immune aging.

### 1) Immune Aging and the Thymus

The most common causes of death amongst people aged 75 and older are influenza and pneumonia, followed by malignant diseases. This reflects the age-related decline in the immune system's self-renewal capacity, which decreases the ability to fight off pathogens and maintain cellular homeostasis. The thymus undergoes a sharp decline in size and function with age, a phenomenon known as thymic involution (Bodey, B., et al. (1997). Involution of the Mammalian Thymus, One of the Leading Regulators of Aging. In Vivo, 11(5), 421-440; Kumar B V, Connors T J, Farber D L. (2018). Human T Cell Development, Localization, and Function throughout Life. Immunity. February 20; 48(2):202-213). Starting in early adulthood, this leads to a decrease in the production of new T cells (Mittelbrunn, M., Kroemer, G. (2021). Hallmarks of T cell aging. Nat Immunol, 22, 687-698). Attempts to reverse thymic involution have achieved only limited success.

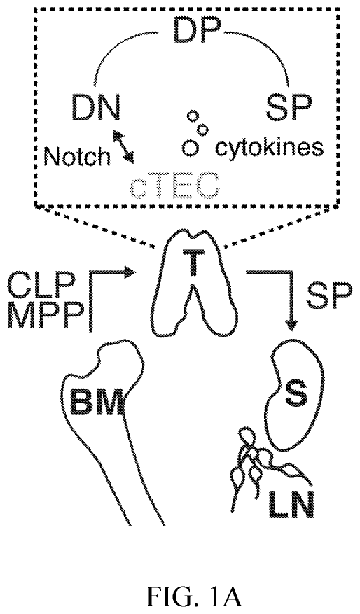

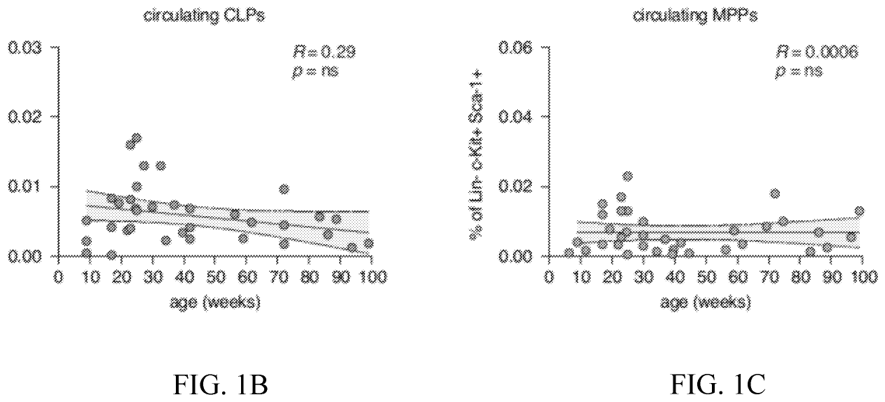

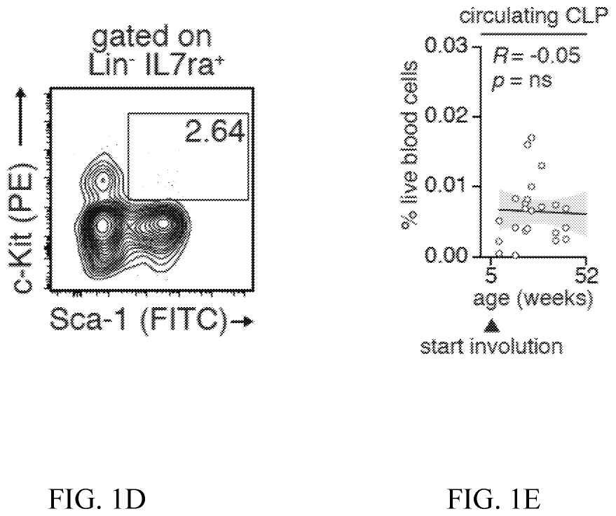

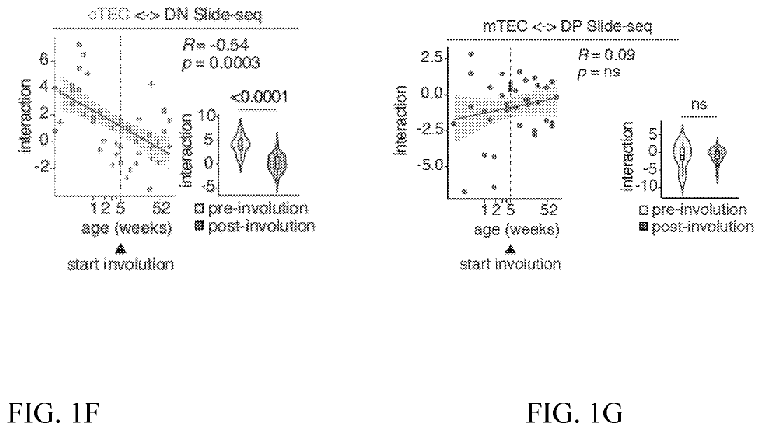

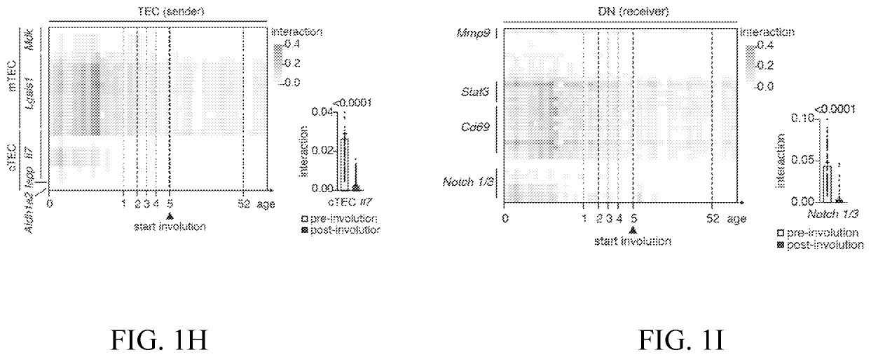

Intrathymic T cell development largely starts from pre-thymic common lymphoid progenitors (CLPs). CLPs have the capacity to differentiate into T cells when Notch signaling is triggered and are considered the major source of thymopoiesis (FIG. 1A). In mice, the frequency of circulating Lin IL7ra⁺ c-Kit⁺ Sca-1⁺ CLPs was not significantly impaired following the beginning of thymic involution at the age of 5 weeks (FIG. 1B-1E). This is in line with studies assessing the abundance of cells with T cell progenitor potential in the bone marrow by multiparametric flow cytometry as well as experimental findings from heterochronic thymus and bone marrow transplantations. Both suggest that an enhancement of T cell-mediated immunity can be achieved by the transplantation of a juvenile thymus into an aged recipient, while an analogous bone marrow transplant does not have this effect. Therefore, improving the functionality of T cells and enhancing the T cell receptor repertoire remain critical yet unmet needs in an ageing population.

Applicants posited that by studying interactions in the aging thymus, mechanisms underlying reduced T cell output could be identified and, potentially, factors that could be used to induce T cell development outside of the thymic microenvironment. Thymocytes and stromal cells across thymic involution in C57BL6/J mice were previously profiled by combining dissociated and spatial single-cell RNA and TCR sequencing (Liu et al., Spatial maps of T cell receptors and transcriptomes reveal distinct immune niches and interactions in the adaptive immune response. Immunity 55, 1940-1952.e5 (2022)). Because T cell production is mainly driven by cell-cell interactions with thymic epithelial cells, shifts in cell-cell interactions between developing thymocytes and thymic epithelial cells during aging were analyzed, using the Squidpy (Palla et al., Squidpy: a scalable framework for spatial omics analysis. Nat. Methods 19, 171-178 (2022)) integration of CellPhoneDB (Efremova et al., CellPhoneDB: inferring cell-cell communication from combined expression of multi-subunit ligand-receptor complexes. Nat. Protoc. 15, 1484-1506 (2020)) and Omnipath (Turei et al., OmniPath: guidelines and gateway for literature-curated signaling pathway resources. Nat. Methods 13, 966-967 (2016)) to incorporate the spatially acquired information. After adjusting for compositional changes, a global reduction in intercellular interactions between cortical thymic epithelial cells (cTECs) and double-negative (DN) T cells following the start of thymic involution was discovered, whereas there was no apparent change in interactions between mTECs and double-positive (DP) T cells with aging (FIGS. 1F-1G). Hence, beyond the reported apoptosis and trans-differentiation of aging cTECs and the depletion of the thymic stromal stem cell pool (Mittelbrunn et al., Hallmarks of T cell aging. Nat. Immunol. 22, 687-698 (2021)), cTEC function is additionally affected by thymic involution, together impairing the early, cTEC-dependent stages of T cell development. On the molecular level, receptor-ligand interaction analyses revealed multiple signaling pathways between thymic epithelial cells and thymocytes that decreased with age (FIG. 1H). Although Interleukin-7 (Il7)-mediated signaling, which has been described as critical for T cell progenitor survival, was found to be reduced in cTECs, DN thymocytes were predicted to additionally receive significantly less CD69 and Notch 1/3 downstream signaling post thymic involution, further underscoring a potential defect in early T cell development (FIG. 1I). Previous research highlighted the importance of Flt3 ligand (FLT3L) for the regeneration of the T cell compartment, particularly in older mice following bone marrow transplantation and confirmed an intrathymic requirement for this factor (Kenins et al., Intrathymic expression of Flt3 ligand enhances thymic recovery after irradiation. J. Exp. Med. 205, 523-531 (2008)). However, this interaction was likely not detected here because FLT3L is primarily produced by perivascular fibroblasts in the adult thymus (Chaudhry et al., Thymus: the next (re)generation. Immunol. Rev. 271, 56-71 (2016)). Based on these results in combination with previous research, three factors, Applicants selected IL-7, FLT3L, and Notch signaling, for further testing in aged mice to investigate if these factors could reverse the effect of ageing on the immune response.

2) Delivery of Dll1, Flt3, and Il7 mRNA to the Liver Improves Immune Response

Cell type-specific delivery of the identified factors to thymic epithelial cells or perivascular fibroblasts by systemic administration is challenging and would not account for the numerous impairments and disrupted tissue architecture caused by thymic involution. Applicants therefore considered a synthetic approach that involved delivery of these factors to an ectopic site, effectively bypassing the need for an intact thymus architecture. Under normal conditions, naïve T cells do not interact with epithelial cells in non-lymphoid tissues due to the endothelial barrier. The one exception to this, however, is the liver. Its unique structure, characterized by slow blood circulation, endothelial fenestrations, and the absence of a basement membrane, permits direct interaction between T cells and hepatocytes (Warren et al., T lymphocytes interact with hepatocytes through fenestrations in murine liver sinusoidal endothelial cells. Hepatology 44, 1182-1190 (2006)). The liver additionally hosts various cell types capable of presenting antigens to T cells (von Andrian et al., Homing and cellular traffic in lymph nodes. Nat. Rev. Immunol. 3, 867-878 (2003), Bertolino et al., Antigen-specific primary activation of CD8+ T cells within the liver. J. Immunol. 166, 5430-5438 (2001)) and maintains a high functional capacity, even in advanced age (Hunt et al., Hallmarks of Aging in the Liver. Comput. Struct. Biotechnol. J. 17, 1151-1161 (2019)). Its sustained adaptability during fetal development and certain adult diseases to support extramedullary hematopoiesis underscores the liver's functional and structural capacity to facilitate the development of blood cells when needed (Mende et al, Unique molecular and functional features of extramedullary hematopoietic stem and progenitor cell reservoirs in humans. Blood 139, 3387-3401 (2022)). Lastly, in vivo delivery of lipid nanoparticle (LNP)-encapsulated molecules to hepatocytes is efficient and robust (Kim et al., Engineered ionizable lipid nanoparticles for targeted delivery of RNA therapeutics into different types of cells in the liver. Sci Adv 7 (2021). For these reasons, the liver was chosen as a site of ectopic T cell maturation.

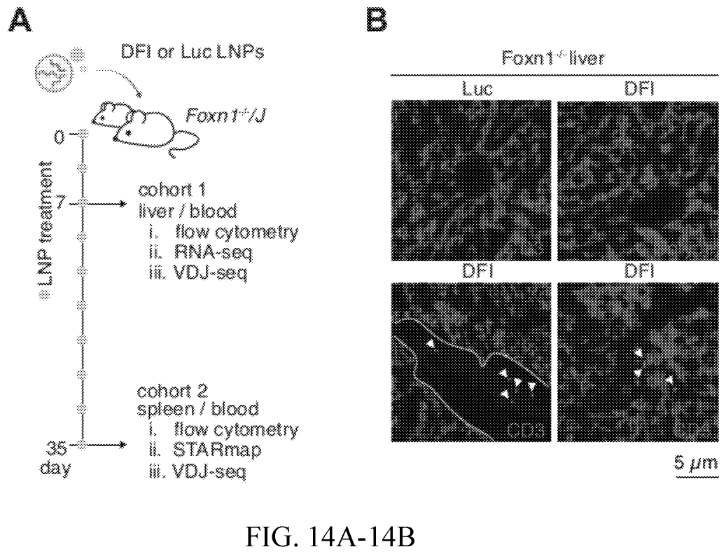

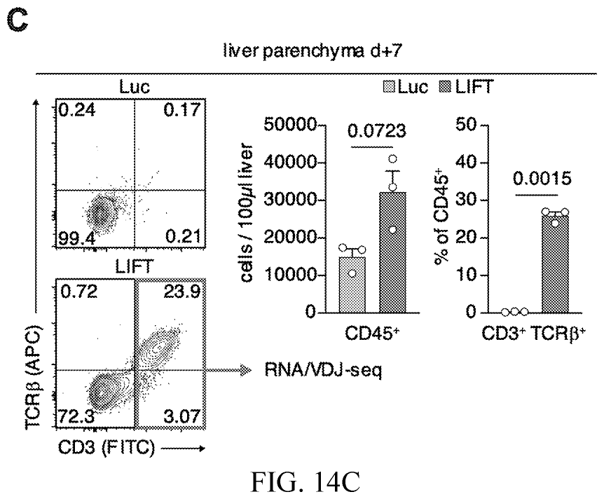

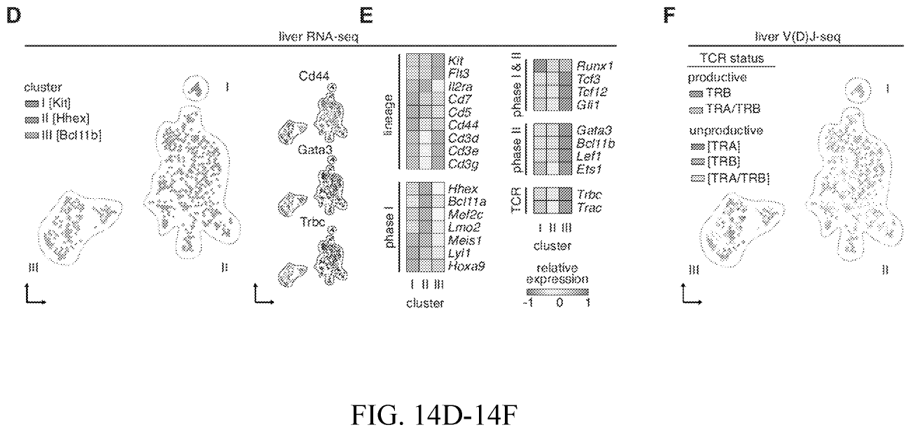

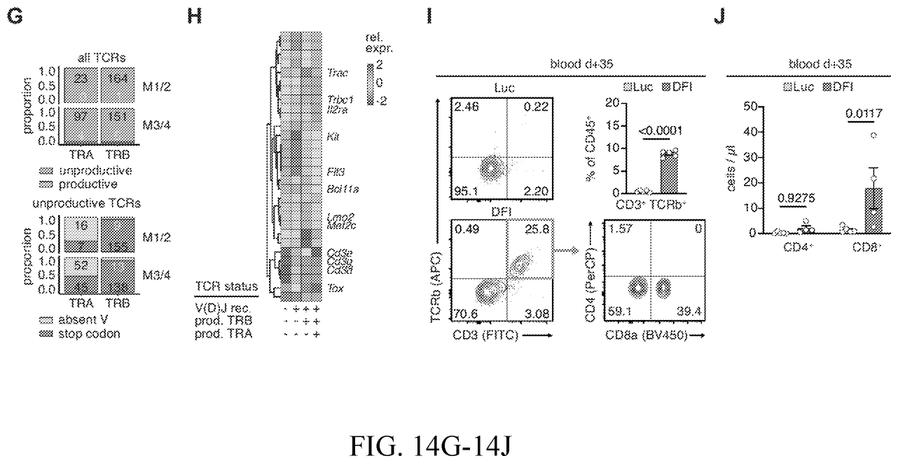

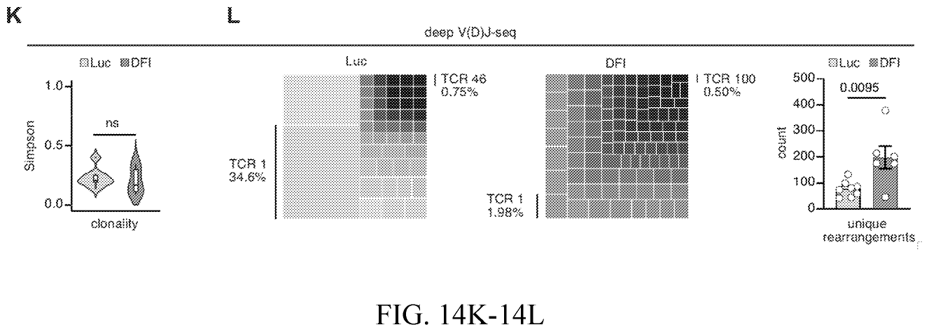

Applicants identified DLL1 (D), FLT-3L (F), and IL-7 (I) (hereinafter DFI) as key factors for T cell development, which are depleted with thymic aging. Applicants engineered a formulation of mRNA-encoded DFI within biodegradable LNPs (FIGS. 2 and 3A) and demonstrated production of biologically active DFI factors by primary murine hepatocytes (FIGS. 3B-3C). FIG. 3B shows fluorescence images (left) and quantification (right) of surface DLL1 protein expression in mouse hepatocytes. β-Catenin staining was used to delineate cell membranes. Scale bar, 100 μm. FIG. 3D shows extracellular concentrations of biologically active IL-7 (left) and FLT3L (right) secreted by mRNA-transfected hepatocytes as determined by IL-7 and FLT3L ELISA, respectively. Data are mean±s.e.m. P values were calculated using two-tailed unpaired t-tests. n=3 independent transfection experiments. Following intravenous administration of DFI LNPs (FIG. 14A), Applicants detected CD3+ cells resembling T cells in the livers of athymic mice (FIG. 14B-14C).

Applicants performed single-cell RNA/TCR sequencing of these liver-parenchymal CD3+ cells and found that Applicants captured a cross-section of T cell progenitors at various stages of T cell development, which Applicants delineated by analyzing canonical transcription factor expression patterns (FIG. 14D-14J). These cells also underwent V(D)J recombination in the absence of a thymus, which resulted in the generation of a primitive, yet diverse TCR repertoire (FIG. 14K-14L).

### 3) DFI Enhances Immune Responses and T Cell Function in Aged Mice

Applicants were interested if DFI treatment enhances the compromised T cell responses of aged animals. Consistent with prior findings, when delivered intravenously, the DFI LNPs primarily target the liver, and mRNA expression was specifically observed in liver cells (FIG. 3H).

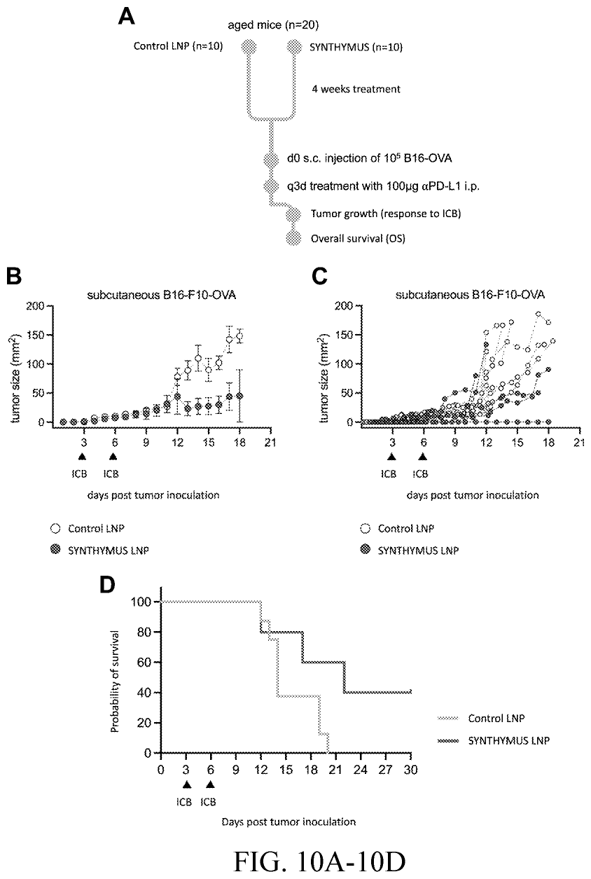

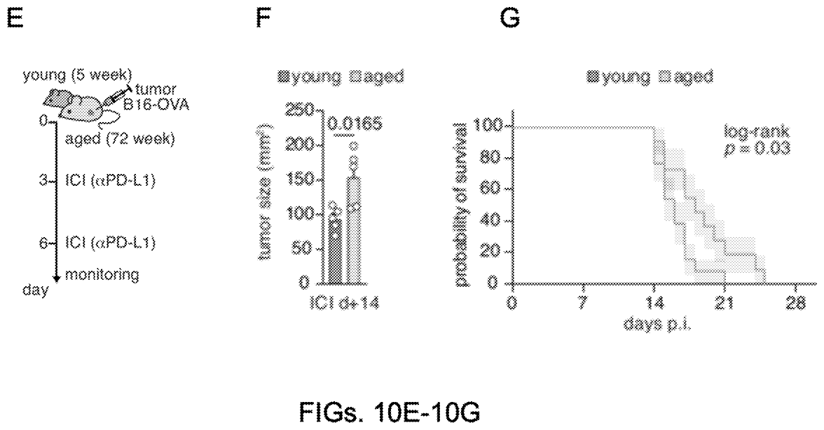

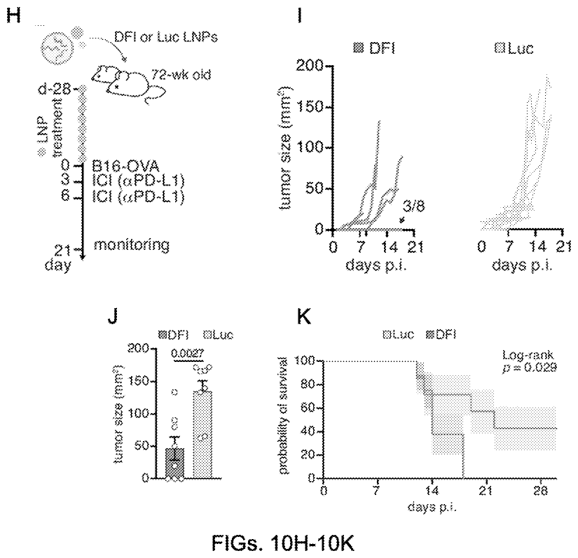

Treatment of aged immunocompetent mice (72 weeks old) with DFI prior to tumor challenge improved anti-tumor responses in a melanoma model treated with immune checkpoint inhibitors in aged mice resulting in increased survival (FIG. 10).

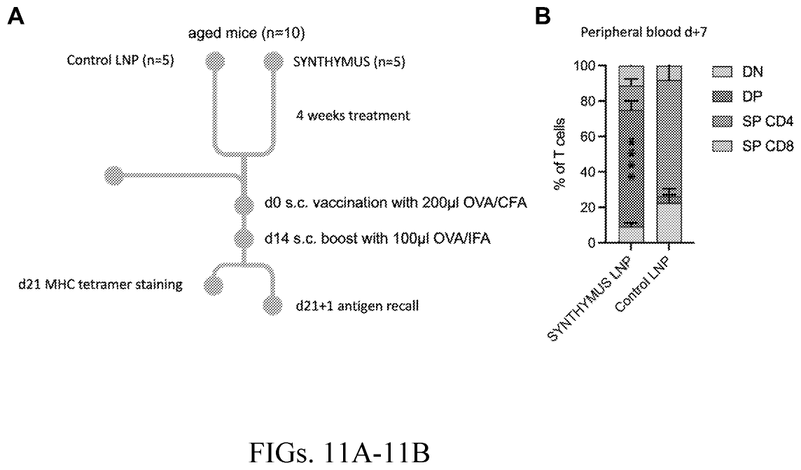

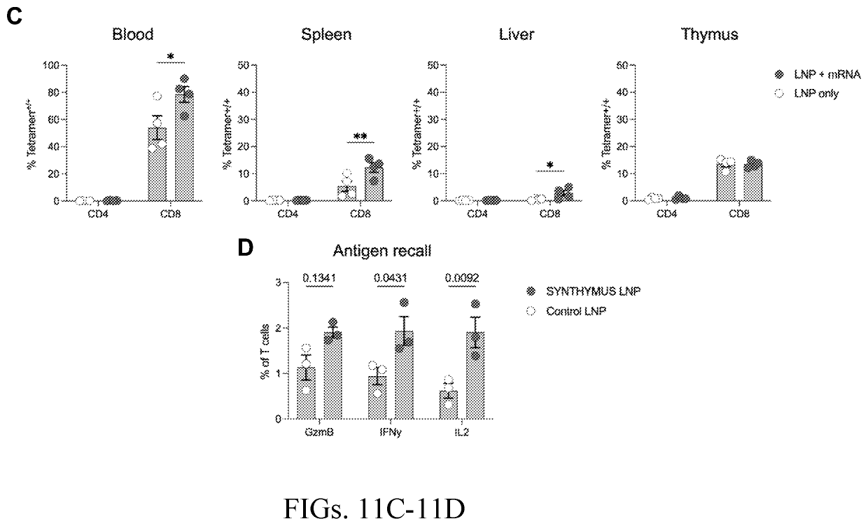

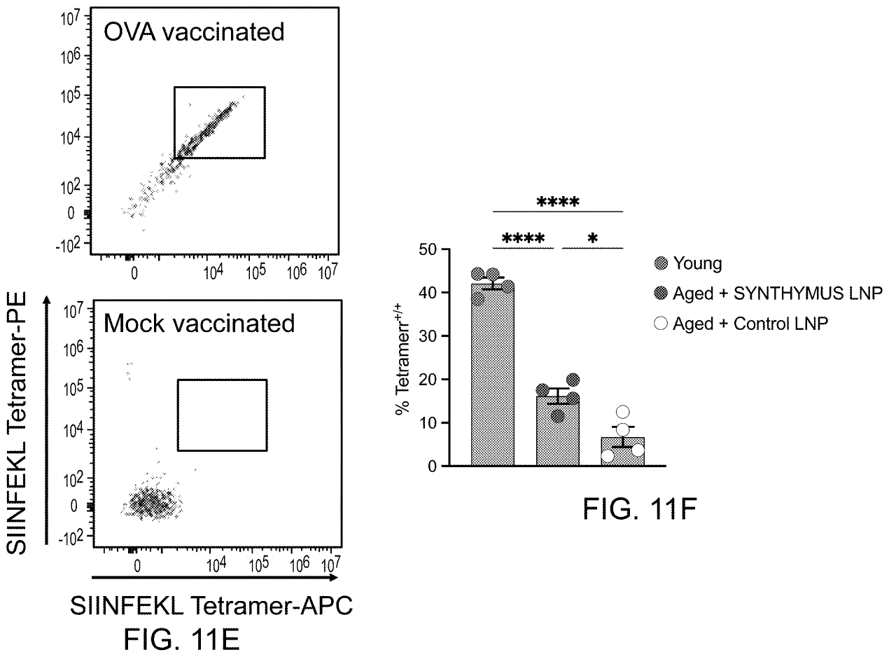

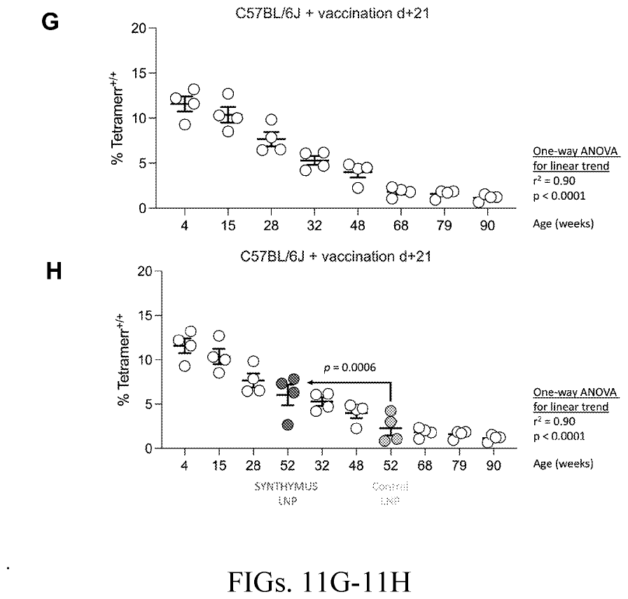

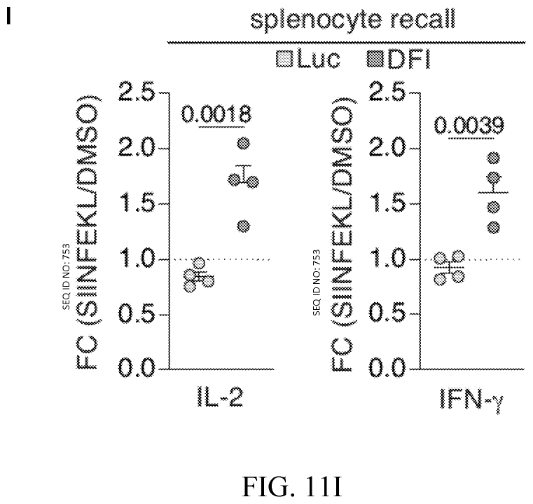

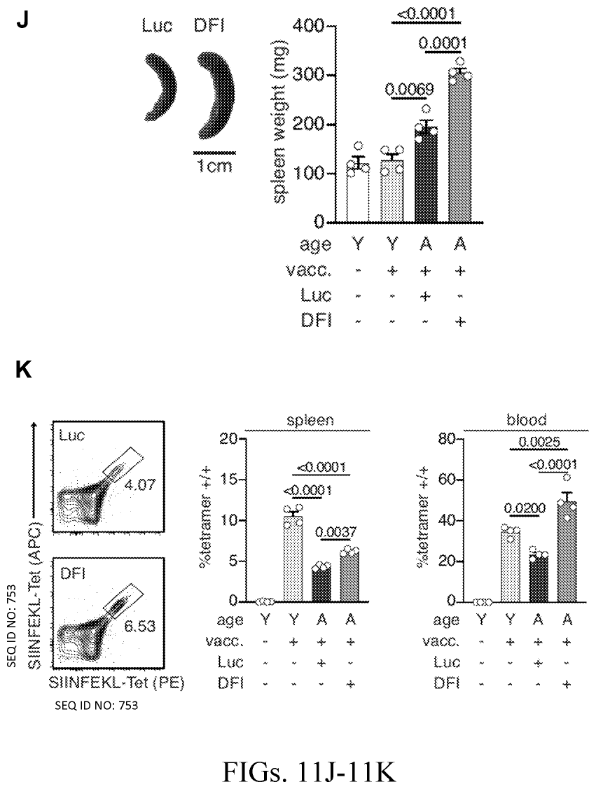

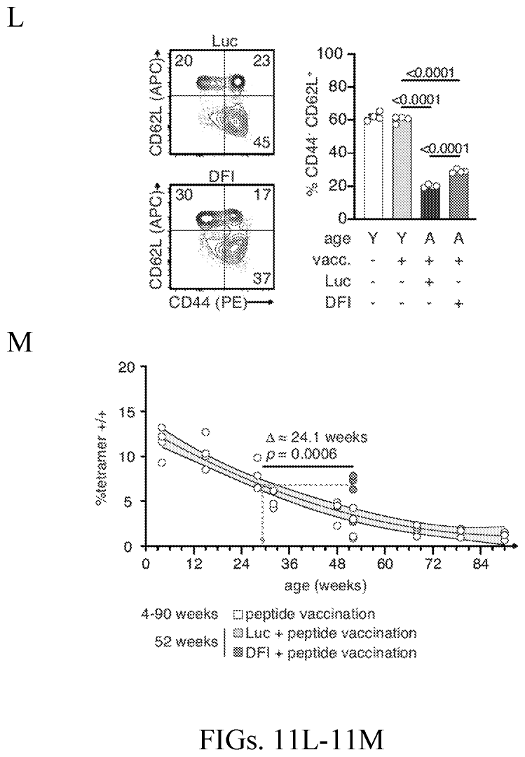

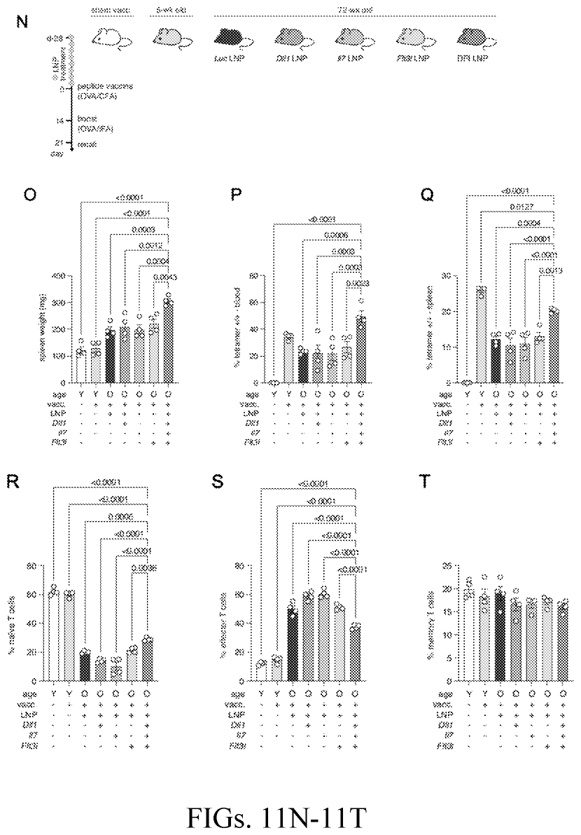

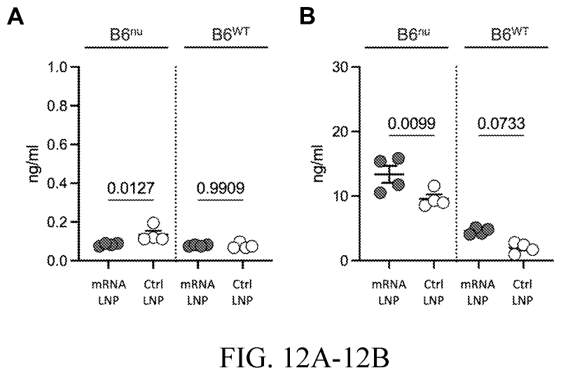

Adjuvant DFI treatment of aged animals challenged with peptide vaccines also resulted in improved T cell responses and an increase in naïve T cells (FIG. 11). Applicants were able to numerically and functionally ‘rejuvenate’ vaccine responses with DFI by 24.1 weeks of age (FIG. 11).

Leveraging the high functional capacity and unique anatomy of the liver, Applicants demonstrate clinically relevant improvements in T cell immunity in aged mice, including enhanced anti-tumor and vaccine responses. Mechanistically, Applicants found that the approach enables the ectopic development of T cell precursors in the liver, ultimately creating a primitive, but diverse T cell repertoire in the absence of a thymus. Underscoring its potential to be deployed safely, this treatment avoids the potentially severe side effects of cytokine treatments or cellular reprogramming and does not trigger autoimmunity in treated animals. The results offer promising evidence that targeted in vivo engineering of T cell immunity can effectively counteract immune aging.

### 4) Increasing Immune Checkpoint Blockade Response in a Murine Model of Age-Related Decline in Anti-Tumor Immunity

a) Subcutaneous Tumor Implantation and Treatment with Immune Checkpoint Inhibitors

OVA-expressing melanoma B16 cell line (B16-OVA) was kindly provided by Michael Kilian (Brigham and Women's Hospital, Boston, USA). Mice were inoculated subcutaneously with 1×10⁵ B16-OVA cells in Matrigel® Matrix (Corning). Tumor growth was monitored daily by measuring with digital calipers using the two largest perpendicular axes until the area (length×width) of the tumor exceeded 200 mm2. 100 μg of anti-PD-1 Ab (clone: 29F.1A12, BioXCell), anti-PD-L1 Ab (10F.9G2, BioXCell), and/or control hamster IgG Ab (BioXCell), and/or control Rat IgG Ab (BioXcell) were injected intraperitoneally 3 and 7 days after tumor inoculation as previously described

To evaluate the efficacy of the compositions described herein in increasing immune checkpoint blockade response in tumor-bearing aged mice, a murine model exhibiting age-related, reduced adaptive immunity was established (see FIGS. 10A to 10K).

Consistent with previous reports in various models, immune checkpoint inhibitor (ICI) with anti-PD-1/PD-L1 antibodies was less effective for tumor eradication in aged mice subcutaneously inoculated with 1×10⁵ ovalbumin (OVA)-expressing B16 melanoma cells (FIGS. 10E-10G). Tumor outgrowth in young mice was substantially attenuated by ICI treatment (FIG. 10F), whereas it did not result in any noticeable clinical activity in aged animals, significantly limiting their survival (FIG. 10G).

One group of aged mice (72-week-old animals) was treated with DFI-LNPs for 28 days (2 doses per week for 4 weeks) (n=10) and a separate control group was treated with empty LNPs (n=10). Following treatment, each group was inoculated with 1×10⁵ B16-OVA melanoma cell line on day 0 and subsequently treated with anti-PD-L1 antibody on day 3 (FIG. 10A-10D) or day 3 and day 6 (FIG. 10H-I0K). Tumor growth in response to immune checkpoint blockade (ICB) was measured daily and overall survival (OS) probability were evaluated (see FIGS. 10B-10D (first experiment) and FIGS. 10I-10K (second experiment)). Tumor rejection was observed in 3 out of 8 animals and long-term s./,./,urvival in 40% of mice treated with DFI-LNPs, a significant improvement over LNP-treated animals (FIGS. 10D and 10K). Taken together, these data demonstrate that the compositions described herein are capable of increasing anti-tumor immune response in aged mice exhibiting diminished adaptive immunity.

### 5) Increasing Vaccination Response in a Murine Model of Age-Related Decline in Adaptive Immunity

To evaluate the efficacy of the compositions described herein in increasing immunization response in aged mice, a murine model exhibiting age-related, reduced adaptive immunity was established.

### a) Subcutaneous Immunization

Young (5 weeks) and aged (72 weeks) mice were immunized with protein emulsified in complete Freund's adjuvant (CFA, BD 210485), followed by one or two booster doses of protein emulsified in incomplete Freund's adjuvant (IFA, BD 210486). Mice were injected with antigen emulsified in CFA subcutaneously at two sites on the chest, injecting 0.05 mL at each site (total of 0.1 per mouse). The needle was kept inserted in the subcutaneous space for 10 to 15 seconds after each injection to avoid leakage of the emulsion. A booster injection of antigen emulsified in IFA is administered 14 days after immunization with antigen/CFA emulsion. The booster is given as a single subcutaneous injection with 0.1 mL of IFA emulsion, at one site on the sternum. Serum, peripheral blood or spleen samples were obtained 21 days after the initial immunization.

### b) Quantification of Vaccination Responses

For all vaccination experiments, at the indicated time points post-vaccination, approximately 100 μL of blood was collected with EDTA-coated capillary tubes from each mouse and then transferred to an EDTA-coated tube. The collected blood samples were centrifuged at 2000 g for 10 min, followed by transferring the resulting plasma into another tube. Anti-OVA mouse IgG antibodies were measured via ELISA using a Mouse Anti-OVA IgG Antibody Assay Kit (Chondrex Inc, 3011), according to the manufacturer's protocol. All samples were processed in technical duplicates, with averaged values of technical duplicates being reported for each biological replicate. All samples were background subtracted using the average of the background of the 0 pg/mL standard (buffer only condition), and the standard curve was fit to a logistic regression that was used for the back-calculation of antibody titers.

At the indicated time point mentioned above, mouse spleen single-cell suspensions were prepared in RPMI 1640 medium by mashing tissue against the surface of a 70-μm cell strainer (BD Falcon, 64752-00). Then, the single-cell suspension was centrifuge at 200 g for 5 minutes and the supernatant was removed. Red blood cells were lysed by adding 3 ml of ACK lysis buffer (ThermoFisher) at 4° C. for 1.5 minutes, followed by centrifugation and removal of the supernatant. The cells were washed once with RPMI 1640 medium and then resuspended with RPMI 1640 medium (10% FBS and 1% Pen-Strep antibiotic). 4×10⁶ of splenocytes from each mouse were cultured in RPMI medium and stimulated with SIINFEKL (SEQ ID NO: 753) peptide (synthesized at 99% purity by GenScript) at a final concentration of 1 μg/ml for each peptide. The GolgiStop transport inhibitor cocktail (BD, 554724) was added according to the manufacturer's instruction 2 hours later. Then, 6 hours later, the cells were collected and washed with FACS buffer (PBS with 2% FBS) prior to Fc block with TruStain FCX (anti-mouse CD16/32, clone 93; BioLegend) (1:50 final dilution) and surface antibody staining for 20 min at 4° C. Cells were washed with a FACS buffer and then fixed and permeabilized using a BD Cytoperm fixation/permeabilization solution kit (BD, 554714) according to the manufacturer's instructions. Cells were washed in perm/wash solution, followed by intracellular staining (45 min, 4° C.) using a cocktail of the respective cytokine antibodies. Finally, the cells were washed in perm/wash solution and suspended in a staining buffer. Samples were washed, resuspended in 200 μl FACS buffer and acquired on a Beckman CytoFLEX LX Flow Cytometer. Analysis was performed using FlowJo v10 software.

One group of aged immunocompetent mice was treated with SYNTHYMUS (n=5) and a separate group of aged immunocompetent mice was treated with empty LNP (n=5). Following treatment, each group was immunized with ovalbumin with complete Freund's adjuvant (OVA/CFA) on day 0 and subsequently provided a booster immunization with ovalbumin with incomplete Freund's adjuvant (OVA/IFA) on day 14. Following immunization, mice were treated with ovalbumin-derived SIINFEKL (SEQ ID NO: 1) and ISQAVHAAHAEINEAGR (ISQ) (SEQ ID NO: 754) tetramer peptides and T cell adaptive response was evaluated (FIG. 11A). Analysis of peripheral blood drawn at day 7 revealed that SYNTHYMUS led to the production of a greater percentage of double positive (DP) T cells (FIG. 11B). The percentage of SIINFEKL (SEQ ID NO: 1) tetramer-specific CD8+ T cells harvested from peripheral blood, spleen, liver, and thymus was measured (FIG. 11C).

A comparison of SIINFEKL (SEQ ID NO: 753) tetramer-specific T cells in young mice (Young, 6 weeks), aged mice (Aged, 72 weeks) treated with DLL1 mRNA in combination with IL-7 mRNA and FLT3L mRNA (Aged+SYNTHYMUS), and aged mice treated with empty lipid nanoparticle (Aged+LNP) is shown in FIGS. 11E-11F. A statistically significantly greater percentage of total T cells and CD8+ T cells specific for SIINFEKL (SEQ ID NO: 753) was observed for the aged group treated with SYNTHYMUS relative to the aged mice group treated with the empty LNP (FIG. 11F). Epitope-specific T cell proliferation and cytokine secretion (granzyme B (GzmB), IFN-γ, and IL-2) were also significantly higher for the aged group treated with SYNTHYMUS (FIG. 11D). Aged mice treated with DLL1 mRNA in combination with IL-7 mRNA and FLT3L mRNA exhibited increased production of SIINFEKL (SEQ ID NO: 753) tetramer-specific CD8+ T cells in the blood, spleen, and liver relative to the control group that did not receive DLL1 mRNA (FIG. 11C). A greater relative proportion of tetramer-specific CD8+ T cells was found in the liver compared to the thymus, with no increase in production of such T cells observed in the latter (FIG. 11C).

As a benchmark for the improved vaccine response observed when aged animals were pre-treated with DFI-LNPs, a time course experiment of vaccine response decline was performed. Challenging wild-type mice of various ages (4 to 90 weeks) with a peptide vaccine showed a clear negative correlation between age and the proportion of vaccine-responsive T cells in the spleen (FIGS. 11G and 11M). Treatment of C57BL/6J mice with SYNTHYMUS was also shown to augment SIINFEKL (SEQ ID NO: 753) tetramer-specific immune response by 20 weeks relative to mice treated with empty LNP (FIG. 11H).

The effect of treatment with DFI-LNPs prior to peptide vaccination, observing significantly enhanced T cell-mediated vaccine responses in older animals compared to Luc-LNP treatment (FIG. 11A-11M). This enhancement was evidenced by increased spleen cellularity and weight, established indicators of immune activation or reconstitution (FIG. 11J). Treatment with DFI-LNPs also increased the presence of vaccine-induced, SIINFEKL-specific T cells in both the spleen and bloodstream (FIG. 11K). Repeated antigen exposures throughout life result in an age-dependent naïve-to-memory shift in the T cell repertoire. In line with this, substantially more CD44⁺ CD62L⁺ naïve T cells were present in the spleen of young mice compared to aged mice. DFI treatment, however, increased the frequency of naïve T cells in the spleen of aged animals (FIG. 11L). To investigate whether these effects were attributable to a single component, these experiments were repeated with either individual mRNAs or the recombinant protein equivalents of IL-7 and FLT-3L (FIGS. 11N-11T). Similar to previous studies on recombinant IL-7 as a vaccine adjuvant, only IL-7 treatment (2 doses of 100 μg per week) significantly increased the abundance of vaccine-responsive T cells in the spleen. However, IL-7 treatment did not result in an increase in naïve T cells or circulating SIINFEKL-specific clones in the peripheral blood, as observed with the triple DFI mRNA combination. This is in agreement with findings from clinical trials, where IL-7 primarily induced proliferation of mature T cell subsets. Other single factors of DFI delivered as mRNA did not recapitulate any of the observed effects (FIGS. 11N-11T). As a benchmark for the improved vaccine response, aged animals were pre-treated with DFI-LNPs. A time course experiment of vaccine response decline was observed. Challenging wild-type mice of various ages (4 to 90 weeks) with a peptide vaccine showed a clear negative correlation between age and the proportion of vaccine-responsive T cells in the spleen (FIG. 11M). One-year-old animals treated with DFI-LNPs exhibited significantly enhanced T cell responses compared to same age wild-type animals, with the response aligning to what Applicants observed in much younger animals (A 24.1 weeks to their biological age, p=0.0006; FIG. 11M). Analysis of splenocytes from these animals revealed that T cells from DFI-LNP-treated groups produced higher levels of IL-2 and IFN-γ upon antigen re-exposure than those from control groups (FIG. 11I). These results indicate that ectopic expression of Dll1, Flt3, and Il-7 in the liver is a potentially viable strategy to enhance the function of the T cell repertoire in older mice, yielding significant improvements in anti-tumor and vaccine-induced immunity. This suggests application of this approach may transiently boost immune responses in immunocompromised individuals.

### Example 4: DFI Treatment Induces Thymus-Independent T Cell Development

Applicants hypothesized that the immunostimulatory effects and increased presence of naïve CD62L⁺ T cells observed upon treatment with DFI may result in part from the lymphopoiesis of novel T cells. To explore this hypothesis, Applicants employed homozygous B6.Cg-Foxn1−/−/J (Foxn1^{-/-}) mice, which lack the thymic anlage, but unlike other models of T cell deficiency (e.g., NSG or Rag2^{-/-} models) do not have any further defects affecting T cell maturation. In fact, some functional mature T cells can be detected, particularly in adult mice, due to abnormal TCR recombination and peripheral expansion. Because of this, prior to each experiment, the absence of all T cells was confirmed. DFI was administered as in the prior experiments and then the liver parenchyma was analyzed at an early treatment stage (day +7, after 2 doses) and the spleens and heart blood after a complete 5-week treatment course (day +35, 10 doses) using flow cytometry and complementary single-cell profiling methods (FIG. 14A). As early as 7 days after beginning treatment, CD3⁺ signals within the liver parenchyma and hepatic vessels were observed (FIG. 14B). Mononuclear immune cell fractions from the perfused liver tissues were then isolated through density centrifugation and analyzed via flow cytometry. In the DFI-treated mice, there was an increase in the absolute number of CD45⁺ cells, driven by the emergence of a new liver intraparenchymal CD3⁺ TCRβ⁺ cell population that lacked CD4 and CD8 expression (FIG. 14C).

### 1) Single-Cell RNA/TCR-Seq of Liver Parenchymal T Cell Progenitors

Freshly isolated immune cells post density centrifugation of liver cell suspensions and red blood cell lysis were blocked with rat anti-mouse CD16/32 (0.5 μg per well, eBioscience). Subsequently, respective antibodies in PBS were added in a total volume of 100 μL and stained for 30 minutes. The antibodies used included anti-mouse CD45-BV510 (clone 30.F11, BioLegend), anti-mouse CD3-FITC (clone 17A2, BioLegend), anti-mouse TCRB-APC (clone H57-597, BD), TruStain FCX (anti-mouse CD16/32, clone 93; BioLegend). eFluor 780 fixable viability dye (eBioscience) was employed per the manufacturer's protocol to exclude dead cells. Cells were stained with respective antibody pools for 20 min at 4° C. Subsequently, cells were washed twice with PBS+2% FBS+5 mM EDTA and sorted on a Sony MA900 sorter using a 100 m nozzle into chilled 1.5 ml microtubes that were pre-coated overnight with 0.4% BSA and kept on ice until processing.

Following the preparation of single-cell suspensions and sorting as described above, 10,000 cells per biological replicate (replicate 1: mice 1+3, replicate 2: mice 2+4) were loaded onto a 10× Chromium controller (Chip G) per the manufacturer's protocol using the Chromium Single Cell V(D)J Reagent Kits (v1.1 Chemistry). The resulting emulsion was recovered and immediately subjected to reverse transcription. Subsequent steps included cDNA isolation and library preparation, adhering to the manufacturer's protocol. Each of the final transcriptome and V(D)J libraries underwent paired-end sequencing (26 and 92 bp) on a single Illumina NovaSeq 6000 S2 lane. Raw sequencing data underwent processing and alignment to the mouse genome (GRCm39-mm39) using the CellRanger pipeline (10x Genomics, version 7.1.0).

Seurat datasets were generated for blood and thymus at each time point. Singlets were identified per the published Seurat vignette (satijalab.org/seurat/articles/hashing vignette.html) and used for downstream analyses. Additionally, only cells with more than 500 and less than 4000 unique features were detected, and less than 5% of mitochondrial counts were considered for further analysis. Using scRepertoire AB-VDJ, information was added per sample using default settings, employing the CDR3 amino acid (“aa”) sequence for clonotype calling.

### 2) Single-Cell RNA/V(D)J Sequencing of the Liver Parenchyma

Single-cell RNA/V(D)J sequencing of the liver parenchyma-infiltrating cells showed three transcriptionally distinct cell clusters, expressing varied sets of lineage marker genes and transcription factors (TFs) indicative of different stages in T cell development (FIG. 14D-14E). Traditionally, pro-T cell stages are divided into three phases based on known precursor-product relationships, extracellular signal requirements, and commitment status. Phase I includes early T cell progenitors (ETP, also known as DN1) and DN2a stages, characterized by the proliferation of uncommitted T cell precursors with a dependency on Notch signaling. Phase II encompasses the T cell lineage-committed DN2b and DN3a stages, where cells exhibit a higher dependency on Notch but proliferate less and undergo TCR gene rearrangement. Phase III spans from DN3b to DP stages, during which T cell lineage-committed cells proliferate in response to pre-TCR or γδ TCR signals and eventually lose Notch dependency. Applicants found cells that express lineage markers and TFs characteristic of each of these developmental phases (FIG. 14E).

The very few cells captured within cluster I highly expressed Kit, resembling DN1 cells, the earliest T cell precursors in the thymus. In cluster II, Applicants observed expression of a number of T cell specific transcription factors (TFs), in line with the natural transition of T cells to phase II. This transition is largely driven by the Notch pathway, and the thymus plays a vital role in providing Notch ligands. Providing DLL-1 mimics these thymic signals in the liver niche, steering the cluster I cells towards the T cell lineage. In phase I of T cell development, no specific set of TFs is recognized to have ‘master-like’ activity. Yet, through their collective actions, overlapping with Notch signaling, a well-defined ensemble of factors establishes T cell identity. Applicants found cluster II cells exhibited a distinct set of these phase I TFs, including Lmo2 (also referred to as Rbtn2), Mef2c, Bcl-11a, Hhex, Lyl1, and, in alignment with the characteristics of early pro-T cells, Hoxa9 and Meis1. Expression of these last two TFs decreases in cells of cluster III, which aligns with the physiological progression of pro-T cells into phase II (FIG. 14E).

During T cell development, Notch signaling is also instrumental in activating a set of genes, referred to as phase II genes. Among these, the coordinated expression of Gata3 and Tcf7 is critical for upregulating genes that define T cell identity and ultimately activating Bcl11b. BCL11B plays a vital role in ensuring that T cell progenitors, moving from phase I to phase II, do not deviate into alternative lineages. This is achieved in part by repressing the expression of Id2 and Zbtb16, which serve essential roles in innate lymphoid cells and natural killer cells. Applicants found that cells in cluster III express these three key TFs but not Id2 or Zbtb16. This distinct expression pattern, which was not observed in clusters I and II, is indicative of lineage commitment as cells transition from phase I to phase II (FIG. 14E).

Applicants also observed that cells in cluster III began co-expressing Trb and Tra genes (FIG. 14E), suggesting these cells might undergo V(D)J recombination, which is essential for generating the TCR repertoire and P-selection. To detect signs of active V(D)J recombination in the CD3+ liver intraparenchymal cells, Applicants incorporated all detected V(D)J sequences into their analysis. Because Applicants anticipated capturing incompletely recombined TCRs during these early developmental stages, the V(D)J repertoire profiles were aligned with the GRCm38 V(D)J reference and subsequently realigned the data with igblast to identify unproductive or partially recombined sequences prior to their inclusion in the single-cell analysis (FIG. 14F, Table 4, Ye et al., IgBLAST: an immunoglobulin variable domain sequence analysis tool. Nucleic Acids Res. 41, W34-40 (2013)).

TABLE 4
Successfully recovered V(D)J rearrangements from single-cell RNA/V(D)J-seq of
liver-parenchymal T cell progenitors in DFI-treated Foxn1-/-mice (clone sequences
selected according to Multi] and productivity status)
TABLE 4

| | SEQ ID | | | | |
|---|---|---|---|---|---|
| CLONE FULL SEQUENCE | NO | productive | locus | MultiJ | class |
| TGGGGACGGTGATTCAATTCTATGGGAAGCCTTTACAAAAACCATTCTGTCTGTCC | 759 | FALSE | TRA; | TRUE; NA | absent V; |
| CAAGGCCCACAGTCCTCTACTCTTCTCCTGGGTCGCAAGCCTAGATGTGTTTAGAT | | | TRB | | premature |
| CCAGAATGCTTTCACGTCACCTCTGCAGCCTGCTAGGCCAAGATTGTGGAGAAGAA | | | | | stop |
| TGGGGCCTTACCAGAGCAGGAGCCTCCTACACTGAATGAACATAAGAAGAATGGCC | | | | | codon |
| TAGTGGCCCTAGCAGCAAAGGATGCAGGGAAATCCTCTTCTGCCAACGTCCATAAA | | | | | |
| ACAGACCACCATCAGTGGATAGGTGAGCCAGAGGTGAACAGTTCTACAGGTTTGTT | | | | | |
| TTGTTTTAAAAGATCTCAGCTCTTGATGAATATCATCATAGGACCAGGAAAACTGT | | | | | |
| TCTCTGACAGGCTACCTCACTTTGATGGACCTTGAGAGAGGAACAATATATATGGG | | | | | |
| GGATTCTTCACAAAAGGGATGTAAGTTCCACTGGAGGGAATCTACCATGTTTGACA | | | | | |
| TTGCCACAAGTCTATCGTCAAAATGTTCTTTTGTGCAATACCTGTGATGCACACAA | | | | | |
| AGCGATTACTCCTCCTATGGTCCTCTAGATACTAGCTGGGAAGAGCCTCTCTTCAC | | | | | |
| CCCTTAAGATTATTTTTCTCCTCATCCTATGGCACTGTGCAAACACAGAAGTCTTC | | | | | |
| TTTGGTAAAGGAACCAGACTCACAGTTGTAGGTAAGATATCTTTCAGGTAAATTTC | | | | | |
| CAGGTCTCTCTTCGGACAAAGCATGTCCTCCGTGTCCATATTCGAGTATCTGTATT | | | | | |
| CTGATGTGCAAACTCCGACTACACCTTCGGCTCAGGGACCAGGCTTTTGGTAATAG | | | | | |
| AGGATCTGAGAAATGTGACTCCACCCAAGGTCTCCTTGTTTGAGCCATCAAAAGCA | | | | | |
| GAGATTGCAAACAAACAAAAGGCTACCCTCGTGTGCTTGGCC_GAGGCATTCAGGC | | | | | |
| AGCGAGAGCAGAGCAGCGTAGAGCAGCACAGCTGAGCTCGTGAGGCAGGAGACTCA | | | | | |
| GCCCGAGGAAATCGCAGATAAGTTTTTAATTAAAAAGATTGAGCAGTAAAAAGAAT | | | | | |
| TAGAACTCTAAACTTAAGCTAATAGAGTAGCTTATCGAAATATTACTTAGTCTTAA | | | | | |
| TAATCTAAGAAGATCTTAAGAGATAACATGAAGGCTTATTTAAACAGTTTGAAAAA | | | | | |
| GGAAATGAGGAGAAAAGTATTTGTACTGTATAATGGAGGCTGACCAGAGCAGTTTA | | | | | |
| GGAGATTGTAAAGGGAGGTTTTGTGAAGTTCTAAAAGGTTCTAGTTTGAAGGTCGG | | | | | |
| CCTTGTAGATTAAAACGAAGGTTACCTAAATAGAATCTAAGTGGCATTTAAAACAG | | | | | |
| TAAAGTTGTAGAGAATAGTTTGAAAATGAGGTGTAGTTTTAAAAGATTGAGAAAAG | | | | | |
| TAGGTTAAGTTGACGGCCGTTATAAAAATCCTTCGACTGGCGCATGTACGTTTGAA | | | | | |
| GGCATGAGTTGGAAACAGGGAAGATGGAAGTGTTAGGC | | | | | |
| TGGGGGCTCTCTCACCAAAGAGACCAGTATCCTGAGAGGAAGCATGTCTAACACTG | 760 | TRUE; FALSE | TRB | FALSE; NA | NA; |
| CCTTCCCTGACCCCGCCTGGAACACCACCCTGCTATCTTGGGTTGCTCTCTTTCTC | | | | | absent V |
| CTGGGAACAAGTTCAGCAAATTCTGGGGTTGTCCAGTCTCCAAGATACATAATCAA | | | | | |
| AGGAAAGGGAGAAAGGTCCATTCTAAAATGTATTCCCATCTCTGGACATCTCTCTG | | | | | |
| TGGCCTGGTATCAACAGACTCAGGGGCAGGAACTAAAGTTCTTCATTCAGCATTAT | | | | | |
| GATAAAATGGAGAGAGATAAAGGAAACCTGCCCAGCAGATTCTCAGTCCAACAGTT | | | | | |
| TGATGACTATCACTCTGAGATGAACATGAGTGCCTTGGAGCTAGAGGACTCTGCCG | | | | | |
| TGTACTTCTGTGCCAGCTCTTCCGGGACAGGGAGATATGAACAGTACTTCGGTCCC | | | | | |
| GGCACCAGGCTCACGGTTTTAGAGGATCTGAGAAATGTGACTCCACCCAAGGTCTC | | | | | |
| CTTGTTTGAGCCATCAAAAGCAGAGATTGCAAACAAACAAAAGGCTACCCTCGTGT | | | | | |
| GCTTGGCC_GGCCAAGCACACGAGGGTAGCCTTTTGTTTGTTTGCAATCTCTGCTT | | | | | |
| TTGATGGCTCAAACAAGGAGACCTTGGGTGGAGTCACATTTCTCAGATCCTCTAGG | | | | | |
| ACGGTGAGTCGTGTCCCTGGTCCGAAGAACTGCTCAGCATAGTTACACAGCAGAAA | | | | | |
| AGGGCTACCAAGAATTCTTCCTCTTTCCTCACCTTCCTCAATACAGACCCGGGGTT | | | | | |
| ACTCACATTCTCCATCTACCTCCAGAACTGGTAAGGCAATCTTGACCTTGCATGAG | | | | | |
| GCCGATCTCAAATTGAGGAGGAGATACCACTTTTGGCTTGGGAACGGATATCCATC | | | | | |
| TATTCACTCAGGGTTTGACAAAGTGAATTTGAAATCTGAAGGCTTCCTGTTGGTGT | | | | | |
| CCAGGTAGCCTCCAATGAGAAAGGACTTGTCCCC | | | | | |
| ACAGTCCTCTACTCTTCTCCTGGGTCGCAAGCCTAGATGTGTTTAGATCCAGAATG | 761 | FALSE | TRA | TRUE | absent V |
| CTTTCACGTCACCTCTGCAGCCTGCTAGGCCAAGATTGTGGAGAAGAATGGGGCCT | | | | | |
| TACCAGAGCAGGAGCCTCCTACACTGAATGAACATAAGAAGAATGGCCTAGTGGCC | | | | | |
| CTAGCAGCAAAGGATGCAGGGAAATCCTCTTCTGCCAACGTCCATAAAACAGACCA | | | | | |
| CCATCAGTGGATAGGTGAGCCAGAGGTGAACAGTTCTACAGGTTTGTTTTGTTTTA | | | | | |
| AAAGATCTCAGCTCTTGATGAATATCATCATAGGACCAGGAAAACTGTTCTCTGAC | | | | | |
| AGGCTACCTCACTTTGATGGACCTTGAGAGAGGAACAATATATATGGGGGATTCTT | | | | | |
| CACAAAAGGGATGTAAGTTCCACTGGAGGGAATCTACCATGTTTGACATTGCCACA | | | | | |
| AGTCTATCGTCAAAATGTTCTTTTGTGCAATACCTGTGATGCACACAAAGCGATTA | | | | | |
| CTCCTCCTATGGTCCTCTAGATACTAGCTGGGAAGAGCCTCTCTTCACCCCTTAAG | | | | | |
| ATTATTTTTCTCCTCATCCTATGGCACTGTGCAAACACAGAAGTCTTCTTTGGTAA | | | | | |
| AGGAACCAGACTCACAGTTGTAGGTAAGATATCTTTCAGGTAAATTTCCAGGTCTC | | | | | |
| TCTTCGGACAAAGCATGTCCTCCGTGTCCATATTCGAGTATCTGTATTCTGATGTG | | | | | |
| CAAACTCCGACTACACCTTCGGCTCAGGGACCAGGCTTTTGGTAATAGGATTGCGG | | | | | |
| CTTTCCTATGCAAGCCACCACAGCTCTCTTACATCTCAGTGTAGGAGTGAATGTGG | | | | | |
| AACATCAGAGGATCTGAGAAATGTGACTCCACCCAAGGTCTCCTTGTTTGAGCCAT | | | | | |
| CAAAAGCAGAGATTGCAAACAAACAAAAGGCTACCCTCGTGTGCTTGGCC | | | | | |
| CATTTGGGTCTTTTTTTTTTGGGGGAAGATATTCAGAGCAGGACAGCTACTCAGCA | 762 | FALSE; TRUE | TRA; | FALSE; NA | premature |
| TCCTCTTGGACTGAGCTTATCTGCCAGAGCAGAAGGATTTCTGGATTGGATGCTGG | | | TRB | | stop |
| GAATAGAGAAACACTCATGGTTGGTGGGGATCTGAGTCAGGGTTGGAGCAGCAGGA | | | | | codon; |
| GCGGGAAGCACTGAGTTTTTGTCCTGAGCCTGGAGGCTGTGAGTGCAGAAACGCTG | | | | | absent |
| TATTTTGGCTCAGGAACCAGACTGACTGTTCTCGAGGATCTGAGAAATGTGACTCC | | | | | V; NA |
| ACCCAAGGTCTCCTTGTTTGAGCCATCAAAAGCAGAGATTGCAAACAAACAAAAGG | | | | | |
| CTACCCTCGTGTGCTTGGCC_GGCCAAGCACACGAGGGTAGCCTTTTGTTTGTTTG | | | | | |
| CAATCTCTGCTTTTGATGGCTCAAACAAGGAGACCTTGGGTGGAGTCACATTTCTC | | | | | |
| AGATCCTCTAAAACCGTGAGCCTGGTGCCGGGACCGAAGTACTGTTCATAGGAGCA | | | | | |
| CAGAGGCTCAACCCCACACACAAACCCGCGGGGTTCAGCGAGGTATTCTCCCATTT | | | | | |
| AGGGAAAATCTCCCTGCTACTCAGCCTCCCTCTCCAGCGCCCGGCTTGGGAACTCA | | | | | |
| GGGTAAAGACGCCAGAAGCCCCAGAACTCCCCGCTGCAGTTGCCAAGAAGCTCGCA | | | | | |
| TAGGCTGTTCAATCGGCTGCCAATCAGTCAGGGCAAGGCCCCA_AGTCAAAGTCGG | | | | | |
| TGAACAGGCAGAGGGTGCTGTCCTGAGACCGAGGATCTTTTAACTGGTACACAGCA | | | | | |
| GGTTCTGGGTTCTGGATGTTTGATTTAACAGAGAGTTTAGTGCCTTTCCCGAAGGT | | | | | |
| TAGCTTTGCATTGCTTCCTCCAGAATTCACAGTCATTTGGGGCCTTACAATAACCA | | | | | |
| GCAGGCAGGAAGGACCGAGAGGCAGACCAAGAAAAGCGACATCATCTTCCCCTCTA | | | | | |
| GTGGAAGCCTTCCTCCTTGCAGCCTTAGTCCCCAGAGTCTGGGAAGGCTTAGAGCA | | | | | |
| TCCTCTCAAAACTAACAGCTGGCTGTGAAGCTACTCTGAGCTTGATCAGAAACCAC | | | | | |
| TCCC_GGGGTCCTAGGTAAGAAGGCAGAGGCCATACAGGTGGGAGAGAAGGTAGGA | | | | | |
| CAGTCAGCCACAAAGACCCCCGGAGCAGAAAACCTTAGCAGAGTGGATATTTAACT | | | | | |
| ATCCAAGGCCCTGTATCAGGAAAGAGGAGGTTTGTGCCAGCATTTCCAGGACTGTG | | | | | |
| CAAACACCGGGCAGCTCTACTTTGGTGAAGGCTCAAAGCTGACAGTGCTGGAGGAT | | | | | |
| CTGAGAAATGTGACTCCACCCAAGGTCTCCTTGTTTGAGCCATCAAAAGCAGAGAT | | | | | |
| TGCAAACAAACAAAAGGCTACCCTCGTGTGCTTGGCC_GTTTCTGATGTGCAGTCA | | | | | |
| GTCAAGCTAGGAGAAACTACATTCCTGCCGTGACCCTACTATGGATATCTGGCTTC | | | | | |
| TAGGTTGGATAATTTTTAGTTTCTTGGAAGCAGGACACACAGGACCCAAAGTCTTA | | | | | |
| CAGATCCCAAGTCATCAAATAATAGATATGGGGCAGATGGTGACCCTCAATTGTGA | | | | | |
| CCCAGTTTCTAATCACCTATATTTTTATTGGTATAAACAGATTTTAGGACAGCAGA | | | | | |
| TGGAGTTTCTGGTTAATTTCTACAATGGTAAAGTCATGGAGAAGTCTAAACTGTTT | | | | | |
| AAGGATCAGTTTTCAGTTGAAAGACCAGATGGTTCATATTTCACTCTGAAAATCCA | | | | | |
| ACCCACAGCACTGGAGGACTCAGCTGTGTACTTCTGTGCCAGCAGCTCGGGACAGG | | | | | |
| AAAACTCCGACTACACCTTCGGCTCAGGGACCAGGCTTTTGGTAATAGAGGATCTG | | | | | |
| AGAAATGTGACTCCACCCAAGGTCTCCTTGTTTGAGCCATCAAAAGCAGAGATTGC | | | | | |
| AAACAAACAAAAGGCTACCCTCGTGTGCTTGGCC_TACGATACCAGGCAGTAAAGC | | | | | |
| TTCGGGTTTCTTATGGGGCTCATTGGAGGCTACCTGGACACCAACAGGAAGCCTTC | | | | | |
| AGATTTCAAATTCACTTTGTCAAACCCTGAGTGAATAGATGGATATCCGTTCCCAA | | | | | |
| GCCAAAAGTGGTATCTCCTCCTCAATTTGAGATCGGCCTCATGCAAGGTCAAGATT | | | | | |
| GCCTTACCAGTTCTGGAGGTAGATGGAGAATGTGAGTAACCCCGGGTCTGTATTGA | | | | | |
| GGAAGGTGAGGAAAGAGGAAGAATTCTTGGTAGCCCTTTTCTGCTGTGTAACTATG | | | | | |
| CTGAGCAGTTCTTCGGACCAGGGACACGACTCACCGTCCTAGAGGATCTGAGAAAT | | | | | |
| GTGACTCCACCCAAGGTCTCCTTGTTTGAGCCATCAAAAGCAGAGATTGCAAACAA | | | | | |
| ACAAAAGGCTACCCTCGTGTGCTTGGCC GGCCAAGCACACGAGGGTAGCCTTTTG | | | | | |
| TTTGTTTGCAATCTCTGCTTTTGATGGCTCAAACAAGGAGACCTTGGGTGGAGTCA | | | | | |
| CATTTCTCAGATCCTCTACAACTGTGAGTCTGGTTCCTTTACCAAAGAAGACTTCT | | | | | |
| GTGTTTGCACAGTGCCATAGGATGAGGAGAAAAATAATCTTAAGGGGTGAAGAGAG | | | | | |
| GCTCTTCCCAGCTAGTATCTAGAGGACCATAGGAGGAGTAATCGCTTTGTGTGCAT | | | | | |
| CACAGGTATTGCACAAAAGAACATTTTGACGATAGACTTGTGGCAATGTCAAACAT | | | | | |
| GGTAGATTCCCTCCAGTGGAACTTACATCCCTTTTGTGAAGAATCCCCCATATATA | | | | | |
| TTGTTCCTCTCTCAAGGTCCATCAAAGTGAGGTAGCCTGTCAGAGAACAGTTTTCC | | | | | |
| TGGTCCTATGATGATATTCATCAAGAGCTGAGATCTTTTAAAACAAAACAAACCTG | | | | | |
| TAGAACTGTTCACCTCTGGCTCCCCCA | | | | | |
| GGGGGATCATTTCAATGACACCCAGCGCCAAGAAAAAAGAACATTCAAAAGAAGAA | 763 | FALSE | TRA | TRUE | absent V |
| CAGGGGGCCCACAGTCCTCTACTCTTCTCCTGGGTCGCAAGCCTAGATGTGTTTAG | | | | | |
| ATCCAGAATGCTTTCACGTCACCTCTGCAGCCTGCTAGGCCAAGATTGTGGAGAAG | | | | | |
| AATGGGGCCTTACCAGAGCAGGAGCCTCCTACACTGAATGAACATAAGAAGAATGG | | | | | |
| CCTAGTGGCCCTAGCAGCAAAGGATGCAGGGAAATCCTCTTCTGCCAACGTCCATA | | | | | |
| AAACAGACCACCATCAGTGGATAGGTGAGCCAGAGGTGAACAGTTCTACAGGTTTG | | | | | |
| TTTTGTTTTAAAAGATCTCAGCTCTTGATGAATATCATCATAGGACCAGGAAAACT | | | | | |
| GTTCTCTGACAGGCTACCTCACTTTGATGGACCTTGAGAGAGGAACAATATATATG | | | | | |
| GGGGATTCTTCACAAAAGGGATGTAAGTTCCACTGGAGGGAATCTACCATGTTTGA | | | | | |
| CATTGCCACAAGTCTATCGTCAAAATGTTCTTTTGTGCAATACCTGTGATGCACAC | | | | | |
| AAAGCGATTACTCCTCCTATGGTCCTCTAGATACTAGCTGGGAAGAGCCTCTCTTC | | | | | |
| ACCCCTTAAGATTATTTTTCTCCTCATCCTATGGCACTGTGCAAACACAGAAGTCT | | | | | |
| TCTTTGGTAAAGGAACCAGACTCACAGTTGTAGGTAAGATATCTTTCAGGTAAATT | | | | | |
| TCCAGGTCTCTCTTCGGACAAAGCATGTCCTCCGTGTCCATATTCGAGTATCTGTA | | | | | |
| TTCTGATGTGCAAACTCCGACTACACCTTCGGCTCAGGGACCAGGCTTTTGGTAAT | | | | | |
| AGAGGATCTGAGAAATGTGACTCCACCCAAGGTCTCCTTGTTTGAGCCATCAAAAG | | | | | |
| CAGAGATTGCAAACAAACAAAAGGCTACCCTCGTGTGCTTGGCC | | | | | |
| GGGGGTCACACACAGAGTCTTGATTGTGGGACAAGGCCCACAGTCCTCTACTCTTC | 764 | FALSE | TRA | TRUE | absent V |
| TCCTGGGTCGCAAGCCTAGATGTGTTTAGATCCAGAATGCTTTCACGTCACCTCTG | | | | | |
| CAGCCTGCTAGGCCAAGATTGTGGAGAAGAATGGGGCCTTACCAGAGCAGGAGCCT | | | | | |
| CCTACACTGAATGAACATAAGAAGAATGGCCTAGTGGCCCTAGCAGCAAAGGATGC | | | | | |
| AGGGAAATCCTCTTCTGCCAACGTCCATAAAACAGACCACCATCAGTGGATAGGTG | | | | | |
| AGCCAGAGGTGAACAGTTCTACAGGTTTGTTTTGTTTTAAAAGATCTCAGCTCTTG | | | | | |
| ATGAATATCATCATAGGACCAGGAAAACTGTTCTCTGACAGGCTACCTCACTTTGA | | | | | |
| TGGACCTTGAGAGAGGAACAATATATATGGGGGATTCTTCACAAAAGGGATGTAAG | | | | | |
| TTCCACTGGAGGGAATCTACCATGTTTGACATTGCCACAAGTCTATCGTCAAAATG | | | | | |
| TTCTTTTGTGCAATACCTGTGATGCACACAAAGCGATTACTCCTCCTATGGTCCTC | | | | | |
| TAGATACTAGCTGGGAAGAGCCTCTCTTCACCCCTTAAGATTATTTTTCTCCTCAT | | | | | |
| CCTATGGCACTGTGCAAACACAGAAGTCTTCTTTGGTAAAGGAACCAGACTCACAG | | | | | |
| TTGTAGGTAAGATATCTTTCAGGTAAATTTCCAGGTCTCTCTTCGGACAAAGCATG | | | | | |
| TCCTCCGTGTCCATATTCGAGTATCTGTATTCTGATGTGCAAACTCCGACTACACC | | | | | |
| TTCGGCTCAGGGACCAGGCTTTTGGTAATAGAGGATCTGAGAAATGTGACTCCACC | | | | | |
| CAAGGTCTCCTTGTTTGAGCCATCAAAAGCAGAGATTGCAAACAAACAAAAGGCTA | | | | | |
| CCCTCGTGTGCTTGGCC | | | | | |
| TGGGGAGGCCTGGAGCTGTATCTCTTGCGATCTGATCTTCATAGGGAAGAATGCAC | 765 | TRUE; FALSE | TRA; | FALSE | NA; |
| AGCCTCCTGGGGTTGTTGTTGTGGCTGCAACTGACAAGGGTGAATAGTCAACTAGC | | | TRB | | premature |
| AGAAGAGAATTCGTGGGCCCTGAGCGTCCACGAGGGTGAAAGTGTCACGGTGAATT | | | | | stop |
| GTAGTTACAAGACATCCATAACTGCCCTACAGTGGTACAGACAGAAGTCAGGCAAA | | | | | codon |
| GGCCCTGCCCAGCTAATCTTAATACGTTCAAATGAGAGAGAGAAGCGCAATGGAAG | | | | | |
| ACTCAGAGCCACCCTTGACACCTCCAGCCAGAGCAGCTCCTTGTCCATCACTGCTA | | | | | |
| CTCGGTGTGAAGACACCGCTGTGTACTTCTGTGCTCCTTATAATGTTGGTGACAAC | | | | | |
| AGTAAGCTGATTTGGGGCTTGGGGACAAGTCTGGTAGTAAATCCAAACATCCAGAA | | | | | |
| CCCAGAACCTGCTGTGTACCAGTTAAAAGATCCTCGGTCTCAGGACAGCACCCTCT | | | | | |
| GCCTGTTCACCGACTTTGACT_TGGGGAAAAGATCATTTCAATGACACCCAGCGCC | | | | | |
| AAGAAAAAAGAACATTCAAAAGAAGAACAGGGGGTAAAGAGGAAACCCCTGCATTA | | | | | |
| GCTCGCATCTTACCACCACCTTGCACAATGGGGGTCGGGGGGGGATGTCACCTTCC | | | | | |
| TTATCTTCAACTCCCCCCCAGAGGAGCAGCTTATCTGGTGGTTTCTTCCAGCCCTC | | | | | |
| AAGGGGTAGACCTATGGGAGGGTCCTTTTTTGTATAAAGCTGTAACATTGTGGGGA | | | | | |
| CAGTGCAGAAACGCTGTATTTTGGCTCAGGAACCAGACTGACTGTTCTCGAGGATC | | | | | |
| TGAGAAATGTGACTCCACCCAAGGTCTCCTTGTTTGAGCCATCAAAAGCAGAGATT | | | | | |
| GCAAACAAACAAAAGGCTACCCTCGTGTGCTTGGCC_TGGGGGAAGATATTCAGAG | | | | | |
| CAGGACAGCTACTCAGCATCCTCTTGGACTGAGCTTATCTGCCAGAGCAGAAGGAT | | | | | |
| TTCTGGATTGGATGCTGGGAATAGAGAAACACTCATGGTTGGTGGGGATCTGAGTC | | | | | |
| AGGGTTGGAGCAGCAGGAGCGGGAAGCACTGAGTTTTTGTCCTGAGCCTGGAGGCT | | | | | |
| GTGAGTGCAGAAACGCTGTATTTTGGCTCAGGAACCAGACTGACTGTTCTCGAGGA | | | | | |
| TCTGAGAAATGTGACTCCACCCAAGGTCTCCTTGTTTGAGCCATCAAAAGCAGAGA | | | | | |
| TTGCAAACAAACAAAAGGCTACCCTCGTGTGCTTGGCC_AATACCCGTCTGGAGCC | | | | | |
| TGATTCCACCATGAGCTGCAGGCTTCTCCTCTATGTTTCCCTATGTCTTGTGGAAA | | | | | |
| CAGCACTCATGAACACTAAAATTACTCAGTCACCAAGATATCTAATCCTGGGAAGA | | | | | |
| GCAAATAAGTCTTTGGAATGTGAGCAACATCTGGGACATAATGCTATGTACTGGTA | | | | | |
| TAAACAGAGCGCTGAGAAGCCGCCAGAGCTCATGTTTCTCTACAATCTTAAACAGT | | | | | |
| TGATTCGAAATGAGACGGTGCCCAGTCGTTTTATACCTGAATGCCCAGACAGCTCC | | | | | |
| AAGCTACTTTTACATATATCTGCCGTGGATCCAGAAGACTCAGCTGTCTATTTTTG | | | | | |
| TGCCAGCAGCCAAGATAGGGACAGGGATGCTGAGCAGTTCTTCGGACCAGGGACAC | | | | | |
| GACTCACCGTCCTAGAGGATCTGAGAAATGTGACTCCACCCAAGGTCTCCTTGTTT | | | | | |
| GAGCCATCAAAAGCAGAGATTGCAAACAAACAAAAGGCTACCCTCGTGTGCTTGGC | | | | | |
| C | | | | | |
| GTGAGTGAGGAGTGTGAGGAGATCCTGCAGGAGGATTGCCCTGGGAGATTGGTGCA | 766 | TRUE; FALSE | TRA; | FALSE; NA | NA; |
| CTCAAGGACCAAGTGTCATTTCTTCCATGAACATGCGTCCTGACACCTGCTCAGTT | | | TRB | | absent V; |
| CTTGTGCTCCTCTTAATGCTCAGAAGGAACAATGGAGACTCTGTGACCCAGACAGA | | | | | premature |
| AGGCCTGGTCACTCTCACCGAGGGGTTGCCTGTGATGCTGAACTGCACCTATCAGA | | | | | stop |
| GTACTTACTCACCTTTCCTTTTCTGGTATGTGCAACATCTCAACGAAGCCCCTAAG | | | | | codon |
| CTACTTTTGAAGAGCTTCACAGACAACAAGAGGCCCGAGCACCAAGGGTTCCACGC | | | | | |
| CACTCTCCATAAGAGCAGCAGCTCCTTCCATCTGCAGAAGTCCTCAGCGCAGCTGT | | | | | |
| CAGACTCTGCCCTGTACTACTGTGCTTTGAGTGATCTTGTTGGTGACAACAGTAAG | | | | | |
| CTGATTTGGGGCTTGGGGACAAGTCTGGTAGTAAATCCAAACATCCAGAACCCAGA | | | | | |
| ACCTGCTGTGTACCAGTTAAAAGATCCTCGGTCTCAGGACAGCACCCTCTGCCTGT | | | | | |
| TCACCGACTTTGACT_TGGGGAGAACATTCAAAAGAAGAACAGGGGGTAAAGAGGA | | | | | |
| AACCCCTGCATTAGCTCGCATCTTACCACCACCTTGCACAATGGGGGTCGGGGGGG | | | | | |
| GATGTCACCTTCCTTATCTTCAACTCCCCCCCAGAGGAGCAGCTTATCTGGTGGTT | | | | | |
| TCTTCCAGCCCTCAAGGGGTAGACCTATGGGAGGGTCCTTTTTTGTATAAAGCTGT | | | | | |
| AACATTGTGGGGACAGGGGGGTTCTGGAAATACGCTCTATTTTGGAGAAGGAAGCC | | | | | |
| GGCTCATTGTTGTAGAGGATCTGAGAAATGTGACTCCACCCAAGGTCTCCTTGTTT | | | | | |
| GAGCCATCAAAAGCAGAGATTGCAAACAAACAAAAGGCTACCCTCGTGTGCTTGGC | | | | | |
| C_AATACCCGTCTGGAGCCTGATTCCACCATGAGCTGCAGGCTTCTCCTCTATGTT | | | | | |
| TCCCTATGTCTTGTGGAAACAGCACTCATGAACACTAAAATTACTCAGTCACCAAG | | | | | |
| ATATCTAATCCTGGGAAGAGCAAATAAGTCTTTGGAATGTGAGCAACATCTGGGAC | | | | | |
| ATAATGCTATGTACTGGTATAAACAGAGCGCTGAGAAGCCGCCAGAGCTCATGTTT | | | | | |
| CTCTACAATCTTAAACAGTTGATTCGAAATGAGACGGTGCCCAGTCGTTTTATACC | | | | | |
| TGAATGCCCAGACAGCTCCAAGCTACTTTTACATATATCTGCCGTGGATCCAGAAG | | | | | |
| ACTCAGCTGTCTATTTTTGTGCCAGCAGCCAAGACCCAAACACAGAAGTCTTCTTT | | | | | |
| GGTAAAGGAACCAGACTCACAGTTGTAGAGGATCTGAGAAATGTGACTCCACCCAA | | | | | |
| GGTCTCCTTGTTTGAGCCATCAAAAGCAGAGATTGCAAACAAACAAAAGGCTACCC | | | | | |
| TCGTGTGCTTGGCC_GGGGATAGATGGATATCCGTTCCCAAGCCAAAAGTGGTATC | | | | | |
| TCCTCCTCAATTTGAGATCGGCCTCATGCAAGGTCAAGATTGCCTTACCAGTTCTG | | | | | |
| GAGGTAGATGGAGAATGTGAGTAACCCCGGGTCTGTATTGAGGAAGGTGAGGAAAG | | | | | |
| AGGAAGAATTCTTGGTAGCCCTTTTCTGCTGTGTAACTATGCTGAGCAGTTCTTCG | | | | | |
| GACCAGGGACACGACTCACCGTCCTAGAGGATCTGAGAAATGTGACTCCACCCAAG | | | | | |
| GTCTCCTTGTTTGAGCCATCAAAAGCAGAGATTGCAAACAAACAAAAGGCTACCCT | | | | | |
| CGTGTGCTTGGCC | | | | | |
| TATGGGGCAATGCTCAGAAACCAACCACCTTGAACAGGCCTGGAGCTGTATCTCTT | 767 | TRUE | TRA; | FALSE | NA |
| GCGATCTGATCTTCATAGGGAAGAATGCACAGCCTCCTGGGGTTGTTGTTGTGGCT | | | TRB | | |
| GCAACTGACAAGGGTGAATAGTCAACTAGCAGAAGAGAATTCGTGGGCCCTGAGCG | | | | | |
| TCCACGAGGGTGAAAGTGTCACGGTGAATTGTAGTTACAAGACATCCATAACTGCC | | | | | |
| CTACAGTGGTACAGACAGAAGTCAGGCAAAGGCCCTGCCCAGCTAATCTTAATACG | | | | | |
| TTCAAATGAGAGAGAGAAGCGCAATGGAAGACTCAGAGCCACCCTTGACACCTCCA | | | | | |
| GCCAGAGCAGCTCCTTGTCCATCACTGCTACTCGGTGTGAAGACACCGCTGTGTAC | | | | | |
| TTCTGTGCTCCTTATAATGTTGGTGACAACAGTAAGCTGATTTGGGGCTTGGGGAC | | | | | |
| AAGTCTGGTAGTAAATCCAAACATCCAGAACCCAGAACCTGCTGTGTACCAGTTAA | | | | | |
| AAGATCCTCGGTCTCAGGACAGCACCCTCTGCCTGTTCACCGACTTTGACT_AATA | | | | | |
| CCCGTCTGGAGCCTGATTCCACCATGAGCTGCAGGCTTCTCCTCTATGTTTCCCTA | | | | | |
| TGTCTTGTGGAAACAGCACTCATGAACACTAAAATTACTCAGTCACCAAGATATCT | | | | | |
| AATCCTGGGAAGAGCAAATAAGTCTTTGGAATGTGAGCAACATCTGGGACATAATG | | | | | |
| CTATGTACTGGTATAAACAGAGCGCTGAGAAGCCGCCAGAGCTCATGTTTCTCTAC | | | | | |
| AATCTTAAACAGTTGATTCGAAATGAGACGGTGCCCAGTCGTTTTATACCTGAATG | | | | | |
| CCCAGACAGCTCCAAGCTACTTTTACATATATCTGCCGTGGATCCAGAAGACTCAG | | | | | |
| CTGTCTATTTTTGTGCCAGCAGCCAAGATAGGGACAGGGATGCTGAGCAGTTCTTC | | | | | |
| GGACCAGGGACACGACTCACCGTCCTAGAGGATCTGAGAAATGTGACTCCACCCAA | | | | | |
| GGTCTCCTTGTTTGAGCCATCAAAAGCAGAGATTGCAAACAAACAAAAGGCTACCC | | | | | |
| TCGTGTGCTTGGCC | | | | | |
| GGGGAGAACATTCAAAAGAAGAACAGGGGGCCCACAGTCCTCTACTCTTCTCCTGG | 768 | FALSE | TRA | TRUE | absent V |
| GTCGCAAGCCTAGATGTGTTTAGATCCAGAATGCTTTCACGTCACCTCTGCAGCCT | | | | | |
| GCTAGGCCAAGATTGTGGAGAAGAATGGGGCCTTACCAGAGCAGGAGCCTCCTACA | | | | | |
| CTGAATGAACATAAGAAGAATGGCCTAGTGGCCCTAGCAGCAAAGGATGCAGGGAA | | | | | |
| ATCCTCTTCTGCCAACGTCCATAAAACAGACCACCATCAGTGGATAGGTGAGCCAG | | | | | |
| AGGTGAACAGTTCTACAGGTTTGTTTTGTTTTAAAAGATCTCAGCTCTTGATGAAT | | | | | |
| ATCATCATAGGACCAGGAAAACTGTTCTCTGACAGGCTACCTCACTTTGATGGACC | | | | | |
| TTGAGAGAGGAACAATATATATGGGGGATTCTTCACAAAAGGGATGTAAGTTCCAC | | | | | |
| TGGAGGGAATCTACCATGTTTGACATTGCCACAAGTCTATCGTCAAAATGTTCTTT | | | | | |
| TGTGCAATACCTGTGATGCACACAAAGCGATTACTCCTCCTATGGTCCTCTAGATA | | | | | |
| CTAGCTGGGAAGAGCCTCTCTTCACCCCTTAAGATTATTTTTCTCCTCATCCTATG | | | | | |
| GCACTGTGCAAACACAGAAGTCTTCTTTGGTAAAGGAACCAGACTCACAGTTGTAG | | | | | |
| GTAAGATATCTTTCAGGTAAATTTCCAGGTCTCTCTTCGGACAAAGCATGTCCTCC | | | | | |
| GTGTCCATATTCGAGTATCTGTATTCTGATGTGCAAACTCCGACTACACCTTCGGC | | | | | |
| TCAGGGACCAGGCTTTTGGTAATAGAGGATCTGAGAAATGTGACTCCACCCAAGGT | | | | | |
| CTCCTTGTTTGAGCCATCAAAAGCAGAGATTGCAAACAAACAAAAGGCTACCCTCG | | | | | |
| TGTGCTTGGCC | | | | | |
| TGGGGCACGGTGATTCAATTCTATGGGAAGCCTTTACAAAAACCATTCTGTCTGTC | 769 | FALSE | TRA | TRUE | absent V |
| CCAAGGCCCACAGTCCTCTACTCTTCTCCTGGGTCGCAAGCCTAGATGTGTTTAGA | | | | | |
| TCCAGAATGCTTTCACGTCACCTCTGCAGCCTGCTAGGCCAAGATTGTGGAGAAGA | | | | | |
| ATGGGGCCTTACCAGAGCAGGAGCCTCCTACACTGAATGAACATAAGAAGAATGGC | | | | | |
| CTAGTGGCCCTAGCAGCAAAGGATGCAGGGAAATCCTCTTCTGCCAACGTCCATAA | | | | | |
| AACAGACCACCATCAGTGGATAGGTGAGCCAGAGGTGAACAGTTCTACAGGTTTGT | | | | | |
| TTTGTTTTAAAAGATCTCAGCTCTTGATGAATATCATCATAGGACCAGGAAAACTG | | | | | |
| TTCTCTGACAGGCTACCTCACTTTGATGGACCTTGAGAGAGGAACAATATATATGG | | | | | |
| GGGATTCTTCACAAAAGGGATGTAAGTTCCACTGGAGGGAATCTACCATGTTTGAC | | | | | |
| ATTGCCACAAGTCTATCGTCAAAATGTTCTTTTGTGCAATACCTGTGATGCACACA | | | | | |
| AAGCGATTACTCCTCCTATGGTCCTCTAGATACTAGCTGGGAAGAGCCTCTCTTCA | | | | | |
| CCCCTTAAGATTATTTTTCTCCTCATCCTATGGCACTGTGCAAACACAGAAGTCTT | | | | | |
| CTTTGGTAAAGGAACCAGACTCACAGTTGTAGGTAAGATATCTTTCAGGTAAATTT | | | | | |
| CCAGGTCTCTCTTCGGACAAAGCATGTCCTCCGTGTCCATATTCGAGTATCTGTAT | | | | | |
| TCTGATGTGCAAACTCCGACTACACCTTCGGCTCAGGGACCAGGCTTTTGGTAATA | | | | | |
| GAGGATCTGAGAAATGTGACTCCACCCAAGGTCTCCTTGTTTGAGCCATCAAAAGC | | | | | |
| AGAGATTGCAAACAAACAAAAGGCTACCCTCGTGTGCTTGGCC | | | | | |
| AAAGCTGTAACATTGTGGGGACAGGGGGCCACGGTGATTCAATTCTATGGGAAGCC | 770 | FALSE | TRA | TRUE | absent V |
| TTTACAAAAACCATTCTGTCTGTCCCAAGGCCCACAGTCCTCTACTCTTCTCCTGG | | | | | |
| GTCGCAAGCCTAGATGTGTTTAGATCCAGAATGCTTTCACGTCACCTCTGCAGCCT | | | | | |
| GCTAGGCCAAGATTGTGGAGAAGAATGGGGCCTTACCAGAGCAGGAGCCTCCTACA | | | | | |
| CTGAATGAACATAAGAAGAATGGCCTAGTGGCCCTAGCAGCAAAGGATGCAGGGAA | | | | | |
| ATCCTCTTCTGCCAACGTCCATAAAACAGACCACCATCAGTGGATAGGTGAGCCAG | | | | | |
| AGGTGAACAGTTCTACAGGTTTGTTTTGTTTTAAAAGATCTCAGCTCTTGATGAAT | | | | | |
| ATCATCATAGGACCAGGAAAACTGTTCTCTGACAGGCTACCTCACTTTGATGGACC | | | | | |
| TTGAGAGAGGAACAATATATATGGGGGATTCTTCACAAAAGGGATGTAAGTTCCAC | | | | | |
| TGGAGGGAATCTACCATGTTTGACATTGCCACAAGTCTATCGTCAAAATGTTCTTT | | | | | |
| TGTGCAATACCTGTGATGCACACAAAGCGATTACTCCTCCTATGGTCCTCTAGATA | | | | | |
| CTAGCTGGGAAGAGCCTCTCTTCACCCCTTAAGATTATTTTTCTCCTCATCCTATG | | | | | |
| GCACTGTGCAAACACAGAAGTCTTCTTTGGTAAAGGAACCAGACTCACAGTTGTAG | | | | | |
| GTAAGATATCTTTCAGGTAAATTTCCAGGTCTCTCTTCGGACAAAGCATGTCCTCC | | | | | |
| GTGTCCATATTCGAGTATCTGTATTCTGATGTGCAAACTCCGACTACACCTTCGGC | | | | | |
| TCAGGGACCAGGCTTTTGGTAATAGAGGATCTGAGAAATGTGACTCCACCCAAGGT | | | | | |
| CTCCTTGTTTGAGCCATCAAAAGCAGAGATTGCAAACAAACAAAAGGCTACCCTCG | | | | | |
| TGTGCTTGGCC | | | | | |
| GAATGACACCCAGCGCCAAGAAAAAAGAACATTCAAAAGAAGAACAGGGGGCCCAC | 771 | FALSE | TRA; | TRUE; NA | absent V; |
| AGTCCTCTACTCTTCTCCTGGGTCGCAAGCCTAGATGTGTTTAGATCCAGAATGCT | | | TRB | | premature |
| TTCACGTCACCTCTGCAGCCTGCTAGGCCAAGATTGTGGAGAAGAATGGGGCCTTA | | | | | stop |
| CCAGAGCAGGAGCCTCCTACACTGAATGAACATAAGAAGAATGGCCTAGTGGCCCT | | | | | codon |
| AGCAGCAAAGGATGCAGGGAAATCCTCTTCTGCCAACGTCCATAAAACAGACCACC | | | | | |
| ATCAGTGGATAGGTGAGCCAGAGGTGAACAGTTCTACAGGTTTGTTTTGTTTTAAA | | | | | |
| AGATCTCAGCTCTTGATGAATATCATCATAGGACCAGGAAAACTGTTCTCTGACAG | | | | | |
| GCTACCTCACTTTGATGGACCTTGAGAGAGGAACAATATATATGGGGGATTCTTCA | | | | | |
| CAAAAGGGATGTAAGTTCCACTGGAGGGAATCTACCATGTTTGACATTGCCACAAG | | | | | |
| TCTATCGTCAAAATGTTCTTTTGTGCAATACCTGTGATGCACACAAAGCGATTACT | | | | | |
| CCTCCTATGGTCCTCTAGATACTAGCTGGGAAGAGCCTCTCTTCACCCCTTAAGAT | | | | | |
| TATTTTTCTCCTCATCCTATGGCACTGTGCAAACACAGAAGTCTTCTTTGGTAAAG | | | | | |
| GAACCAGACTCACAGTTGTAGGTAAGATATCTTTCAGGTAAATTTCCAGGTCTCTC | | | | | |
| TTCGGACAAAGCATGTCCTCCGTGTCCATATTCGAGTATCTGTATTCTGATGTGCA | | | | | |
| AACTCCGACTACACCTTCGGCTCAGGGACCAGGCTTTTGGTAATAGAGGATCTGAG | | | | | |
| AAATGTGACTCCACCCAAGGTCTCCTTGTTTGAGCCATCAAAAGCAGAGATTGCAA | | | | | |
| ACAAACAAAAGGCTACCCTCGTGTGCTTGGCC_CTCGTGAGGCAGGAGACTCAGCC | | | | | |
| CGAGGAGATCGCAGATAAGTTTTTAATTAAAAAGATTGAGCAGTAAAAAGAATTAG | | | | | |
| AACTCTAAACTTAAGCTAATAGAGTAGCTTATCGAAATATTACTTAGTCTTAATAA | | | | | |
| TCTAAGAAGATCTTAAGAGATAACATGAAGGCTTATTTAAACAGTTTGAAAAAGGA | | | | | |
| AATGAGGAGAAAAGTATTTGTACTGTATAATGGAGGCTGACCAGAGCAGTTTAGGA | | | | | |
| GATTGTAAAGGGAGGTTTTGTGAAGTTCTAAAAGGTTCTAGTTTGAAGGTCGGCCT | | | | | |
| TGTAGATTAAAACGAAGGTTACCTAAATAGAATCTAAGTGGCATTTAAAACAGTAA | | | | | |
| AGTTGTAGAGAATAGTTTGAAAATGAGGTGTAGTTTTAAAAGATTGAGAAAAG | | | | | |
| CTCTGGTTCTCGGACGATTTTGTTCATTTCTTATATGGGATCCACAAAGGTGGATT | 772 | FALSE | TRA | FALSE; | absent V |
| CATCATAGGTCCAGGAATGTTTATGGTGGAGTCAAGATGAACCTCGGGTGGAAGTC | | | | TRUE | |
| AGGAAAGAGAGAGGAGAAAGGCTCACAGAGAGGGAGGGGGTTTTGAAGTGGACCCG | | | | | |
| GGAGGCTGTGTTCTGGAAATACGCTCTATTTTGGAGAAGGAAGCCGGCTCATTGTT | | | | | |
| GTAGAGGATCTGAGAAATGTGACTCCACCCAAGGTCTCCTTGTTTGAGCCATCAAA | | | | | |
| AGCAGAGATTGCAAACAAACAAAAGGCTACCCTCGTGTGCTTGGCC_GGGTACACT | | | | | |
| GAATGAACATAAGAAGAATGGCCTAGTGGCCCTAGCAGCAAAGGATGCAGGGAAAT | | | | | |
| CCTCTTCTGCCAACGTCCATAAAACAGACCACCATCAGTGGATAGGTGAGCCAGAG | | | | | |
| GTGAACAGTTCTACAGGTTTGTTTTGTTTTAAAAGATCTCAGCTCTTGATGAATAT | | | | | |
| CATCATAGGACCAGGAAAACTGTTCTCTGACAGGCTACCTCACTTTGATGGACCTT | | | | | |
| GAGAGAGGAACAATATATATGGGGGATTCTTCACAAAAGGGATGTAAGTTCCACTG | | | | | |
| GAGGGAATCTACCATGTTTGACATTGCCACAAGTCTATCGTCAAAATGTTCTTTTG | | | | | |
| TGCAATACCTGTGATGCACACAAAGCGATTACTCCTCCTATGGTCCTCTAGATACT | | | | | |
| AGCTGGGAAGAGCCTCTCTTCACCCCTTAAGATTATTTTTCTCCTCATCCTATGGC | | | | | |
| ACTGTGCAAACACAGAAGTCTTCTTTGGTAAAGGAACCAGACTCACAGTTGTAGGT | | | | | |
| AAGATATCTTTCAGGTAAATTTCCAGGTCTCTCTTCGGACAAAGCATGTCCTCCGT | | | | | |
| GTCCATATTCGAGTATCTGTATTCTGATGTGCAAACTCCGACTACACCTTCGGCTC | | | | | |
| AGGGACCAGGCTTTTGGTAATAGGATTGCGGCTTTCCTATGCAAGCCACCACAGCT | | | | | |
| CTCTTACATCTCAGTGTAGGAGTGAATGTGGAACATCAGAGGATCTGAGAAATGTG | | | | | |
| ACTCCACCCAAGGTCTCCTTGTTTGAGCCATCAAAAGCAGAGATTGCAAACAAACA | | | | | |
| AAAGGCTACCCTCGTGTGCTTGGCC | | | | | |
| TCGTTTGATTTCTTTTCTGGGGACGGTGATTCAATTCTATGGGAAGCCTTTACAAA | | FALSE | TRA | TRUE | absent V |
| AACCATTCTGTCTGTCCCAAGGCCCACAGTCCTCTACTCTTCTCCTGGGTCGCAAG | | | | | |
| CCTAGATGTGTTTAGATCCAGAATGCTTTCACGTCACCTCTGCAGCCTGCTAGGCC | | | | | |
| AAGATTGTGGAGAAGAATGGGGCCTTACCAGAGCAGGAGCCTCCTACACTGAATGA | | | | | |
| ACATAAGAAGAATGGCCTAGTGGCCCTAGCAGCAAAGGATGCAGGGAAATCCTCTT | | | | | |
| CTGCCAACGTCCATAAAACAGACCACCATCAGTGGATAGGTGAGCCAGAGGTGAAC | | | | | |
| AGTTCTACAGGTTTGTTTTGTTTTAAAAGATCTCAGCTCTTGATGAATATCATCAT | | | | | |
| AGGACCAGGAAAACTGTTCTCTGACAGGCTACCTCACTTTGATGGACCTTGAGAGA | | | | | |
| GGAACAATATATATGGGGGATTCTTCACAAAAGGGATGTAAGTTCCACTGGAGGGA | | | | | |
| ATCTACCATGTTTGACATTGCCACAAGTCTATCGTCAAAATGTTCTTTTGTGCAAT | | | | | |
| ACCTGTGATGCACACAAAGCGATTACTCCTCCTATGGTCCTCTAGATACTAGCTGG | | | | | |
| GAAGAGCCTCTCTTCACCCCTTAAGATTATTTTTCTCCTCATCCTATGGCACTGTG | | | | | |
| CAAACACAGAAGTCTTCTTTGGTAAAGGAACCAGACTCACAGTTGTAGGTAAGATA | | | | | |
| TCTTTCAGGTAAATTTCCAGGTCTCTCTTCGGACAAAGCATGTCCTCCGTGTCCAT | | | | | |
| ATTCGAGTATCTGTATTCTGATGTGCAAACTCCGACTACACCTTCGGCTCAGGGAC | | | | | |
| CAGGCTTTTGGTAATAGGATTGCGGCTTTCCTATGCAAGCCACCACAGCTCTCTTA | | | | | |
| CATCTCAGTGTAGGAGTGAATGTGGAACATCAGAGGATCTGAGAAATGTGACTCCA | | | | | |
| CCCAAGGTCTCCTTGTTTGAGCCATCAAAAGCAGAGATTGCAAACAAACAAAAGGC | | | | | |
| TACCCTCGTGTGCTTGGCC | | | | | |
| AGAAACCAACCACCTTGAACAGGCCTGGAGCTGTATCTCTTGCGATCTGATCTTCA | 774 | TRUE; FALSE | TRA; | FALSE | NA; |
| TAGGGAAGAATGCACAGCCTCCTGGGGTTGTTGTTGTGGCTGCAACTGACAAGGGT | | | TRB | | premature |
| GAATAGTCAACTAGCAGAAGAGAATTCGTGGGCCCTGAGCGTCCACGAGGGTGAAA | | | | | stop |
| GTGTCACGGTGAATTGTAGTTACAAGACATCCATAACTGCCCTACAGTGGTACAGA | | | | | codon |
| CAGAAGTCAGGCAAAGGCCCTGCCCAGCTAATCTTAATACGTTCAAATGAGAGAGA | | | | | |
| GAAGCGCAATGGAAGACTCAGAGCCACCCTTGACACCTCCAGCCAGAGCAGCTCCT | | | | | |
| TGTCCATCACTGCTACTCGGTGTGAAGACACCGCTGTGTACTTCTGTGCTCCTTAT | | | | | |
| AATGTTGGTGACAACAGTAAGCTGATTTGGGGCTTGGGGACAAGTCTGGTAGTAAA | | | | | |
| TCCAAACATCCAGAACCCAGAACCTGCTGTGTACCAGTTAAAAGATCCTCGGTCTC | | | | | |
| AGGACAGCACCCTCTGCCTGTTCACCGACTTTGACT_GCATTTCTTATATGGGGAG | | | | | |
| ATATTCAGAGCAGGACAGCTACTCAGCATCCTCTTGGACTGAGCTTATCTGCCAGA | | | | | |
| GCAGAAGGATTTCTGGATTGGATGCTGGGAATAGAGAAACACTCATGGTTGGTGGG | | | | | |
| GATCTGAGTCAGGGTTGGAGCAGCAGGAGCGGGAAGCACTGAGTTTTTGTCCTGAG | | | | | |
| CCTGGAGGCTGTGAGTGCAGAAACGCTGTATTTTGGCTCAGGAACCAGACTGACTG | | | | | |
| TTCTCGAGGATCTGAGAAATGTGACTCCACCCAAGGTCTCCTTGTTTGAGCCATCA | | | | | |
| AAAGCAGAGATTGCAAACAAACAAAAGGCTACCCTCGTGTGCTTGGCC_AATACCC | | | | | |
| GTCTGGAGCCTGATTCCACCATGAGCTGCAGGCTTCTCCTCTATGTTTCCCTATGT | | | | | |
| CTTGTGGAAACAGCACTCATGAACACTAAAATTACTCAGTCACCAAGATATCTAAT | | | | | |
| CCTGGGAAGAGCAAATAAGTCTTTGGAATGTGAGCAACATCTGGGACATAATGCTA | | | | | |
| TGTACTGGTATAAACAGAGCGCTGAGAAGCCGCCAGAGCTCATGTTTCTCTACAAT | | | | | |
| CTTAAACAGTTGATTCGAAATGAGACGGTGCCCAGTCGTTTTATACCTGAATGCCC | | | | | |
| AGACAGCTCCAAGCTACTTTTACATATATCTGCCGTGGATCCAGAAGACTCAGCTG | | | | | |
| TCTATTTTTGTGCCAGCAGCCAAGATAGGGACAGGGATGCTGAGCAGTTCTTCGGA | | | | | |
| CCAGGGACACGACTCACCGTCCTAGAGGATCTGAGAAATGTGACTCCACCCAAGGT | | | | | |
| CTCCTTGTTTGAGCCATCAAAAGCAGAGATTGCAAACAAACAAAAGGCTACCCTCG | | | | | |
| TGTGCTTGGCC | | | | | |
| GGTGATTCAATTCTATGGGAAGCCTTTACAAAAACCATTCTGTCTGTCCCAAGGCC | 775 | FALSE | TRA; | TRUE; NA | absent V; |
| CACAGTCCTCTACTCTTCTCCTGGGTCGCAAGCCTAGATGTGTTTAGATCCAGAAT | | | TRB | | premature |
| GCTTTCACGTCACCTCTGCAGCCTGCTAGGCCAAGATTGTGGAGAAGAATGGGGCC | | | | | stop |
| TTACCAGAGCAGGAGCCTCCTACACTGAATGAACATAAGAAGAATGGCCTAGTGGC | | | | | codon |
| CCTAGCAGCAAAGGATGCAGGGAAATCCTCTTCTGCCAACGTCCATAAAACAGACC | | | | | |
| ACCATCAGTGGATAGGTGAGCCAGAGGTGAACAGTTCTACAGGTTTGTTTTGTTTT | | | | | |
| AAAAGATCTCAGCTCTTGATGAATATCATCATAGGACCAGGAAAACTGTTCTCTGA | | | | | |
| CAGGCTACCTCACTTTGATGGACCTTGAGAGAGGAACAATATATATGGGGGATTCT | | | | | |
| TCACAAAAGGGATGTAAGTTCCACTGGAGGGAATCTACCATGTTTGACATTGCCAC | | | | | |
| AAGTCTATCGTCAAAATGTTCTTTTGTGCAATACCTGTGATGCACACAAAGCGATT | | | | | |
| ACTCCTCCTATGGTCCTCTAGATACTAGCTGGGAAGAGCCTCTCTTCACCCCTTAA | | | | | |
| GATTATTTTTCTCCTCATCCTATGGCACTGTGCAAACACAGAAGTCTTCTTTGGTA | | | | | |
| AAGGAACCAGACTCACAGTTGTAGGTAAGATATCTTTCAGGTAAATTTCCAGGTCT | | | | | |
| CTCTTCGGACAAAGCATGTCCTCCGTGTCCATATTCGAGTATCTGTATTCTGATGT | | | | | |
| GCAAACTCCGACTACACCTTCGGCTCAGGGACCAGGCTTTTGGTAATAGAGGATCT | | | | | |
| GAGAAATGTGACTCCACCCAAGGTCTCCTTGTTTGAGCCATCAAAAGCAGAGATTG | | | | | |
| CAAACAAACAAAAGGCTACCCTCGTGTGCTTGGCC_CAGCGAGAGCAGAGCAGCGT | | | | | |
| AGAGCAGCACAGCTGAGCTCGTGAGGCAGGAGACTCAGCCCGAGGAAATCGCAGAT | | | | | |
| AAGTTTTTAATTAAAAAGATTGAGCAGTAAAAAGAATTAGAACTCTAAACTTAAGC | | | | | |
| TAATAGAGTAGCTTATCGAAATATTACTTAGTCTTAATAATCTAAGAAGATCTTAA | | | | | |
| GAGATAACATGAAGGCTTATTTAAACAGTTTGAAAAAGGAAATGAGGAGAAAAGTA | | | | | |
| TTTGTACTGTATAATGGAGGCTGACCAGAGCAGTTTAGGAGATTGTAAAGGGAGGT | | | | | |
| TTTGTGAAGTTCTAAAAGGTTCTAGTTTGAAGGTCGGCCTTGTAGATTAAAACGAA | | | | | |
| GGTTACCTAAATAGAATCTAAGTGGCATTTAAAACAGTAAAGTTGTAGAGAATAGT | | | | | |
| TTGAAAATGAGGTGTAGTTTTAAAAGATTGAGAAAAGTAGGTTAAGTTGACGGCCG | | | | | |
| TTATAAAAATCCTTC | | | | | |
| GCCCACAGTCCTCTACTCTTCTCCTGGGTCGCAAGCCTAGATGTGTTTAGATCCAG | 776 | FALSE | TRA | TRUE | absent V |
| AATGCTTTCACGTCACCTCTGCAGCCTGCTAGGCCAAGATTGTGGAGAAGAATGGG | | | | | |
| GCCTTACCAGAGCAGGAGCCTCCTACACTGAATGAACATAAGAAGAATGGCCTAGT | | | | | |
| GGCCCTAGCAGCAAAGGATGCAGGGAAATCCTCTTCTGCCAACGTCCATAAAACAG | | | | | |
| ACCACCATCAGTGGATAGGTGAGCCAGAGGTGAACAGTTCTACAGGTTTGTTTTGT | | | | | |
| TTTAAAAGATCTCAGCTCTTGATGAATATCATCATAGGACCAGGAAAACTGTTCTC | | | | | |
| TGACAGGCTACCTCACTTTGATGGACCTTGAGAGAGGAACAATATATATGGGGGAT | | | | | |
| TCTTCACAAAAGGGATGTAAGTTCCACTGGAGGGAATCTACCATGTTTGACATTGC | | | | | |
| CACAAGTCTATCGTCAAAATGTTCTTTTGTGCAATACCTGTGATGCACACAAAGCG | | | | | |
| ATTACTCCTCCTATGGTCCTCTAGATACTAGCTGGGAAGAGCCTCTCTTCACCCCT | | | | | |
| TAAGATTATTTTTCTCCTCATCCTATGGCACTGTGCAAACACAGAAGTCTTCTTTG | | | | | |
| GTAAAGGAACCAGACTCACAGTTGTAGGTAAGATATCTTTCAGGTAAATTTCCAGG | | | | | |
| TCTCTCTTCGGACAAAGCATGTCCTCCGTGTCCATATTCGAGTATCTGTATTCTGA | | | | | |
| TGTGCAAACTCCGACTACACCTTCGGCTCAGGGACCAGGCTTTTGGTAATAGAGGA | | | | | |
| TCTGAGAAATGTGACTCCACCCAAGGTCTCCTTGTTTGAGCCATCAAAAGCAGAGA | | | | | |
| TTGCAAACAAACAAAAGGCTACCCTCGTGTGCTTGGCC | | | | | |
| AGCGCCAAGAAAAAAGAACATTCAAAAGAAGAACAGGGGGCCCACAGTCCTCTACT | 777 | FALSE | TRA | TRUE | absent V |
| CTTCTCCTGGGTCGCAAGCCTAGATGTGTTTAGATCCAGAATGCTTTCACGTCACC | | | | | |
| TCTGCAGCCTGCTAGGCCAAGATTGTGGAGAAGAATGGGGCCTTACCAGAGCAGGA | | | | | |
| GCCTCCTACACTGAATGAACATAAGAAGAATGGCCTAGTGGCCCTAGCAGCAAAGG | | | | | |
| ATGCAGGGAAATCCTCTTCTGCCAACGTCCATAAAACAGACCACCATCAGTGGATA | | | | | |
| GGTGAGCCAGAGGTGAACAGTTCTACAGGTTTGTTTTGTTTTAAAAGATCTCAGCT | | | | | |
| CTTGATGAATATCATCATAGGACCAGGAAAACTGTTCTCTGACAGGCTACCTCACT | | | | | |
| TTGATGGACCTTGAGAGAGGAACAATATATATGGGGGATTCTTCACAAAAGGGATG | | | | | |
| TAAGTTCCACTGGAGGGAATCTACCATGTTTGACATTGCCACAAGTCTATCGTCAA | | | | | |
| AATGTTCTTTTGTGCAATACCTGTGATGCACACAAAGCGATTACTCCTCCTATGGT | | | | | |
| CCTCTAGATACTAGCTGGGAAGAGCCTCTCTTCACCCCTTAAGATTATTTTTCTCC | | | | | |
| TCATCCTATGGCACTGTGCAAACACAGAAGTCTTCTTTGGTAAAGGAACCAGACTC | | | | | |
| ACAGTTGTAGGTAAGATATCTTTCAGGTAAATTTCCAGGTCTCTCTTCGGACAAAG | | | | | |
| CATGTCCTCCGTGTCCATATTCGAGTATCTGTATTCTGATGTGCAAACTCCGACTA | | | | | |
| CACCTTCGGCTCAGGGACCAGGCTTTTGGTAATAGAGGATCTGAGAAATGTGACTC | | | | | |
| CACCCAAGGTCTCCTTGTTTGAGCCATCAAAAGCAGAGATTGCAAACAAACAAAAG | | | | | |
| GCTACCCTCGTGTGCTTGGCC | | | | | |
| ACGGTGATTCAATTCTATGGGAAGCCTTTACAAAAACCATTCTGTCTGTCCCAAGG | 778 | FALSE | TRA | TRUE | absent V |
| CCCACAGTCCTCTACTCTTCTCCTGGGTCGCAAGCCTAGATGTGTTTAGATCCAGA | | | | | |
| ATGCTTTCACGTCACCTCTGCAGCCTGCTAGGCCAAGATTGTGGAGAAGAATGGGG | | | | | |
| CCTTACCAGAGCAGGAGCCTCCTACACTGAATGAACATAAGAAGAATGGCCTAGTG | | | | | |
| GCCCTAGCAGCAAAGGATGCAGGGAAATCCTCTTCTGCCAACGTCCATAAAACAGA | | | | | |
| CCACCATCAGTGGATAGGTGAGCCAGAGGTGAACAGTTCTACAGGTTTGTTTTGTT | | | | | |
| TTAAAAGATCTCAGCTCTTGATGAATATCATCATAGGACCAGGAAAACTGTTCTCT | | | | | |
| GACAGGCTACCTCACTTTGATGGACCTTGAGAGAGGAACAATATATATGGGGGATT | | | | | |
| CTTCACAAAAGGGATGTAAGTTCCACTGGAGGGAATCTACCATGTTTGACATTGCC | | | | | |
| ACAAGTCTATCGTCAAAATGTTCTTTTGTGCAATACCTGTGATGCACACAAAGCGA | | | | | |
| TTACTCCTCCTATGGTCCTCTAGATACTAGCTGGGAAGAGCCTCTCTTCACCCCTT | | | | | |
| AAGATTATTTTTCTCCTCATCCTATGGCACTGTGCAAACACAGAAGTCTTCTTTGG | | | | | |
| TAAAGGAACCAGACTCACAGTTGTAGGTAAGATATCTTTCAGGTAAATTTCCAGGT | | | | | |
| CTCTCTTCGGACAAAGCATGTCCTCCGTGTCCATATTCGAGTATCTGTATTCTGAT | | | | | |
| GTGCAAACTCCGACTACACCTTCGGCTCAGGGACCAGGCTTTTGGTAATAGAGGAT | | | | | |
| CTGAGAAATGTGACTCCACCCAAGGTCTCCTTGTTTGAGCCATCAAAAGCAGAGAT | | | | | |
| TGCAAACAAACAAAAGGCTACCCTCGTGTGCTTGGCC | | | | | |
| GGGGAATGACACCCAGCGCCAAGAAAAAAGAACATTCAAAAGAAGAACAGGGGGCC | 779 | FALSE | TRA | TRUE | absent V |
| CACAGTCCTCTACTCTTCTCCTGGGTCGCAAGCCTAGATGTGTTTAGATCCAGAAT | | | | | |
| GCTTTCACGTCACCTCTGCAGCCTGCTAGGCCAAGATTGTGGAGAAGAATGGGGCC | | | | | |
| TTACCAGAGCAGGAGCCTCCTACACTGAATGAACATAAGAAGAATGGCCTAGTGGC | | | | | |
| CCTAGCAGCAAAGGATGCAGGGAAATCCTCTTCTGCCAACGTCCATAAAACAGACC | | | | | |
| ACCATCAGTGGATAGGTGAGCCAGAGGTGAACAGTTCTACAGGTTTGTTTTGTTTT | | | | | |
| AAAAGATCTCAGCTCTTGATGAATATCATCATAGGACCAGGAAAACTGTTCTCTGA | | | | | |
| CAGGCTACCTCACTTTGATGGACCTTGAGAGAGGAACAATATATATGGGGGATTCT | | | | | |
| TCACAAAAGGGATGTAAGTTCCACTGGAGGGAATCTACCATGTTTGACATTGCCAC | | | | | |
| AAGTCTATCGTCAAAATGTTCTTTTGTGCAATACCTGTGATGCACACAAAGCGATT | | | | | |
| ACTCCTCCTATGGTCCTCTAGATACTAGCTGGGAAGAGCCTCTCTTCACCCCTTAA | | | | | |
| GATTATTTTTCTCCTCATCCTATGGCACTGTGCAAACACAGAAGTCTTCTTTGGTA | | | | | |
| AAGGAACCAGACTCACAGTTGTAGGTAAGATATCTTTCAGGTAAATTTCCAGGTCT | | | | | |
| CTCTTCGGACAAAGCATGTCCTCCGTGTCCATATTCGAGTATCTGTATTCTGATGT | | | | | |
| GCAAACTCCGACTACACCTTCGGCTCAGGGACCAGGCTTTTGGTAATAGGATTGCG | | | | | |
| GCTTTCCTATGCAAGCCACCACAGCTCTCTTACATCTCAGTGTAGGAGTGAATGTG | | | | | |
| GAACATCAGAGGATCTGAGAAATGTGACTCCACCCAAGGTCTCCTTGTTTGAGCCA | | | | | |
| TCAAAAGCAGAGATTGCAAACAAACAAAAGGCTACCCTCGTGTGCTTGGCC | | | | | |
| ACACCAGGAAGCTCCTTTTGAACATCTCAAGAGTCTTCCTACCATTCTAGTCATCA | 780 | TRUE | TRB | FALSE | NA |
| TAGTTAGCCAGGTGGAGCCCCTGCATAGCGCGTCTCCTTGTTTCTCTTTTAACTAA | | | | | |
| TGCCCAGAGCCAAAGAAAGTCCCTCCAAACTATGAACAAGTGGGTTTTCTGCTGGG | | | | | |
| TAACCCTTTGTCTCCTTACTGTAGAGACCACACATGGTGATGGTGGCATCATTACT | | | | | |
| CAGACACCCAAATTCCTGATTGGTCAGGAAGGGCAAAAACTGACCTTGAAATGTCA | | | | | |
| ACAGAATTTCAATCATGATACAATGTACTGGTACCGACAGGATTCAGGGAAAGGAT | | | | | |
| TGAGACTGATCTACTATTCAATAACTGAAAACGATCTTCAAAAAGGCGATCTATCT | | | | | |
| GAAGGCTATGATGCGTCTCGAGAGAAGAAGTCATCTTTTTCTCTCACTGTGACATC | | | | | |
| TGCCCAGAAGAACGAGATGGCCGTTTTTCTCTGTGCCAGCAGTATGCACAGCAACG | | | | | |
| AAAGATTATTTTTCGGTCATGGAACCAAGCTGTCTGTCCTGGAGGATCTGAGAAAT | | | | | |
| GTGACTCCACCCAAGGTCTCCTTGTTTGAGCCATCAAAAGCAGAGATTGCAAACAA | | | | | |
| ACAAAAGGCTACCCTCGTGTGCTTGGCC | | | | | |

### 3) Realignment of V(D)J Sequences to the GRCm38 VDJ Reference

Immune repertoire profiles were aligned using Cell Ranger v7.2.0 and the GRCm38 VDJ reference. In order to capture the unproductive or partially recombined sequences, the outputted files were realigned with igblast v1.22.0 using the dandelion python package (v0.3.5) 11 with reannotate genes and the flavor set to “original”. Applicants compared strain and non-strain specific alignments and had improved performance with the non-strain-specific realignment. Contig designations were assigned as previously describe). Single-cell contig information for TRA and TRB locus was attached to the single-cell object using a modified form of combine TCR( ) from scRepertoire v2.0.0 allowing for sequences designated as unproductive or with NAs in the junctional assignments.

Applicants' analysis predominantly identified unproductive or germline T cell receptor beta (TRB) rearrangements within cells from clusters I and II. By contrast, although the majority of cells in cluster III also presented with non-functional TRB and T cell receptor alpha (TRA) rearrangements, a subset began to express fully recombined and functional TRB or combined TRA/TRB chains (FIG. 14F, Table 4). This heterogeneity may reflect the early analysis time point (7-days post treatment onset), resulting in only 2.5% of all sequenced clones exhibiting a functional TCR configuration. Notably, Applicants also detected TCR germline transcription alongside missing V genes in several instances of non-productive TRA and TRB sequences across clusters. This suggests that the TRA and TRB loci acquire an accessible state, poised for active V(D)J recombination (FIG. 14G).

By reassigning the transcriptional clusters based on the sequence of their V(D)J genes, Applicants achieved a clearer segregation that aligns with the established sequential trajectory of T cell maturation (FIG. 14H). This progression is further delineated by the co-expression of phase II TFs and, ultimately, the strong expression of the CD3/TCR complex alongside an increase in Thymocyte selection-associated high mobility group box protein (Tox), indicative of successful (3-selection (FIG. 14H).

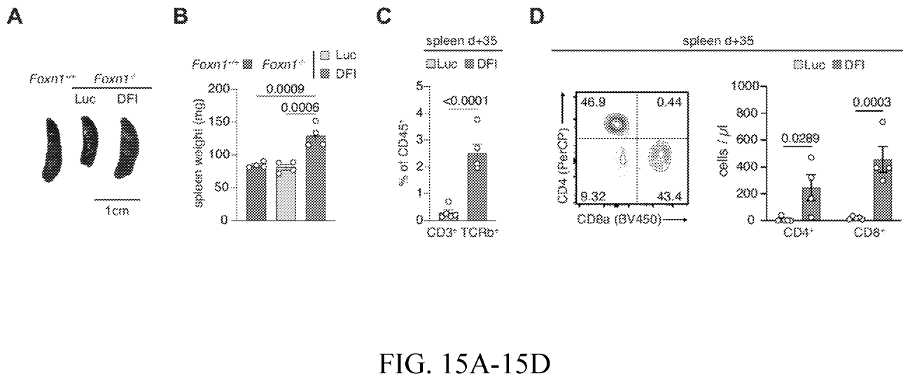

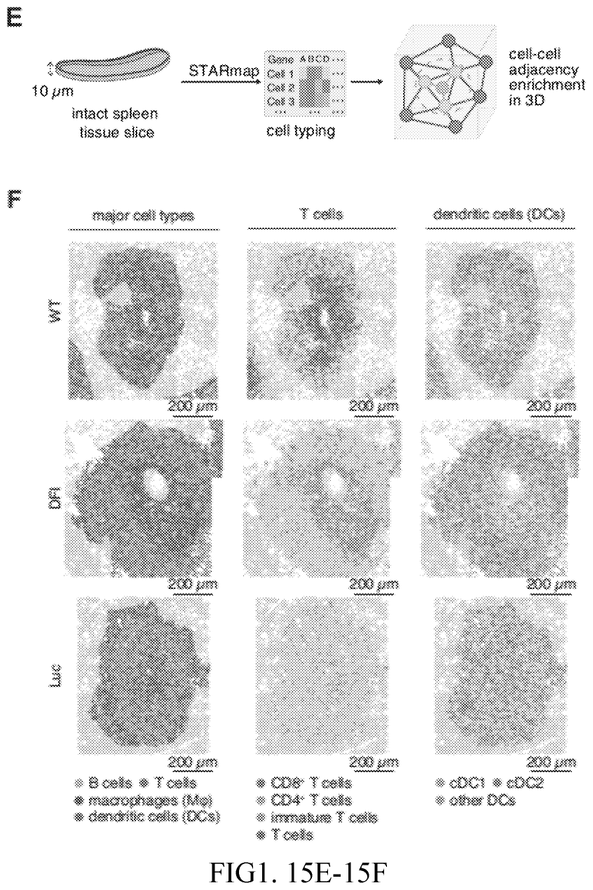

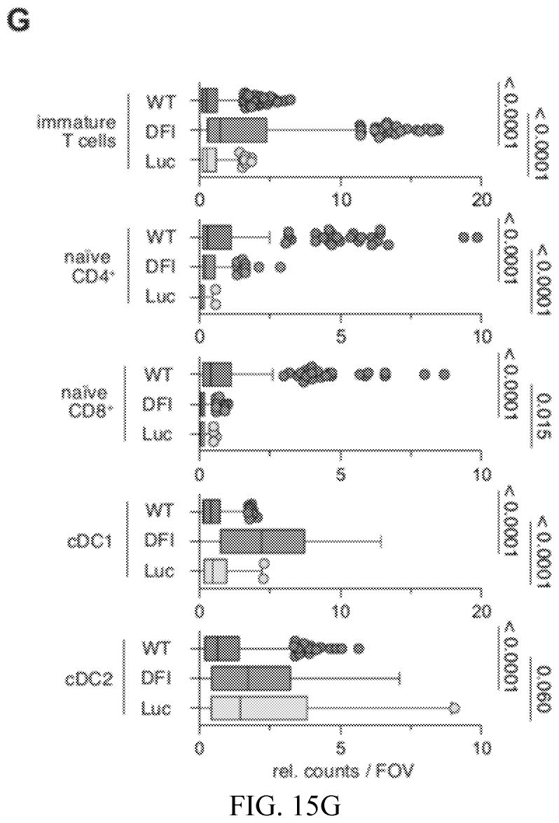

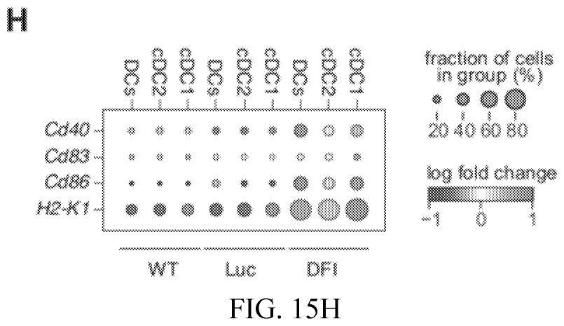

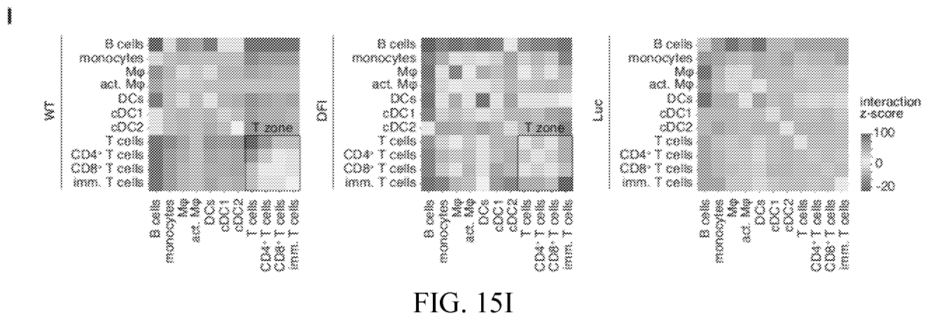

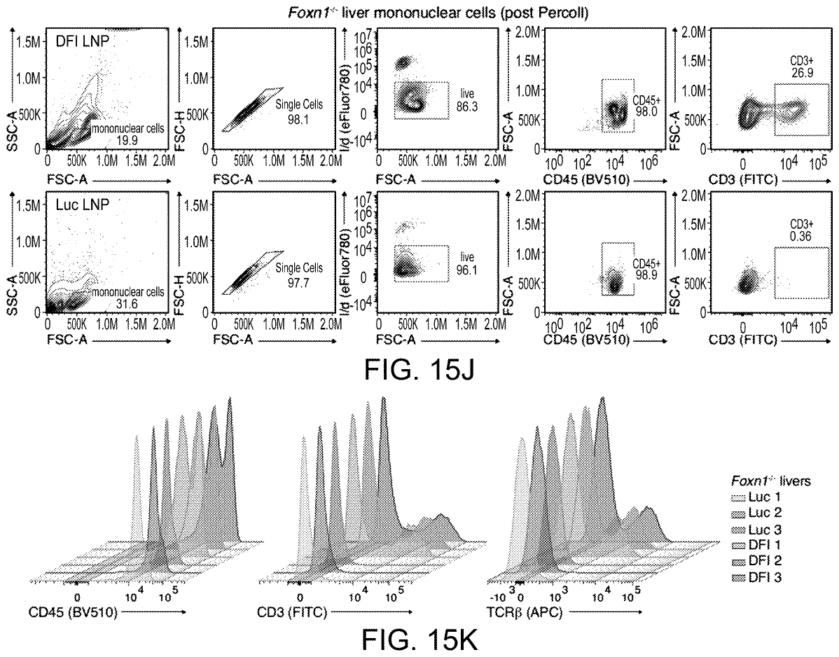

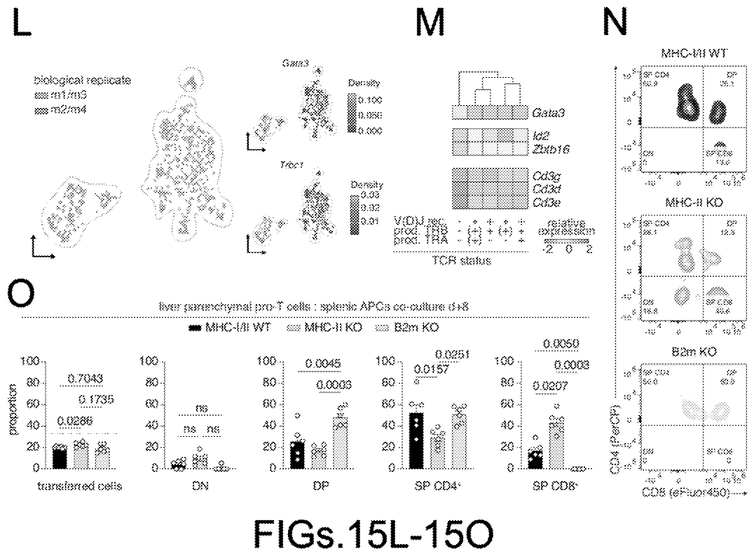

Encouraged by the detection of rare, but productively recombined TCRab T cells in the liver parenchyma, Applicants tested if animals treated for 35 days built a TCR repertoire in the absence of a thymus by analyzing the peripheral blood by flow cytometry and deep V(D)J sequencing. Flow cytometry analysis revealed that CD3⁺ TCRβ⁺ cells constituted approximately 10% of the CD45⁺ cell population (compared to around 20% T cells typically observed in C57BL/6J wild-type mice; FIG. 14I). Interestingly, although many of these cells were CD4⁻/CD8⁻ about 40% were CD4⁻/CD8⁺. This suggests they had successfully undergone positive selection, a key process in T cell maturation following successful V(D)J recombination (FIG. 14I-J). In the peripheral blood of these athymic animals, Applicants observed predominantly CD4⁻/CD8⁺ T cells, but no CD4⁺ T cells (FIG. 14J). Although both Notch and IL-7 signaling favor differentiation of CD8⁺ T cells, this observation aligns with previous studies in in vitro T cell differentiation cultures, which attributed limitations in generating CD4⁺ cells to a lack of Major Histocompatibility Complex (MHC) class II expression or inefficient TCR-MHC interactions in such experimental setups. Nevertheless, CD4⁻/CD8⁺ T cells can be positively selected in vitro presumably because the feeder cells also express ubiquitous MHC class I ligands, which can be recognized by MHC class I restricted TCRs. Applicants therefore tested if a similar mechanism could achieve positive selection in athymic DFI-treated animals using ex vivo differentiation co-cultures with different MHC-transgenic microenvironments. Co-culture of CD3⁺ cells isolated from the livers of DFI-treated mice at d+7 with T cell-depleted splenocytes from B2M^{-/-} mice (which lack MHC class I expression) showed no formation of CD4⁻/CD8⁺ T cells (FIGS. 15N-150). However, maturation to CD4⁻/CD8⁺ T cells occurred when co-cultured with wild-type splenocytes, while a notable increase in CD4⁺/CD8⁺ (double positive, DP) T cells was observed in B2M^{-/-} co-cultures, indicating a block in maturation beyond the DP stage in the absence of MHC class I (FIGS. 15N-150). Applicants conclude positive selection and successful maturation of CD4⁻/CD8⁺ T cells from pro-T cells generated outside the thymus, specifically in the DFI-reprogrammed liver niche, depends on MHC class I molecules.

In contrast, in the peripheral blood, ex vivo co-cultures produced CD4⁺/CD8⁻ T cells, suggesting that liver-generated pro-T cells can in principle differentiate into the CD4⁺ T cell lineage. However, co-culture with splenocytes lacking all murine MHC class II molecules (2-Abl^{-/-}, H2-Aa^{-/-}, H2-Eb1^{-/-}, H2-Eb2^{-/-}, H2-Ea^{-/-}) resulted in a significant reduction of these events (FIG. 15N-15O).

Considering that MHC class II expression is restricted to professional antigen-presenting cells (APCs) and lowly abundant in the non-inflamed non-thymic environments, it is plausible that the maturation of CD4⁺ T cells in DFI-treated athymic mice will only occur within secondary lymphoid organs, such as lymph nodes and the spleen, both of which have a specialized role in supporting the maturation and differentiation of immune cells.

Athymic mice have been reported to possess a very limited quantity of mature T cells, a condition that is particularly pronounced in adult specimens. In scenarios lacking sufficient T cells, naïve T cells are known to undergo spontaneous homeostatic proliferation upon encountering self-MHC/peptide ligands, a process that depends on the presence of IL-7. To ascertain whether the observed increase in T cell numbers stemmed from the actual creation of new T cell clonotypes rather than from homeostatic proliferation triggered by Il7 mRNA delivery, blood samples from Foxn1^{-/-}+Luc and Foxn1^{-/-}+DFI treated mice were analyzed by deep V(D)J sequencing. To quantitatively assess the diversity of TCRs in these groups, Applicants computed two metrics: clonality, which utilizes Simpson indices for normalization across sample sizes, and power law distribution slopes. The Simpson clonality index, favored over the Shannon clonality due to its lower sensitivity to sequence count disparities, ranges from 0 to 1, where 1 indicates minimal diversity (a single clone population) and 0 signifies maximum diversity (all clones present at equal frequencies). The assumption that DFI results in increased homeostatic T cell proliferation was not supported by the findings; the Simpson clonality scores were similar across both treatment groups, suggesting the introduction of new TCRs rather than mere expansion of existing clones (FIG. 14L).

### 4) VDJ Deep Sequencing

Genomic DNA was isolated from snap-frozen spleen samples using QIAamp DNA isolation kit (QIAGEN) as per the manufacturer's instructions. TCR beta chain (TCRB) deep sequencing was performed on purified DNA to detect rearranged TCRβ gene sequences using mTCRB Kit (Adaptive Biotechnologies) according to the manufacturer's protocol. The prepared library was sequenced by Adaptive Biotechnologies. Data processing (demultiplexing, trimming, gene mapping) was done using the Adaptive Biotechnologies proprietary platform as previously described.

Consistent with prior studies in Foxn1^{-/-} mice, Applicants identified a scarce number of TCR rearrangements, with the average count of unique clonotypes being fewer than 50 and the repertoire being predominantly occupied by a few highly expanded clones (FIG. 14K). By contrast, mice treated for 35 days with DFI showed a significant increase in the number of TCR rearrangements, with up to 400 unique rearrangements (FIG. 14K-L). Together, these data align with previous findings that DFI treatment, but not Il7 mRNA alone, elevates naïve T cell counts in immunocompetent, but aged animals.

Overall, the V(D)J sequencing analysis corroborates the development of a rudimentary, yet diverse TCR repertoire in DFI-treated athymic mice by the addition of novel TCR clonotypes, further characterized by the absence of a dominant CDR3 length or Vβ/Jβ family preference.

### Example 5—Accumulation of Functional T Cells in the Spleen

Applicant's prior experiments indicated that full maturation of T cell progenitors induced by DFI-exposed hepatocytes requires MHC class I and II-competent APCs. Applicants therefore hypothesized that the ectopically generated T cell progenitors that Applicants found circulating in the blood might either die of neglect or home to secondary lymphoid organs to mature. Given that the spleen is essential for T cell immunity, playing a critical role in the migration and activation of T cells, and that it is the primary location where T cells interact with MHC class II-competent APCs, such as dendritic cells (DCs), Applicants tested if the T cell progenitors in DFI-treated animals can be detected in the spleen. Applicants explanted spleens after 35 days of DFI treatment and 24 hours after the last injection. Applicants observed a substantial weight and size increase of spleens from DFI-treated animals relative to Luc LNP treated controls as well as age-matched Foxn1^{-/-} animals (FIG. 15A-15B). Applicants next performed flow cytometry and found an increase in overall CD45⁺ immune cells and specifically CD3⁺ TCRβ⁺ cells in DFI-treated animals (FIG. 15C). Phenotyping the CD3⁺ TCR⁺ cell fraction revealed a significantly increased abundance of both single-positive CD4⁺ and CD8⁺ T cells in DFI-treated animals relative to Luc-mRNA-treated controls (FIG. 15D).

To better understand how the DFI affects the splenic architecture and maturation of T cells, Applicants performed in situ sequencing using STARmap to characterize the types of immune cells present in wild-type, athymic DFI-treated, and athymic Luc-treated animals.

### 1) STARmap Protocol

STARmap padlock and primer probes were designed as described in Wang et al (Wang et al., Science 361 (2018)). For the 64-gene STARmap data collection, 2-6 pairs of primer and padlock probes (Tables 2 and 3) were designed for each gene. The mice used in this study were anesthetized with isoflurane and rapidly decapitated. The mouse spleen tissue was collected and placed in Tissue-Tek O.C.T. Compound. Two biological replicates 7 were collected for each condition (Luc, DFI, and WT). Then the spleen tissue in O.C.T. was frozen in liquid nitrogen and stored at −80° C. For mouse spleen tissue sectioning, the spleen tissue was transferred to cryostat (Leica CM1950) and cut into 20 μm cross sections at −20° C. The slices were transferred and attached to glass-bottom 24-well plates pretreated with 3-(Trimethoxysilyl)propyl methacrylate and poly-D-lysine.

The STARmap procedure was conducted as described in Wang (Wang et al., Science 361 (2018). Briefly, the spleen slices were fixed with 400 μl 4% PFA in 1×PBS at room temperature for 15 min, then permeabilized with 600 μl prechilled methanol at −20° C. for an hour. The samples were then taken from −20° C. freezer to room temperature for 5 min, then quenched with 400 μl quenching solution (0.1 mg/ml Yeast tRNA, 0.1 U/μl SUPERase In RNase Inhibitor, 100 mM Glycine, 0.1% Tween-20 in PBS) at room temperature for 10 min. After quenching, the samples were rinsed with 600 μl PBSTR (1×PBS supplemented with 0.1% Tween-20 and 0.02 U/μl SUPERase In RNase Inhibitor) twice. Then the samples were incubated with 200 μl of 1× hybridization buffer (2×SSC, 10% formamide, 20 mM Ribonucleoside vanadyl complex, 0.1 mg/ml yeast tRNA, 0.1 U/μl SUPERase In, 1% Triton-X-100, pooled padlock and primer probes at a concentration of 10 nM per oligo) at 40° C. in a humidified oven with parafilm wrapping and shaking for 20 h. The samples were washed with 300 μl PBSTR twice and 300 μl high-salt washing buffer (4×SSC dissolved in PBSTR) once, for 20 min at 37° C. for each wash. Finally, the samples were rinsed once with 300 μl PBSTR at room temperature.

The samples were then incubated with a 200 μligation mixture (0.1 Weiss U/μl T4 DNA ligase, 0.5 mg/ml BSA, and 0.2 U/μl of SUPERase In RNase inhibitor in 1×T4 DNA ligase buffer) at room temperature for three hours with gentle shaking. After the ligation reaction, the samples were washed twice with 300 μl PBSTR and then incubated with 200 μl rolling circle amplification (RCA) mixture (0.2 Weiss U/μl Phi29 DNA polymerase, 250 μM dNTP, 20 μM 5-(3-aminoallyl)-dUTP, 0.5 mg/ml BSA and 0.2 U/μl of SUPERase In RNase inhibitor in 1×Phi29 buffer) at 4° C. for 30 min then 30° C. for three hours with gentle shaking. The samples were washed twice with PBST (0.1% Tween-20 in PBS) before being temporarily stored at 4° C. overnight.

The next day, the samples were treated with 300 μl freshly prepared modification mixture (20 mM methacrylic acid NHS ester in 100 mM sodium bicarbonate buffer) at room temperature for one hour and then washed once by PBST for 5 min. The samples were rinsed once and then incubated with 200 μl monomer buffer (4% acrylamide, 0.2% bis-acrylamide, 0.2% tetramethylethylenediamine in 2×SSC) at 4° C. for 15 min. Then the buffer was aspirated, and 50 μl polymerization mixture (0.2% ammonium persulfate dissolved in pre-cooled monomer buffer) was added to the center of the sample and immediately covered by Gel Slick-coated glass coverslip (Electron Microscopy Sciences, 72226-01). The polymerization reaction was performed for one hour at room temperature in an N2 box, then washed by PBST twice for 5 min each. The samples embedded in hydrogel were digested with 300 μl Proteinase K mixture (0.5 mg/ml Proteinase K, 1% SDS in 2×SSC) at 37° C. for three hours, then washed by PBST three times for 5 min each. Subsequently, the samples were treated with 200 μl dephosphorylation mixture (0.25 U/μl Antarctic Phosphatase, 0.5 mg/mL BSA in 1× Antarctic Phosphatase buffer) at 37° C. overnight and washed by PBST three times for 5 min each.

For SEDAL sequencing, each sequencing cycle began with treating the sample with 800 μl stripping buffer (60% formamide, 0.1% Triton X-100 in H2O) at room temperature twice for 10 min each, followed by washing with 1 mL PBST three times for 5 min each. Then the samples were incubated with a250 μl sequencing mixture (0.1875 U/μl T4 DNA ligase, 0.5 mg/ml BSA, 10 μM reading probe, and 5 μM decoding probes in 1×T4 DNA ligase buffer) at room temperature 8 for at least 5 hours. The samples were washed with 900 μl washing and imaging buffer (10% formamide in 2×SSC buffer) three times for 10 min each, then immersed in washing and imaging buffer for imaging. Images were acquired using Leica TCS SP8 confocal microscopy with 40× oil immersion objective (NA 1.3) and voxel size of 194 nm×194 nm×350 nm (x×y×z). DAPI staining was performed before the first cycle using 5×DAPI in PBST for 3 hours at room temperature. The DAPI signal was collected at the first cycle of imaging. Four cycles of imaging were performed to decode 64 genes.

After SEDAL sequencing, the samples were treated with 800 μl stripping buffer three times at room temperature for 10 min each. The samples were then washed with 1 mL PBST three times for 5 min each. To visualize the plasma membrane and aid cell segmentation, the samples were treated with 300 μl Flamingo staining mixture (1× Flamingo Fluorescent Gel Stain and 5×DAPI in PBS) at room temperature overnight, then washed with 300 μl PBST three times for 5 min each. The Flamingo signal was collected and the DAPI signal was reimaged while the samples were immersed in PBST with 0.1× Flamingo Fluorescent Gel Stain.

### 2) STARmap Primer Probes

TABLE 2

| Target | | | | |
|---|---|---|---|---|
| No | Gene Symbol | Probe index | Probe sequence | SEQ ID NO: |
| 1 | Cd8a | 1 | AGCAGAAATAGTAGCCTTCGTTTTTATTTTTATCTT | 1 |
| 1 | Cd8a | 2 | TCTTGTTGTGGGATGAAGCCTATTITTATCTT | 2 |
| 1 | Cd8a | 3 | GTCCACCTTCTGACCAAGTTTATTTTTATCTT | 3 |
| 1 | Cd8a | 4 | CCTCATGGCAGAAAACAGTTTTATTTTTATCTT | 4 |
| 1 | Cd8a | 5 | CTAGCGGCTGTGGTAGCAGTATTTTTATCTT | 5 |
| 1 | Cd8a | 6 | AGAGCAAGTTGAGATACTCTCCTATTTTTATCTT | 6 |
| 2 | Cd3d | 1 | GGCCTTCCGGTCTCATGTCCTATTGTTATCTT | 7 |
| 2 | Cd3d | 2 | GGGCCAGTTCCCTCCAAGATATTGTTATCTT | 8 |
| 2 | Cd3d | 3 | CCTCGTGGGTCCAGAACGCTATTGTTATCTT | 9 |
| 2 | Cd3d | 4 | TGCAAACCATCCTTCCACCTATTGTTATCTT | 10 |
| 2 | Cd3d | 5 | TGCAGGTCACAAATACTTTGTATTGTTATCTT | 11 |
| 2 | Cd3d | 6 | CTTCCAGAGGGAATCATGCATATTGTTATCTT | 12 |
| 3 | Lef1 | 1 | GCAGCTGTCATTCTGGGACCTTATTCTTATCTT | 13 |
| 3 | Lef1 | 2 | ATGAAAGCATTCAGAGGCTTCTTTATTCTTATCTT | 14 |
| 3 | Lef1 | 3 | GTCGCTGTTCATATTGGGCTATTCTTATCTT | 15 |
| 3 | Lef1 | 4 | GCTCCTGTTTGACCTGAGGTATTCTTATCTT | 16 |
| 3 | Lef1 | 5 | GGTCGCTGGCTTTCTAGTTGTATTCTTATCTT | 17 |
| 3 | Lef1 | 6 | AGTGAGGATGGGTAGGGTTGCTATTCTTATCTT | 18 |
| 4 | Cd44 | 1 | ATTTCCTTCTATGAACCCATACCTATTATTATCTT | 19 |
| 4 | Cd44 | 2 | AGATAGCGTTGGGATGAATCTATTATTATCTT | 20 |
| 4 | Cd44 | 3 | GGTAGGTGTGCAAAATGTAGCTTTCTATTATTATCTT | 21 |
| 4 | Cd44 | 4 | CTGGTTCGCACTTGAGTGTTATTATTATCTT | 22 |
| 4 | Cd44 | 5 | GGAGGTGTTGGACGTGACGTATTATTATCTT | 23 |
| 4 | Cd44 | 6 | ACGTCTCCAATCGTGCTGTTATTATTATCTT | 24 |
| 5 | Il2ra | 1 | CTGCAGTGACCTGTAAGGTTATGTTTATCTT | 25 |
| 5 | Il2ra | 2 | CTGTAGAGCCTTGTATCCCGTATGTTTATCTT | 26 |
| 5 | Il2ra | 3 | ACCTCTCTTGCATTCACAGTTTAGGTATGTTTATCTT | 27 |
| 5 | Il2ra | 4 | ACTCCATTGTGAGCACAAATTATGTTTATCTT | 28 |
| 5 | Il2ra | 5 | CCACCCCGTTTTCCCACACTATGTTTATCTT | 29 |
| 5 | Il2ra | 6 | TCCAGTGTTTTCACGGTATGTTATGTTTATCTT | 30 |
| 6 | Kit | 1 | TGGCATAACACATGAACACTCTATGGTTATCTT | 31 |
| 6 | Kit | 2 | GCATCAGAGTTGGACACCAGTATGGTTATCTT | 32 |
| 6 | Kit | 3 | TCTCCTCGACAACCTTCCATATGGTTATCTT | 33 |
| 6 | Kit | 4 | GGAGCATGCCATTTATGAGCCTATGGTTATCTT | 34 |
| 6 | Kit | 5 | AATGTGTCCCCTTTCTTAAGTATGGTTATCTT | 35 |
| 6 | Kit | 6 | ATCCTGTAAGGACAACACATGATATGGTTATCTT | 36 |
| 7 | Cd28 | 1 | AGATTCCAGAGACGGAACGTCATATGCTTATCTT | 37 |
| 7 | Cd28 | 2 | TGCCCGGAATTCCTTTGCGTATGCTTATCTT | 38 |
| 7 | Cd28 | 3 | ATAGTTCCATTGCTCCTCTCGTATGCTTATCTT | 39 |
| 7 | Cd28 | 4 | AGCCACACAGACTTTATCCTATGCTTATCTT | 40 |
| 7 | Cd28 | 5 | CGAAACTGGGGCTGATAGGTATGCTTATCTT | 41 |
| 7 | Cd28 | 6 | CTCCAGCAACCACGACCAGTATGCTTATCTT | 42 |
| 8 | Cd3g | 1 | AGTCTTGTCAGTCAAGCCACATATGATTATCTT | 43 |
| 8 | Cd3g | 2 | TGTCCCGCAATGAGATATACTATGATTATCTT | 44 |
| 8 | Cd3g | 3 | CTGCTTGTCTGAAGCTCTTGACTATGATTATCTT | 45 |
| 8 | Cd3g | 4 | TGGTGCCTATGTTTAGCTCTATGATTATCTT | 46 |
| 8 | Cd3g | 5 | ATGCTGATGACCTCAGCGAAGATTATGATTATCTT | 47 |
| 8 | Cd3g | 6 | CCTTGGAGATGGCTGTACTGGTCATATGATTATCTT | 48 |
| 9 | Cd3e | 1 | CTCGTCACTGTCTAGAGGGCTATCTTTATCTT | 49 |
| 9 | Cd3e | 2 | GCCCAGAGTGATACAGATGTTATCTTTATCTT | 50 |
| 9 | Cd3e | 3 | TCAGACTGCTCTCTGATTCATATCTTTATCTT | 51 |
| 9 | Cd3e | 4 | TGGAGATGACCATCCGAGGATATCTTTATCTT | 52 |
| 9 | Cd3e | 5 | GCTGTAGGAAAGGGCTTAGCTATCTTTATCTT | 53 |
| 9 | Cd3e | 6 | GGGTCCACAGAAGGCGATGTATCTTTATCTT | 54 |
| 10 | Cd247 | 1 | TCCTCTCTTCGCCCTAGATTGAGTATCGTTATCTT | 55 |
| 10 | Cd247 | 2 | GGCTGTGATGATGACTCCGTATCGTTATCTT | 56 |
| 10 | Cd247 | 3 | TGCTCACTTCTGAGTAGAGCTATCGTTATCTT | 57 |
| 10 | Cd247 | 4 | GGCGGGCTCTTAACCCAAGTATCGTTATCTT | 58 |
| 10 | Cd247 | 5 | GCAAGTAGCAGAGTTTGGGATCCTATCGTTATCTT | 59 |
| 10 | Cd247 | 6 | ACCCTGGTAAAGGCCATCGTGCCCTATCGTTATCTT | 60 |
| 11 | Rag1 | 1 | ACTCACAAACTGCGTCTACAGTCTATCCTTATCTT | 61 |
| 11 | Rag1 | 2 | AGTTCCGCGAAACGCTGTGTATCCTTATCTT | 62 |
| 11 | Rag1 | 3 | GGCCCCGATAGAGCCATCCTATCCTTATCTT | 63 |
| 11 | Rag1 | 4 | CCTGGCATTCATTTTCCGAAATATCCTTATCTT | 64 |
| 11 | Rag1 | 5 | GCATCTATGGAAGGGACTGTCTCTATCCTTATCTT | 65 |
| 11 | Rag1 | 6 | AGAGGGACTCTGGACACTCTTATCCTTATCTT | 66 |
| 12 | Rag2 | 1 | CCATCTCACTGATTTCAATCGTGTTTATCATTATCTT | 67 |
| 12 | Rag2 | 2 | ACCACGTCAATGGAATGGCTATCATTATCTT | 68 |
| 12 | Rag2 | 3 | GGAAGGATTTCTTGGCAGGAGTATCATTATCTT | 69 |
| 12 | Rag2 | 4 | CTCATGGCCATGCTTTCTTATATCATTATCTT | 70 |
| 12 | Rag2 | 5 | ATGGCGGGTTTATTGAGCTCTATCATTATCTT | 71 |
| 12 | Rag2 | 6 | AGACAGAGATTCCTCCTGGCAAGATATCATTATCTT | 72 |
| 13 | Hes1 | 1 | AGAGGCCGTCTTTGGTTTGTCCTATATTTATCTT | 73 |
| 13 | Hes1 | 2 | CCTCTTCTCCATGATAGGCTTATATTTATCTT | 74 |
| 13 | Hes1 | 3 | AGGGCTACTTAGTGATCGGTTATATTTATCTT | 75 |
| 13 | Hes1 | 4 | CCGGGAGCTATCTTTCTTAAGTATATTTATCTT | 76 |
| 13 | Hes1 | 5 | ACCGGGATGACCGGGCCGCTATATTTATCTT | 77 |
| 13 | Hes1 | 6 | CGCCACGGTCTCCACATGGAGTATATTTATCTT | 78 |
| 14 | Tcf7 | 1 | CTGCCTGTGTTTTCAGGTTTATAGTTATCTT | 79 |
| 14 | Tcf7 | 2 | GTGGACTGCTGAAATGTTCGTATAGTTATCTT | 80 |
| 14 | Tcf7 | 3 | ATCTCCTTCATGTAAAGCATTATAGTTATCTT | 81 |
| 14 | Tcf7 | 4 | TGACCACAGAGTTATTTTGTGTGATATAGTTATCTT | 82 |
| 14 | Tcf7 | 5 | GAAGGCAGAGTAGACAGTCTTATAGTTATCTT | 83 |
| 14 | Tcf7 | 6 | GGTGCACACTGGGTTTAGGTATAGTTATCTT | 84 |
| 15 | Gata3 | 1 | AGTTTCCGTAGTAGGACGGGACTATACTTATCTT | 85 |
| 15 | Gata3 | 2 | CGGATGCATGGGCGTCGGTTATACTTATCTT | 86 |
| 15 | Gata3 | 3 | GGTGGTGGTGGTCTGACAGTTATACTTATCTT | 87 |
| 15 | Gata3 | 4 | ACGGTTGCTCTTCCGATCACTATACTTATCTT | 88 |
| 15 | Gata3 | 5 | AAAACACAGATCTGTCGCTTTATACTTATCTT | 89 |
| 15 | Gata3 | 6 | CCCGGTCAGATTGCGTAGCTATACTTATCTT | 90 |
| 16 | Bcl11b | 1 | GCTGGAAGGCTCATCTTTACGTATAATTATCTT | 91 |
| 16 | Bcl11b | 2 | GCCTGCTCGATTTTGACATCATTATAATTATCTT | 92 |
| 16 | Bcl11b | 3 | CGAGCCACTGCGAGTACACTATAATTATCTT | 93 |
| 16 | Bcl11b | 4 | GTTGAAGGGCTGCTTGCATTATAATTATCTT | 94 |
| 16 | Bcl11b | 5 | ACTGCCTTAATCAACCCTCATATAATTATCTT | 95 |
| 16 | Bcl11b | 6 | TCCCTCTACGTCACTTGCGTATAATTATCTT | 96 |
| 17 | Ets1 | 1 | ACCACTGAAAGTAGCTTTCAAGTAGTITTATCTT | 97 |
| 17 | Ets1 | 2 | AGGGACAGGAGTTCTTCCGATAGTTTTATCTT | 98 |
| 17 | Ets1 | 3 | GCATGCTCGATACCGTAGCTAGTITTATCTT | 99 |
| 17 | Ets1 | 4 | ACCTCTTGCTTGATGGCAAAGTAGTTTTATCTT | 100 |
| 17 | Ets1 | 5 | TTCTTTGCTGCTCGGAGTTATAGTTTTATCTT | 101 |
| 17 | Ets1 | 6 | AAAGAGCATCAATGCAGCAATAGTTTTATCTT | 102 |
| 18 | Lyl1 | 1 | CTGGAGGCACATACCATCTCAGTAGTGTTATCTT | 103 |
| 18 | Lyl1 | 2 | ACAGGATCATGGATCAAGCGCTAGTGTTATCTT | 104 |
| 18 | Lyl1 | 3 | TGGACCCCACGGATAGAATGTAGTGTTATCTT | 105 |
| 18 | Lyl1 | 4 | AAGAGTGCCTGAGTCTTCGTTAGTGTTATCTT | 106 |
| 18 | Lyl1 | 5 | CCCAAGGCAGTTAGCTGCAGCAAGTAGTGTTATCTT | 107 |
| 18 | Lyl1 | 6 | GCCAAGTCCAGCTCACTATGGCTTGTAGTGTTATCTT | 108 |
| 19 | Hhex | 1 | ACTGAGCTGTAACATCTTGGCCTAGTCTTATCTT | 109 |
| 19 | Hhex | 2 | AGTTAAGGTCTCAACGCAATGTAGTCTTATCTT | 110 |
| 19 | Hhex | 3 | AGTGCACCTCTAAAGTCGATAGTCTTATCTT | 111 |
| 19 | Hhex | 4 | AGGCAGCAATGTAAGATGCTAGTCTTATCTT | 112 |
| 19 | Hhex | 5 | CTCCAGCTCGACGGTCTGGTCTAGTCTTATCTT | 113 |
| 19 | Hhex | 6 | GTGCGTGTAGTCGTTCACCGTCCTAGTCTTATCTT | 114 |
| 20 | Bcl11a | 1 | GGGGCAAATTCCTCTAGATTAGTATTATCTT | 115 |
| 20 | Bcl11a | 2 | CCCCTCCAGTGCAGAAGTTTAGTATTATCTT | 116 |
| 20 | Bcl11a | 3 | GGGTCATGCTCATTTCCACAGTAGTATTATCTT | 117 |
| 20 | Bcl11a | 4 | ACATCACAATCTAGCAGGATATAGTATTATCTT | 118 |
| 20 | Bcl11a | 5 | GGTCAGGGGACTTCCGTGTTAGTATTATCTT | 119 |
| 20 | Bcl11a | 6 | AGCTTCCATCCGAAAACTGCCATAGTATTATCTT | 120 |
| 21 | Hoxa9 | 1 | GGACTGGAAGCTGCAAGGACTAGGTTTATCTT | 121 |
| 21 | Hoxa9 | 2 | AGTCAGGGACAAAGTGTGAGTGTAGGTTTATCTT | 122 |
| 21 | Hoxa9 | 3 | TCACCAAGGATACAAATCTACAGTATAGGTTTATCTT | 123 |
| 21 | Hoxa9 | 4 | GGAAAGTCAGGAGCGACCATAGGTTTATCTT | 124 |
| 21 | Hoxa9 | 5 | GCACATACAGCCAATAGCGCTAGGTTTATCTT | 125 |
| 21 | Hoxa9 | 6 | TCTGCACACACGTATAGCTTAGGTTTATCTT | 126 |
| 22 | Mef2c | 1 | ACAGGATTGCTGTACACCAAATAGGGTTATCTT | 127 |
| 22 | Mef2c | 2 | ACCGCCTGTGTTACCTGCATAGGGTTATCTT | 128 |
| 22 | Mef2c | 3 | TGTTGAAATGGCTGATGGATATAGGGTTATCTT | 129 |
| 22 | Mef2c | 4 | TCCTTGTCCTGGTAAAGTAGGTAGGGTTATCTT | 130 |
| 22 | Mef2c | 5 | AGAGGCGGGAACAGATGAAGTTAGGGTTATCTT | 131 |
| 22 | Mef2c | 6 | AGTTCCCAAATCCCTGCATTCTAGGGTTATCTT | 132 |
| 23 | Lmo2 | 1 | ATCTTGGTCCACTCGTAGATGTTAGGCTTATCTT | 133 |
| 23 | Lmo2 | 2 | CGGAGTTGATGAGAAGGTATCTAGGCTTATCTT | 134 |
| 23 | Lmo2 | 3 | GGTGATACACTTTGTCTTTCACCTAGGCTTATCTT | 135 |
| 23 | Lmo2 | 4 | CCATGAACACCAAGGTCACATCTAGGCTTATCTT | 136 |
| 23 | Lmo2 | 5 | CTCTTTCCTTTCCTAGTGTGAATAGGCTTATCTT | 137 |
| 23 | Lmo2 | 6 | TGGCTTTCAGGAAGTAGCGGTTAGGCTTATCTT | 138 |
| 24 | Meis1 | 1 | GGCGAACACCGCTATATCTTCTAGGATTATCTT | 139 |
| 24 | Meis1 | 2 | ATCTCTCTTTAAAGCGTCATTAGGATTATCTT | 140 |
| 24 | Meis1 | 3 | GCGTACCCCACTCAGTTAACTAGGATTATCTT | 141 |
| 24 | Meis1 | 4 | ACTTGTGCACTCATTGTCGGGTAGGATTATCTT | 142 |
| 24 | Meis1 | 5 | GGCGAGGTTGTCAACGTGGTAGGATTATCTT | 143 |
| 24 | Meis1 | 6 | CGGGGTTCCTCCTGAACGATAGGATTATCTT | 144 |
| 25 | Tcf3 | 1 | TGTCCGGCTAGGGTCAAAGTAGCTTTATCTT | 145 |
| 25 | Tcf3 | 2 | GTTGAGGGGCTAGGTGAGATAGCTTTATCTT | 146 |
| 25 | Tcf3 | 3 | AGACAGTCGCTTTGTCGCTGTTTAGCTTTATCTT | 147 |
| 25 | Tcf3 | 4 | GCTTTGTTTCCAGTTCTGGTTAGCTTTATCTT | 148 |
| 25 | Tcf3 | 5 | CCCCTAATGCATGACACCCAAGTATAGCTTTATCTT | 149 |
| 25 | Tcf3 | 6 | ATGCCGCCCGCCCAGTGACTAGCTTTATCTT | 150 |
| 26 | Ikzf1 | 1 | CTCTGCTCCTATCTTGCACAGGTAGCGTTATCTT | 151 |
| 26 | Ikzf1 | 2 | TGGAAAGGCCGTTCACCCATAGCGTTATCTT | 152 |
| 26 | Ikzf1 | 3 | CCAAGAAATTTCTGAGGCATTAGCGTTATCTT | 153 |
| 26 | Ikzf1 | 4 | ATGTGAATGGTATACATGACTAGCGTTATCTT | 154 |
| 26 | Ikzf1 | 5 | ACTTGGGACATGTCTTGACTAGCGTTATCTT | 155 |
| 26 | Ikzf1 | 6 | ACCTGGGAAGACAGTTAGGTTAGCGTTATCTT | 156 |
| 27 | Runx1 | 1 | CGCGGTAGCATTTCTCAGTTCTAGCCTTATCTT | 157 |
| 27 | Runx1 | 2 | GGTGGCAACTTGTGGCGGATAGCCTTATCTT | 158 |
| 27 | Runx1 | 3 | CCGCCCGACAAACCTGAGGTAGCCTTATCTT | 159 |
| 27 | Runx1 | 4 | GCCCCGTGGAATCTATCTCTAGCCTTATCTT | 160 |
| 27 | Runx1 | 5 | ATTTACAGTGACACGATGCATAGCCTTATCTT | 161 |
| 27 | Runx1 | 6 | ACTCAGCCTTTAGGCCTCATAGCCTTATCTT | 162 |
| 28 | Tcf12 | 1 | AGGCTGCCATAACTACTGGACTTAGCATTATCTT | 163 |
| 28 | Tcf12 | 2 | CCACTGACTGGTACCTGTGATAGCATTATCTT | 164 |
| 28 | Tcf12 | 3 | ACTGACTGAATCTTCCCGATGTGTTAGCATTATCTT | 165 |
| 28 | Tcf12 | 4 | ACTGGTGTTGATGGGTTTGATAGCATTATCTT | 166 |
| 28 | Tcf12 | 5 | AGGATGCTATCTGATCCATTGATAGCATTATCTT | 167 |
| 28 | Tcf12 | 6 | GGCTCATCCCATTCGATGAACTTAGCATTATCTT | 168 |
| 29 | Gfi1 | 1 | GGTCAGACCCAGCAAGACCGTAGATTTATCTT | 169 |
| 29 | Gfi1 | 2 | AGATGTGTGGACAGCGTGGTAGATTTATCTT | 170 |
| 29 | Gfi1 | 3 | TGCCACAGATCTTACAGTCAATAGATTTATCTT | 171 |
| 29 | Gfi1 | 4 | GGCTCTCCCATACCTAGCACATAGATTTATCTT | 172 |
| 29 | Gfi1 | 5 | GCTTGATGGTGCATCTGATGATTTCTAGATTTATCTT | 173 |
| 29 | Gfi1 | 6 | CCGCTGGACGAATGTTTGGTAGATTTATCTT | 174 |
| 30 | Cd4 | 1 | CAGCGTCTTCCCTTGAGTGACTAGAGTTATCTT | 175 |
| 30 | Cd4 | 2 | TGCACTCTGTCAAGGGGTTATAGAGTTATCTT | 176 |
| 30 | Cd4 | 3 | CTCCCTCACTCTTATAGGCCGTAGAGTTATCTT | 177 |
| 30 | Cd4 | 4 | ACCAGCAAACTGAAGCGAGACCTAGAGTTATCTT | 178 |
| 30 | Cd4 | 5 | AGCTTGAGGTCTTTGGTGGTAGAGTTATCTT | 179 |
| 30 | Cd4 | 6 | AGTCCATCTTGACCTTATCACCTAGAGTTATCTT | 180 |
| 31 | Foxp3 | 1 | ACAGTGGAGAGCTGATGCATGATAGACTTATCTT | 181 |
| 31 | Foxp3 | 2 | GGCTCCTCTTCTTGCGAAATAGACTTATCTT | 182 |
| 31 | Foxp3 | 3 | AGCACACTGCCCTGAGTACTAGACTTATCTT | 183 |
| 31 | Foxp3 | 4 | AAGTAGGCGAACATGCGAGTAGACTTATCTT | 184 |
| 31 | Foxp3 | 5 | ATGGCTTGCTGATATCTCGTAGACTTATCTT | 185 |
| 31 | Foxp3 | 6 | ACACAGGCACTATCATGAGGGATAGACTTATCTT | 186 |
| 32 | Ifng | 1 | AGAAGGTAGTAATCAGGTGTGATTTAGAATTATCTT | 187 |
| 32 | Ifng | 2 | TTGGTCAGTGAAGTAAAGGTACTAGAATTATCTT | 188 |
| 32 | Ifng | 3 | AGGCTGGATTCCGGCAACAGTAGAATTATCTT | 189 |
| 32 | Ifng | 4 | AAAGAGTCTGAGGTAGAAAGTAGAATTATCTT | 190 |
| 32 | Ifng | 5 | ATAGTTATTCAGACTTTCTAGGTAGAATTATCTT | 191 |
| 32 | Ifng | 6 | GCAAAGCCAAGATGCAGTGTTAGAATTATCTT | 192 |
| 33 | Il4 | 1 | CTGTGGTGTTCTTCGTTGCTGTTACTTTTATCTT | 193 |
| 33 | Il4 | 2 | ATATGCGAAGCACCTTGGAATACTITTATCTT | 194 |
| 33 | Il4 | 3 | GGTGCAGCTTATCGATGAATCCATACTTTTATCTT | 195 |
| 33 | Il4 | 4 | GGGTTGAGACCCATCAATAGCTCTTACTTTTATCTT | 196 |
| 33 | Il4 | 5 | TGGTGGCTCAGTACTACGATACTTTTATCTT | 197 |
| 33 | Il4 | 6 | TGCAGCTCCATGAGAACACTACTTTTATCTT | 198 |
| 34 | 1117a | 1 | GGGTGACGTGGAACGGTTGTACTGTTATCTT | 199 |
| 34 | Il17a | 2 | CGCCAAGGGAGTTAAAGACTACTGTTATCTT | 200 |
| 34 | Il17a | 3 | CAGCATCTTCTCGACCCTGATACTGTTATCTT | 201 |
| 34 | Il17a | 4 | ACGAAGCAGTTTGGGACCCTACTGTTATCTT | 202 |
| 34 | Il17a | 5 | GCTTCCCAGATCACAGAGGGATACTGTTATCTT | 203 |
| 34 | Il17a | 6 | CTCTTGCTGGATGAGAACAGATACTGTTATCTT | 204 |
| 35 | Tbx21 | 1 | GGGACACTCGTATCAACAGATGTACTCTTATCTT | 205 |
| 35 | Tbx21 | 2 | TGGTGAGCTTTAGCTTCCCTACTCTTATCTT | 206 |
| 35 | Tbx21 | 3 | GGTCTCTTGGAAAGTAAAGACGTTACTCTTATCTT | 207 |
| 35 | Tbx21 | 4 | TCCTCCTCCTCTTAGTGTCTCCTACTCTTATCTT | 208 |
| 35 | Tbx21 | 5 | ACAGCTCGGAACTCCGCTTTACTCTTATCTT | 209 |
| 35 | Tbx21 | 6 | CCTCGCCTGGTGAAATGTGTACTCTTATCTT | 210 |
| 36 | Ccr7 | 1 | AGATGCCCTTACACAGGTAGATACTATTATCTT | 211 |
| 36 | Ccr7 | 2 | GCCGATGTAGTCATCGGTGACTACTATTATCTT | 212 |
| 36 | Ccr7 | 3 | GGAGCAAGGTACGGATGATAATTACTATTATCTT | 213 |
| 36 | Ccr7 | 4 | CATCTGGGCCACTTGGATGTACTATTATCTT | 214 |
| 36 | Ccr7 | 5 | GCTGCGGAACTTGACGCCGTACTATTATCTT | 215 |
| 36 | Ccr7 | 6 | AGCAGAACATGAGGAAAGGTATTACTATTATCTT | 216 |
| 37 | Sell | 1 | AGAGAGCTGCCTTTCGTTTGTTACGTTTATCTT | 217 |
| 37 | Sell | 2 | AGCCCCGAGAATGCGGTGATACGTTTATCTT | 218 |
| 37 | Sell | 3 | TCTCTGCTTCTTTAGTGAGATACGTTTATCTT | 219 |
| 37 | Sell | 4 | TTCCCAGAGTCTCGTTCCCTACGTTTATCTT | 220 |
| 37 | Sell | 5 | AGCGAGAAAATGAGATAGCCTACATACGTTTATCTT | 221 |
| 37 | Sell | 6 | AGGGGAAATTAGGAGGAGCTACGTTTATCTT | 222 |
| 38 | Adgre1 | 1 | GCACGAGGGAGACACTTGTAGTACGGTTATCTT | 223 |
| 38 | Adgre1 | 2 | GCTTCCCACAGAGTTAGAGCATACGGTTATCTT | 224 |
| 38 | Adgre1 | 3 | CAGGAGACACAGATAGGCCTACGGTTATCTT | 225 |
| 38 | Adgre1 | 4 | TTCATCCCGTACCTGACGGTACGGTTATCTT | 226 |
| 38 | Adgre1 | 5 | AGTCCTGAGTTGCACGTACAATAATTACGGTTATCTT | 227 |
| 38 | Adgre1 | 6 | AGCAAGAAAGGATGTTAACCCTACGGTTATCTT | 228 |
| 39 | Cd68 | 1 | GTTCCTTGGGCTATAAGCGTACGCTTATCTT | 229 |
| 39 | Cd68 | 2 | TCCACCCTGAATTGGGTATATACGCTTATCTT | 230 |
| 39 | Cd68 | 3 | TGGAGCTCTCGAAGAGATGATACGCTTATCTT | 231 |
| 39 | Cd68 | 4 | GGTTTTGTTGGGATTCAAAACTACGCTTATCTT | 232 |
| 39 | Cd68 | 5 | ACCAGCTAGGCTACACCAGTACGCTTATCTT | 233 |
| 39 | Cd68 | 6 | GGTGGTGGCAGGGTTATGAGTGACTACGCTTATCTT | 234 |
| 40 | Csf1r | 1 | TCCGAGGGAACTCCCACTTTACGATTATCTT | 235 |
| 40 | Csf1r | 2 | GGACCTAGGTAGGTCCAGTTGTTACGATTATCTT | 236 |
| 40 | Csf1r | 3 | CCATGGGGTCTTCAAGCTCTACGATTATCTT | 237 |
| 40 | Csf1r | 4 | TGGTCTTGCACACGTAGGTATTGTACGATTATCTT | 238 |
| 40 | Csf1r | 5 | ATGGCGAGAAGAAGTAGACCTACGATTATCTT | 239 |
| 40 | Csf1r | 6 | TGAGAGACTTAGGAAACCACCTACGATTATCTT | 240 |
| 41 | Lyz2 | 1 | TTCAGAGTTCTGGCAAACTTACCTTTATCTT | 241 |
| 41 | Lyz2 | 2 | TGAAGTCCTGTGACTTAGAGTACCTTTATCTT | 242 |
| 42 | Itgax | 1 | GGCTGCTGATCATGGTGTACTACCGTTATCTT | 243 |
| 42 | Itgax | 2 | CGAGGCTGTGTATGTGTACCTACCGTTATCTT | 244 |
| 42 | Itgax | 3 | ACACTGTGTCCGAACTCAGCTACCGTTATCTT | 245 |
| 42 | Itgax | 4 | TGTGTGATAGCCACATTTGTAGATACCGTTATCTT | 246 |
| 42 | Itgax | 5 | ACGCTGAATACGTATTCATGGGTACCGTTATCTT | 247 |
| 42 | Itgax | 6 | ATCTTTGTGTCCTACCCCAATTTACCGTTATCTT | 248 |
| 43 | Itgam | 1 | CTGGGAGAAGATGACCTGGGTACCCTTATCTT | 249 |
| 43 | Itgam | 2 | AGGGCAAACGCAGAGTCATTATACCCTTATCTT | 250 |
| 43 | Itgam | 3 | AGGTCATAAGTGACAGTGCTCTACCCTTATCTT | 251 |
| 43 | Itgam | 4 | CCATGGCACTCATGGTGATTACCCTTATCTT | 252 |
| 43 | Itgam | 5 | ACCAAGCTTTGGACACGGTTACCCTTATCTT | 253 |
| 43 | Itgam | 6 | ACCATGAGAAGTCTTGATGTACTACCCTTATCTT | 254 |
| 44 | Flt3 | 1 | AGGCTTGAGAGAAGATCCACGTGTACCATTATCTT | 255 |
| 44 | Flt3 | 2 | TCTGGGATTTCCTCCGTGCATACCATTATCTT | 256 |
| 44 | Flt3 | 3 | AGAGCAGGTGTAATATCCGGTGTACCATTATCTT | 257 |
| 44 | Flt3 | 4 | AGACGTGCCCATAGAATTGTACGTACCATTATCTT | 258 |
| 44 | Flt3 | 5 | AGGACCTTCCCAAACTCTAAGTACCATTATCTT | 259 |
| 44 | Flt3 | 6 | AGCGTGAACATTTTGGTGAACTACCATTATCTT | 260 |
| 45 | Sirpa | 1 | TCCAGTTGTGGGTAGCATTATTATCTACATTTATCTT | 261 |
| 45 | Sirpa | 2 | CTGGACACTAGCATACTCTGTACATTTATCTT | 262 |
| 45 | Sirpa | 3 | TGCTGTGCTCCTAACATCCCATACATTTATCTT | 263 |
| 45 | Sirpa | 4 | TGTCCCCTCAATGTGGAATGTACATTTATCTT | 264 |
| 45 | Sirpa | 5 | GTTGGCTCTGCTTACATGAATTACATTTATCTT | 265 |
| 45 | Sirpa | 6 | TGTTCTCTTCTTTGTACCAGGTACATTTATCTT | 266 |
| 46 | Cd19 | 1 | TCCTCGGAGACAATAATAGGTTACAGTTATCTT | 267 |
| 46 | Cd19 | 2 | CTCCAGTATCCTGGACTTGAATTACAGTTATCTT | 268 |
| 46 | Cd19 | 3 | ATGTAGGAAGGGAGAGCACATACAGTTATCTT | 269 |
| 46 | Cd19 | 4 | GGTCCTGCCCAAGGTTGGATACAGTTATCTT | 270 |
| 46 | Cd19 | 5 | CGGCTGGACATATTTGCATCATACAGTTATCTT | 271 |
| 46 | Cd19 | 6 | CACCACTGGGACTATCCATCTACAGTTATCTT | 272 |
| 47 | Cd79a | 1 | GCACCACTGCACAGAACAGTACACTTATCTT | 273 |
| 47 | Cd79a | 2 | CCGGGACCCAAACAGGCGTTACACTTATCTT | 274 |
| 47 | Cd79a | 3 | GCAGCGATTAAGGGCTCATTATACACTTATCTT | 275 |
| 47 | Cd79a | 4 | ACTTGGGGAAGGACAAGATTTACACTTATCTT | 276 |
| 47 | Cd79a | 5 | TTTCACAGGTGAGGCGGGCTACACTTATCTT | 277 |
| 47 | Cd79a | 6 | AGGGCCTAGGGACTGGATTGCTACACTTATCTT | 278 |
| 48 | Ms4a1 | 1 | CCCCAAAGCCTTTGATTCCCTTACAATTATCTT | 279 |
| 48 | Ms4a1 | 2 | AGGAGCGATCTCATTTTCCACTTACAATTATCTT | 280 |
| 48 | Ms4a1 | 3 | AAGGCAGAGATCAGCATCGTACAATTATCTT | 281 |
| 48 | Ms4a1 | 4 | AGGTTGCATGGCGAGGGGATACAATTATCTT | 282 |
| 48 | Ms4a1 | 5 | CCAGAGAGGGTACCATACACTCAAATACAATTATCT | 283 |
| | | | T | |
| 48 | Ms4a1 | 6 | ACACACATCCAAGGAAAAGGTTACAATTATCTT | 284 |
| 49 | Cd22 | 1 | GGTGACGTCTGGATTGCTGTAATTTTATCTT | 285 |
| 49 | Cd22 | 2 | AGAGGATGTCATACCCAAAGCTAATTTTATCTT | 286 |
| 49 | Cd22 | 3 | AGGAGTGCTGGACTTGGCACTAATTTTATCTT | 287 |
| 49 | Cd22 | 4 | AGCGCAAGATGGCATAACTAATAATTTTATCTT | 288 |
| 49 | Cd22 | 5 | GCGCGGATCTCTGATGCGGTAATTTTATCTT | 289 |
| 49 | Cd22 | 6 | AAGGTTGGAACGGTTTCTCTAATTTTATCTT | 290 |
| 50 | Bcl2 | 1 | AGCTGACTGGACATCTCTGCGATAATGTTATCTT | 291 |
| 50 | Bcl2 | 2 | ACCAGGGGTGACATCTCCCTAATGTTATCTT | 292 |
| 50 | Bcl2 | 3 | CCGGTTCAGGTACTCAGTCATTAATGTTATCTT | 293 |
| 50 | Bcl2 | 4 | TGGATGAAGGGGTGTCTTTAGTTAATGTTATCTT | 294 |
| 50 | Bcl2 | 5 | ACACTCCGGCTTCACTGAGTAATGTTATCTT | 295 |
| 50 | Bcl2 | 6 | CGGGACGACCTAGGAAGTTATAATGTTATCTT | 296 |
| 51 | 117r | 1 | GTTTCCAGAGTTTGGCAGCATAATCTTATCTT | 297 |
| 51 | 117r | 2 | TGTGTGGAATAAAGATACATGCGTTAATCTTATCTT | 298 |
| 51 | Il7r | 3 | ACCATCTCTGTAGTCAGGGGTAATCTTATCTT | 299 |
| 51 | 117r | 4 | CAGTCCAGGAAACTTTCGGTAATCTTATCTT | 300 |
| 51 | Il7r | 5 | ATCATTTGCTTCTTTGCGATATAATCTTATCTT | 301 |
| 51 | 117r | 6 | GGAGATTGGTTATACACAGCATAATCTTATCTT | 302 |
| 52 | Pdcd1 | 1 | CTGGCCTCTGACATACTTGTTGAGTAATATTATCTT | 303 |
| 52 | Pdcd1 | 2 | TCCAGTTCAGCATAAGATCCTCCTAATATTATCTT | 304 |
| 52 | Pdcd1 | 3 | CCACCAGACAGCTAGACTCTTAATATTATCTT | 305 |
| 52 | Pdcd1 | 4 | TGCTCATTTCAGAGTCCTAGGTAATATTATCTT | 306 |
| 52 | Pdcd1 | 5 | ATCCTGGACGGGTTGGCTCTAATATTATCTT | 307 |
| 52 | Pdcd1 | 6 | GGCCATTCAAAGCAGGCTGTAATATTATCTT | 308 |
| 53 | Xcr1 | 1 | ACCAACTCCATTGTGCTGAGATAAGTTTATCTT | 309 |
| 53 | Xcr1 | 2 | AGATGAGCCTGACTGTTCGGTTAAGTTTATCTT | 310 |
| 53 | Xcr1 | 3 | AGGCTGAGGAGAAATACCATAAGTTTATCTT | 311 |
| 53 | Xcr1 | 4 | GGGGCTCACTACAGACAGGTAAGTTTATCTT | 312 |
| 53 | Xcr1 | 5 | TCAGTAGAAGGAGGGTCCCTAAGTTTATCTT | 313 |
| 53 | Xcr1 | 6 | AGTCCATGTCAGGGTTTCAAGTAAGTTTATCTT | 314 |
| 54 | Irf8 | 1 | CGGGGACAATTCGGTAAACTAAGGTTATCTT | 315 |
| 54 | Irf8 | 2 | AGCTGAATGGTGTGTGTCATAGTAAGGTTATCTT | 316 |
| 54 | Irf8 | 3 | AGGTGACATGTGACAAGGGGTAAGGTTATCTT | 317 |
| 54 | Irf8 | 4 | ACCGTGGTCCTTAGCTTTTCATAAGGTTATCTT | 318 |
| 54 | Irf8 | 5 | AGAGTCTGTGTGAATCAATGATAAGGTTATCTT | 319 |
| 54 | Irf8 | 6 | TTTCCCCAGAGCAACAGTCATAAGGTTATCTT | 320 |
| 55 | Irf4 | 1 | TCCTCTGTCCATTGTCGTCCTAAGCTTATCTT | 321 |
| 55 | Irf4 | 2 | AGCGGTGGTAATCTGGAGTGGTAAGCTTATCTT | 322 |
| 55 | Irf4 | 3 | ACACTTGCAGCTCTGATAGAAACTAAGCTTATCTT | 323 |
| 55 | Irf4 | 4 | GCTAGCAGAGGTTCCACATGTAAGCTTATCTT | 324 |
| 55 | Irf4 | 5 | CGTGGGCCAAACGTCACAGTAAGCTTATCTT | 325 |
| 55 | Irf4 | 6 | AGCAGCAATTAGCAAGGACTAAGTAAGCTTATCTT | 326 |
| 56 | H2-Aa | 1 | GGGTGGAATTTGACCTCTTAGTAAGATTATCTT | 327 |
| 56 | H2-Aa | 2 | AGGAATAGTCACGGTTGACGAATAAGATTATCTT | 328 |
| 56 | H2-Aa | 3 | CCAGGAGACTGATATACACTTATAAGATTATCTT | 329 |
| 56 | H2-Aa | 4 | AGGTGCCACCTGATCGCAGTAAGATTATCTT | 330 |
| 56 | H2-Aa | 5 | TCCTTTCCAGGGTGTGACTCATAAGATTATCTT | 331 |
| 57 | Cd40 | 1 | AGGGATAACACTTTTCGAAAAGTTAACTTTATCTT | 332 |
| 57 | Cd40 | 2 | AGGTACTGTTTGTCACTGCACGTTAACTTTATCTT | 333 |
| 57 | Cd40 | 3 | AGTGTTGTCCTTCCTTACAGGTACTAACTTTATCTT | 334 |
| 57 | Cd40 | 4 | ACTCGTTCCTTTCTGTAGGACTAACTTTATCTT | 335 |
| 57 | Cd40 | 5 | AGCCAGGGATACAGGGCGTGTAACTTTATCTT | 336 |
| 57 | Cd40 | 6 | ACTGGGCTGAGAATTCGCCTTAACTTTATCTT | 337 |
| 58 | Cd83 | 1 | GGTGACTGGAAGAAAAGCTTAACGTTATCTT | 338 |
| 58 | Cd83 | 2 | CCCTGTACTTCCTGAAAGTTTAACGTTATCTT | 339 |
| 58 | Cd83 | 3 | ATGGTAGTGTTTTGGATCGTCTAACGTTATCTT | 340 |
| 58 | Cd83 | 4 | TGTGGCCAGACTTCTGGTTTAACGTTATCTT | 341 |
| 58 | Cd83 | 5 | ACTGAAAAGCACTCACGAGGTAACGTTATCTT | 342 |
| 58 | Cd83 | 6 | AGAAGCGGTGATACTCCATGTAACGTTATCTT | 343 |
| 59 | Cd86 | 1 | TGCAGTCCCATTGAAATAAGCTTTAACCTTATCTT | 344 |
| 59 | Cd86 | 2 | GTGCCCAAATAGTGCTCGTACTAACCTTATCTT | 345 |
| 59 | Cd86 | 3 | ACCATCCGGGAATGAAAGAGATAACCTTATCTT | 346 |
| 59 | Cd86 | 4 | AGGAAATCTTCATTGACTCCGTTAACCTTATCTT | 347 |
| 59 | Cd86 | 5 | CCTGCTAGGCTGATTCGGCTAACCTTATCTT | 348 |
| 59 | Cd86 | 6 | ATGAGCTGAAAGAGTCCTAGCTAACCTTATCTT | 349 |
| 60 | H2-K1 | 1 | GTGCACGGTACCATCGCACCTTAACATTATCTT | 350 |
| 60 | H2-K1 | 2 | TTTACAATCTGGGAGAGACATAACATTATCTT | 351 |
| 60 | H2-K1 | 3 | CGATGTAATCGCAGCCGTCGTAACATTATCTT | 352 |
| 60 | H2-K1 | 4 | TGACAGGCTACAGATGTCCTAACATTATCTT | 353 |
| 61 | Ccl17 | 1 | AGGTCTTATACCAGCTCACCAACTAAATTTATCTT | 354 |
| 61 | Ccl17 | 2 | ACAGTCAGAAACACGATGGCATCTAAATTTATCTT | 355 |
| 61 | Ccl17 | 3 | ACCAATCTGATGGCCTTCTTCATAAATTTATCTT | 356 |
| 61 | Ccl17 | 4 | AGCATCTGAAGTGACCTCATGTAAATTTATCTT | 357 |
| 61 | Ccl17 | 5 | CTCTGCTGGTCACAGGCCGTAAATTTATCTT | 358 |
| 62 | Ccl22 | 1 | CGGCACAGATATCTCGGTTCTTAAAGTTATCTT | 359 |
| 62 | Ccl22 | 2 | AGGTCCAGAAGAACTCCTTCTAAAGTTATCTT | 360 |
| 62 | Ccl22 | 3 | GTCCTCCTCCCTAGGACAGTAAAGTTATCTT | 361 |
| 62 | Ccl22 | 4 | ACACCATAGCATAACTTCGACTATAAAGTTATCTT | 362 |
| 62 | Ccl22 | 5 | TCAAAACAACGCCAGGCTTGTAAAGTTATCTT | 363 |
| 62 | Ccl22 | 6 | GGGGCAGAGAAAGTAAAAGACCCTAAAGTTATCTT | 364 |
| 63 | Ccr4 | 1 | GGAGCTGAGGACTTTCCACGTTAAACTTATCTT | 365 |
| 63 | Ccr4 | 2 | CACCAGGTACATCCATGAAATAAACTTATCTT | 366 |
| 63 | Ccr4 | 3 | TACAAAGCGTCACGGAAGTCTAAACTTATCTT | 367 |
| 63 | Ccr4 | 4 | ATTTCTCCCCGAGAAAGAAGTAAACTTATCTT | 368 |
| 63 | Ccr4 | 5 | AGGGATTGTCCCAACACCCTAAACTTATCTT | 369 |
| 63 | Ccr4 | 6 | AGGTGAGTACCGTCATGCCTAAACTTATCTT | 370 |
| 64 | Ccl25 | 1 | ACTACTTTCTGGCGGAAGTAGAATTAAAATTATCTT | 371 |
| 64 | Ccl25 | 2 | GCTGGTGATAATTCCTAGCATAAAATTATCTT | 372 |
| 64 | Ccl25 | 3 | TCCCTCAGCAATCATCAATAGTAAAATTATCTT | 373 |
| 64 | Ccl25 | 4 | GTGCCTGCTTGTAGCGTGTTAAAATTATCTT | 374 |
| 64 | Ccl25 | 5 | GGGAGGGCCTTTAGCGCAGTAAAATTATCTT | 375 |
| 64 | Ccl25 | 6 | TCTCATCGCCCTCTTCACATAAAATTATCTT | 376 |

### 3) STARmap Padlock Probes

TABLE 3

| Target | | | | |
|---|---|---|---|---|
| no | Gene Symbol | Probe index | Probe sequence | SEQ ID NO: |
| 1 | Cd8a | 1 | /5Phos/AAAATATTGCTGAACTTGTTCAGGGTAATT | 377 |
| | | | ATTACAAAACATACACTAAAGATA | |
| 1 | Cd8a | 2 | /5Phos/AAAATAATAGACAACGAAGGTGGGCTAATT | 378 |
| | | | ATTACAAAACATACACTAAAGATA | |
| 1 | Cd8a | 3 | /5Phos/AAAATAGGCGTCCATTTTCTTTGGAAATTAT | 379 |
| | | | TACAAAACATACACTAAAGATA | |
| 1 | Cd8a | 4 | /5Phos/AAAATAGACGAATTCAGCTTCTCGTAATTAT | 380 |
| | | | TACAAAACATACACTAAAGATA | |
| 1 | Cd8a | 5 | /5Phos/AAAATATGAGAGTGATGATCAAGGACAATT | 381 |
| | | | ATTACAAAACATACACTAAAGATA | |
| 1 | Cd8a | 6 | /5Phos/AAAATAATAAATGGGGATTCTCCAGACAAA | 382 |
| | | | TTATTACAAAACATACACTAAAGATA | |
| 2 | Cd3d | 1 | /5Phos/ACAATAGCAAAGCAGTAGACGCCCAAATTA | 383 |
| | | | TTACACAACATACACTAAAGATA | |
| 2 | Cd3d | 2 | /5Phos/ACAATAGCTGTACTGGGTATCTTCACGATCT | 384 |
| | | | AATTATTACACAACATACACTAAAGATA | |
| 2 | Cd3d | 3 | /5Phos/ACAATATTTGCCCAAGTTGAGTGTTTAATTA | 385 |
| | | | TTACACAACATACACTAAAGATA | |
| 2 | Cd3d | 4 | /5Phos/ACAATATCCATCTAGATGCATGACGCTAATT | 386 |
| | | | ATTACACAACATACACTAAAGATA | |
| 2 | Cd3d | 5 | /5Phos/ACAATACTCATATTCGGTCACTTGTATCTAA | 387 |
| | | | TTATTACACAACATACACTAAAGATA | |
| 2 | Cd3d | 6 | /5Phos/ACAATACAAATAGAGGCAAGCCACAAATTA | 388 |
| | | | TTACACAACATACACTAAAGATA | |
| 3 | Lef1 | 1 | /5Phos/AGAATAACCTGAAGTCGACTCCTGTAGCTA | 389 |
| | | | ATTATTACAGAACATACACTAAAGATA | |
| 3 | Lef1 | 2 | /5Phos/AGAATATATGAGGTCTTTTGGGCTCCTAATT | 390 |
| | | | ATTACAGAACATACACTAAAGATA | |
| 3 | Lef1 | 3 | /5Phos/AGAATACATTATGTAGCCAGAGTAACTGGA | 391 |
| | | | ATTATTACAGAACATACACTAAAGATA | |
| 3 | Lef1 | 4 | /5Phos/AGAATAGTTACAATAGCTGGATGAGGGAA | 392 |
| | | | TTATTACAGAACATACACTAAAGATA | |
| 3 | Lef1 | 5 | /5Phos/AGAATAACGTGACAAGGCAAACACAAATTA | 393 |
| | | | TTACAGAACATACACTAAAGATA | |
| 3 | Lef1 | 6 | /5Phos/AGAATATGAATCCACCCGTGATGGGAATTA | 394 |
| | | | TTACAGAACATACACTAAAGATA | |
| 4 | Cd44 | 1 | /5Phos/ATAATACATGTTTCAAAACCCTTGCTAATTA | 395 |
| | | | TTACATAACATACACTAAAGATA | |
| 4 | Cd44 | 2 | /5Phos/ATAATATCGGAATTACCACATTTCCTAATTA | 396 |
| | | | TTACATAACATACACTAAAGATA | |
| 4 | Cd44 | 3 | /5Phos/ATAATAGGGTGCTCTTCTCGATGGTAATTAT | 397 |
| | | | TACATAACATACACTAAAGATA | |
| 4 | Cd44 | 4 | /5Phos/ATAATAAGCTAATTCGGATCCATGAGTAAT | 398 |
| | | | TATTACATAACATACACTAAAGATA | |
| 4 | Cd44 | 5 | /5Phos/ATAATAGATATATACTCCTGTGTGGTTAATT | 399 |
| | | | ATTACATAACATACACTAAAGATA | |
| 4 | Cd44 | 6 | /5Phos/ATAATATTTCAAGTTAATGGCGTAGGCACT | 400 |
| | | | AATTATTACATAACATACACTAAAGATA | |
| 5 | Il2ra | 1 | /5Phos/AACATACTCTTGGTGCATAGACTGTGTAATT | 401 |
| | | | ATTACAACACATACACTAAAGATA | |
| 5 | Il2ra | 2 | /5Phos/AACATAATACACTCGTAGTGAACACTCTGT | 402 |
| | | | AATTATTACAACACATACACTAAAGATA | |
| 5 | Il2ra | 3 | /5Phos/AACATAGGTGCCGTTCTTGTAGGAGAATTA | 403 |
| | | | TTACAACACATACACTAAAGATA | |
| 5 | Il2ra | 4 | /5Phos/AACATATCTCCGTCATTGCAGTTGTAATTAT | 404 |
| | | | TACAACACATACACTAAAGATA | |
| 5 | Il2ra | 5 | /5Phos/AACATACATCTTGCAGATGCTAATAGCAGG | 405 |
| | | | AAATTATTACAACACATACACTAAAGATA | |
| 5 | Il2ra | 6 | /5Phos/AACATACAAGTCCATACCTGTGCCTAATTAT | 406 |
| | | | TACAACACATACACTAAAGATA | |
| 6 | Kit | 1 | /5Phos/ACCATAGAATCGTCAACTCTTGCCGAATTAT | 407 |
| | | | TACACCACATACACTAAAGATA | |
| 6 | Kit | 2 | /5Phos/ACCATAAGGTATAAGTGCCTCCTTCTGTGA | 408 |
| | | | ATTATTACACCACATACACTAAAGATA | |
| 6 | Kit | 3 | /5Phos/ACCATAGTACTTCATACATGGGTTTCTGCAA | 409 |
| | | | ATTATTACACCACATACACTAAAGATA | |
| 6 | Kit | 4 | /5Phos/ACCATATCGTACGTCAGGATTTCTGGTAATT | 410 |
| | | | ATTACACCACATACACTAAAGATA | |
| 6 | Kit | 5 | /5Phos/ACCATAAGGTGACTTGTTTCAGGCAAATTA | 411 |
| | | | TTACACCACATACACTAAAGATA | |
| 6 | Kit | 6 | /5Phos/ACCATAACGTACACACTGAACCCTTAATTAT | 412 |
| | | | TACACCACATACACTAAAGATA | |
| 7 | Cd28 | 1 | /5Phos/AGCATAGTTTCGTTGTCGAAATCCCCGTAAT | 413 |
| | | | TATTACAGCACATACACTAAAGATA | |
| 7 | Cd28 | 2 | /5Phos/AGCATAAAGGTTGTAGGAATACCTGCAGCT | 414 |
| | | | GAATTATTACAGCACATACACTAAAGATA | |
| 7 | Cd28 | 3 | /5Phos/AGCATATGTCTAGGTAAGGCGGAGGAATT | 415 |
| | | | ATTACAGCACATACACTAAAGATA | |
| 7 | Cd28 | 4 | /5Phos/AGCATAGCCATAGAAGCTTCTGTCATAATT | 416 |
| | | | ATTACAGCACATACACTAAAGATA | |
| 7 | Cd28 | 5 | /5Phos/AGCATAAAAATTCCCATTCCCGACACAATTA | 417 |
| | | | TTACAGCACATACACTAAAGATA | |
| 7 | Cd28 | 6 | /5Phos/AGCATACCCAAAACAGCTTAGGAGATGACT | 418 |
| | | | GAATTATTACAGCACATACACTAAAGATA | |
| 8 | Cd3g | 1 | /5Phos/ATCATATCAGAAGTACAGAACCGTCTCCTA | 419 |
| | | | ATTATTACATCACATACACTAAAGATA | |
| 8 | Cd3g | 2 | /5Phos/ATCATACAAGAGCAAGGAAGAAGATGAAT | 420 |
| | | | TATTACATCACATACACTAAAGATA | |
| 8 | Cd3g | 3 | /5Phos/ATCATAGCGAACTCCATCCTGTCCCAATTAT | 421 |
| | | | TACATCACATACACTAAAGATA | |
| 8 | Cd3g | 4 | /5Phos/ATCATATGCAGTTTTCACACATTCTGTAATT | 422 |
| | | | ATTACATCACATACACTAAAGATA | |
| 8 | Cd3g | 5 | /5Phos/ATCATAAGCCGGATATGGTGCCTATGTAAT | 423 |
| | | | TATTACATCACATACACTAAAGATA | |
| 8 | Cd3g | 6 | /5Phos/ATCATATTCCCGGTCCTTGAGGGGCAATTA | 424 |
| | | | TTACATCACATACACTAAAGATA | |
| 9 | Cd3e | 1 | /5Phos/AAGATAGTCAACTCTACACTGGTTCCTGAG | 425 |
| | | | AATTATTACAAGACATACACTAAAGATA | |
| 9 | Cd3e | 2 | /5Phos/AAGATAACAATGATGATTATGGCTACTGCT | 426 |
| | | | AATTATTACAAGACATACACTAAAGATA | |
| 9 | Cd3e | 3 | /5Phos/AAGATAGCCAGAATACAGGTCCCGCAATTA | 427 |
| | | | TTACAAGACATACACTAAAGATA | |
| 9 | Cd3e | 4 | /5Phos/AAGATATAAGGGGAAATCTGGAGAGGCAA | 428 |
| | | | ATTATTACAAGACATACACTAAAGATA | |
| 9 | Cd3e | 5 | /5Phos/AAGATATGAGGGCCAATTAGGAGAGGAAT | 429 |
| | | | TATTACAAGACATACACTAAAGATA | |
| 9 | Cd3e | 6 | /5Phos/AAGATATCTCCTATCTGTCAGACTGCTAATT | 430 |
| | | | ATTACAAGACATACACTAAAGATA | |
| 10 | Cd247 | 1 | /5Phos/ACGATACATTGTAGAGCTGGTTGGGGTAAT | 431 |
| | | | TATTACACGACATACACTAAAGATA | |
| 10 | Cd247 | 2 | /5Phos/ACGATAGATGAAGAGGATTCCATCTAGCAA | 432 |
| | | | ATTATTACACGACATACACTAAAGATA | |
| 10 | Cd247 | 3 | /5Phos/ACGATAGCATGAAAGAAATTGGAACATCA | 433 |
| | | | GAATTATTACACGACATACACTAAAGATA | |
| 10 | Cd247 | 4 | /5Phos/ACGATATCCTTGTGAGTTCTGAACCTTCTAA | 434 |
| | | | TTATTACACGACATACACTAAAGATA | |
| 10 | Cd247 | 5 | /5Phos/ACGATACAGACCAAAGCTCTGTGCCTAATT | 435 |
| | | | ATTACACGACATACACTAAAGATA | |
| 10 | Cd247 | 6 | /5Phos/ACGATATGCCTCTCCGCCTCTCGCCTAATTA | 436 |
| | | | TTACACGACATACACTAAAGATA | |
| 11 | Rag1 | 1 | /5Phos/AGGATATTGGGTCATAAGCTTCCGGGAATT | 437 |
| | | | ATTACAGGACATACACTAAAGATA | |
| 11 | Rag1 | 2 | /5Phos/AGGATATTGAAACTGTACTGACAGAGGGA | 438 |
| | | | CTAATTATTACAGGACATACACTAAAGATA | |
| 11 | Rag1 | 3 | /5Phos/AGGATATTCAATAATTTCAGGGACATGTAA | 439 |
| | | | TTATTACAGGACATACACTAAAGATA | |
| 11 | Rag1 | 4 | /5Phos/AGGATAGTCTAAACAGCTTGTTACCCGAAT | 440 |
| | | | TATTACAGGACATACACTAAAGATA | |
| 11 | Rag1 | 5 | /5Phos/AGGATATGAAAGGTTTGGCAGAGACCCAA | 441 |
| | | | TTATTACAGGACATACACTAAAGATA | |
| 11 | Rag1 | 6 | /5Phos/AGGATATAGCGGGACATGAAGAGCGCAAT | 442 |
| | | | TATTACAGGACATACACTAAAGATA | |
| 12 | Rag2 | 1 | /5Phos/ATGATACCCCTAGAGAGACAAGGCTAATTA | 443 |
| | | | TTACATGACATACACTAAAGATA | |
| 12 | Rag2 | 2 | /5Phos/ATGATATATCTGGGTTCAGGGACATCTAAT | 444 |
| | | | TATTACATGACATACACTAAAGATA | |
| 12 | Rag2 | 3 | /5Phos/ATGATACAAGACTTTCCCAGAGCCTAATTAT | 445 |
| | | | TACATGACATACACTAAAGATA | |
| 12 | Rag2 | 4 | /5Phos/ATGATACTCCTAGCTCATGGCATCTAATTAT | 446 |
| | | | TACATGACATACACTAAAGATA | |
| 12 | Rag2 | 5 | /5Phos/ATGATATTGAATAGAACGGAACCCAGGTAA | 447 |
| | | | TTATTACATGACATACACTAAAGATA | |
| 12 | Rag2 | 6 | /5Phos/ATGATAGTGCAATTCACTGCTGGGGTAATT | 448 |
| | | | ATTACATGACATACACTAAAGATA | |
| 13 | Hes1 | 1 | /5Phos/AATATATGTCGTGTTGACACTGGCTGGAAT | 449 |
| | | | TATTACAATACATACACTAAAGATA | |
| 13 | Hes1 | 2 | /5Phos/AATATATGATGACTTTCTGTGCTCAGAATTA | 450 |
| | | | TTACAATACATACACTAAAGATA | |
| 13 | Hes1 | 3 | /5Phos/AATATAGCACTATTCCAGGACCAAGGAATT | 451 |
| | | | ATTACAATACATACACTAAAGATA | |
| 13 | Hes1 | 4 | /5Phos/AATATACATCCAAAATCAGTGTTTTCAGTAA | 452 |
| | | | TTATTACAATACATACACTAAAGATA | |
| 13 | Hes1 | 5 | /5Phos/AATATATGAGCGAAGGCCCCGTTGGGGAT | 453 |
| | | | AATTATTACAATACATACACTAAAGATA | |
| 13 | Hes1 | 6 | /5Phos/AATATACGAAGTGAGCGAGGAGCCACTGG | 454 |
| | | | AATTATTACAATACATACACTAAAGATA | |
| 14 | Tcf7 | 1 | /5Phos/ACTATACTATCATATGGCTGCAGCTCAATTA | 455 |
| | | | TTACACTACATACACTAAAGATA | |
| 14 | Tcf7 | 2 | /5Phos/ACTATAAGAGTGGAGAAAGCTGGGGAATT | 456 |
| | | | ATTACACTACATACACTAAAGATA | |
| 14 | Tcf7 | 3 | /5Phos/ACTATAAACGCATTGAGGGGTTTCTAATTA | 457 |
| | | | TTACACTACATACACTAAAGATA | |
| 14 | Tcf7 | 4 | /5Phos/ACTATAAACCAGGTATTCTTGGTCCAAATTA | 458 |
| | | | TTACACTACATACACTAAAGATA | |
| 14 | Tcf7 | 5 | /5Phos/ACTATATTTGTACATGCCACTGGCAAATTAT | 459 |
| | | | TACACTACATACACTAAAGATA | |
| 14 | Tcf7 | 6 | /5Phos/ACTATACAACTATATTGACTAGTTGTAATTA | 460 |
| | | | TTACACTACATACACTAAAGATA | |
| 15 | Gata3 | 1 | /5Phos/AGTATAGGTTGCCTTGACCATCGATGTAAT | 461 |
| | | | TATTACAGTACATACACTAAAGATA | |
| 15 | Gata3 | 2 | /5Phos/AGTATATGGTCAGCATGTGGCTGGAGTAAT | 462 |
| | | | TATTACAGTACATACACTAAAGATA | |
| 15 | Gata3 | 3 | /5Phos/AGTATACGCGCAGGATGTCCCTGCTAATTA | 463 |
| | | | TTACAGTACATACACTAAAGATA | |
| 15 | Gata3 | 4 | /5Phos/AGTATAGAGTAGCAAGGAGCGTAGAGGAA | 464 |
| | | | TTATTACAGTACATACACTAAAGATA | |
| 15 | Gata3 | 5 | /5Phos/AGTATACGGGCTTCATGATACTGCTAATTAT | 465 |
| | | | TACAGTACATACACTAAAGATA | |
| 15 | Gata3 | 6 | /5Phos/AGTATATCTATGTCTCGCTCTCTCAGTAATT | 466 |
| | | | ATTACAGTACATACACTAAAGATA | |
| 16 | Bcl11b | 1 | /5Phos/ATTATATGATGAGTTCCCTCTGGGAAATTAT | 467 |
| | | | TACATTACATACACTAAAGATA | |
| 16 | Bcl11b | 2 | /5Phos/ATTATAGTCAGCAAGTGTTCACCGTAATTAT | 468 |
| | | | TACATTACATACACTAAAGATA | |
| 16 | Bcl11b | 3 | /5Phos/ATTATATTCTCAGATGGGATGAGGGCGAAT | 469 |
| | | | TATTACATTACATACACTAAAGATA | |
| 16 | Bcl11b | 4 | /5Phos/ATTATATTGTGCAAATGTAGCTGGAAGAAT | 470 |
| | | | TATTACATTACATACACTAAAGATA | |
| 16 | Bcl11b | 5 | /5Phos/ATTATATTTCCATAGGACTTCGCAGAAATTA | 471 |
| | | | TTACATTACATACACTAAAGATA | |
| 16 | Bcl11b | 6 | /5Phos/ATTATATCATTGCTCAGACTACTACTGGGTA | 472 |
| | | | ATTATTACATTACATACACTAAAGATA | |
| 17 | Ets1 | 1 | /5Phos/AAACTATTGGGACATCATTTCTTTGCTAATT | 473 |
| | | | ATTACAAACCATACACTAAAGATA | |
| 17 | Ets1 | 2 | /5Phos/AAACTATGATAGGATGCAGCGTCTGATAGG | 474 |
| | | | AATTATTACAAACCATACACTAAAGATA | |
| 17 | Ets1 | 3 | /5Phos/AAACTAATGAAGTAATCCGAGGTGTAACAG | 475 |
| | | | GAATTATTACAAACCATACACTAAAGATA | |
| 17 | Ets1 | 4 | /5Phos/AAACTAGTCTGTCTGCAAGGTGTCTGTAAT | 476 |
| | | | TATTACAAACCATACACTAAAGATA | |
| 17 | Ets1 | 5 | /5Phos/AAACTACAGCGGGACATCTGCACATAATTA | 477 |
| | | | TTACAAACCATACACTAAAGATA | |
| 17 | Ets1 | 6 | /5Phos/AAACTAGCTGCTACATTTCCAGTGTAATTAT | 478 |
| | | | TACAAACCATACACTAAAGATA | |
| 18 | Lyl1 | 1 | /5Phos/ACACTATTCTCAGTCATGGCGGAACCAATT | 479 |
| | | | ATTACACACCATACACTAAAGATA | |
| 18 | Lyl1 | 2 | /5Phos/ACACTAACCAGTTGGGCTTCATGGGAATTA | 480 |
| | | | TTACACACCATACACTAAAGATA | |
| 18 | Lyl1 | 3 | /5Phos/ACACTAAACCCTGTAGCAGTTTCCACAGAA | 481 |
| | | | TTATTACACACCATACACTAAAGATA | |
| 18 | Lyl1 | 4 | /5Phos/ACACTAGTCGCGGTTAACAGGATCAAATTA | 482 |
| | | | TTACACACCATACACTAAAGATA | |
| 18 | Lyl1 | 5 | /5Phos/ACACTAGGGCCGAAAGGCACTGAGCTAATT | 483 |
| | | | ATTACACACCATACACTAAAGATA | |
| 18 | Lyl1 | 6 | /5Phos/ACACTACTGCGCTTCAGCCGGCTGTAATTAT | 484 |
| | | | TACACACCATACACTAAAGATA | |
| 19 | Hhex | 1 | /5Phos/AGACTAACGCTTTCTCTCGGGTGGGAATTA | 485 |
| | | | TTACAGACCATACACTAAAGATA | |
| 19 | Hhex | 2 | /5Phos/AGACTATGTTTGTTAATTAGCCAGTGCAAA | 486 |
| | | | TTATTACAGACCATACACTAAAGATA | |
| 19 | Hhex | 3 | /5Phos/AGACTAGAATAGCTACAGACTTAGGAAAGT | 487 |
| | | | AATTATTACAGACCATACACTAAAGATA | |
| 19 | Hhex | 4 | /5Phos/AGACTATGTAAAAGTTGGGTATACCCTAAT | 488 |
| | | | TATTACAGACCATACACTAAAGATA | |
| 19 | Hhex | 5 | /5Phos/AGACTATGGAGAACCTCACTTGACCGCCTA | 489 |
| | | | ATTATTACAGACCATACACTAAAGATA | |
| 19 | Hhex | 6 | /5Phos/AGACTAGGGAACGGGTACAGAGGGCCTAA | 490 |
| | | | TTATTACAGACCATACACTAAAGATA | |
| 20 | Bcl11a | 1 | /5Phos/ATACTACGTTGATAAACAATCGTCATCCTAA | 491 |
| | | | TTATTACATACCATACACTAAAGATA | |
| 20 | Bcl11a | 2 | /5Phos/ATACTAATCTGCTATGTGTTCCTGTTAATTA | 492 |
| | | | TTACATACCATACACTAAAGATA | |
| 20 | Bcl11a | 3 | /5Phos/ATACTACGGGTAATTAGGTTGGCTGGTAAT | 493 |
| | | | TATTACATACCATACACTAAAGATA | |
| 20 | Bcl11a | 4 | /5Phos/ATACTACGGAGCACGTTGACGTCGGAATTA | 494 |
| | | | TTACATACCATACACTAAAGATA | |
| 20 | Bcl11a | 5 | /5Phos/ATACTACACTTTCTAAGTAGATTCTTAATTA | 495 |
| | | | TTACATACCATACACTAAAGATA | |
| 20 | Bcl11a | 6 | /5Phos/ATACTACATCTTGAGCTCGCGGGGTAATTA | 496 |
| | | | TTACATACCATACACTAAAGATA | |
| 21 | Hoxa9 | 1 | /5Phos/AACCTAGAAGTCGGGGTGTTCGGCCAATTA | 497 |
| | | | TTACAACCCATACACTAAAGATA | |
| 21 | Hoxa9 | 2 | /5Phos/AACCTAAAGCGTGGGACAGTCACCCAATTA | 498 |
| | | | TTACAACCCATACACTAAAGATA | |
| 21 | Hoxa9 | 3 | /5Phos/AACCTACCAAGAGCAGTTTCATGGTAATTA | 499 |
| | | | TTACAACCCATACACTAAAGATA | |
| 21 | Hoxa9 | 4 | /5Phos/AACCTACAGATTCGTTTGCCTCGCTGAATTA | 500 |
| | | | TTACAACCCATACACTAAAGATA | |
| 21 | Hoxa9 | 5 | /5Phos/AACCTAAATGCCCTCCCTTGTGTGTAATTAT | 501 |
| | | | TACAACCCATACACTAAAGATA | |
| 21 | Hoxa9 | 6 | /5Phos/AACCTACGAATTTCCTCACTTCTCCTAATTAT | 502 |
| | | | TACAACCCATACACTAAAGATA | |
| 22 | Mef2c | 1 | /5Phos/ACCCTATGTTATGGCTGGACACTGGAATTA | 503 |
| | | | TTACACCCCATACACTAAAGATA | |
| 22 | Mef2c | 2 | /5Phos/ACCCTATGGAGGTCTATGTGTTACACCAGG | 504 |
| | | | AATTATTACACCCCATACACTAAAGATA | |
| 22 | Mef2c | 3 | /5Phos/ACCCTACTCCCATTCCTTGTCCTGGAATTAT | 505 |
| | | | TACACCCCATACACTAAAGATA | |
| 22 | Mef2c | 4 | /5Phos/ACCCTAGTTGCTACGGAAACCACCGAATTA | 506 |
| | | | TTACACCCCATACACTAAAGATA | |
| 22 | Mef2c | 5 | /5Phos/ACCCTAGCCATGTTTAGGCAACAGCCTAAT | 507 |
| | | | TATTACACCCCATACACTAAAGATA | |
| 22 | Mef2c | 6 | /5Phos/ACCCTATTCGCGTAAAAGCCACGATAATTA | 508 |
| | | | TTACACCCCATACACTAAAGATA | |
| 23 | Lmo2 | 1 | /5Phos/AGCCTATGTTCACACACTATGTCGGAGTAA | 509 |
| | | | TTATTACAGCCCATACACTAAAGATA | |
| 23 | Lmo2 | 2 | /5Phos/AGCCTATCACCTACACAGAAATGCTAATTAT | 510 |
| | | | TACAGCCCATACACTAAAGATA | |
| 23 | Lmo2 | 3 | /5Phos/AGCCTACATCGTCATCTCATAGGCAAATTAT | 511 |
| | | | TACAGCCCATACACTAAAGATA | |
| 23 | Lmo2 | 4 | /5Phos/AGCCTAATCGATAGTTACCTGGGGAAGCAA | 512 |
| | | | TTATTACAGCCCATACACTAAAGATA | |
| 23 | Lmo2 | 5 | /5Phos/AGCCTAACTGTGACTAGAACAGGGTAATTA | 513 |
| | | | TTACAGCCCATACACTAAAGATA | |
| 23 | Lmo2 | 6 | /5Phos/AGCCTACCTATGTTCTGCTGGCAGCCAATTA | 514 |
| | | | TTACAGCCCATACACTAAAGATA | |
| 24 | Meis1 | 1 | /5Phos/ATCCTATGAATGACTCTGACGAGCAGAAAT | 515 |
| | | | TATTACATCCCATACACTAAAGATA | |
| 24 | Meis1 | 2 | /5Phos/ATCCTAGACCGAGGAACCCATGCTGAATTA | 516 |
| | | | TTACATCCCATACACTAAAGATA | |
| 24 | Meis1 | 3 | /5Phos/ATCCTATATATGCATGCCACCTTGCAAATTA | 517 |
| | | | TTACATCCCATACACTAAAGATA | |
| 24 | Meis1 | 4 | /5Phos/ATCCTATCCTGTGTTAAGAACCGAGGGGAA | 518 |
| | | | TTATTACATCCCATACACTAAAGATA | |
| 24 | Meis1 | 5 | /5Phos/ATCCTAAATGAATGATCGTCCGTTCTGTCTA | 519 |
| | | | ATTATTACATCCCATACACTAAAGATA | |
| 24 | Meis1 | 6 | /5Phos/ATCCTAGGATGCCGTGTCATCATGGTCTAA | 520 |
| | | | TTATTACATCCCATACACTAAAGATA | |
| 25 | Tcf3 | 1 | /5Phos/AAGCTAAGAACTGTTCTGGTCACTGCTAAT | 521 |
| | | | TATTACAAGCCATACACTAAAGATA | |
| 25 | Tcf3 | 2 | /5Phos/AAGCTAATTATTGCTGGAGTGATCCGGAAT | 522 |
| | | | TATTACAAGCCATACACTAAAGATA | |
| 25 | Tcf3 | 3 | /5Phos/AAGCTAGAATACCCAGGTCATGGCCTGTAA | 523 |
| | | | TTATTACAAGCCATACACTAAAGATA | |
| 25 | Tcf3 | 4 | /5Phos/AAGCTAAACCGAGAGGAAGGAACTTTAATT | 524 |
| | | | ATTACAAGCCATACACTAAAGATA | |
| 25 | Tcf3 | 5 | /5Phos/AAGCTAGAGGACCATGAGGACGGCCAATT | 525 |
| | | | ATTACAAGCCATACACTAAAGATA | |
| 25 | Tcf3 | 6 | /5Phos/AAGCTAGGGGCCGGTGAAGCTCGTGGTAA | 526 |
| | | | TTATTACAAGCCATACACTAAAGATA | |
| 26 | Ikzf1 | 1 | /5Phos/ACGCTATTCTGCCATCTCGTTGTGGTAATTA | 527 |
| | | | TTACACGCCATACACTAAAGATA | |
| 26 | Ikzf1 | 2 | /5Phos/ACGCTAGCCTCGATCACTCTTGGAGTAATT | 528 |
| | | | ATTACACGCCATACACTAAAGATA | |
| 26 | Ikzf1 | 3 | /5Phos/ACGCTAGAGCTCTTACGTTTGGCGAAATTA | 529 |
| | | | TTACACGCCATACACTAAAGATA | |
| 26 | Ikzf1 | 4 | /5Phos/ACGCTATGATCCAGGAAGAGCACGCAATTA | 530 |
| | | | TTACACGCCATACACTAAAGATA | |
| 26 | Ikzf1 | 5 | /5Phos/ACGCTACTCATCGACATCCATTGTCTAATTA | 531 |
| | | | TTACACGCCATACACTAAAGATA | |
| 26 | Ikzf1 | 6 | /5Phos/ACGCTATCAGCTAGAATGTTCCTGGCAATT | 532 |
| | | | ATTACACGCCATACACTAAAGATA | |
| 27 | Runx1 | 1 | /5Phos/AGGCTACCGAGTAGTTTTCATCGTTGCCTA | 533 |
| | | | ATTATTACAGGCCATACACTAAAGATA | |
| 27 | Runx1 | 2 | /5Phos/AGGCTATGTAAAGACGGTGATGGTCAGAG | 534 |
| | | | TAATTATTACAGGCCATACACTAAAGATA | |
| 27 | Runx1 | 3 | /5Phos/AGGCTACGTTGAATCTCGCTACCTGGTAAT | 535 |
| | | | TATTACAGGCCATACACTAAAGATA | |
| 27 | Runx1 | 4 | /5Phos/AGGCTAAGACTACATGTGCCTGATGGATAA | 536 |
| | | | TTATTACAGGCCATACACTAAAGATA | |
| 27 | Runx1 | 5 | /5Phos/AGGCTAATCTGAACCGTAGCCATGGAATTA | 537 |
| | | | TTACAGGCCATACACTAAAGATA | |
| 27 | Runx1 | 6 | /5Phos/AGGCTAAGACACCTGGTCTTAACTCTAATT | 538 |
| | | | ATTACAGGCCATACACTAAAGATA | |
| 28 | Tcf12 | 1 | /5Phos/ATGCTAGACATGTGGGATGTGGAGGTCAA | 539 |
| | | | TTATTACATGCCATACACTAAAGATA | |
| 28 | Tcf12 | 2 | /5Phos/ATGCTAAGGTGAAGGAGATCCCACTAATTA | 540 |
| | | | TTACATGCCATACACTAAAGATA | |
| 28 | Tcf12 | 3 | /5Phos/ATGCTACGACCATCGAAGCTGATCGAAATT | 541 |
| | | | ATTACATGCCATACACTAAAGATA | |
| 28 | Tcf12 | 4 | /5Phos/ATGCTAGAAAACTACTGCTTGTGTGGTAAT | 542 |
| | | | TATTACATGCCATACACTAAAGATA | |
| 28 | Tcf12 | 5 | /5Phos/ATGCTAGGAGGAGTATGTGAGGCGGAATT | 543 |
| | | | ATTACATGCCATACACTAAAGATA | |
| 28 | Tcf12 | 6 | /5Phos/ATGCTAAAAGGTCAGAAGAACTGTGGGTA | 544 |
| | | | ATTATTACATGCCATACACTAAAGATA | |
| 29 | Gfi1 | 1 | /5Phos/AATCTACCATGCATAGGGCTTGAAAGGCAA | 545 |
| | | | TTATTACAATCCATACACTAAAGATA | |
| 29 | Gfi1 | 2 | /5Phos/AATCTATGACCTCTTGAAGCTCTTGCAATTA | 546 |
| | | | TTACAATCCATACACTAAAGATA | |
| 29 | Gfi1 | 3 | /5Phos/AATCTACTGCGTTCCTGGGAGTGCAAATTA | 547 |
| | | | TTACAATCCATACACTAAAGATA | |
| 29 | Gfi1 | 4 | /5Phos/AATCTATCCTTACTCTGATAACCTGCGGCAA | 548 |
| | | | TTATTACAATCCATACACTAAAGATA | |
| 29 | Gfi1 | 5 | /5Phos/AATCTAAGGAACGAGGTTCCTTCCCAATTA | 549 |
| | | | TTACAATCCATACACTAAAGATA | |
| 29 | Gfi1 | 6 | /5Phos/AATCTACCTCGGATACTCTGAGTTCTCGTAA | 550 |
| | | | TTATTACAATCCATACACTAAAGATA | |
| 30 | Cd4 | 1 | /5Phos/ACTCTACTAGGAGTTGTGACAGCTGCAGAA | 551 |
| | | | TTATTACACTCCATACACTAAAGATA | |
| 30 | Cd4 | 2 | /5Phos/ACTCTAGACCTTAGAGTTGCTATCCAAGGT | 552 |
| | | | AATTATTACACTCCATACACTAAAGATA | |
| 30 | Cd4 | 3 | /5Phos/ACTCTAATAGCTGTGCTCTGAAAACCCAATT | 553 |
| | | | ATTACACTCCATACACTAAAGATA | |
| 30 | Cd4 | 4 | /5Phos/ACTCTAGGGTATCTTGAGGGTGAGTGGGA | 554 |
| | | | ATTATTACACTCCATACACTAAAGATA | |
| 30 | Cd4 | 5 | /5Phos/ACTCTACTTTTGTACGGACACCTCTTAATTA | 555 |
| | | | TTACACTCCATACACTAAAGATA | |
| 30 | Cd4 | 6 | /5Phos/ACTCTACACTCAGTAGACACTGCCAAATTAT | 556 |
| | | | TACACTCCATACACTAAAGATA | |
| 31 | Foxp3 | 1 | /5Phos/AGTCTATGTGGTCTGTCCTGGAGAAGGAAT | 557 |
| | | | TATTACAGTCCATACACTAAAGATA | |
| 31 | Foxp3 | 2 | /5Phos/AGTCTACAAATTCATCTACGGTCCACACTAA | 558 |
| | | | TTATTACAGTCCATACACTAAAGATA | |
| 31 | Foxp3 | 3 | /5Phos/AGTCTAGGTGGCTACGATGCAGCAAGAATT | 559 |
| | | | ATTACAGTCCATACACTAAAGATA | |
| 31 | Foxp3 | 4 | /5Phos/AGTCTAAAACCAATGGTAGATTTCATAATT | 560 |
| | | | ATTACAGTCCATACACTAAAGATA | |
| 31 | Foxp3 | 5 | /5Phos/AGTCTAGATCCCCTTTGTCTTATCAGGAATT | 561 |
| | | | ATTACAGTCCATACACTAAAGATA | |
| 31 | Foxp3 | 6 | /5Phos/AGTCTATTCGAGATCTTGAGGAGCAGGTAA | 562 |
| | | | TTATTACAGTCCATACACTAAAGATA | |
| 32 | Ifng | 1 | /5Phos/ATTCTAATGACGCTTATGTTGTTGCTAATTA | 563 |
| | | | TTACATTCCATACACTAAAGATA | |
| 32 | Ifng | 2 | /5Phos/ATTCTAGCTACAATCTGAGTTCAGTCAGAAT | 564 |
| | | | TATTACATTCCATACACTAAAGATA | |
| 32 | Ifng | 3 | /5Phos/ATTCTAGGTGGACCACTCGGATGAGCTAAT | 565 |
| | | | TATTACATTCCATACACTAAAGATA | |
| 32 | Ifng | 4 | /5Phos/ATTCTAGATAATCTGGCTCTGCAGGAATTAT | 566 |
| | | | TACATTCCATACACTAAAGATA | |
| 32 | Ifng | 5 | /5Phos/ATTCTATTCAATGACTGTGCCGTGGAATTAT | 567 |
| | | | TACATTCCATACACTAAAGATA | |
| 32 | Ifng | 6 | /5Phos/ATTCTAAGCGTTCATTGTCTCAGAGCTAATT | 568 |
| | | | ATTACATTCCATACACTAAAGATA | |
| 33 | 114 | 1 | /5Phos/AAAGTAGGACGTTTGGCACATCCATCTAAT | 569 |
| | | | TATTACAAAGCATACACTAAAGATA | |
| 33 | 114 | 2 | /5Phos/AAAGTACCTACAGACGAGCTCACTCTAATT | 570 |
| | | | ATTACAAAGCATACACTAAAGATA | |
| 33 | 114 | 3 | /5Phos/AAAGTACATCGAAAAGCCCGAAAGAGTAA | 571 |
| | | | TTATTACAAAGCATACACTAAAGATA | |
| 33 | 114 | 4 | /5Phos/AAAGTAGCCGTCAGTGACGAGAGACAAAT | 572 |
| | | | TATTACAAAGCATACACTAAAGATA | |
| 33 | 114 | 5 | /5Phos/AAAGTAAATCCATTTGCATGATGCTCTTAAT | 573 |
| | | | TATTACAAAGCATACACTAAAGATA | |
| 33 | 114 | 6 | /5Phos/AAAGTAGAGTTCTTCTTCAAGCATGGAGTA | 574 |
| | | | ATTATTACAAAGCATACACTAAAGATA | |
| 34 | Il17a | 1 | /5Phos/ACAGTAGGTAGTCTGAGGGCCTTCTGAATT | 575 |
| | | | ATTACACAGCATACACTAAAGATA | |
| 34 | Il17a | 2 | /5Phos/ACAGTATTGAGGTTGACCTTCACATTAATTA | 576 |
| | | | TTACACAGCATACACTAAAGATA | |
| 34 | Il17a | 3 | /5Phos/ACAGTAAGTGAAGGGGCAGCTCTCAAATTA | 577 |
| | | | TTACACAGCATACACTAAAGATA | |
| 34 | Il17a | 4 | /5Phos/ACAGTATTACACCTTCTTTTCATTGTGGAAT | 578 |
| | | | TATTACACAGCATACACTAAAGATA | |
| 34 | Il17a | 5 | /5Phos/ACAGTATCTATCAGGGTCTTCATTGCGGTA | 579 |
| | | | ATTATTACACAGCATACACTAAAGATA | |
| 34 | Il17a | 6 | /5Phos/ACAGTATTCATGTGGTGGTCCAGCTAATTA | 580 |
| | | | TTACACAGCATACACTAAAGATA | |
| 35 | Tbx21 | 1 | /5Phos/AGAGTATACATGGACTCAAAGTTCTCCCAA | 581 |
| | | | TTATTACAGAGCATACACTAAAGATA | |
| 35 | Tbx21 | 2 | /5Phos/AGAGTAAATGAAACTTCCTGGCGCATAATT | 582 |
| | | | ATTACAGAGCATACACTAAAGATA | |
| 35 | Tbx21 | 3 | /5Phos/AGAGTAGTGTTAGAAGCACTGCAGGAATT | 583 |
| | | | ATTACAGAGCATACACTAAAGATA | |
| 35 | Tbx21 | 4 | /5Phos/AGAGTACGCCTAGTCCTGAGTCGCTAATTA | 584 |
| | | | TTACAGAGCATACACTAAAGATA | |
| 35 | Tbx21 | 5 | /5Phos/AGAGTAATAACTGTGTTCCCGAGGTGTAAT | 585 |
| | | | TATTACAGAGCATACACTAAAGATA | |
| 35 | Tbx21 | 6 | /5Phos/AGAGTAACCCTTCAAACCCTTCCTCTAATTA | 586 |
| | | | TTACAGAGCATACACTAAAGATA | |
| 36 | Ccr7 | 1 | /5Phos/ATAGTACCAAAGATCCAGGACTTGGCAATT | 587 |
| | | | ATTACATAGCATACACTAAAGATA | |
| 36 | Ccr7 | 2 | /5Phos/ATAGTACATCTTGGCAGAAGCACACCTAAT | 588 |
| | | | TATTACATAGCATACACTAAAGATA | |
| 36 | Ccr7 | 3 | /5Phos/ATAGTAGGTAGCAGAAACTCATAGCCAGAA | 589 |
| | | | TTATTACATAGCATACACTAAAGATA | |
| 36 | Ccr7 | 4 | /5Phos/ATAGTATGATCAAGGCCTCCACTTGGAATT | 590 |
| | | | ATTACATAGCATACACTAAAGATA | |
| 36 | Ccr7 | 5 | /5Phos/ATAGTAGAAGGCATACAAGAAAGGGTTGA | 591 |
| | | | CGAATTATTACATAGCATACACTAAAGATA | |
| 36 | Ccr7 | 6 | /5Phos/ATAGTAGTGGCTGACAGAGAACAAAGAAT | 592 |
| | | | TATTACATAGCATACACTAAAGATA | |
| 37 | Sell | 1 | /5Phos/AACGTAACAGGCGTCATCGTTCCATAATTA | 593 |
| | | | TTACAACGCATACACTAAAGATA | |
| 37 | Sell | 2 | /5Phos/AACGTAATGACGGCTACAGGAATGAAGAG | 594 |
| | | | GAATTATTACAACGCATACACTAAAGATA | |
| 37 | Sell | 3 | /5Phos/AACGTATTTTGTTGGTTCCCACCCAAATTAT | 595 |
| | | | TACAACGCATACACTAAAGATA | |
| 37 | Sell | 4 | /5Phos/AACGTACTTGATATAGATCTCCACACAATTA | 596 |
| | | | TTACAACGCATACACTAAAGATA | |
| 37 | Sell | 5 | /5Phos/AACGTATTGGGCAAGTTAAGGAGCAAATTA | 597 |
| | | | TTACAACGCATACACTAAAGATA | |
| 37 | Sell | 6 | /5Phos/AACGTAGTTGGTCATGAAGAAATCCTAATT | 598 |
| | | | ATTACAACGCATACACTAAAGATA | |
| 38 | Adgre1 | 1 | /5Phos/ACCGTAGCACTCTGTAAGGACACTGGGAAT | 599 |
| | | | TATTACACCGCATACACTAAAGATA | |
| 38 | Adgre1 | 2 | /5Phos/ACCGTATGGAATACTTAGGGCAGATCCCAA | 600 |
| | | | TTATTACACCGCATACACTAAAGATA | |
| 38 | Adgre1 | 3 | /5Phos/ACCGTACTCAGACTTCTGCTTTGGCTAATTA | 601 |
| | | | TTACACCGCATACACTAAAGATA | |
| 38 | Adgre1 | 4 | /5Phos/ACCGTATGAGCAGACAGTGAATGAGGAAT | 602 |
| | | | TATTACACCGCATACACTAAAGATA | |
| 38 | Adgre1 | 5 | /5Phos/ACCGTACTTCCGAGAGTGTTGTGGCAATTA | 603 |
| | | | TTACACCGCATACACTAAAGATA | |
| 38 | Adgre1 | 6 | /5Phos/ACCGTATCTTGGAAGTGGATGGCATAATTA | 604 |
| | | | TTACACCGCATACACTAAAGATA | |
| 39 | Cd68 | 1 | /5Phos/AGCGTATCCTAGCAAGATCAGACACAAATT | 605 |
| | | | ATTACAGCGCATACACTAAAGATA | |
| 39 | Cd68 | 2 | /5Phos/AGCGTAATTCGGATTTGAATTTGGGCTAAT | 606 |
| | | | TATTACAGCGCATACACTAAAGATA | |
| 39 | Cd68 | 3 | /5Phos/AGCGTATTCTGCGCCATGAATGTCCAATTAT | 607 |
| | | | TACAGCGCATACACTAAAGATA | |
| 39 | Cd68 | 4 | /5Phos/AGCGTAATATGCCCCAAGCCTTTCTAATTAT | 608 |
| | | | TACAGCGCATACACTAAAGATA | |
| 39 | Cd68 | 5 | /5Phos/AGCGTATCCTTCCTCAATCCAATCCCAATTA | 609 |
| | | | TTACAGCGCATACACTAAAGATA | |
| 39 | Cd68 | 6 | /5Phos/AGCGTATTGTGGGTCCGGAGCTGGTAATTA | 610 |
| | | | TTACAGCGCATACACTAAAGATA | |
| 40 | Csf1r | 1 | /5Phos/ATCGTACATTGTAGGGCAACTGAGTAGGGT | 611 |
| | | | AATTATTACATCGCATACACTAAAGATA | |
| 40 | Csf1r | 2 | /5Phos/ATCGTATGCTGTATGCTAGGGTAGGCATAA | 612 |
| | | | TTATTACATCGCATACACTAAAGATA | |
| 40 | Csf1r | 3 | /5Phos/ATCGTAGTACAACGGTAGGTCCCAGTAATT | 613 |
| | | | ATTACATCGCATACACTAAAGATA | |
| 40 | Csf1r | 4 | /5Phos/ATCGTAGTCAAGGACTTTAGCCTTGCGAAT | 614 |
| | | | TATTACATCGCATACACTAAAGATA | |
| 40 | Csf1r | 5 | /5Phos/ATCGTATTTTGCGTAAGACCTGCCTAATTAT | 615 |
| | | | TACATCGCATACACTAAAGATA | |
| 40 | Csf1r | 6 | /5Phos/ATCGTATAAGGACACTTAGGAGAGGAGTA | 616 |
| | | | ATTATTACATCGCATACACTAAAGATA | |
| 41 | Lyz2 | 1 | /5Phos/AAGGTAACAACGTTCATAGACCTTGGAATT | 617 |
| | | | ATTACAAGGCATACACTAAAGATA | |
| 41 | Lyz2 | 2 | /5Phos/AAGGTAGGAAATCGAGGGAATGTGAAATT | 618 |
| | | | ATTACAAGGCATACACTAAAGATA | |
| 42 | Itgax | 1 | /5Phos/ACGGTACGCATATTTTACTGGAAGCTCCAA | 619 |
| | | | AATTATTACACGGCATACACTAAAGATA | |
| 42 | Itgax | 2 | /5Phos/ACGGTACTTAGCTGCCTTACAGAACCCAAT | 620 |
| | | | TATTACACGGCATACACTAAAGATA | |
| 42 | Itgax | 3 | /5Phos/ACGGTACGTCCATGTGAAAATGTGTCAGCT | 621 |
| | | | AATTATTACACGGCATACACTAAAGATA | |
| 42 | Itgax | 4 | /5Phos/ACGGTACCACCTATTTGGTTAGTGGCTAATT | 622 |
| | | | ATTACACGGCATACACTAAAGATA | |
| 42 | Itgax | 5 | /5Phos/ACGGTAGGCATCGATGCAATGGCCTAATTA | 623 |
| | | | TTACACGGCATACACTAAAGATA | |
| 42 | Itgax | 6 | /5Phos/ACGGTAATAACGAATGATGCTTGCAGCAAT | 624 |
| | | | TATTACACGGCATACACTAAAGATA | |
| 43 | Itgam | 1 | /5Phos/AGGGTATGATCCCATACGGTCACATTGTAA | 625 |
| | | | TTATTACAGGGCATACACTAAAGATA | |
| 43 | Itgam | 2 | /5Phos/AGGGTAACAGGATCTCAGTGCTGCTAATTA | 626 |
| | | | TTACAGGGCATACACTAAAGATA | |
| 43 | Itgam | 3 | /5Phos/AGGGTAGGATATCTCCTTCGCGCAGAATTA | 627 |
| | | | TTACAGGGCATACACTAAAGATA | |
| 43 | Itgam | 4 | /5Phos/AGGGTACTGAGATCGTCTTGGCAGATAATT | 628 |
| | | | ATTACAGGGCATACACTAAAGATA | |
| 43 | Itgam | 5 | /5Phos/AGGGTACCTCAAGATGACTGCAGAAGCAAT | 629 |
| | | | TATTACAGGGCATACACTAAAGATA | |
| 43 | Itgam | 6 | /5Phos/AGGGTAGTCAAATGATAGGTTGCCCTAATT | 630 |
| | | | ATTACAGGGCATACACTAAAGATA | |
| 44 | Flt3 | 1 | /5Phos/ATGGTATCGGATTCGTGGGTACGCTAATTA | 631 |
| | | | TTACATGGCATACACTAAAGATA | |
| 44 | Flt3 | 2 | /5Phos/ATGGTATGGGAGATTTGTCCGAACACTTCT | 632 |
| | | | AATTATTACATGGCATACACTAAAGATA | |
| 44 | Flt3 | 3 | /5Phos/ATGGTAGTTCCTTCCCACGGAAGACAAATT | 633 |
| | | | ATTACATGGCATACACTAAAGATA | |
| 44 | Flt3 | 4 | /5Phos/ATGGTACAGCATTTGACCAGAAGCCCTAAT | 634 |
| | | | TATTACATGGCATACACTAAAGATA | |
| 44 | Flt3 | 5 | /5Phos/ATGGTATCTCTCTCGGGAACTCCCAAATTAT | 635 |
| | | | TACATGGCATACACTAAAGATA | |
| 44 | Flt3 | 6 | /5Phos/ATGGTAGGGCGTCATCATTTTCTGCAATTAT | 636 |
| | | | TACATGGCATACACTAAAGATA | |
| 45 | Sirpa | 1 | /5Phos/AATGTATACAGAAACAGGACGCGGAAATT | 637 |
| | | | ATTACAATGCATACACTAAAGATA | |
| 45 | Sirpa | 2 | /5Phos/AATGTAGAAAGATGGCTCAGGCTTGAATTA | 638 |
| | | | TTACAATGCATACACTAAAGATA | |
| 45 | Sirpa | 3 | /5Phos/AATGTACGCACCTACAGAAATAGGCAGGTA | 639 |
| | | | ATTATTACAATGCATACACTAAAGATA | |
| 45 | Sirpa | 4 | /5Phos/AATGTACAACATTAGGTCAGCATTGGGTAA | 640 |
| | | | TTATTACAATGCATACACTAAAGATA | |
| 45 | Sirpa | 5 | /5Phos/AATGTAGCTGGAATCAGGAATCCCCAATTA | 641 |
| | | | TTACAATGCATACACTAAAGATA | |
| 45 | Sirpa | 6 | /5Phos/AATGTATTCCATAGGCTTGGGAGCCAATTA | 642 |
| | | | TTACAATGCATACACTAAAGATA | |
| 46 | Cd19 | 1 | /5Phos/ACTGTACTTCATCTAAAGCTGTGGCTAATTA | 643 |
| | | | TTACACTGCATACACTAAAGATA | |
| 46 | Cd19 | 2 | /5Phos/ACTGTAGTGGATTTCCATAGGACCCAATTA | 644 |
| | | | TTACACTGCATACACTAAAGATA | |
| 46 | Cd19 | 3 | /5Phos/ACTGTATCCCGTACTGGTTCTGGGTAATTAT | 645 |
| | | | TACACTGCATACACTAAAGATA | |
| 46 | Cd19 | 4 | /5Phos/ACTGTACGTTCTCATAGAATTCAGAGCCCTA | 646 |
| | | | ATTATTACACTGCATACACTAAAGATA | |
| 46 | Cd19 | 5 | /5Phos/ACTGTAGGATGTTAGGTGGCTAAATGCCAA | 647 |
| | | | TTATTACACTGCATACACTAAAGATA | |
| 46 | Cd19 | 6 | /5Phos/ACTGTAACCAGTTCTCAACAGCCAGAATTA | 648 |
| | | | TTACACTGCATACACTAAAGATA | |
| 47 | Cd79a | 1 | /5Phos/AGTGTAAGATGATCCCTTCTGCTGTGATGA | 649 |
| | | | ATTATTACAGTGCATACACTAAAGATA | |
| 47 | Cd79a | 2 | /5Phos/AGTGTATGACAAGAAGAGGAGGAGAGGA | 650 |
| | | | ATTATTACAGTGCATACACTAAAGATA | |
| 47 | Cd79a | 3 | /5Phos/AGTGTACACTAGAGAGGGAGCTGGAAATT | 651 |
| | | | ATTACAGTGCATACACTAAAGATA | |
| 47 | Cd79a | 4 | /5Phos/AGTGTAGGTGGGAGAATAGGGTGGGAATT | 652 |
| | | | ATTACAGTGCATACACTAAAGATA | |
| 47 | Cd79a | 5 | /5Phos/AGTGTATCCTCGCCCAAGTTCACCGTAATTA | 653 |
| | | | TTACAGTGCATACACTAAAGATA | |
| 47 | Cd79a | 6 | /5Phos/AGTGTAACGCGGAGGTAAGTACCACAGGA | 654 |
| | | | ATTATTACAGTGCATACACTAAAGATA | |
| 48 | Ms4a1 | 1 | /5Phos/ATTGTATGAAGAAGCTTTGTGTGGGGCAAT | 655 |
| | | | TATTACATTGCATACACTAAAGATA | |
| 48 | Ms4a1 | 2 | /5Phos/ATTGTACAAGGATTCCTGCTCTTGGGAATT | 656 |
| | | | ATTACATTGCATACACTAAAGATA | |
| 48 | Ms4a1 | 3 | /5Phos/ATTGTACGACAGAATGCCCAAGAACAAATT | 657 |
| | | | ATTACATTGCATACACTAAAGATA | |
| 48 | Ms4a1 | 4 | /5Phos/ATTGTATTTTGTAGGCTCTGCTGGGAAAGG | 658 |
| | | | TAATTATTACATTGCATACACTAAAGATA | |
| 48 | Ms4a1 | 5 | /5Phos/ATTGTAGATGGGTGCGAAGACCCCTAATTA | 659 |
| | | | TTACATTGCATACACTAAAGATA | |
| 48 | Ms4a1 | 6 | /5Phos/ATTGTAATGAGAATTGGATTCTTCAGCGTA | 660 |
| | | | ATTATTACATTGCATACACTAAAGATA | |
| 49 | Cd22 | 1 | /5Phos/AAATTAGTTGTACCTACAGACCAGGGTAAT | 661 |
| | | | TATTACAAATCATACACTAAAGATA | |
| 49 | Cd22 | 2 | /5Phos/AAATTAGAGAAATTCAGTCCACAGGTCAGA | 662 |
| | | | ATTATTACAAATCATACACTAAAGATA | |
| 49 | Cd22 | 3 | /5Phos/AAATTACACACTCTTTCCGTGGTCCGTAATT | 663 |
| | | | ATTACAAATCATACACTAAAGATA | |
| 49 | Cd22 | 4 | /5Phos/AAATTAGTGTCATCCATTGCCGGATAATTAT | 664 |
| | | | TACAAATCATACACTAAAGATA | |
| 49 | Cd22 | 5 | /5Phos/AAATTACTTACCTTCAGTACCTTCACGTCTA | 665 |
| | | | ATTATTACAAATCATACACTAAAGATA | |
| 49 | Cd22 | 6 | /5Phos/AAATTAGAGACATTGAGGTGAATGGGAAT | 666 |
| | | | TATTACAAATCATACACTAAAGATA | |
| 50 | Bcl2 | 1 | /5Phos/ACATTATCACGACGGTAGCGACGAGAGAAT | 667 |
| | | | TATTACACATCATACACTAAAGATA | |
| 50 | Bcl2 | 2 | /5Phos/ACATTAGTTGACGCTCTCCACACACAAATTA | 668 |
| | | | TTACACATCATACACTAAAGATA | |
| 50 | Bcl2 | 3 | /5Phos/ACATTACACAGGGCGATGTTGTCCAAATTA | 669 |
| | | | TTACACATCATACACTAAAGATA | |
| 50 | Bcl2 | 4 | /5Phos/ACATTACACCGAACACTTGATTCTGGTAATT | 670 |
| | | | ATTACACATCATACACTAAAGATA | |
| 50 | Bcl2 | 5 | /5Phos/ACATTAATGTCAATCCGTAGGAATCCAATT | 671 |
| | | | ATTACACATCATACACTAAAGATA | |
| 50 | Bcl2 | 6 | /5Phos/ACATTAAATCAGGTGTGTTTTCCTTAATTAT | 672 |
| | | | TACACATCATACACTAAAGATA | |
| 51 | 117r | 1 | /5Phos/AGATTAGTCTTGATACACAGGAGGCCAATT | 673 |
| | | | ATTACAGATCATACACTAAAGATA | |
| 51 | 117r | 2 | /5Phos/AGATTAAGTTGCTTTCACCCCTTGCAATTAT | 674 |
| | | | TACAGATCATACACTAAAGATA | |
| 51 | 117r | 3 | /5Phos/AGATTACTAGAGGAAAGGAGTGGAGGAAT | 675 |
| | | | TATTACAGATCATACACTAAAGATA | |
| 51 | 117r | 4 | /5Phos/AGATTAATTGAAACTCACATTCAGACTAATT | 676 |
| | | | ATTACAGATCATACACTAAAGATA | |
| 51 | 117r | 5 | /5Phos/AGATTACGACTTTCAGGTCAGAGGGAATTA | 677 |
| | | | TTACAGATCATACACTAAAGATA | |
| 51 | 117r | 6 | /5Phos/AGATTAGATTCAAAATTGTGACTTGCAGTA | 678 |
| | | | ATTATTACAGATCATACACTAAAGATA | |
| 52 | Pdcd1 | 1 | /5Phos/ATATTAGAAGACAGCTAGGGCCCAGAATTA | 679 |
| | | | TTACATATCATACACTAAAGATA | |
| 52 | Pdcd1 | 2 | /5Phos/ATATTACCAGTTGGACAAGCTGCAGAATTA | 680 |
| | | | TTACATATCATACACTAAAGATA | |
| 52 | Pdcd1 | 3 | /5Phos/ATATTAGCTACCCTGACTGTCTGAAGAATTA | 681 |
| | | | TTACATATCATACACTAAAGATA | |
| 52 | Pdcd1 | 4 | /5Phos/ATATTAGTGAAGGAGAGCCAGAACCAATTA | 682 |
| | | | TTACATATCATACACTAAAGATA | |
| 52 | Pdcd1 | 5 | /5Phos/ATATTAAACCATTACAGAAGGCGGCCAATT | 683 |
| | | | ATTACATATCATACACTAAAGATA | |
| 52 | Pdcd1 | 6 | /5Phos/ATATTACTCAGATCTCTCCATTTCCAGAATT | 68 |
| | | | ATTACATATCATACACTAAAGATA | |
| 53 | Xcr1 | 1 | /5Phos/AACTTAAACACAGGCAGTAGACAGGAGAA | 685 |
| | | | TTATTACAACTCATACACTAAAGATA | |
| 53 | Xcr1 | 2 | /5Phos/AACTTACTCTGTCTGGACCTTGTGCGAATTA | 686 |
| | | | TTACAACTCATACACTAAAGATA | |
| 53 | Xcr1 | 3 | /5Phos/AACTTAGAGTACAGGACAATGGTAGAGAA | 687 |
| | | | TTATTACAACTCATACACTAAAGATA | |
| 53 | Xcr1 | 4 | /5Phos/AACTTAATCGGTGGATGGTCATGATGAATT | 688 |
| | | | ATTACAACTCATACACTAAAGATA | |
| 53 | Xcr1 | 5 | /5Phos/AACTTACATATGTAAAGGTACCAGGGGAAT | 689 |
| | | | TATTACAACTCATACACTAAAGATA | |
| 53 | Xcr1 | 6 | /5Phos/AACTTATGTACGCTTTGTGCTTCCTAATTAT | 690 |
| | | | TACAACTCATACACTAAAGATA | |
| 54 | Irf8 | 1 | /5Phos/ACCTTATATATGGCTCAGAAATGTCCAGAA | 691 |
| | | | TTATTACACCTCATACACTAAAGATA | |
| 54 | Irf8 | 2 | /5Phos/ACCTTAGGCATATCCGGTCACCAGTAATTAT | 692 |
| | | | TACACCTCATACACTAAAGATA | |
| 54 | Irf8 | 3 | /5Phos/ACCTTATTGTGAACAGGTTGTCCTGGCAATT | 693 |
| | | | ATTACACCTCATACACTAAAGATA | |
| 54 | Irf8 | 4 | /5Phos/ACCTTATTGTAAGGTATGGCCTGTAGCTAA | 694 |
| | | | TTATTACACCTCATACACTAAAGATA | |
| 54 | Irf8 | 5 | /5Phos/ACCTTACACAACCAAAATTCCTGCGAATTAT | 695 |
| | | | TACACCTCATACACTAAAGATA | |
| 54 | Irf8 | 6 | /5Phos/ACCTTACAGGTAAGGCTTTCCTGCAAATTAT | 696 |
| | | | TACACCTCATACACTAAAGATA | |
| 55 | Irf4 | 1 | /5Phos/AGCTTAGTAGGGAAACAGGACCTGGTAATT | 697 |
| | | | ATTACAGCTCATACACTAAAGATA | |
| 55 | Irf4 | 2 | /5Phos/AGCTTAACGTGTTCAGGTAACTCGTAGCCA | 698 |
| | | | ATTATTACAGCTCATACACTAAAGATA | |
| 55 | Irf4 | 3 | /5Phos/AGCTTACTGTGTGTCAAAGAGCTTGCAAAT | 699 |
| | | | TATTACAGCTCATACACTAAAGATA | |
| 55 | Irf4 | 4 | /5Phos/AGCTTACTGTGATGAGCTTCCTCTGTAATTA | 700 |
| | | | TTACAGCTCATACACTAAAGATA | |
| 55 | Irf4 | 5 | /5Phos/AGCTTACATTGATATGGGATTTCTGGGAAT | 701 |
| | | | TATTACAGCTCATACACTAAAGATA | |
| 55 | Irf4 | 6 | /5Phos/AGCTTAAGTTTCACTTACGGTGCAGGAATT | 702 |
| | | | ATTACAGCTCATACACTAAAGATA | |
| 56 | H2-Aa | 1 | /5Phos/ATCTTACAAGACTCCCAAGTTGTGTAATTAT | 703 |
| | | | TACATCTCATACACTAAAGATA | |
| 56 | H2-Aa | 2 | /5Phos/ATCTTAAGCTGGTCTCATAAACACCGTAATT | 704 |
| | | | ATTACATCTCATACACTAAAGATA | |
| 56 | H2-Aa | 3 | /5Phos/ATCTTACCATAGGTGCCTACGTGGTAATTAT | 705 |
| | | | TACATCTCATACACTAAAGATA | |
| 56 | H2-Aa | 4 | /5Phos/ATCTTACTTGAATGATGAAGATGGTGCCCA | 706 |
| | | | AATTATTACATCTCATACACTAAAGATA | |
| 56 | H2-Aa | 5 | /5Phos/ATCTTAAAAGGCCCTGGGTGTCTGGAATTA | 707 |
| | | | TTACATCTCATACACTAAAGATA | |
| 57 | Cd40 | 1 | /5Phos/AAGTTATGACTGATTGGAGAAGAAGCAATT | 708 |
| | | | ATTACAAGTCATACACTAAAGATA | |
| 57 | Cd40 | 2 | /5Phos/AAGTTACACACTGCCCTAGATGGACCGAAT | 709 |
| | | | TATTACAAGTCATACACTAAAGATA | |
| 57 | Cd40 | 3 | /5Phos/AAGTTAACAGTGTCTGATTCTGCGGTAATT | 710 |
| | | | ATTACAAGTCATACACTAAAGATA | |
| 57 | Cd40 | 4 | /5Phos/AAGTTACCAAGTTCTTATCCTCACAGCTAAT | 711 |
| | | | TATTACAAGTCATACACTAAAGATA | |
| 57 | Cd40 | 5 | /5Phos/AAGTTACTGAGCACATGCCTCGCAATCCTA | 712 |
| | | | ATTATTACAAGTCATACACTAAAGATA | |
| 57 | Cd40 | 6 | /5Phos/AAGTTAGTCACATGGGTGGCATTGGGAATT | 713 |
| | | | ATTACAAGTCATACACTAAAGATA | |
| 58 | Cd83 | 1 | /5Phos/ACGTTATTCCGTACCAGGTTTAGAAATAATT | 714 |
| | | | ATTACACGTCATACACTAAAGATA | |
| 58 | Cd83 | 2 | /5Phos/ACGTTAACTCTGTAGCTTCCTTGGGAATTAT | 715 |
| | | | TACACGTCATACACTAAAGATA | |
| 58 | Cd83 | 3 | /5Phos/ACGTTAGGGAATAGGCCCTTCTCCTAATTAT | 716 |
| | | | TACACGTCATACACTAAAGATA | |
| 58 | Cd83 | 4 | /5Phos/ACGTTACTCAGTCCATTGTCTGCCCTAATTA | 717 |
| | | | TTACACGTCATACACTAAAGATA | |
| 58 | Cd83 | 5 | /5Phos/ACGTTAGACCAGATAGCCACATGAAACATA | 718 |
| | | | ATTATTACACGTCATACACTAAAGATA | |
| 58 | Cd83 | 6 | /5Phos/ACGTTATCGTGATCATATGATGGTGCTAATT | 719 |
| | | | ATTACACGTCATACACTAAAGATA | |
| 59 | Cd86 | 1 | /5Phos/AGGTTAGTCTCCACGGAAACAGCATAATTA | 720 |
| | | | TTACAGGTCATACACTAAAGATA | |
| 59 | Cd86 | 2 | /5Phos/AGGTTAAACCAACTTTTGCTGGTCCTAATTA | 721 |
| | | | TTACAGGTCATACACTAAAGATA | |
| 59 | Cd86 | 3 | /5Phos/AGGTTAGGCTGTTGGAGATACTGAACAGTA | 722 |
| | | | ATTATTACAGGTCATACACTAAAGATA | |
| 59 | Cd86 | 4 | /5Phos/AGGTTACCAGAACACACACAACGGTAATTA | 723 |
| | | | TTACAGGTCATACACTAAAGATA | |
| 59 | Cd86 | 5 | /5Phos/AGGTTACTTGTGACATACAATGATGAGCAG | 724 |
| | | | CAATTATTACAGGTCATACACTAAAGATA | |
| 59 | Cd86 | 6 | /5Phos/AGGTTATTCATCAATGTGACTGCTCTCTAAT | 725 |
| | | | TATTACAGGTCATACACTAAAGATA | |
| 60 | H2-K1 | 1 | /5Phos/ATGTTACGGCGATTCGCGACTTCTGAGTAA | 726 |
| | | | TTATTACATGTCATACACTAAAGATA | |
| 60 | H2-K1 | 2 | /5Phos/ATGTTAATCAGAGGTCTGGGAGCCTAATTA | 727 |
| | | | TTACATGTCATACACTAAAGATA | |
| 60 | H2-K1 | 3 | /5Phos/ATGTTAAGGCGTACTGCTGGTACCCAATTA | 728 |
| | | | TTACATGTCATACACTAAAGATA | |
| 60 | H2-K1 | 4 | /5Phos/ATGTTATCAGTGTTTGGCTGAACCAGAATT | 729 |
| | | | ATTACATGTCATACACTAAAGATA | |
| 61 | Ccl17 | 1 | /5Phos/AATTTACCTGATAGGAATGGCCCCTAATTAT | 730 |
| | | | TACAATTCATACACTAAAGATA | |
| 61 | Ccl17 | 2 | /5Phos/AATTTATGGAACACTCCACTGAGGTCTAATT | 731 |
| | | | ATTACAATTCATACACTAAAGATA | |
| 61 | Ccl17 | 3 | /5Phos/AATTTATGTTTGTCTTTGGGGTCTGCAAATT | 732 |
| | | | ATTACAATTCATACACTAAAGATA | |
| 61 | Ccl17 | 4 | /5Phos/AATTTAACCAAAAGCTGAGGTGAAGGAATT | 733 |
| | | | ATTACAATTCATACACTAAAGATA | |
| 61 | Ccl17 | 5 | /5Phos/AATTTATTATGTTGAAACCATGGACAGCAG | 734 |
| | | | AATTATTACAATTCATACACTAAAGATA | |
| 62 | Ccl22 | 1 | /5Phos/ACTTTAACGGTTATCAAAACAACGCCAGAA | 735 |
| | | | TTATTACACTTCATACACTAAAGATA | |
| 62 | Ccl22 | 2 | /5Phos/ACTTTACTAAACGTGATGGCAGAGGAATTA | 736 |
| | | | TTACACTTCATACACTAAAGATA | |
| 62 | Ccl22 | 3 | /5Phos/ACTTTATATGGAGTAGCTTCTTCACCCAATT | 737 |
| | | | ATTACACTTCATACACTAAAGATA | |
| 62 | Ccl22 | 4 | /5Phos/ACTTTAAGCTACACAGGCAAGGAGTAATTA | 738 |
| | | | TTACACTTCATACACTAAAGATA | |
| 62 | Ccl22 | 5 | /5Phos/ACTTTAGGCAGGATTTTGAGGTCCAGAATT | 739 |
| | | | ATTACACTTCATACACTAAAGATA | |
| 62 | Ccl22 | 6 | /5Phos/ACTTTAGCATGTGGTTGAAGAGGGGTAATT | 740 |
| | | | ATTACACTTCATACACTAAAGATA | |
| 63 | Ccr4 | 1 | /5Phos/AGTTTATCGAGTTGACCGAGTACTGGGTAA | 741 |
| | | | TTATTACAGTTCATACACTAAAGATA | |
| 63 | Ccr4 | 2 | /5Phos/AGTTTAGATCTTGCACAGACCTAGTAATTAT | 742 |
| | | | TACAGTTCATACACTAAAGATA | |
| 63 | Ccr4 | 3 | /5Phos/AGTTTATGATCCACAGTGGACTGCGAATTA | 743 |
| | | | TTACAGTTCATACACTAAAGATA | |
| 63 | Ccr4 | 4 | /5Phos/AGTTTAAAATGACGGGGTTAAGGCAAATTA | 744 |
| | | | TTACAGTTCATACACTAAAGATA | |
| 63 | Ccr4 | 5 | /5Phos/AGTTTAATCTCTACGCTTGTAACCAGGCTAA | 745 |
| | | | TTATTACAGTTCATACACTAAAGATA | |
| 63 | Ccr4 | 6 | /5Phos/AGTTTAAACTGTTCTAACTTTTAATACAACT | 746 |
| | | | AATTATTACAGTTCATACACTAAAGATA | |
| 64 | Ccl25 | 1 | /5Phos/ATTTTACACAGCACGTAGGTTGCAGAATTA | 747 |
| | | | TTACATTTCATACACTAAAGATA | |
| 64 | Ccl25 | 2 | /5Phos/ATTTTACCGGAGAACATTCCATTTGATAATT | 748 |
| | | | ATTACATTTCATACACTAAAGATA | |
| 64 | Ccl25 | 3 | /5Phos/ATTTTACAATAGCTCCTTGGTGAGTAATTAT | 749 |
| | | | TACATTTCATACACTAAAGATA | |
| 64 | Ccl25 | 4 | /5Phos/ATTTTATGCCTATGGGATCAGTGAACTAATT | 750 |
| | | | ATTACATTTCATACACTAAAGATA | |
| 64 | Ccl25 | 5 | /5Phos/ATTTTAAGTTCCTTCCTCATACATGCTAATTA | 751 |
| | | | TTACATTTCATACACTAAAGATA | |
| 64 | Ccl25 | 6 | /5Phos/ATTTTATCATGTCCTCTGGATTCCCAAATTA | 752 |
| | | | TTACATTTCATACACTAAAGATA | |

### 4) STARmap Data Analysis

STARmap data analysis was performed similarly to Wang and Shi (Wang et al., Science 361 (2018) and Shi et al Nature 622, 552-561 (2023)). Briefly, image deconvolution was achieved with Huygens Essential version 21.04 (Scientific Volume Imaging, The Netherlands, http://svi.nl), using the CMLE algorithm, with SNR:10 and 10 iterations. Image registration, spot calling, and barcode filtering were performed as previously described.

### a) 3D Cell Segmentation:

A synthetic image with improved contrast between cell nuclei was generated by multiplying the inverted Flamingo staining image and DAPI staining image after enhancing contrast using Fiji for each field of view (FOV). A StarDist 3D segmentation model was then trained using a manually labeled training dataset created from the synthetic data. Subsequently, the model was applied to predict segmentation for each FOV.

### b) Tissue Region Identification:

To isolate low-frequency (i.e. large length scale) transcriptional patterns over the tissue, Laplacian smoothing over a spatial Delaunay triangulation (i.e. a nearest neighbor mesh of cells) was performed. The specific low-pass filter used was a heat kernel with time t=10. PCA was then performed on the resulting low-pass filtered cell-by-gene matrix to identify region features, followed by clustering in PC space to produce categorical region labels. K-means was used for clustering to avoid smoothing-induced spatial autocorrelation artifacts.

### c) Quality Control & Cell Type Classification:

Cells with less than 2 reads and expressed fewer than 2 genes were excluded. Gene expression profiles were normalized and scaled using standard Scanpy procedures. A hierarchical clustering approach was then utilized to create a three-level celltype annotation. Initially, 24 clusters were identified through k-means clustering of the preprocessed gene expression profile containing 19 genes, which were further categorized into four level 1 cell types (T cells, B cells, Macrophages, and Dendritic cells). Cells lacking expression of any of the selected 19 gene markers were removed from subsequent analysis. Each level 1 cell type underwent additional k-means clustering to establish level 2 annotations. Specifically, T cells were subdivided into ectopically generated T cells (Ccr7+), CD4+ T cells (Cd4+), and CD8+ T cells (Cd8a+). Macrophages were classified as Activated Macrophages (Cd68+) and Monocytes (Csf1r+, Lyz2+), while Dendritic cells were differentiated into cDC1 (Irf8+) and cDC2 (Irf4+). 9 Level 2 CD4+/CD8+ T cells and Synthetic T cells then underwent a level-3 k-means clustering using a panel of 15 markers for further refinement in classification.

### d) Neighborhood Enrichment Analysis:

A permutation-based approach from Squidpy was used to quantify localized cell neighborhoods in each tissue section. For each pair of cell types, the analysis calculates an enrichment score that reflects how often cells of these two types are found in close proximity compared to what would be expected by chance. This involves comparing the observed frequency of each cell type pair within neighborhoods against a null model that assumes a random distribution of cell types. The significance of the enrichment scores is assessed using permutation tests as the p-values shown in the corresponding heatmaps.

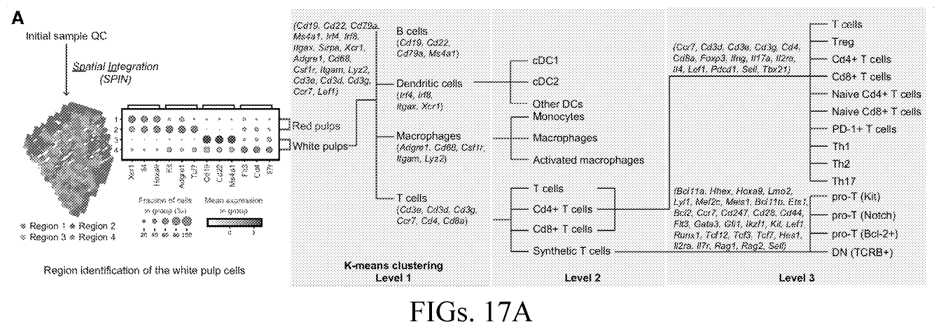

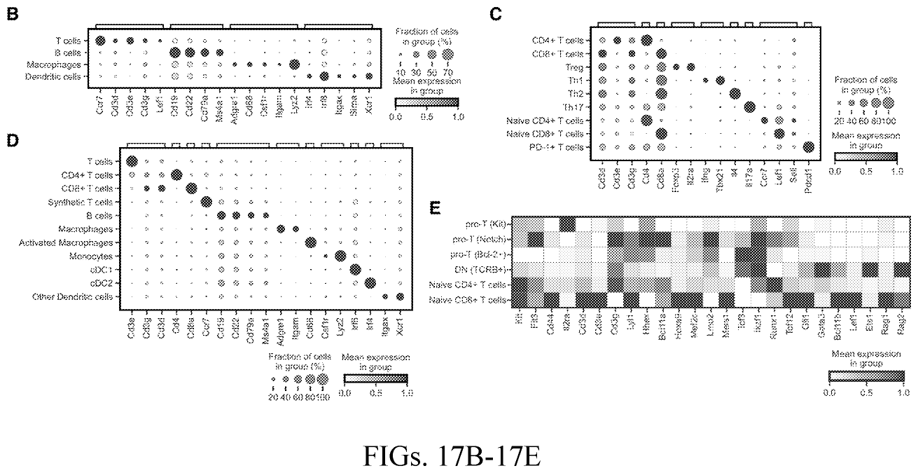

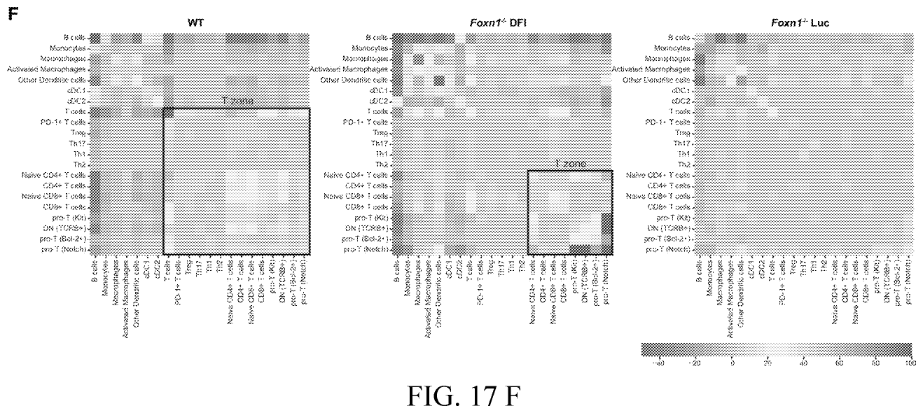

A panel of 64-gene probes were designed (Tables 2 and 3) to accurately subtype splenocytes, assess their activation levels, and detail any immature T cell states based on TF expression patterns (FIG. 17A). Cells in the white pulp were spatially organized (the lymphoid tissue component of the spleen) into distinct regions informed by the native spleen histology (such as B cell follicles and periarteriolar lymphoid sheath (PALS)) using SPIN and performed subtyping with k-means clustering. Furthermore, Applicants mapped out cell-cell interactions based on their proximity in 3D tissue reconstructions (FIG. 15E, FIGS. 17A-17B).

Based on the functionally defined splenic architecture, Applicants 3D-segmented and quantified immune cells in all captured PALS (FIG. 15F-15G; FIG. 17A). In Luc-treated athymic mice, Applicants found that the physiological PALS architecture and segregation into a periarteriolar T cell zone surrounded by B cells and other APCs (Macrophages, Mφ) and DCs was disrupted by the severe reduction of T cells (FIG. 15F). DFI-treated mice, however, demonstrated a regeneration of this architecture that was driven by the addition of T cell clusters not found in Luc-treated athymic animals (FIG. 15F). Quantifying the cells in these regions showed that these additional T cells are largely immature, with some of them having progressed to naïve single-positive CD4⁺ and CD8⁺ T cells (FIG. 15G, FIG. 17E).

Applicants also observed a substantial increase in cDC1 cells in the STARmap data (FIG. 15G). Previous work has shown that Flt3L administration increased DC numbers in the spleen from Flt3-expressing progenitors. DFI administration led to a systemic increase in Flt3L levels (FIG. 3G). Applicants therefore hypothesized that the observed increase in splenic cDC1 populations is an additional mechanism of the Flt3l mRNA-containing DFI approach, which induces development of DCs from progenitors. In addition, Applicants observed enhanced activation of DC subtypes in the PALS (FIG. 15H). As neither treatment with Flt3l mRNA alone or recombinant Flt3L protein was able to recapitulate the increase in vaccination-specific or naïve T cells Applicants observed with full DFI-treatment in aged animals, Applicants reasoned that these two mechanisms of DFI treatment are likely complementary.

T cells are guided into the spleen through a process involving chemokines and their receptors, directing them to areas within the spleen for effective interaction with antigen presenting cells (APCs). The CCR7 chemokine receptor is particularly important for T cell migration to secondary lymphoid organs like the spleen. This CCR7-dependent pathway is vital for correctly positioning T cells, allowing them to efficiently encounter and respond to antigens. Applicants found that in addition to being present at higher numbers (FIG. 15F-G), immature T cells expressed high levels of Ccr7, potentially explaining their homing to the spleen (FIG. 17B).

Because T cell maturation depends on interaction with APCs, Applicants analyzed the cellular neighborhood of immature T cells using Squidpy. For each pair of cell types, the analysis calculates an enrichment score that reflects how often cells of two types are found in close proximity compared to what would be expected by chance. Spatial profiling studies of mouse spleens have revealed a strong association between cells of the same phenotypic class, suggesting that similar cell types tend to cluster together, a principle referred to as homotypic association, which appears to be a key driver in the organization of immune tissue. Applicants found this pattern is consistent across major cell types in the white pulp of wild-type (Foxn1^{+/+}) animals, including T and B cells, and extends to less common cells such as monocytes (FIG. 15I). Although cell-cell interactions were largely limited to the myeloid cell compartment in Luc-treated Foxn1^{-/-} animals, mirroring their lack of T cells, in DFI-treated animals Applicants observed the re-emergence of cellular interactions among maturing T cells as well as between T cells and DCs in the PALS (FIG. 15I, FIG. 17F).

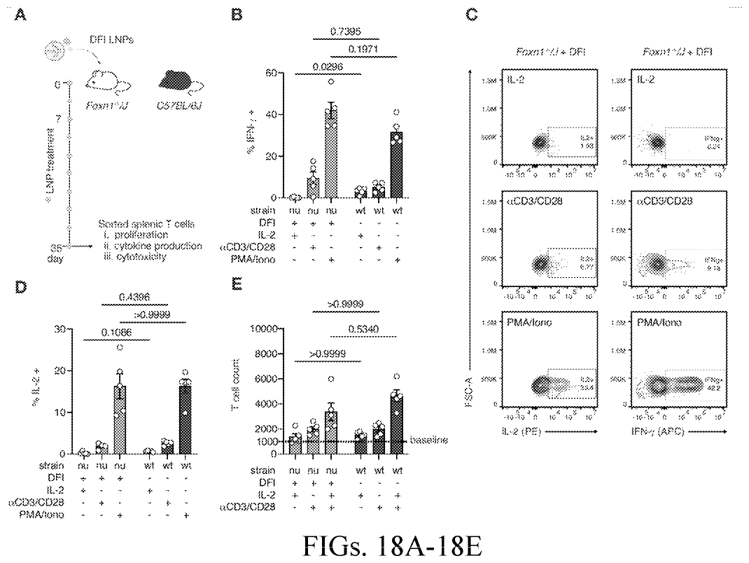

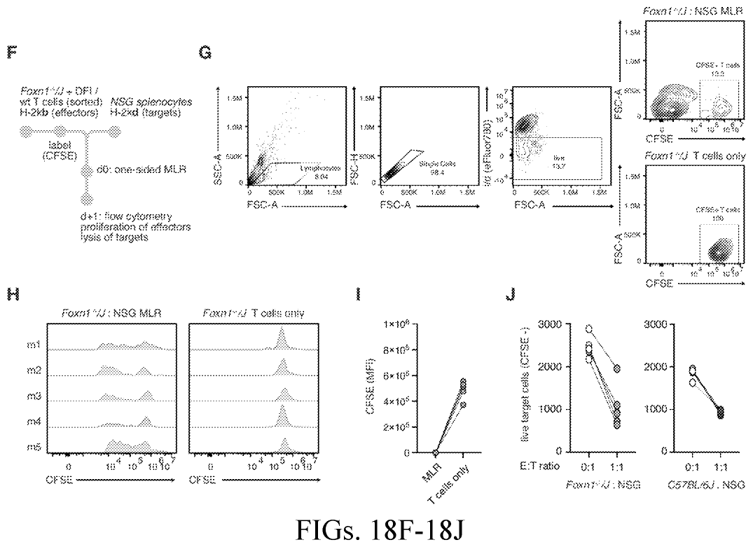

To verify that the ectopically generated naïve T cells generated by DFI treatment are functional, Applicants extracted all CD3⁺ T cells from the spleens of Foxn1^{-/-} animals treated for 35 days with DFI and evaluated their functionality using three metrics: 1) capacity to produce two key T cell cytokines, IL-2 and IFN-γ, upon activation, 2) ability to proliferate following engagement of the TCR, and 3) competence in promoting cellular cytotoxicity (FIG. 18A).

Applicants found that ectopically in vivo-produced T cells produced IL-2 and IFN-γ upon stimulation with PMA/ionomycin. However, stimulation with these compounds bypasses the TCR complex, so Applicants also tested if treatment with aCD3/CD28 antibodies, which engage the TCR complex, resulted in IL-2 and IFN-γ production as well. Even with this assay, Applicants found that the T cells from DFI-treated animals produced similar levels to those produced by T cells isolated from wild-type mice (FIG. 18B-18D). Furthermore, DFI-derived T cells demonstrated wild-type levels of proliferation activity in response to TCR activation by αCD3/CD28 engagement, which was further increased with the addition of 100 ng/ml IL-2 (FIG. 18E).

To assess TCR-driven cytotoxicity, Applicants performed a one-sided mixed lymphocyte reaction (MLR), which is widely used to assess T cellular function, particularly in the context of allogeneic responses (FIG. 18F). Foxn1^{-/-} mice express the MHC class I H-2^{Kb} allele, while NSG mice, which lack T cells, are on the H-2Kd genetic background. This mismatch will in principle elicit alloreactivity. Applicants co-cultured sorted and Carboxyfluorescein succinimidyl ester (CFSE)-labeled DFI-derived T cells and splenocytes from NSG mice and then analyzed cells by flow cytometry (FIG. 18G). After 24 hours of co-culture, Applicants found a significant loss of NSG (CFSE⁻) splenocytes in the MLR setups with both DFI-derived and wild-type effector T cells relative to the non-MLR controls. This was accompanied by a significant proliferation of effector T cells, which Applicants were able to track by CFSE dilution (FIGS. 18H-18J).

Together, these results show that DFI-treatment induces the accumulation of mature T cells and DCs in the spleens of athymic animals. Ectopically generated T cells exhibited comparable cellular functions relative to physiologically developed T cells from wild-type animals, underscoring the potential of these cells to contribute to adaptive immune responses. Importantly, the multifaceted assessments of T cell functionality revealed that mature T cells generated by DFI treatment did not exhibit unnaturally high levels of activity or abnormal proliferation.

### Example 6: DFI Factors Demonstrate Favorable Toxicity and Immunological Safety

### Spontaneous Type-1 Model and Clinical and Molecular Monitoring

NOD/ShiLtJ (NOD) mice were treated with DFI mRNA or Luc mRNA encapsulated in SM-102 LNPs twice per week for 4 weeks followed by clinical and molecular monitoring for the development of type 1 (T1D). NOD.Cg-Tg (TcraTcrbNY8.3)1Pesa/DvsJ (NY8.3) mice transgenic for the autoreactive TCR recognizing NRP-V7 included as positive control group. Experimental endpoint (Onset of T1D) was reached in animals with blood glucose levels >200 mg/dL in two independent measurements or in animals developing glucosuria.

The safety of DFI treatment was investigated. Applicants confirmed that DFI treatment as an LNP did not affect the weight of aged animals relative to treatment with Luc LNPs over the course of 35 days of bi-weekly treatments (FIGS. 13A-13B). Applicants then assessed the relative safety of DFI mRNA (packaged in LNPs) treatment relative to systemic protein delivery. Applicants observed no significant changes in liver function parameters such as AST, ALT, CK, or in hepatic steatosis indicators GGT and bilirubin in mice treated with DFI mRNA-LNPs compared to those receiving recombinant proteins or a saline control. However, Applicants saw an elevation in triglyceride levels, which could indicate the presence or breakdown of LNPs, but this did not lead to increased serum cholesterol or LDL levels (FIG. 13C). Furthermore, DFI mRNA did not induce a significant inflammatory response compared to the control group, except for a moderate increase in IL-6 levels, which aligns with previous preclinical evaluations of SM-102 LNP formulations (FIG. 13D). Conversely, mice treated with recombinant FLT-3L and IL-7 exhibited significant elevations in GM-CSF, IL-10, IL-12, and IL-1β levels (FIG. 13D).

A key question raised by these findings is whether DFI treatment leads to autoimmune disease via the generation of autoreactive T cells, which are typically eliminated in the thymus through the process of negative selection. To answer this, Applicants first turned to the T cell-driven non-obese diabetic (NOD) mouse model to assess if DFI treatment affects the susceptibility to Type 1 diabetes (T1D). In NOD mice, T1D is brought on by autoreactive T cells, the main TCR clonotype (NRP-V7) of which is detectable in the bloodstream via peptide-H2^{kd} tetramer staining of its mimotope.

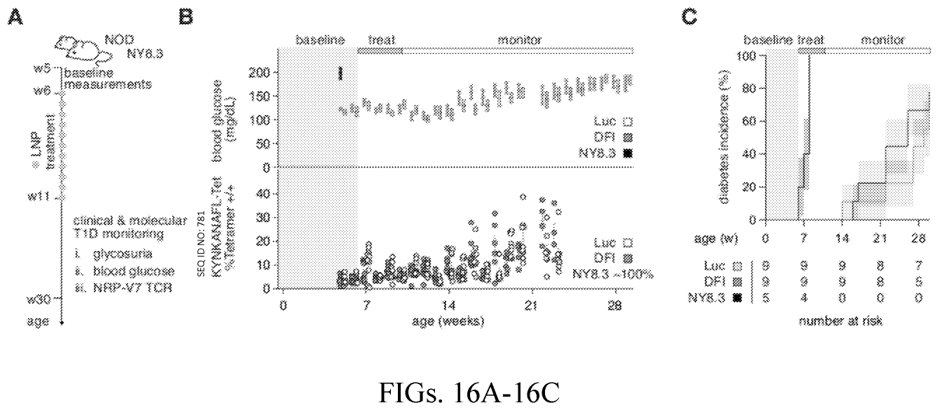

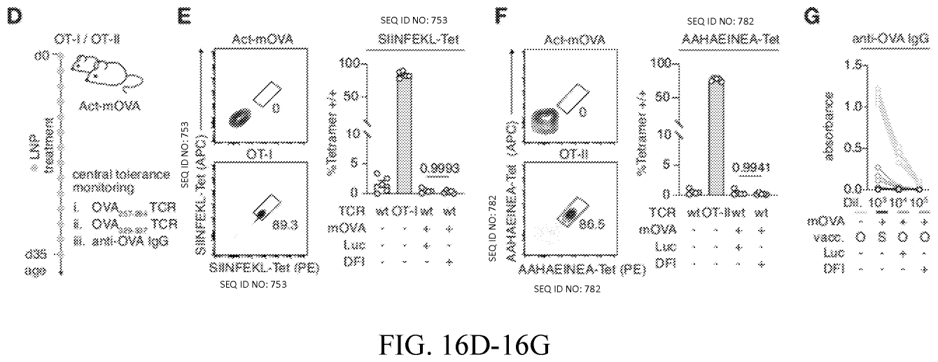

To define a threshold for exacerbated T1D, Applicants used NOD mice genetically modified to express only the NRP-V7 TCR (NOD NY8.3). After establishing baseline levels of serum glucose, glycosuria, and autoreactive T cell levels, NOD mice were treated with Luc or DFI twice weekly for five weeks (FIG. 16A). In parallel, Applicants monitored the onset of T1D via blood and urine glucose testing. Applicants also performed weekly flow cytometry from peripheral blood of these animals to quantify the frequency of circulating autoreactive T cells carrying the TCRs that recognize the NRP-V7 mimotope KYNKANVFL (SEQ ID NO: 758). Applicants found no significant difference in blood glucose levels (FIG. 16B, top) or diabetes incidence (FIG. 16C) between littermate mice treated with DFI or Luc LNPs throughout the 6-month observation period. By contrast, all NY8.3 mice developed T1D by 8 weeks of age (FIG. 16C). Flow cytometry analysis revealed the presence of NRP-V7-specific TCRs in all tested animals as early as 5 weeks of age, with a progressive increase to about 28% NRP-V7-specific T cells (compared to nearly 100% in NY8.3 mice). However, despite the rising variability among individuals over time—correlating with the emergence of clinically evident T1D—the difference in NRP-V7-specific T cells between DFI- and Luc-treated mice did not reach statistical significance at any point of the study (FIG. 16B, bottom).

Although Applicants were encouraged by the results in the NOD mice, central tolerance in these animals is compromised. Applicants therefore established a second model to test if DFI affects the maintenance of negative selection against a germline encoded antigen. Applicants used Act-mOVA mice, which possess a transgenic construct that drives the expression of chicken ovalbumin, along with elements from the H-2^{Kb} and the H-2^{Db} alleles, under the regulation of a chicken beta actin promoter and the cytomegalovirus (CMV) immediate-early enhancer. This configuration ensures robust and widespread germline expression of the OVA 254-267-K^{b} peptide:MHC complex, recognizable by OT-I TCRs, and the OVA 323-339-I-A^{b} complex, recognizable by OT-II TCRs. In the Act-mOVA mice, OVA is constitutively expressed from an early developmental stage, in contrast to being introduced externally as performed in the previous vaccination experiments. This internal expression subjects OVA to the organism's mechanisms of central tolerance, leading to a physiological absence of T cells or immunoglobulins that recognize OVA. Applicants treated these mice with two doses of either Luc or DFI LNPs, administered twice weekly over a five-week period. To assess whether central tolerance was disrupted, Applicants utilized OVA tetramer staining to detect autoreactive TCRs and an anti-OVA pan-IgG ELISA for autoreactive antibodies (FIG. 16D). Following a five-week DFI treatment, Applicants did not detect any T cells specific for OVA peptides 254-267 in CD8⁺ T cells or 323-339 in CD4⁺ T cells in the peripheral blood of these mice. Importantly, compared to wild-type mice, Act-mOVA mice showed near-zero levels of T cells binding to the OVA 254-267-K^{b} peptide throughout the treatment, indicating a continuous negative selection against these TCRs (FIG. 16E-F). In addition, Applicants challenged these animals with an exogenous OVA vaccine (with the strong adjuvant CFA). In this challenge experiment, wild-type mice developed robust anti-OVA IgG serum levels, but neither DFI nor Luc treated Act-mOVA animals showed any significant antibody response (FIG. 16G). Finally, Applicants transferred genetically labeled CD45.2 pro-T cells into immunocompetent, but aged CD45.1 animals to better understand the fate of immature, but systemically circulating T cells in the presence of a thymus (FIGS. 13F-13H). Applicants detected the transferred cells in the thymus as early as 24 hours after i.v. injection (FIG. 13G). After 7 days, Applicants still detected immature CD45.2 T cells in the thymus of recipient animals. However, the spleen of recipients only contained single-positive CD45.2 T cells, suggesting that further maturation and negative selection by the recipient's thymus can in principle occur in this context (FIG. 13H).

Together, Applicants results suggest that while DFI enhances immunity against foreign antigens in aged animals, it does not interfere with self-tolerance. This underscores the potential for DFI's safe application. Additionally, it avoids the potentially severe adverse effects and costs associated with recombinant cytokine treatments or cellular reprogramming approaches.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

1. A composition comprising:
(a) one or more polynucleotides encoding one or more activating proteins that induce T cell differentiation of T cell progenitor cells, and
(b) a delivery vehicle configured to deliver the one or more polynucleotides to a target tissue where T cell progenitors differentiate.

2. The composition of claim 1, wherein the one or more activating proteins is an agonist of a Notch signaling pathway comprising DLL1, DLL4, Jagged1, Jagged2, or a combination thereof, or of a T cell receptor (TCR) signaling pathway comprising a major histocompatibility complex (MHC) class I ligand comprising HLA-A, HLA-B, HLA-C, or a homolog thereof, or an MHC class II ligand comprising HLA-DP, HLA-DQ, HLA-DR, or a homolog thereof.

3. (canceled)

4. (canceled)

5. (canceled)

6. (canceled)

7. The composition of claim 1, further comprising one or more cytokines, or one or more polynucleotides encoding said cytokines.

8. The composition of claim 7, wherein the one or more cytokines comprise IL-7, FLT3L, KIT ligand, IL-2, IL-4, IL-15, CCL25, or a combination thereof, and/or wherein the one or more cytokines induce expression of the transcription factor RUNX3.

9. (canceled)

10. The composition of claim 1, wherein the one or more polynucleotides comprise one or more plasmid DNAs encoding the one or more activating proteins and/or cytokines; one or more mRNA molecules encoding the one or more activating proteins and/or cytokines; wherein the one or more mRNAs comprise one or more modifications that increase mRNA stability, reduce immunogenicity, and/or improve translation efficiency; or a combination thereof.

11. (canceled)

12. The composition of claim 1, wherein the delivery vehicle comprises a lipid nanoparticle, an exosome, a microvesicle, a viral vector comprising a retroviral, lentiviral, adenoviral, adeno-associated, or herpes simplex viral vector, an engineered retroelement vector, a polynucleotide-based nanostructure, or an extracellular contractile injection system.

13. (canceled)

14. The composition of claim 1, wherein the target tissue is the liver, spleen, small intestine, thymus, or a combination thereof.

15. A method for inducing T cell progenitor differentiation comprising:
delivering, to a target tissue where T cell progenitor differentiation occurs, one or more polynucleotides encoding one or more activating proteins that induce differentiation of T cell progenitor cells.

16. The method of claim 15, wherein the one or more polynucleotides are formulated such that the one or more polynucleotides are delivered to endothelial cells in the target tissue, and wherein the activating proteins are displayed on a surface of the endothelial cells.

17. The method of claim 15, wherein the target tissue is the liver, spleen, small intestine, thymus, or a combination thereof.

18. The method of claim 15, wherein the one or more activating proteins induce differentiation of T cell progenitors to double positive (DP) T cells.

19. The method of claim 15, wherein the one or more activating proteins is an agonist of a Notch signaling pathway or of a T cell receptor (TCR) signaling pathway.

20. The method of claim 19, wherein the agonist of the Notch signaling pathway comprises DLL1, DLL4, Jagged1, Jagged2, or a combination thereof, or wherein the agonist of the TCR signaling pathway is a major histocompatibility complex (MHC) class I ligand comprising HLA-A, HLA-B, HLA-C, or a homolog thereof, or an MHC class II ligand comprising HLA-DP, HLA-DQ, HLA-DR, or a homolog thereof.

21. (canceled)

22. (canceled)

23. (canceled)

24. The method of claim 15, wherein the one or more polynucleotides comprise one or more plasmid DNAs encoding the one or more activating proteins; one or more mRNA molecules encoding the one or more activating proteins, wherein the one or more mRNA molecules comprise one or more modifications that increase mRNA stability, reduce immunogenicity, and/or improve translation efficiency; or a combination thereof.

25. (canceled)

26. The method of claim 15, to wherein the one or more polynucleotides are delivered a lipid nanoparticle, an exosome, a microvesicle, a viral vector comprising a retroviral, lentiviral, adenoviral, adeno-associated, or herpes simplex viral vector, an engineered retroelement vector, a polynucleotide-based nanostructure, or an extracellular contractile injection system.

27. (canceled)

28. The method of claim 15, further comprising delivering one or more cytokines, or one or more polynucleotides encoding said cytokines.

29. The method of claim 28, wherein the one or more cytokines comprise IL-7, FLT3L, KIT ligand, IL-2, IL-4, IL-15, CCL25, or a combination thereof, and/or wherein the one or more cytokines induce expression of the transcription factor RUNX3.

30. (canceled)

31. The method of claim 28, wherein the one or more cytokines are co-delivered with the one or more polynucleotides encoding the one or more activating proteins, or wherein the one or more cytokines are delivered separately, either temporally or spatially, from the one or more polynucleotides encoding the one or more activating proteins, and wherein the one or more cytokines induce differentiation of double positive (DP) T cells into single positive (SP) T cells.

32. (canceled)

33. (canceled)

34. A method for identifying candidate activating proteins, or combinations of activating proteins, that induce T cell proliferation and differentiation, said method comprising:
(a) delivering one or more polynucleotides encoding one or more candidate activating proteins that induce T cell differentiation of T cell progenitor cells to a population of cells derived from a tissue of interest,
(b) selecting a sample of single clone cells expressing the candidate activating proteins from the population of cells derived from the tissue of interest,
(c) treating a population of T cell progenitors with the sample of single clone cells expressing the candidate activating proteins,
(d) quantifying the percentage of T cells differentiated from the population of T cell progenitors, and
(e) selecting the polynucleotides encoding the candidate activating proteins that resulted in T cell differentiation for evaluation in vivo, wherein the percentage of differentiated T cells is at or above 50% of the total cell population consisting of the differentiated T cells and T cell progenitors.

35. The method of claim 34, wherein the tissue of interest is the liver, spleen, small intestine, thymus, or a combination thereof, and wherein the sample of single clone cells is selected using flow cytometry.

36. (canceled)


SEQUENCE LISTING (782 sequences)

SEQ ID NO: 1 - DNA, 36 bp, synthetic construct
  source 1..36: mol_type = other DNA
  misc_feature 1..36: note = Synthetic
agcagaaatagtagccttcgtttttatttttatctt

SEQ ID NO: 2 - DNA, 32 bp, synthetic construct
  source 1..32: mol_type = other DNA
  misc_feature 1..32: note = Synthetic
tcttgttgtgggatgaagcctatttttatctt

SEQ ID NO: 3 - DNA, 32 bp, synthetic construct
  source 1..32: mol_type = other DNA
  misc_feature 1..32: note = Synthetic
gtccaccttctgaccaagtttatttttatctt

SEQ ID NO: 4 - DNA, 33 bp, synthetic construct
  misc_feature 1..33: note = Synthetic
  source 1..33: mol_type = other DNA
cctcatggcagaaaacagttttatttttatctt

SEQ ID NO: 5 - DNA, 31 bp, synthetic construct
  source 1..31: mol_type = other DNA
  misc_feature 1..31: note = Synthetic
ctagcggctgtggtagcagtatttttatctt

SEQ ID NO: 6 - DNA, 34 bp, synthetic construct
  source 1..34: mol_type = other DNA
  misc_feature 1..34: note = Synthetic
agagcaagttgagatactctcctatttttatctt

SEQ ID NO: 7 - DNA, 32 bp, synthetic construct
  source 1..32: mol_type = other DNA
  misc_feature 1..32: note = Synthetic
ggccttccggtctcatgtcctattgttatctt

SEQ ID NO: 8 - DNA, 31 bp, synthetic construct
  misc_feature 1..31: note = Synthetic
  source 1..31: mol_type = other DNA
gggccagttccctccaagatattgttatctt

SEQ ID NO: 9 - DNA, 31 bp, synthetic construct
  misc_feature 1..31: note = Synthetic
  source 1..31: mol_type = other DNA
cctcgtgggtccagaacgctattgttatctt

SEQ ID NO: 10 - DNA, 31 bp, synthetic construct
  misc_feature 1..31: note = Synthetic
  source 1..31: mol_type = other DNA
tgcaaaccatccttccacctattgttatctt

SEQ ID NO: 11 - DNA, 32 bp, synthetic construct
  source 1..32: mol_type = other DNA
  misc_feature 1..32: note = Synthetic
tgcaggtcacaaatactttgtattgttatctt

SEQ ID NO: 12 - DNA, 32 bp, synthetic construct
  misc_feature 1..32: note = Synthetic
  source 1..32: mol_type = other DNA
cttccagagggaatcatgcatattgttatctt

SEQ ID NO: 13 - DNA, 33 bp, synthetic construct
  source 1..33: mol_type = other DNA
  misc_feature 1..33: note = Synthetic
gcagctgtcattctgggaccttattcttatctt

SEQ ID NO: 14 - DNA, 35 bp, synthetic construct
  source 1..35: mol_type = other DNA
  misc_feature 1..35: note = Synthetic
atgaaagcattcagaggcttctttattcttatctt

SEQ ID NO: 15 - DNA, 31 bp, synthetic construct
  misc_feature 1..31: note = Synthetic
  source 1..31: mol_type = other DNA
gtcgctgttcatattgggctattcttatctt

SEQ ID NO: 16 - DNA, 31 bp, synthetic construct
  source 1..31: mol_type = other DNA
  misc_feature 1..31: note = Synthetic
gctcctgtttgacctgaggtattcttatctt

SEQ ID NO: 17 - DNA, 32 bp, synthetic construct
  source 1..32: mol_type = other DNA
  misc_feature 1..32: note = Synthetic
ggtcgctggctttctagttgtattcttatctt

SEQ ID NO: 18 - DNA, 33 bp, synthetic construct
  source 1..33: mol_type = other DNA
  misc_feature 1..33: note = Synthetic
agtgaggatgggtagggttgctattcttatctt

SEQ ID NO: 19 - DNA, 35 bp, synthetic construct
  misc_feature 1..35: note = Synthetic
  source 1..35: mol_type = other DNA
atttccttctatgaacccatacctattattatctt

SEQ ID NO: 20 - DNA, 32 bp, synthetic construct
  source 1..32: mol_type = other DNA
  misc_feature 1..32: note = Synthetic
agatagcgttgggatgaatctattattatctt

SEQ ID NO: 21 - DNA, 37 bp, synthetic construct
  source 1..37: mol_type = other DNA
  misc_feature 1..37: note = Synthetic
ggtaggtgtgcaaaatgtagctttctattattatctt

SEQ ID NO: 22 - DNA, 31 bp, synthetic construct
  source 1..31: mol_type = other DNA
  misc_feature 1..31: note = Synthetic
ctggttcgcacttgagtgttattattatctt

SEQ ID NO: 23 - DNA, 31 bp, synthetic construct
  misc_feature 1..31: note = Synthetic
  source 1..31: mol_type = other DNA
ggaggtgttggacgtgacgtattattatctt

SEQ ID NO: 24 - DNA, 31 bp, synthetic construct
  source 1..31: mol_type = other DNA
  misc_feature 1..31: note = Synthetic
acgtctccaatcgtgctgttattattatctt

SEQ ID NO: 25 - DNA, 31 bp, synthetic construct
  misc_feature 1..31: note = Synthetic
  source 1..31: mol_type = other DNA
ctgcagtgacctgtaaggttatgtttatctt

SEQ ID NO: 26 - DNA, 32 bp, synthetic construct
  source 1..32: mol_type = other DNA
  misc_feature 1..32: note = Synthetic
ctgtagagccttgtatcccgtatgtttatctt

SEQ ID NO: 27 - DNA, 37 bp, synthetic construct
  source 1..37: mol_type = other DNA
  misc_feature 1..37: note = Synthetic
acctctcttgcattcacagtttaggtatgtttatctt

SEQ ID NO: 28 - DNA, 32 bp, synthetic construct
  misc_feature 1..32: note = Synthetic
  source 1..32: mol_type = other DNA
actccattgtgagcacaaattatgtttatctt

SEQ ID NO: 29 - DNA, 31 bp, synthetic construct
  misc_feature 1..31: note = Synthetic
  source 1..31: mol_type = other DNA
ccaccccgttttcccacactatgtttatctt

SEQ ID NO: 30 - DNA, 33 bp, synthetic construct
  source 1..33: mol_type = other DNA
  misc_feature 1..33: note = Synthetic
tccagtgttttcacggtatgttatgtttatctt

SEQ ID NO: 31 - DNA, 33 bp, synthetic construct
  misc_feature 1..33: note = Synthetic
  source 1..33: mol_type = other DNA
tggcataacacatgaacactctatggttatctt

SEQ ID NO: 32 - DNA, 32 bp, synthetic construct
  source 1..32: mol_type = other DNA
  misc_feature 1..32: note = Synthetic
gcatcagagttggacaccagtatggttatctt

SEQ ID NO: 33 - DNA, 31 bp, synthetic construct
  misc_feature 1..31: note = Synthetic
  source 1..31: mol_type = other DNA
tctcctcgacaaccttccatatggttatctt

SEQ ID NO: 34 - DNA, 33 bp, synthetic construct
  misc_feature 1..33: note = Synthetic
  source 1..33: mol_type = other DNA
ggagcatgccatttatgagcctatggttatctt

SEQ ID NO: 35 - DNA, 32 bp, synthetic construct
  source 1..32: mol_type = other DNA
  misc_feature 1..32: note = Synthetic
aatgtgtcccctttcttaagtatggttatctt

SEQ ID NO: 36 - DNA, 34 bp, synthetic construct
  misc_feature 1..34: note = Synthetic
  source 1..34: mol_type = other DNA
atcctgtaaggacaacacatgatatggttatctt

SEQ ID NO: 37 - DNA, 34 bp, synthetic construct
  source 1..34: mol_type = other DNA
  misc_feature 1..34: note = Synthetic
agattccagagacggaacgtcatatgcttatctt

SEQ ID NO: 38 - DNA, 31 bp, synthetic construct
  misc_feature 1..31: note = Synthetic
  source 1..31: mol_type = other DNA
tgcccggaattcctttgcgtatgcttatctt

SEQ ID NO: 39 - DNA, 33 bp, synthetic construct
  source 1..33: mol_type = other DNA
  misc_feature 1..33: note = Synthetic
atagttccattgctcctctcgtatgcttatctt

SEQ ID NO: 40 - DNA, 31 bp, synthetic construct
  misc_feature 1..31: note = Synthetic
  source 1..31: mol_type = other DNA
agccacacagactttatcctatgcttatctt

SEQ ID NO: 41 - DNA, 31 bp, synthetic construct
  source 1..31: mol_type = other DNA
  misc_feature 1..31: note = Synthetic
cgaaactggggctgataggtatgcttatctt

SEQ ID NO: 42 - DNA, 31 bp, synthetic construct
  misc_feature 1..31: note = Synthetic
  source 1..31: mol_type = other DNA
ctccagcaaccacgaccagtatgcttatctt

SEQ ID NO: 43 - DNA, 33 bp, synthetic construct
  misc_feature 1..33: note = Synthetic
  source 1..33: mol_type = other DNA
agtcttgtcagtcaagccacatatgattatctt

SEQ ID NO: 44 - DNA, 32 bp, synthetic construct
  misc_feature 1..32: note = Synthetic
  source 1..32: mol_type = other DNA
tgtcccgcaatgagatatactatgattatctt

SEQ ID NO: 45 - DNA, 34 bp, synthetic construct
  source 1..34: mol_type = other DNA
  misc_feature 1..34: note = Synthetic
ctgcttgtctgaagctcttgactatgattatctt

SEQ ID NO: 46 - DNA, 31 bp, synthetic construct
  misc_feature 1..31: note = Synthetic
  source 1..31: mol_type = other DNA
tggtgcctatgtttagctctatgattatctt

SEQ ID NO: 47 - DNA, 35 bp, synthetic construct
  misc_feature 1..35: note = Synthetic
  source 1..35: mol_type = other DNA
atgctgatgacctcagcgaagattatgattatctt

SEQ ID NO: 48 - DNA, 36 bp, synthetic construct
  misc_feature 1..36: note = Synthetic
  source 1..36: mol_type = other DNA
ccttggagatggctgtactggtcatatgattatctt

SEQ ID NO: 49 - DNA, 32 bp, synthetic construct
  source 1..32: mol_type = other DNA
  misc_feature 1..32: note = Synthetic
ctcgtcactgtctagagggctatctttatctt

SEQ ID NO: 50 - DNA, 32 bp, synthetic construct
  source 1..32: mol_type = other DNA
  misc_feature 1..32: note = Synthetic
gcccagagtgatacagatgttatctttatctt

SEQ ID NO: 51 - DNA, 32 bp, synthetic construct
  misc_feature 1..32: note = Synthetic
  source 1..32: mol_type = other DNA
tcagactgctctctgattcatatctttatctt

SEQ ID NO: 52 - DNA, 32 bp, synthetic construct
  source 1..32: mol_type = other DNA
  misc_feature 1..32: note = Synthetic
tggagatgaccatccgaggatatctttatctt

SEQ ID NO: 53 - DNA, 32 bp, synthetic construct
  misc_feature 1..32: note = Synthetic
  source 1..32: mol_type = other DNA
gctgtaggaaagggcttagctatctttatctt

SEQ ID NO: 54 - DNA, 31 bp, synthetic construct
  misc_feature 1..31: note = Synthetic
  source 1..31: mol_type = other DNA
gggtccacagaaggcgatgtatctttatctt

SEQ ID NO: 55 - DNA, 35 bp, synthetic construct
  source 1..35: mol_type = other DNA
  misc_feature 1..35: note = Synthetic
tcctctcttcgccctagattgagtatcgttatctt

SEQ ID NO: 56 - DNA, 31 bp, synthetic construct
  source 1..31: mol_type = other DNA
  misc_feature 1..31: note = Synthetic
ggctgtgatgatgactccgtatcgttatctt

SEQ ID NO: 57 - DNA, 32 bp, synthetic construct
  source 1..32: mol_type = other DNA
  misc_feature 1..32: note = Synthetic
tgctcacttctgagtagagctatcgttatctt

SEQ ID NO: 58 - DNA, 31 bp, synthetic construct
  misc_feature 1..31: note = Synthetic
  source 1..31: mol_type = other DNA
ggcgggctcttaacccaagtatcgttatctt

SEQ ID NO: 59 - DNA, 35 bp, synthetic construct
  source 1..35: mol_type = other DNA
  misc_feature 1..35: note = Synthetic
gcaagtagcagagtttgggatcctatcgttatctt

SEQ ID NO: 60 - DNA, 36 bp, synthetic construct
  source 1..36: mol_type = other DNA
  misc_feature 1..36: note = Synthetic
accctggtaaaggccatcgtgccctatcgttatctt

SEQ ID NO: 61 - DNA, 35 bp, synthetic construct
  source 1..35: mol_type = other DNA
  misc_feature 1..35: note = Synthetic
actcacaaactgcgtctacagtctatccttatctt

SEQ ID NO: 62 - DNA, 31 bp, synthetic construct
  misc_feature 1..31: note = Synthetic
  source 1..31: mol_type = other DNA
agttccgcgaaacgctgtgtatccttatctt

SEQ ID NO: 63 - DNA, 31 bp, synthetic construct
  misc_feature 1..31: note = Synthetic
  source 1..31: mol_type = other DNA
ggccccgatagagccatcctatccttatctt

SEQ ID NO: 64 - DNA, 33 bp, synthetic construct
  misc_feature 1..33: note = Synthetic
  source 1..33: mol_type = other DNA
cctggcattcattttccgaaatatccttatctt

SEQ ID NO: 65 - DNA, 35 bp, synthetic construct
  misc_feature 1..35: note = Synthetic
  source 1..35: mol_type = other DNA
gcatctatggaagggactgtctctatccttatctt

SEQ ID NO: 66 - DNA, 32 bp, synthetic construct
  misc_feature 1..32: note = Synthetic
  source 1..32: mol_type = other DNA
agagggactctggacactcttatccttatctt

SEQ ID NO: 67 - DNA, 37 bp, synthetic construct
  source 1..37: mol_type = other DNA
  misc_feature 1..37: note = Synthetic
ccatctcactgatttcaatcgtgtttatcattatctt

SEQ ID NO: 68 - DNA, 31 bp, synthetic construct
  misc_feature 1..31: note = Synthetic
  source 1..31: mol_type = other DNA
accacgtcaatggaatggctatcattatctt

SEQ ID NO: 69 - DNA, 33 bp, synthetic construct
  source 1..33: mol_type = other DNA
  misc_feature 1..33: note = Synthetic
ggaaggatttcttggcaggagtatcattatctt

SEQ ID NO: 70 - DNA, 32 bp, synthetic construct
  misc_feature 1..32: note = Synthetic
  source 1..32: mol_type = other DNA
ctcatggccatgctttcttatatcattatctt

SEQ ID NO: 71 - DNA, 32 bp, synthetic construct
  source 1..32: mol_type = other DNA
  misc_feature 1..32: note = Synthetic
atggcgggtttattgagctctatcattatctt

SEQ ID NO: 72 - DNA, 36 bp, synthetic construct
  misc_feature 1..36: note = Synthetic
  source 1..36: mol_type = other DNA
agacagagattcctcctggcaagatatcattatctt

SEQ ID NO: 73 - DNA, 34 bp, synthetic construct
  source 1..34: mol_type = other DNA
  misc_feature 1..34: note = Synthetic
agaggccgtctttggtttgtcctatatttatctt

SEQ ID NO: 74 - DNA, 32 bp, synthetic construct
  misc_feature 1..32: note = Synthetic
  source 1..32: mol_type = other DNA
cctcttctccatgataggcttatatttatctt

SEQ ID NO: 75 - DNA, 32 bp, synthetic construct
  misc_feature 1..32: note = Synthetic
  source 1..32: mol_type = other DNA
agggctacttagtgatcggttatatttatctt

SEQ ID NO: 76 - DNA, 33 bp, synthetic construct
  misc_feature 1..33: note = Synthetic
  source 1..33: mol_type = other DNA
ccgggagctatctttcttaagtatatttatctt

SEQ ID NO: 77 - DNA, 31 bp, synthetic construct
  misc_feature 1..31: note = Synthetic
  source 1..31: mol_type = other DNA
accgggatgaccgggccgctatatttatctt

SEQ ID NO: 78 - DNA, 33 bp, synthetic construct
  source 1..33: mol_type = other DNA
  misc_feature 1..33: note = Synthetic
cgccacggtctccacatggagtatatttatctt

SEQ ID NO: 79 - DNA, 31 bp, synthetic construct
  misc_feature 1..31: note = Synthetic
  source 1..31: mol_type = other DNA
ctgcctgtgttttcaggtttatagttatctt

SEQ ID NO: 80 - DNA, 32 bp, synthetic construct
  misc_feature 1..32: note = Synthetic
  source 1..32: mol_type = other DNA
gtggactgctgaaatgttcgtatagttatctt

SEQ ID NO: 81 - DNA, 32 bp, synthetic construct
  source 1..32: mol_type = other DNA
  misc_feature 1..32: note = Synthetic
atctccttcatgtaaagcattatagttatctt

SEQ ID NO: 82 - DNA, 36 bp, synthetic construct
  source 1..36: mol_type = other DNA
  misc_feature 1..36: note = Synthetic
tgaccacagagttattttgtgtgatatagttatctt

SEQ ID NO: 83 - DNA, 32 bp, synthetic construct
  misc_feature 1..32: note = Synthetic
  source 1..32: mol_type = other DNA
gaaggcagagtagacagtcttatagttatctt

SEQ ID NO: 84 - DNA, 31 bp, synthetic construct
  misc_feature 1..31: note = Synthetic
  source 1..31: mol_type = other DNA
ggtgcacactgggtttaggtatagttatctt

SEQ ID NO: 85 - DNA, 34 bp, synthetic construct
  misc_feature 1..34: note = Synthetic
  source 1..34: mol_type = other DNA
agtttccgtagtaggacgggactatacttatctt

SEQ ID NO: 86 - DNA, 31 bp, synthetic construct
  misc_feature 1..31: note = Synthetic
  source 1..31: mol_type = other DNA
cggatgcatgggcgtcggttatacttatctt

SEQ ID NO: 87 - DNA, 32 bp, synthetic construct
  source 1..32: mol_type = other DNA
  misc_feature 1..32: note = Synthetic
ggtggtggtggtctgacagttatacttatctt

SEQ ID NO: 88 - DNA, 32 bp, synthetic construct
  source 1..32: mol_type = other DNA
  misc_feature 1..32: note = Synthetic
acggttgctcttccgatcactatacttatctt

SEQ ID NO: 89 - DNA, 32 bp, synthetic construct
  source 1..32: mol_type = other DNA
  misc_feature 1..32: note = Synthetic
aaaacacagatctgtcgctttatacttatctt

SEQ ID NO: 90 - DNA, 31 bp, synthetic construct
  source 1..31: mol_type = other DNA
  misc_feature 1..31: note = Synthetic
cccggtcagattgcgtagctatacttatctt

SEQ ID NO: 91 - DNA, 33 bp, synthetic construct
  source 1..33: mol_type = other DNA
  misc_feature 1..33: note = Synthetic
gctggaaggctcatctttacgtataattatctt

SEQ ID NO: 92 - DNA, 34 bp, synthetic construct
  misc_feature 1..34: note = Synthetic
  source 1..34: mol_type = other DNA
gcctgctcgattttgacatcattataattatctt

SEQ ID NO: 93 - DNA, 31 bp, synthetic construct
  misc_feature 1..31: note = Synthetic
  source 1..31: mol_type = other DNA
cgagccactgcgagtacactataattatctt

SEQ ID NO: 94 - DNA, 31 bp, synthetic construct
  source 1..31: mol_type = other DNA
  misc_feature 1..31: note = Synthetic
gttgaagggctgcttgcattataattatctt

SEQ ID NO: 95 - DNA, 32 bp, synthetic construct
  misc_feature 1..32: note = Synthetic
  source 1..32: mol_type = other DNA
actgccttaatcaaccctcatataattatctt

SEQ ID NO: 96 - DNA, 31 bp, synthetic construct
  source 1..31: mol_type = other DNA
  misc_feature 1..31: note = Synthetic
tccctctacgtcacttgcgtataattatctt

SEQ ID NO: 97 - DNA, 34 bp, synthetic construct
  source 1..34: mol_type = other DNA
  misc_feature 1..34: note = Synthetic
accactgaaagtagctttcaagtagttttatctt

SEQ ID NO: 98 - DNA, 32 bp, synthetic construct
  source 1..32: mol_type = other DNA
  misc_feature 1..32: note = Synthetic
agggacaggagttcttccgatagttttatctt

SEQ ID NO: 99 - DNA, 31 bp, synthetic construct
  misc_feature 1..31: note = Synthetic
  source 1..31: mol_type = other DNA
gcatgctcgataccgtagctagttttatctt

SEQ ID NO: 100 - DNA, 33 bp, synthetic construct
  misc_feature 1..33: note = Synthetic
  source 1..33: mol_type = other DNA
acctcttgcttgatggcaaagtagttttatctt

SEQ ID NO: 101 - DNA, 32 bp, synthetic construct
  misc_feature 1..32: note = Synthetic
  source 1..32: mol_type = other DNA
ttctttgctgctcggagttatagttttatctt

SEQ ID NO: 102 - DNA, 32 bp, synthetic construct
  misc_feature 1..32: note = Synthetic
  source 1..32: mol_type = other DNA
aaagagcatcaatgcagcaatagttttatctt

SEQ ID NO: 103 - DNA, 34 bp, synthetic construct
  misc_feature 1..34: note = Synthetic
  source 1..34: mol_type = other DNA
ctggaggcacataccatctcagtagtgttatctt

SEQ ID NO: 104 - DNA, 33 bp, synthetic construct
  source 1..33: mol_type = other DNA
  misc_feature 1..33: note = Synthetic
acaggatcatggatcaagcgctagtgttatctt

SEQ ID NO: 105 - DNA, 32 bp, synthetic construct
  misc_feature 1..32: note = Synthetic
  source 1..32: mol_type = other DNA
tggaccccacggatagaatgtagtgttatctt

SEQ ID NO: 106 - DNA, 32 bp, synthetic construct
  misc_feature 1..32: note = Synthetic
  source 1..32: mol_type = other DNA
aagagtgcctgagtcttcgttagtgttatctt

SEQ ID NO: 107 - DNA, 36 bp, synthetic construct
  misc_feature 1..36: note = Synthetic
  source 1..36: mol_type = other DNA
cccaaggcagttagctgcagcaagtagtgttatctt

SEQ ID NO: 108 - DNA, 37 bp, synthetic construct
  source 1..37: mol_type = other DNA
  misc_feature 1..37: note = Synthetic
gccaagtccagctcactatggcttgtagtgttatctt

SEQ ID NO: 109 - DNA, 34 bp, synthetic construct
  source 1..34: mol_type = other DNA
  misc_feature 1..34: note = Synthetic
actgagctgtaacatcttggcctagtcttatctt

SEQ ID NO: 110 - DNA, 33 bp, synthetic construct
  misc_feature 1..33: note = Synthetic
  source 1..33: mol_type = other DNA
agttaaggtctcaacgcaatgtagtcttatctt

SEQ ID NO: 111 - DNA, 31 bp, synthetic construct
  source 1..31: mol_type = other DNA
  misc_feature 1..31: note = Synthetic
agtgcacctctaaagtcgatagtcttatctt

SEQ ID NO: 112 - DNA, 31 bp, synthetic construct
  misc_feature 1..31: note = Synthetic
  source 1..31: mol_type = other DNA
aggcagcaatgtaagatgctagtcttatctt

SEQ ID NO: 113 - DNA, 33 bp, synthetic construct
  source 1..33: mol_type = other DNA
  misc_feature 1..33: note = Synthetic
ctccagctcgacggtctggtctagtcttatctt

SEQ ID NO: 114 - DNA, 35 bp, synthetic construct
  source 1..35: mol_type = other DNA
  misc_feature 1..35: note = Synthetic
gtgcgtgtagtcgttcaccgtcctagtcttatctt

SEQ ID NO: 115 - DNA, 31 bp, synthetic construct
  source 1..31: mol_type = other DNA
  misc_feature 1..31: note = Synthetic
ggggcaaattcctctagattagtattatctt

SEQ ID NO: 116 - DNA, 31 bp, synthetic construct
  misc_feature 1..31: note = Synthetic
  source 1..31: mol_type = other DNA
cccctccagtgcagaagtttagtattatctt

SEQ ID NO: 117 - DNA, 33 bp, synthetic construct
  source 1..33: mol_type = other DNA
  misc_feature 1..33: note = Synthetic
gggtcatgctcatttccacagtagtattatctt

SEQ ID NO: 118 - DNA, 33 bp, synthetic construct
  misc_feature 1..33: note = Synthetic
  source 1..33: mol_type = other DNA
acatcacaatctagcaggatatagtattatctt

SEQ ID NO: 119 - DNA, 31 bp, synthetic construct
  source 1..31: mol_type = other DNA
  misc_feature 1..31: note = Synthetic
ggtcaggggacttccgtgttagtattatctt

SEQ ID NO: 120 - DNA, 34 bp, synthetic construct
  misc_feature 1..34: note = Synthetic
  source 1..34: mol_type = other DNA
agcttccatccgaaaactgccatagtattatctt

SEQ ID NO: 121 - DNA, 32 bp, synthetic construct
  source 1..32: mol_type = other DNA
  misc_feature 1..32: note = Synthetic
ggactggaagctgcaaggactaggtttatctt

SEQ ID NO: 122 - DNA, 34 bp, synthetic construct
  source 1..34: mol_type = other DNA
  misc_feature 1..34: note = Synthetic
agtcagggacaaagtgtgagtgtaggtttatctt

SEQ ID NO: 123 - DNA, 37 bp, synthetic construct
  source 1..37: mol_type = other DNA
  misc_feature 1..37: note = Synthetic
tcaccaaggatacaaatctacagtataggtttatctt

SEQ ID NO: 124 - DNA, 31 bp, synthetic construct
  misc_feature 1..31: note = Synthetic
  source 1..31: mol_type = other DNA
ggaaagtcaggagcgaccataggtttatctt

SEQ ID NO: 125 - DNA, 32 bp, synthetic construct
  misc_feature 1..32: note = Synthetic
  source 1..32: mol_type = other DNA
gcacatacagccaatagcgctaggtttatctt

SEQ ID NO: 126 - DNA, 31 bp, synthetic construct
  source 1..31: mol_type = other DNA
  misc_feature 1..31: note = Synthetic
tctgcacacacgtatagcttaggtttatctt

SEQ ID NO: 127 - DNA, 33 bp, synthetic construct
  source 1..33: mol_type = other DNA
  misc_feature 1..33: note = Synthetic
acaggattgctgtacaccaaatagggttatctt

SEQ ID NO: 128 - DNA, 31 bp, synthetic construct
  misc_feature 1..31: note = Synthetic
  source 1..31: mol_type = other DNA
accgcctgtgttacctgcatagggttatctt

SEQ ID NO: 129 - DNA, 33 bp, synthetic construct
  misc_feature 1..33: note = Synthetic
  source 1..33: mol_type = other DNA
tgttgaaatggctgatggatatagggttatctt

SEQ ID NO: 130 - DNA, 33 bp, synthetic construct
  source 1..33: mol_type = other DNA
  misc_feature 1..33: note = Synthetic
tccttgtcctggtaaagtaggtagggttatctt

SEQ ID NO: 131 - DNA, 33 bp, synthetic construct
  source 1..33: mol_type = other DNA
  misc_feature 1..33: note = Synthetic
agaggcgggaacagatgaagttagggttatctt

SEQ ID NO: 132 - DNA, 33 bp, synthetic construct
  source 1..33: mol_type = other DNA
  misc_feature 1..33: note = Synthetic
agttcccaaatccctgcattctagggttatctt

SEQ ID NO: 133 - DNA, 34 bp, synthetic construct
  source 1..34: mol_type = other DNA
  misc_feature 1..34: note = Synthetic
atcttggtccactcgtagatgttaggcttatctt

SEQ ID NO: 134 - DNA, 33 bp, synthetic construct
  misc_feature 1..33: note = Synthetic
  source 1..33: mol_type = other DNA
cggagttgatgagaaggtatctaggcttatctt

SEQ ID NO: 135 - DNA, 35 bp, synthetic construct
  source 1..35: mol_type = other DNA
  misc_feature 1..35: note = Synthetic
ggtgatacactttgtctttcacctaggcttatctt

SEQ ID NO: 136 - DNA, 34 bp, synthetic construct
  misc_feature 1..34: note = Synthetic
  source 1..34: mol_type = other DNA
ccatgaacaccaaggtcacatctaggcttatctt

SEQ ID NO: 137 - DNA, 34 bp, synthetic construct
  misc_feature 1..34: note = Synthetic
  source 1..34: mol_type = other DNA
ctctttcctttcctagtgtgaataggcttatctt

SEQ ID NO: 138 - DNA, 33 bp, synthetic construct
  source 1..33: mol_type = other DNA
  misc_feature 1..33: note = Synthetic
tggctttcaggaagtagcggttaggcttatctt

SEQ ID NO: 139 - DNA, 33 bp, synthetic construct
  misc_feature 1..33: note = Synthetic
  source 1..33: mol_type = other DNA
ggcgaacaccgctatatcttctaggattatctt

SEQ ID NO: 140 - DNA, 32 bp, synthetic construct
  source 1..32: mol_type = other DNA
  misc_feature 1..32: note = Synthetic
atctctctttaaagcgtcattaggattatctt

SEQ ID NO: 141 - DNA, 32 bp, synthetic construct
  misc_feature 1..32: note = Synthetic
  source 1..32: mol_type = other DNA
gcgtaccccactcagttaactaggattatctt

SEQ ID NO: 142 - DNA, 33 bp, synthetic construct
  source 1..33: mol_type = other DNA
  misc_feature 1..33: note = Synthetic
acttgtgcactcattgtcgggtaggattatctt

SEQ ID NO: 143 - DNA, 31 bp, synthetic construct
  misc_feature 1..31: note = Synthetic
  source 1..31: mol_type = other DNA
ggcgaggttgtcaacgtggtaggattatctt

SEQ ID NO: 144 - DNA, 31 bp, synthetic construct
  source 1..31: mol_type = other DNA
  misc_feature 1..31: note = Synthetic
cggggttcctcctgaacgataggattatctt

SEQ ID NO: 145 - DNA, 31 bp, synthetic construct
  source 1..31: mol_type = other DNA
  misc_feature 1..31: note = Synthetic
tgtccggctagggtcaaagtagctttatctt

SEQ ID NO: 146 - DNA, 31 bp, synthetic construct
  misc_feature 1..31: note = Synthetic
  source 1..31: mol_type = other DNA
gttgaggggctaggtgagatagctttatctt

SEQ ID NO: 147 - DNA, 34 bp, synthetic construct
  source 1..34: mol_type = other DNA
  misc_feature 1..34: note = Synthetic
agacagtcgctttgtcgctgtttagctttatctt

SEQ ID NO: 148 - DNA, 32 bp, synthetic construct
  source 1..32: mol_type = other DNA
  misc_feature 1..32: note = Synthetic
gctttgtttccagttctggttagctttatctt

SEQ ID NO: 149 - DNA, 36 bp, synthetic construct
  source 1..36: mol_type = other DNA
  misc_feature 1..36: note = Synthetic
cccctaatgcatgacacccaagtatagctttatctt

SEQ ID NO: 150 - DNA, 31 bp, synthetic construct
  misc_feature 1..31: note = Synthetic
  source 1..31: mol_type = other DNA
atgccgcccgcccagtgactagctttatctt

SEQ ID NO: 151 - DNA, 34 bp, synthetic construct
  source 1..34: mol_type = other DNA
  misc_feature 1..34: note = Synthetic
ctctgctcctatcttgcacaggtagcgttatctt

SEQ ID NO: 152 - DNA, 31 bp, synthetic construct
  source 1..31: mol_type = other DNA
  misc_feature 1..31: note = Synthetic
tggaaaggccgttcacccatagcgttatctt

SEQ ID NO: 153 - DNA, 32 bp, synthetic construct
  source 1..32: mol_type = other DNA
  misc_feature 1..32: note = Synthetic
ccaagaaatttctgaggcattagcgttatctt

SEQ ID NO: 154 - DNA, 32 bp, synthetic construct
  misc_feature 1..32: note = Synthetic
  source 1..32: mol_type = other DNA
atgtgaatggtatacatgactagcgttatctt

SEQ ID NO: 155 - DNA, 31 bp, synthetic construct
  source 1..31: mol_type = other DNA
  misc_feature 1..31: note = Synthetic
acttgggacatgtcttgactagcgttatctt

SEQ ID NO: 156 - DNA, 32 bp, synthetic construct
  misc_feature 1..32: note = Synthetic
  source 1..32: mol_type = other DNA
acctgggaagacagttaggttagcgttatctt

SEQ ID NO: 157 - DNA, 33 bp, synthetic construct
  misc_feature 1..33: note = Synthetic
  source 1..33: mol_type = other DNA
cgcggtagcatttctcagttctagccttatctt

SEQ ID NO: 158 - DNA, 31 bp, synthetic construct
  misc_feature 1..31: note = Synthetic
  source 1..31: mol_type = other DNA
ggtggcaacttgtggcggatagccttatctt

SEQ ID NO: 159 - DNA, 31 bp, synthetic construct
  source 1..31: mol_type = other DNA
  misc_feature 1..31: note = Synthetic
ccgcccgacaaacctgaggtagccttatctt

SEQ ID NO: 160 - DNA, 31 bp, synthetic construct
  source 1..31: mol_type = other DNA
  misc_feature 1..31: note = Synthetic
gccccgtggaatctatctctagccttatctt

SEQ ID NO: 161 - DNA, 32 bp, synthetic construct
  source 1..32: mol_type = other DNA
  misc_feature 1..32: note = Synthetic
atttacagtgacacgatgcatagccttatctt

SEQ ID NO: 162 - DNA, 31 bp, synthetic construct
  misc_feature 1..31: note = Synthetic
  source 1..31: mol_type = other DNA
actcagcctttaggcctcatagccttatctt

SEQ ID NO: 163 - DNA, 34 bp, synthetic construct
  source 1..34: mol_type = other DNA
  misc_feature 1..34: note = Synthetic
aggctgccataactactggacttagcattatctt

SEQ ID NO: 164 - DNA, 32 bp, synthetic construct
  misc_feature 1..32: note = Synthetic
  source 1..32: mol_type = other DNA
ccactgactggtacctgtgatagcattatctt

SEQ ID NO: 165 - DNA, 36 bp, synthetic construct
  source 1..36: mol_type = other DNA
  misc_feature 1..36: note = Synthetic
actgactgaatcttcccgatgtgttagcattatctt

SEQ ID NO: 166 - DNA, 32 bp, synthetic construct
  misc_feature 1..32: note = Synthetic
  source 1..32: mol_type = other DNA
actggtgttgatgggtttgatagcattatctt

SEQ ID NO: 167 - DNA, 34 bp, synthetic construct
  misc_feature 1..34: note = Synthetic
  source 1..34: mol_type = other DNA
aggatgctatctgatccattgatagcattatctt

SEQ ID NO: 168 - DNA, 34 bp, synthetic construct
  misc_feature 1..34: note = Synthetic
  source 1..34: mol_type = other DNA
ggctcatcccattcgatgaacttagcattatctt

SEQ ID NO: 169 - DNA, 32 bp, synthetic construct
  source 1..32: mol_type = other DNA
  misc_feature 1..32: note = Synthetic
ggtcagacccagcaagaccgtagatttatctt

SEQ ID NO: 170 - DNA, 31 bp, synthetic construct
  source 1..31: mol_type = other DNA
  misc_feature 1..31: note = Synthetic
agatgtgtggacagcgtggtagatttatctt

SEQ ID NO: 171 - DNA, 33 bp, synthetic construct
  misc_feature 1..33: note = Synthetic
  source 1..33: mol_type = other DNA
tgccacagatcttacagtcaatagatttatctt

SEQ ID NO: 172 - DNA, 33 bp, synthetic construct
  misc_feature 1..33: note = Synthetic
  source 1..33: mol_type = other DNA
ggctctcccatacctagcacatagatttatctt

SEQ ID NO: 173 - DNA, 37 bp, synthetic construct
  misc_feature 1..37: note = Synthetic
  source 1..37: mol_type = other DNA
gcttgatggtgcatctgatgatttctagatttatctt

SEQ ID NO: 174 - DNA, 31 bp, synthetic construct
  misc_feature 1..31: note = Synthetic
  source 1..31: mol_type = other DNA
ccgctggacgaatgtttggtagatttatctt

SEQ ID NO: 175 - DNA, 33 bp, synthetic construct
  source 1..33: mol_type = other DNA
  misc_feature 1..33: note = Synthetic
cagcgtcttcccttgagtgactagagttatctt

SEQ ID NO: 176 - DNA, 32 bp, synthetic construct
  source 1..32: mol_type = other DNA
  misc_feature 1..32: note = Synthetic
tgcactctgtcaaggggttatagagttatctt

SEQ ID NO: 177 - DNA, 33 bp, synthetic construct
  misc_feature 1..33: note = Synthetic
  source 1..33: mol_type = other DNA
ctccctcactcttataggccgtagagttatctt

SEQ ID NO: 178 - DNA, 34 bp, synthetic construct
  misc_feature 1..34: note = Synthetic
  source 1..34: mol_type = other DNA
accagcaaactgaagcgagacctagagttatctt

SEQ ID NO: 179 - DNA, 31 bp, synthetic construct
  misc_feature 1..31: note = Synthetic
  source 1..31: mol_type = other DNA
agcttgaggtctttggtggtagagttatctt

SEQ ID NO: 180 - DNA, 34 bp, synthetic construct
  misc_feature 1..34: note = Synthetic
  source 1..34: mol_type = other DNA
agtccatcttgaccttatcacctagagttatctt

SEQ ID NO: 181 - DNA, 34 bp, synthetic construct
  source 1..34: mol_type = other DNA
  misc_feature 1..34: note = Synthetic
acagtggagagctgatgcatgatagacttatctt

SEQ ID NO: 182 - DNA, 31 bp, synthetic construct
  source 1..31: mol_type = other DNA
  misc_feature 1..31: note = Synthetic
ggctcctcttcttgcgaaatagacttatctt

SEQ ID NO: 183 - DNA, 31 bp, synthetic construct
  source 1..31: mol_type = other DNA
  misc_feature 1..31: note = Synthetic
agcacactgccctgagtactagacttatctt

SEQ ID NO: 184 - DNA, 31 bp, synthetic construct
  misc_feature 1..31: note = Synthetic
  source 1..31: mol_type = other DNA
aagtaggcgaacatgcgagtagacttatctt

SEQ ID NO: 185 - DNA, 31 bp, synthetic construct
  misc_feature 1..31: note = Synthetic
  source 1..31: mol_type = other DNA
atggcttgctgatatctcgtagacttatctt

SEQ ID NO: 186 - DNA, 34 bp, synthetic construct
  misc_feature 1..34: note = Synthetic
  source 1..34: mol_type = other DNA
acacaggcactatcatgagggatagacttatctt

SEQ ID NO: 187 - DNA, 36 bp, synthetic construct
  misc_feature 1..36: note = Synthetic
  source 1..36: mol_type = other DNA
agaaggtagtaatcaggtgtgatttagaattatctt

SEQ ID NO: 188 - DNA, 34 bp, synthetic construct
  misc_feature 1..34: note = Synthetic
  source 1..34: mol_type = other DNA
ttggtcagtgaagtaaaggtactagaattatctt

SEQ ID NO: 189 - DNA, 32 bp, synthetic construct
  source 1..32: mol_type = other DNA
  misc_feature 1..32: note = Synthetic
aggctggattccggcaacagtagaattatctt

SEQ ID NO: 190 - DNA, 32 bp, synthetic construct
  source 1..32: mol_type = other DNA
  misc_feature 1..32: note = Synthetic
aaagagtctgaggtagaaagtagaattatctt

SEQ ID NO: 191 - DNA, 34 bp, synthetic construct
  misc_feature 1..34: note = Synthetic
  source 1..34: mol_type = other DNA
atagttattcagactttctaggtagaattatctt

SEQ ID NO: 192 - DNA, 32 bp, synthetic construct
  source 1..32: mol_type = other DNA
  misc_feature 1..32: note = Synthetic
gcaaagccaagatgcagtgttagaattatctt

SEQ ID NO: 193 - DNA, 34 bp, synthetic construct
  source 1..34: mol_type = other DNA
  misc_feature 1..34: note = Synthetic
ctgtggtgttcttcgttgctgttacttttatctt

SEQ ID NO: 194 - DNA, 32 bp, synthetic construct
  source 1..32: mol_type = other DNA
  misc_feature 1..32: note = Synthetic
atatgcgaagcaccttggaatacttttatctt

SEQ ID NO: 195 - DNA, 35 bp, synthetic construct
  source 1..35: mol_type = other DNA
  misc_feature 1..35: note = Synthetic
ggtgcagcttatcgatgaatccatacttttatctt

SEQ ID NO: 196 - DNA, 36 bp, synthetic construct
  misc_feature 1..36: note = Synthetic
  source 1..36: mol_type = other DNA
gggttgagacccatcaatagctcttacttttatctt

SEQ ID NO: 197 - DNA, 31 bp, synthetic construct
  misc_feature 1..31: note = Synthetic
  source 1..31: mol_type = other DNA
tggtggctcagtactacgatacttttatctt

SEQ ID NO: 198 - DNA, 31 bp, synthetic construct
  source 1..31: mol_type = other DNA
  misc_feature 1..31: note = Synthetic
tgcagctccatgagaacactacttttatctt

SEQ ID NO: 199 - DNA, 31 bp, synthetic construct
  source 1..31: mol_type = other DNA
  misc_feature 1..31: note = Synthetic
gggtgacgtggaacggttgtactgttatctt

SEQ ID NO: 200 - DNA, 31 bp, synthetic construct
  source 1..31: mol_type = other DNA
  misc_feature 1..31: note = Synthetic
cgccaagggagttaaagactactgttatctt

SEQ ID NO: 201 - DNA, 32 bp, synthetic construct
  source 1..32: mol_type = other DNA
  misc_feature 1..32: note = Synthetic
cagcatcttctcgaccctgatactgttatctt

SEQ ID NO: 202 - DNA, 31 bp, synthetic construct
  source 1..31: mol_type = other DNA
  misc_feature 1..31: note = Synthetic
acgaagcagtttgggaccctactgttatctt

SEQ ID NO: 203 - DNA, 33 bp, synthetic construct
  misc_feature 1..33: note = Synthetic
  source 1..33: mol_type = other DNA
gcttcccagatcacagagggatactgttatctt

SEQ ID NO: 204 - DNA, 33 bp, synthetic construct
  misc_feature 1..33: note = Synthetic
  source 1..33: mol_type = other DNA
ctcttgctggatgagaacagatactgttatctt

SEQ ID NO: 205 - DNA, 34 bp, synthetic construct
  source 1..34: mol_type = other DNA
  misc_feature 1..34: note = Synthetic
gggacactcgtatcaacagatgtactcttatctt

SEQ ID NO: 206 - DNA, 31 bp, synthetic construct
  source 1..31: mol_type = other DNA
  misc_feature 1..31: note = Synthetic
tggtgagctttagcttccctactcttatctt

SEQ ID NO: 207 - DNA, 35 bp, synthetic construct
  source 1..35: mol_type = other DNA
  misc_feature 1..35: note = Synthetic
ggtctcttggaaagtaaagacgttactcttatctt

SEQ ID NO: 208 - DNA, 34 bp, synthetic construct
  misc_feature 1..34: note = Synthetic
  source 1..34: mol_type = other DNA
tcctcctcctcttagtgtctcctactcttatctt

SEQ ID NO: 209 - DNA, 31 bp, synthetic construct
  misc_feature 1..31: note = Synthetic
  source 1..31: mol_type = other DNA
acagctcggaactccgctttactcttatctt

SEQ ID NO: 210 - DNA, 31 bp, synthetic construct
  misc_feature 1..31: note = Synthetic
  source 1..31: mol_type = other DNA
cctcgcctggtgaaatgtgtactcttatctt

SEQ ID NO: 211 - DNA, 33 bp, synthetic construct
  source 1..33: mol_type = other DNA
  misc_feature 1..33: note = Synthetic
agatgcccttacacaggtagatactattatctt

SEQ ID NO: 212 - DNA, 33 bp, synthetic construct
  source 1..33: mol_type = other DNA
  misc_feature 1..33: note = Synthetic
gccgatgtagtcatcggtgactactattatctt

SEQ ID NO: 213 - DNA, 34 bp, synthetic construct
  misc_feature 1..34: note = Synthetic
  source 1..34: mol_type = other DNA
ggagcaaggtacggatgataattactattatctt

SEQ ID NO: 214 - DNA, 31 bp, synthetic construct
  misc_feature 1..31: note = Synthetic
  source 1..31: mol_type = other DNA
catctgggccacttggatgtactattatctt

SEQ ID NO: 215 - DNA, 31 bp, synthetic construct
  source 1..31: mol_type = other DNA
  misc_feature 1..31: note = Synthetic
gctgcggaacttgacgccgtactattatctt

SEQ ID NO: 216 - DNA, 34 bp, synthetic construct
  source 1..34: mol_type = other DNA
  misc_feature 1..34: note = Synthetic
agcagaacatgaggaaaggtattactattatctt

SEQ ID NO: 217 - DNA, 33 bp, synthetic construct
  misc_feature 1..33: note = Synthetic
  source 1..33: mol_type = other DNA
agagagctgcctttcgtttgttacgtttatctt

SEQ ID NO: 218 - DNA, 31 bp, synthetic construct
  source 1..31: mol_type = other DNA
  misc_feature 1..31: note = Synthetic
agccccgagaatgcggtgatacgtttatctt

SEQ ID NO: 219 - DNA, 32 bp, synthetic construct
  source 1..32: mol_type = other DNA
  misc_feature 1..32: note = Synthetic
tctctgcttctttagtgagatacgtttatctt

SEQ ID NO: 220 - DNA, 31 bp, synthetic construct
  source 1..31: mol_type = other DNA
  misc_feature 1..31: note = Synthetic
ttcccagagtctcgttccctacgtttatctt

SEQ ID NO: 221 - DNA, 36 bp, synthetic construct
  source 1..36: mol_type = other DNA
  misc_feature 1..36: note = Synthetic
agcgagaaaatgagatagcctacatacgtttatctt

SEQ ID NO: 222 - DNA, 31 bp, synthetic construct
  source 1..31: mol_type = other DNA
  misc_feature 1..31: note = Synthetic
aggggaaattaggaggagctacgtttatctt

SEQ ID NO: 223 - DNA, 33 bp, synthetic construct
  misc_feature 1..33: note = Synthetic
  source 1..33: mol_type = other DNA
gcacgagggagacacttgtagtacggttatctt

SEQ ID NO: 224 - DNA, 33 bp, synthetic construct
  misc_feature 1..33: note = Synthetic
  source 1..33: mol_type = other DNA
gcttcccacagagttagagcatacggttatctt

SEQ ID NO: 225 - DNA, 31 bp, synthetic construct
  misc_feature 1..31: note = Synthetic
  source 1..31: mol_type = other DNA
caggagacacagataggcctacggttatctt

SEQ ID NO: 226 - DNA, 31 bp, synthetic construct
  source 1..31: mol_type = other DNA
  misc_feature 1..31: note = Synthetic
ttcatcccgtacctgacggtacggttatctt

SEQ ID NO: 227 - DNA, 37 bp, synthetic construct
  misc_feature 1..37: note = Synthetic
  source 1..37: mol_type = other DNA
agtcctgagttgcacgtacaataattacggttatctt

SEQ ID NO: 228 - DNA, 33 bp, synthetic construct
  misc_feature 1..33: note = Synthetic
  source 1..33: mol_type = other DNA
agcaagaaaggatgttaaccctacggttatctt

SEQ ID NO: 229 - DNA, 31 bp, synthetic construct
  misc_feature 1..31: note = Synthetic
  source 1..31: mol_type = other DNA
gttccttgggctataagcgtacgcttatctt

SEQ ID NO: 230 - DNA, 32 bp, synthetic construct
  source 1..32: mol_type = other DNA
  misc_feature 1..32: note = Synthetic
tccaccctgaattgggtatatacgcttatctt

SEQ ID NO: 231 - DNA, 32 bp, synthetic construct
  source 1..32: mol_type = other DNA
  misc_feature 1..32: note = Synthetic
tggagctctcgaagagatgatacgcttatctt

SEQ ID NO: 232 - DNA, 33 bp, synthetic construct
  source 1..33: mol_type = other DNA
  misc_feature 1..33: note = Synthetic
ggttttgttgggattcaaaactacgcttatctt

SEQ ID NO: 233 - DNA, 31 bp, synthetic construct
  misc_feature 1..31: note = Synthetic
  source 1..31: mol_type = other DNA
accagctaggctacaccagtacgcttatctt

SEQ ID NO: 234 - DNA, 36 bp, synthetic construct
  misc_feature 1..36: note = Synthetic
  source 1..36: mol_type = other DNA
ggtggtggcagggttatgagtgactacgcttatctt

SEQ ID NO: 235 - DNA, 31 bp, synthetic construct
  source 1..31: mol_type = other DNA
  misc_feature 1..31: note = Synthetic
tccgagggaactcccactttacgattatctt

SEQ ID NO: 236 - DNA, 34 bp, synthetic construct
  source 1..34: mol_type = other DNA
  misc_feature 1..34: note = Synthetic
ggacctaggtaggtccagttgttacgattatctt

SEQ ID NO: 237 - DNA, 31 bp, synthetic construct
  misc_feature 1..31: note = Synthetic
  source 1..31: mol_type = other DNA
ccatggggtcttcaagctctacgattatctt

SEQ ID NO: 238 - DNA, 35 bp, synthetic construct
  misc_feature 1..35: note = Synthetic
  source 1..35: mol_type = other DNA
tggtcttgcacacgtaggtattgtacgattatctt

SEQ ID NO: 239 - DNA, 32 bp, synthetic construct
  misc_feature 1..32: note = Synthetic
  source 1..32: mol_type = other DNA
atggcgagaagaagtagacctacgattatctt

SEQ ID NO: 240 - DNA, 33 bp, synthetic construct
  source 1..33: mol_type = other DNA
  misc_feature 1..33: note = Synthetic
tgagagacttaggaaaccacctacgattatctt

SEQ ID NO: 241 - DNA, 31 bp, synthetic construct
  source 1..31: mol_type = other DNA
  misc_feature 1..31: note = Synthetic
ttcagagttctggcaaacttacctttatctt

SEQ ID NO: 242 - DNA, 32 bp, synthetic construct
  source 1..32: mol_type = other DNA
  misc_feature 1..32: note = Synthetic
tgaagtcctgtgacttagagtacctttatctt

SEQ ID NO: 243 - DNA, 32 bp, synthetic construct
  source 1..32: mol_type = other DNA
  misc_feature 1..32: note = Synthetic
ggctgctgatcatggtgtactaccgttatctt

SEQ ID NO: 244 - DNA, 32 bp, synthetic construct
  source 1..32: mol_type = other DNA
  misc_feature 1..32: note = Synthetic
cgaggctgtgtatgtgtacctaccgttatctt

SEQ ID NO: 245 - DNA, 32 bp, synthetic construct
  misc_feature 1..32: note = Synthetic
  source 1..32: mol_type = other DNA
acactgtgtccgaactcagctaccgttatctt

SEQ ID NO: 246 - DNA, 35 bp, synthetic construct
  misc_feature 1..35: note = Synthetic
  source 1..35: mol_type = other DNA
tgtgtgatagccacatttgtagataccgttatctt

SEQ ID NO: 247 - DNA, 34 bp, synthetic construct
  misc_feature 1..34: note = Synthetic
  source 1..34: mol_type = other DNA
acgctgaatacgtattcatgggtaccgttatctt

SEQ ID NO: 248 - DNA, 34 bp, synthetic construct
  source 1..34: mol_type = other DNA
  misc_feature 1..34: note = Synthetic
atctttgtgtcctaccccaatttaccgttatctt

SEQ ID NO: 249 - DNA, 32 bp, synthetic construct
  source 1..32: mol_type = other DNA
  misc_feature 1..32: note = Synthetic
ctgggagaagatgacctgggtacccttatctt

SEQ ID NO: 250 - DNA, 33 bp, synthetic construct
  source 1..33: mol_type = other DNA
  misc_feature 1..33: note = Synthetic
agggcaaacgcagagtcattatacccttatctt

SEQ ID NO: 251 - DNA, 33 bp, synthetic construct
  misc_feature 1..33: note = Synthetic
  source 1..33: mol_type = other DNA
aggtcataagtgacagtgctctacccttatctt

SEQ ID NO: 252 - DNA, 31 bp, synthetic construct
  source 1..31: mol_type = other DNA
  misc_feature 1..31: note = Synthetic
ccatggcactcatggtgattacccttatctt

SEQ ID NO: 253 - DNA, 31 bp, synthetic construct
  source 1..31: mol_type = other DNA
  misc_feature 1..31: note = Synthetic
accaagctttggacacggttacccttatctt

SEQ ID NO: 254 - DNA, 34 bp, synthetic construct
  source 1..34: mol_type = other DNA
  misc_feature 1..34: note = Synthetic
accatgagaagtcttgatgtactacccttatctt

SEQ ID NO: 255 - DNA, 35 bp, synthetic construct
  source 1..35: mol_type = other DNA
  misc_feature 1..35: note = Synthetic
aggcttgagagaagatccacgtgtaccattatctt

SEQ ID NO: 256 - DNA, 32 bp, synthetic construct
  misc_feature 1..32: note = Synthetic
  source 1..32: mol_type = other DNA
tctgggatttcctccgtgcataccattatctt

SEQ ID NO: 257 - DNA, 34 bp, synthetic construct
  misc_feature 1..34: note = Synthetic
  source 1..34: mol_type = other DNA
agagcaggtgtaatatccggtgtaccattatctt

SEQ ID NO: 258 - DNA, 35 bp, synthetic construct
  misc_feature 1..35: note = Synthetic
  source 1..35: mol_type = other DNA
agacgtgcccatagaattgtacgtaccattatctt

SEQ ID NO: 259 - DNA, 33 bp, synthetic construct
  source 1..33: mol_type = other DNA
  misc_feature 1..33: note = Synthetic
aggaccttcccaaactctaagtaccattatctt

SEQ ID NO: 260 - DNA, 33 bp, synthetic construct
  misc_feature 1..33: note = Synthetic
  source 1..33: mol_type = other DNA
agcgtgaacattttggtgaactaccattatctt

SEQ ID NO: 261 - DNA, 37 bp, synthetic construct
  source 1..37: mol_type = other DNA
  misc_feature 1..37: note = Synthetic
tccagttgtgggtagcattattatctacatttatctt

SEQ ID NO: 262 - DNA, 32 bp, synthetic construct
  source 1..32: mol_type = other DNA
  misc_feature 1..32: note = Synthetic
ctggacactagcatactctgtacatttatctt

SEQ ID NO: 263 - DNA, 33 bp, synthetic construct
  source 1..33: mol_type = other DNA
  misc_feature 1..33: note = Synthetic
tgctgtgctcctaacatcccatacatttatctt

SEQ ID NO: 264 - DNA, 32 bp, synthetic construct
  misc_feature 1..32: note = Synthetic
  source 1..32: mol_type = other DNA
tgtcccctcaatgtggaatgtacatttatctt

SEQ ID NO: 265 - DNA, 33 bp, synthetic construct
  source 1..33: mol_type = other DNA
  misc_feature 1..33: note = Synthetic
gttggctctgcttacatgaattacatttatctt

SEQ ID NO: 266 - DNA, 33 bp, synthetic construct
  misc_feature 1..33: note = Synthetic
  source 1..33: mol_type = other DNA
tgttctcttctttgtaccaggtacatttatctt

SEQ ID NO: 267 - DNA, 33 bp, synthetic construct
  misc_feature 1..33: note = Synthetic
  source 1..33: mol_type = other DNA
tcctcggagacaataataggttacagttatctt

SEQ ID NO: 268 - DNA, 34 bp, synthetic construct
  source 1..34: mol_type = other DNA
  misc_feature 1..34: note = Synthetic
ctccagtatcctggacttgaattacagttatctt

SEQ ID NO: 269 - DNA, 32 bp, synthetic construct
  misc_feature 1..32: note = Synthetic
  source 1..32: mol_type = other DNA
atgtaggaagggagagcacatacagttatctt

SEQ ID NO: 270 - DNA, 31 bp, synthetic construct
  misc_feature 1..31: note = Synthetic
  source 1..31: mol_type = other DNA
ggtcctgcccaaggttggatacagttatctt

SEQ ID NO: 271 - DNA, 33 bp, synthetic construct
  source 1..33: mol_type = other DNA
  misc_feature 1..33: note = Synthetic
cggctggacatatttgcatcatacagttatctt

SEQ ID NO: 272 - DNA, 32 bp, synthetic construct
  misc_feature 1..32: note = Synthetic
  source 1..32: mol_type = other DNA
caccactgggactatccatctacagttatctt

SEQ ID NO: 273 - DNA, 31 bp, synthetic construct
  misc_feature 1..31: note = Synthetic
  source 1..31: mol_type = other DNA
gcaccactgcacagaacagtacacttatctt

SEQ ID NO: 274 - DNA, 31 bp, synthetic construct
  source 1..31: mol_type = other DNA
  misc_feature 1..31: note = Synthetic
ccgggacccaaacaggcgttacacttatctt

SEQ ID NO: 275 - DNA, 33 bp, synthetic construct
  misc_feature 1..33: note = Synthetic
  source 1..33: mol_type = other DNA
gcagcgattaagggctcattatacacttatctt

SEQ ID NO: 276 - DNA, 32 bp, synthetic construct
  source 1..32: mol_type = other DNA
  misc_feature 1..32: note = Synthetic
acttggggaaggacaagatttacacttatctt

SEQ ID NO: 277 - DNA, 31 bp, synthetic construct
  misc_feature 1..31: note = Synthetic
  source 1..31: mol_type = other DNA
tttcacaggtgaggcgggctacacttatctt

SEQ ID NO: 278 - DNA, 33 bp, synthetic construct
  source 1..33: mol_type = other DNA
  misc_feature 1..33: note = Synthetic
agggcctagggactggattgctacacttatctt

SEQ ID NO: 279 - DNA, 33 bp, synthetic construct
  misc_feature 1..33: note = Synthetic
  source 1..33: mol_type = other DNA
ccccaaagcctttgattcccttacaattatctt

SEQ ID NO: 280 - DNA, 34 bp, synthetic construct
  misc_feature 1..34: note = Synthetic
  source 1..34: mol_type = other DNA
aggagcgatctcattttccacttacaattatctt

SEQ ID NO: 281 - DNA, 31 bp, synthetic construct
  misc_feature 1..31: note = Synthetic
  source 1..31: mol_type = other DNA
aaggcagagatcagcatcgtacaattatctt

SEQ ID NO: 282 - DNA, 31 bp, synthetic construct
  misc_feature 1..31: note = Synthetic
  source 1..31: mol_type = other DNA
aggttgcatggcgaggggatacaattatctt

SEQ ID NO: 283 - DNA, 37 bp, synthetic construct
  source 1..37: mol_type = other DNA
  misc_feature 1..37: note = Synthetic
ccagagagggtaccatacactcaaatacaattatctt

SEQ ID NO: 284 - DNA, 33 bp, synthetic construct
  source 1..33: mol_type = other DNA
  misc_feature 1..33: note = Synthetic
acacacatccaaggaaaaggttacaattatctt

SEQ ID NO: 285 - DNA, 31 bp, synthetic construct
  source 1..31: mol_type = other DNA
  misc_feature 1..31: note = Synthetic
ggtgacgtctggattgctgtaattttatctt

SEQ ID NO: 286 - DNA, 33 bp, synthetic construct
  source 1..33: mol_type = other DNA
  misc_feature 1..33: note = Synthetic
agaggatgtcatacccaaagctaattttatctt

SEQ ID NO: 287 - DNA, 32 bp, synthetic construct
  source 1..32: mol_type = other DNA
  misc_feature 1..32: note = Synthetic
aggagtgctggacttggcactaattttatctt

SEQ ID NO: 288 - DNA, 33 bp, synthetic construct
  source 1..33: mol_type = other DNA
  misc_feature 1..33: note = Synthetic
agcgcaagatggcataactaataattttatctt

SEQ ID NO: 289 - DNA, 31 bp, synthetic construct
  misc_feature 1..31: note = Synthetic
  source 1..31: mol_type = other DNA
gcgcggatctctgatgcggtaattttatctt

SEQ ID NO: 290 - DNA, 31 bp, synthetic construct
  source 1..31: mol_type = other DNA
  misc_feature 1..31: note = Synthetic
aaggttggaacggtttctctaattttatctt

SEQ ID NO: 291 - DNA, 34 bp, synthetic construct
  source 1..34: mol_type = other DNA
  misc_feature 1..34: note = Synthetic
agctgactggacatctctgcgataatgttatctt

SEQ ID NO: 292 - DNA, 31 bp, synthetic construct
  source 1..31: mol_type = other DNA
  misc_feature 1..31: note = Synthetic
accaggggtgacatctccctaatgttatctt

SEQ ID NO: 293 - DNA, 33 bp, synthetic construct
  misc_feature 1..33: note = Synthetic
  source 1..33: mol_type = other DNA
ccggttcaggtactcagtcattaatgttatctt

SEQ ID NO: 294 - DNA, 34 bp, synthetic construct
  source 1..34: mol_type = other DNA
  misc_feature 1..34: note = Synthetic
tggatgaaggggtgtctttagttaatgttatctt

SEQ ID NO: 295 - DNA, 31 bp, synthetic construct
  source 1..31: mol_type = other DNA
  misc_feature 1..31: note = Synthetic
acactccggcttcactgagtaatgttatctt

SEQ ID NO: 296 - DNA, 32 bp, synthetic construct
  source 1..32: mol_type = other DNA
  misc_feature 1..32: note = Synthetic
cgggacgacctaggaagttataatgttatctt

SEQ ID NO: 297 - DNA, 32 bp, synthetic construct
  misc_feature 1..32: note = Synthetic
  source 1..32: mol_type = other DNA
gtttccagagtttggcagcataatcttatctt

SEQ ID NO: 298 - DNA, 36 bp, synthetic construct
  source 1..36: mol_type = other DNA
  misc_feature 1..36: note = Synthetic
tgtgtggaataaagatacatgcgttaatcttatctt

SEQ ID NO: 299 - DNA, 32 bp, synthetic construct
  misc_feature 1..32: note = Synthetic
  source 1..32: mol_type = other DNA
accatctctgtagtcaggggtaatcttatctt

SEQ ID NO: 300 - DNA, 31 bp, synthetic construct
  misc_feature 1..31: note = Synthetic
  source 1..31: mol_type = other DNA
cagtccaggaaactttcggtaatcttatctt

SEQ ID NO: 301 - DNA, 33 bp, synthetic construct
  source 1..33: mol_type = other DNA
  misc_feature 1..33: note = Synthetic
atcatttgcttctttgcgatataatcttatctt

SEQ ID NO: 302 - DNA, 33 bp, synthetic construct
  misc_feature 1..33: note = Synthetic
  source 1..33: mol_type = other DNA
ggagattggttatacacagcataatcttatctt

SEQ ID NO: 303 - DNA, 36 bp, synthetic construct
  source 1..36: mol_type = other DNA
  misc_feature 1..36: note = Synthetic
ctggcctctgacatacttgttgagtaatattatctt

SEQ ID NO: 304 - DNA, 35 bp, synthetic construct
  misc_feature 1..35: note = Synthetic
  source 1..35: mol_type = other DNA
tccagttcagcataagatcctcctaatattatctt

SEQ ID NO: 305 - DNA, 32 bp, synthetic construct
  source 1..32: mol_type = other DNA
  misc_feature 1..32: note = Synthetic
ccaccagacagctagactcttaatattatctt

SEQ ID NO: 306 - DNA, 33 bp, synthetic construct
  source 1..33: mol_type = other DNA
  misc_feature 1..33: note = Synthetic
tgctcatttcagagtcctaggtaatattatctt

SEQ ID NO: 307 - DNA, 31 bp, synthetic construct
  misc_feature 1..31: note = Synthetic
  source 1..31: mol_type = other DNA
atcctggacgggttggctctaatattatctt

SEQ ID NO: 308 - DNA, 31 bp, synthetic construct
  source 1..31: mol_type = other DNA
  misc_feature 1..31: note = Synthetic
ggccattcaaagcaggctgtaatattatctt

SEQ ID NO: 309 - DNA, 33 bp, synthetic construct
  misc_feature 1..33: note = Synthetic
  source 1..33: mol_type = other DNA
accaactccattgtgctgagataagtttatctt

SEQ ID NO: 310 - DNA, 33 bp, synthetic construct
  misc_feature 1..33: note = Synthetic
  source 1..33: mol_type = other DNA
agatgagcctgactgttcggttaagtttatctt

SEQ ID NO: 311 - DNA, 31 bp, synthetic construct
  source 1..31: mol_type = other DNA
  misc_feature 1..31: note = Synthetic
aggctgaggagaaataccataagtttatctt

SEQ ID NO: 312 - DNA, 31 bp, synthetic construct
  misc_feature 1..31: note = Synthetic
  source 1..31: mol_type = other DNA
ggggctcactacagacaggtaagtttatctt

SEQ ID NO: 313 - DNA, 31 bp, synthetic construct
  misc_feature 1..31: note = Synthetic
  source 1..31: mol_type = other DNA
tcagtagaaggagggtccctaagtttatctt

SEQ ID NO: 314 - DNA, 33 bp, synthetic construct
  source 1..33: mol_type = other DNA
  misc_feature 1..33: note = Synthetic
agtccatgtcagggtttcaagtaagtttatctt

SEQ ID NO: 315 - DNA, 31 bp, synthetic construct
  misc_feature 1..31: note = Synthetic
  source 1..31: mol_type = other DNA
cggggacaattcggtaaactaaggttatctt

SEQ ID NO: 316 - DNA, 34 bp, synthetic construct
  source 1..34: mol_type = other DNA
  misc_feature 1..34: note = Synthetic
agctgaatggtgtgtgtcatagtaaggttatctt

SEQ ID NO: 317 - DNA, 32 bp, synthetic construct
  misc_feature 1..32: note = Synthetic
  source 1..32: mol_type = other DNA
aggtgacatgtgacaaggggtaaggttatctt

SEQ ID NO: 318 - DNA, 33 bp, synthetic construct
  source 1..33: mol_type = other DNA
  misc_feature 1..33: note = Synthetic
accgtggtccttagcttttcataaggttatctt

SEQ ID NO: 319 - DNA, 33 bp, synthetic construct
  source 1..33: mol_type = other DNA
  misc_feature 1..33: note = Synthetic
agagtctgtgtgaatcaatgataaggttatctt

SEQ ID NO: 320 - DNA, 32 bp, synthetic construct
  source 1..32: mol_type = other DNA
  misc_feature 1..32: note = Synthetic
tttccccagagcaacagtcataaggttatctt

SEQ ID NO: 321 - DNA, 32 bp, synthetic construct
  misc_feature 1..32: note = Synthetic
  source 1..32: mol_type = other DNA
tcctctgtccattgtcgtcctaagcttatctt

SEQ ID NO: 322 - DNA, 33 bp, synthetic construct
  source 1..33: mol_type = other DNA
  misc_feature 1..33: note = Synthetic
agcggtggtaatctggagtggtaagcttatctt

SEQ ID NO: 323 - DNA, 35 bp, synthetic construct
  misc_feature 1..35: note = Synthetic
  source 1..35: mol_type = other DNA
acacttgcagctctgatagaaactaagcttatctt

SEQ ID NO: 324 - DNA, 32 bp, synthetic construct
  source 1..32: mol_type = other DNA
  misc_feature 1..32: note = Synthetic
gctagcagaggttccacatgtaagcttatctt

SEQ ID NO: 325 - DNA, 31 bp, synthetic construct
  source 1..31: mol_type = other DNA
  misc_feature 1..31: note = Synthetic
cgtgggccaaacgtcacagtaagcttatctt

SEQ ID NO: 326 - DNA, 35 bp, synthetic construct
  misc_feature 1..35: note = Synthetic
  source 1..35: mol_type = other DNA
agcagcaattagcaaggactaagtaagcttatctt

SEQ ID NO: 327 - DNA, 33 bp, synthetic construct
  source 1..33: mol_type = other DNA
  misc_feature 1..33: note = Synthetic
gggtggaatttgacctcttagtaagattatctt

SEQ ID NO: 328 - DNA, 34 bp, synthetic construct
  source 1..34: mol_type = other DNA
  misc_feature 1..34: note = Synthetic
aggaatagtcacggttgacgaataagattatctt

SEQ ID NO: 329 - DNA, 34 bp, synthetic construct
  misc_feature 1..34: note = Synthetic
  source 1..34: mol_type = other DNA
ccaggagactgatatacacttataagattatctt

SEQ ID NO: 330 - DNA, 31 bp, synthetic construct
  misc_feature 1..31: note = Synthetic
  source 1..31: mol_type = other DNA
aggtgccacctgatcgcagtaagattatctt

SEQ ID NO: 331 - DNA, 33 bp, synthetic construct
  source 1..33: mol_type = other DNA
  misc_feature 1..33: note = Synthetic
tcctttccagggtgtgactcataagattatctt

SEQ ID NO: 332 - DNA, 35 bp, synthetic construct
  source 1..35: mol_type = other DNA
  misc_feature 1..35: note = Synthetic
agggataacacttttcgaaaagttaactttatctt

SEQ ID NO: 333 - DNA, 35 bp, synthetic construct
  misc_feature 1..35: note = Synthetic
  source 1..35: mol_type = other DNA
aggtactgtttgtcactgcacgttaactttatctt

SEQ ID NO: 334 - DNA, 36 bp, synthetic construct
  misc_feature 1..36: note = Synthetic
  source 1..36: mol_type = other DNA
agtgttgtccttccttacaggtactaactttatctt

SEQ ID NO: 335 - DNA, 33 bp, synthetic construct
  misc_feature 1..33: note = Synthetic
  source 1..33: mol_type = other DNA
actcgttcctttctgtaggactaactttatctt

SEQ ID NO: 336 - DNA, 32 bp, synthetic construct
  misc_feature 1..32: note = Synthetic
  source 1..32: mol_type = other DNA
agccagggatacagggcgtgtaactttatctt

SEQ ID NO: 337 - DNA, 32 bp, synthetic construct
  misc_feature 1..32: note = Synthetic
  source 1..32: mol_type = other DNA
actgggctgagaattcgccttaactttatctt

SEQ ID NO: 338 - DNA, 31 bp, synthetic construct
  source 1..31: mol_type = other DNA
  misc_feature 1..31: note = Synthetic
ggtgactggaagaaaagcttaacgttatctt

SEQ ID NO: 339 - DNA, 32 bp, synthetic construct
  source 1..32: mol_type = other DNA
  misc_feature 1..32: note = Synthetic
ccctgtacttcctgaaagtttaacgttatctt

SEQ ID NO: 340 - DNA, 33 bp, synthetic construct
  misc_feature 1..33: note = Synthetic
  source 1..33: mol_type = other DNA
atggtagtgttttggatcgtctaacgttatctt

SEQ ID NO: 341 - DNA, 31 bp, synthetic construct
  misc_feature 1..31: note = Synthetic
  source 1..31: mol_type = other DNA
tgtggccagacttctggtttaacgttatctt

SEQ ID NO: 342 - DNA, 32 bp, synthetic construct
  source 1..32: mol_type = other DNA
  misc_feature 1..32: note = Synthetic
actgaaaagcactcacgaggtaacgttatctt

SEQ ID NO: 343 - DNA, 32 bp, synthetic construct
  source 1..32: mol_type = other DNA
  misc_feature 1..32: note = Synthetic
agaagcggtgatactccatgtaacgttatctt

SEQ ID NO: 344 - DNA, 35 bp, synthetic construct
  misc_feature 1..35: note = Synthetic
  source 1..35: mol_type = other DNA
tgcagtcccattgaaataagctttaaccttatctt

SEQ ID NO: 345 - DNA, 33 bp, synthetic construct
  misc_feature 1..33: note = Synthetic
  source 1..33: mol_type = other DNA
gtgcccaaatagtgctcgtactaaccttatctt

SEQ ID NO: 346 - DNA, 33 bp, synthetic construct
  misc_feature 1..33: note = Synthetic
  source 1..33: mol_type = other DNA
accatccgggaatgaaagagataaccttatctt

SEQ ID NO: 347 - DNA, 34 bp, synthetic construct
  source 1..34: mol_type = other DNA
  misc_feature 1..34: note = Synthetic
aggaaatcttcattgactccgttaaccttatctt

SEQ ID NO: 348 - DNA, 31 bp, synthetic construct
  misc_feature 1..31: note = Synthetic
  source 1..31: mol_type = other DNA
cctgctaggctgattcggctaaccttatctt

SEQ ID NO: 349 - DNA, 33 bp, synthetic construct
  source 1..33: mol_type = other DNA
  misc_feature 1..33: note = Synthetic
atgagctgaaagagtcctagctaaccttatctt

SEQ ID NO: 350 - DNA, 33 bp, synthetic construct
  misc_feature 1..33: note = Synthetic
  source 1..33: mol_type = other DNA
gtgcacggtaccatcgcaccttaacattatctt

SEQ ID NO: 351 - DNA, 32 bp, synthetic construct
  misc_feature 1..32: note = Synthetic
  source 1..32: mol_type = other DNA
tttacaatctgggagagacataacattatctt

SEQ ID NO: 352 - DNA, 32 bp, synthetic construct
  misc_feature 1..32: note = Synthetic
  source 1..32: mol_type = other DNA
cgatgtaatcgcagccgtcgtaacattatctt

SEQ ID NO: 353 - DNA, 31 bp, synthetic construct
  source 1..31: mol_type = other DNA
  misc_feature 1..31: note = Synthetic
tgacaggctacagatgtcctaacattatctt

SEQ ID NO: 354 - DNA, 35 bp, synthetic construct
  source 1..35: mol_type = other DNA
  misc_feature 1..35: note = Synthetic
aggtcttataccagctcaccaactaaatttatctt

SEQ ID NO: 355 - DNA, 35 bp, synthetic construct
  source 1..35: mol_type = other DNA
  misc_feature 1..35: note = Synthetic
acagtcagaaacacgatggcatctaaatttatctt

SEQ ID NO: 356 - DNA, 34 bp, synthetic construct
  misc_feature 1..34: note = Synthetic
  source 1..34: mol_type = other DNA
accaatctgatggccttcttcataaatttatctt

SEQ ID NO: 357 - DNA, 33 bp, synthetic construct
  source 1..33: mol_type = other DNA
  misc_feature 1..33: note = Synthetic
agcatctgaagtgacctcatgtaaatttatctt

SEQ ID NO: 358 - DNA, 31 bp, synthetic construct
  source 1..31: mol_type = other DNA
  misc_feature 1..31: note = Synthetic
ctctgctggtcacaggccgtaaatttatctt

SEQ ID NO: 359 - DNA, 33 bp, synthetic construct
  source 1..33: mol_type = other DNA
  misc_feature 1..33: note = Synthetic
cggcacagatatctcggttcttaaagttatctt

SEQ ID NO: 360 - DNA, 32 bp, synthetic construct
  source 1..32: mol_type = other DNA
  misc_feature 1..32: note = Synthetic
aggtccagaagaactccttctaaagttatctt

SEQ ID NO: 361 - DNA, 31 bp, synthetic construct
  misc_feature 1..31: note = Synthetic
  source 1..31: mol_type = other DNA
gtcctcctccctaggacagtaaagttatctt

SEQ ID NO: 362 - DNA, 35 bp, synthetic construct
  misc_feature 1..35: note = Synthetic
  source 1..35: mol_type = other DNA
acaccatagcataacttcgactataaagttatctt

SEQ ID NO: 363 - DNA, 32 bp, synthetic construct
  source 1..32: mol_type = other DNA
  misc_feature 1..32: note = Synthetic
tcaaaacaacgccaggcttgtaaagttatctt

SEQ ID NO: 364 - DNA, 35 bp, synthetic construct
  misc_feature 1..35: note = Synthetic
  source 1..35: mol_type = other DNA
ggggcagagaaagtaaaagaccctaaagttatctt

SEQ ID NO: 365 - DNA, 33 bp, synthetic construct
  source 1..33: mol_type = other DNA
  misc_feature 1..33: note = Synthetic
ggagctgaggactttccacgttaaacttatctt

SEQ ID NO: 366 - DNA, 32 bp, synthetic construct
  misc_feature 1..32: note = Synthetic
  source 1..32: mol_type = other DNA
caccaggtacatccatgaaataaacttatctt

SEQ ID NO: 367 - DNA, 32 bp, synthetic construct
  source 1..32: mol_type = other DNA
  misc_feature 1..32: note = Synthetic
tacaaagcgtcacggaagtctaaacttatctt

SEQ ID NO: 368 - DNA, 32 bp, synthetic construct
  source 1..32: mol_type = other DNA
  misc_feature 1..32: note = Synthetic
atttctccccgagaaagaagtaaacttatctt

SEQ ID NO: 369 - DNA, 31 bp, synthetic construct
  misc_feature 1..31: note = Synthetic
  source 1..31: mol_type = other DNA
agggattgtcccaacaccctaaacttatctt

SEQ ID NO: 370 - DNA, 31 bp, synthetic construct
  source 1..31: mol_type = other DNA
  misc_feature 1..31: note = Synthetic
aggtgagtaccgtcatgcctaaacttatctt

SEQ ID NO: 371 - DNA, 36 bp, synthetic construct
  misc_feature 1..36: note = Synthetic
  source 1..36: mol_type = other DNA
actactttctggcggaagtagaattaaaattatctt

SEQ ID NO: 372 - DNA, 32 bp, synthetic construct
  misc_feature 1..32: note = Synthetic
  source 1..32: mol_type = other DNA
gctggtgataattcctagcataaaattatctt

SEQ ID NO: 373 - DNA, 33 bp, synthetic construct
  source 1..33: mol_type = other DNA
  misc_feature 1..33: note = Synthetic
tccctcagcaatcatcaatagtaaaattatctt

SEQ ID NO: 374 - DNA, 31 bp, synthetic construct
  misc_feature 1..31: note = Synthetic
  source 1..31: mol_type = other DNA
gtgcctgcttgtagcgtgttaaaattatctt

SEQ ID NO: 375 - DNA, 31 bp, synthetic construct
  misc_feature 1..31: note = Synthetic
  source 1..31: mol_type = other DNA
gggagggcctttagcgcagtaaaattatctt

SEQ ID NO: 376 - DNA, 31 bp, synthetic construct
  misc_feature 1..31: note = Synthetic
  source 1..31: mol_type = other DNA
tctcatcgccctcttcacataaaattatctt

SEQ ID NO: 377 - DNA, 54 bp, synthetic construct
  misc_feature 1..54: note = Synthetic
  misc_feature <1: note = /5Phos/
  source 1..54: mol_type = other DNA
aaaatattgctgaacttgttcagggtaattattacaaaacatacactaaagata

SEQ ID NO: 378 - DNA, 54 bp, synthetic construct
  misc_feature 1..54: note = Synthetic
  misc_feature <1: note = /5Phos/
  source 1..54: mol_type = other DNA
aaaataatagacaacgaaggtgggctaattattacaaaacatacactaaagata

SEQ ID NO: 379 - DNA, 53 bp, synthetic construct
  misc_feature 1..53: note = Synthetic
  misc_feature <1: note = /5Phos/
  source 1..53: mol_type = other DNA
aaaataggcgtccattttctttggaaattattacaaaacatacactaaagata

SEQ ID NO: 380 - DNA, 53 bp, synthetic construct
  misc_feature <1: note = /5Phos/
  source 1..53: mol_type = other DNA
  misc_feature 1..53: note = Synthetic
aaaatagacgaattcagcttctcgtaattattacaaaacatacactaaagata

SEQ ID NO: 381 - DNA, 54 bp, synthetic construct
  source 1..54: mol_type = other DNA
  misc_feature 1..54: note = Synthetic
  misc_feature <1: note = /5Phos/
aaaatatgagagtgatgatcaaggacaattattacaaaacatacactaaagata

SEQ ID NO: 382 - DNA, 56 bp, synthetic construct
  misc_feature 1..56: note = Synthetic
  source 1..56: mol_type = other DNA
  misc_feature <1: note = /5Phos/
aaaataataaatggggattctccagacaaattattacaaaacatacactaaagata

SEQ ID NO: 383 - DNA, 53 bp, synthetic construct
  misc_feature <1: note = /5Phos/
  source 1..53: mol_type = other DNA
  misc_feature 1..53: note = Synthetic
acaatagcaaagcagtagacgcccaaattattacacaacatacactaaagata

SEQ ID NO: 384 - DNA, 59 bp, synthetic construct
  misc_feature <1: note = /5Phos/
  source 1..59: mol_type = other DNA
  misc_feature 1..59: note = Synthetic
acaatagctgtactgggtatcttcacgatctaattattacacaacatacactaaagata

SEQ ID NO: 385 - DNA, 54 bp, synthetic construct
  misc_feature 1..54: note = Synthetic
  source 1..54: mol_type = other DNA
  misc_feature <1: note = /5Phos/
acaatatttgcccaagttgagtgtttaattattacacaacatacactaaagata

SEQ ID NO: 386 - DNA, 55 bp, synthetic construct
  misc_feature 1..55: note = Synthetic
  misc_feature <1: note = /5Phos/
  source 1..55: mol_type = other DNA
acaatatccatctagatgcatgacgctaattattacacaacatacactaaagata

SEQ ID NO: 387 - DNA, 57 bp, synthetic construct
  source 1..57: mol_type = other DNA
  misc_feature 1..57: note = Synthetic
  misc_feature <1: note = /5Phos/
acaatactcatattcggtcacttgtatctaattattacacaacatacactaaagata

SEQ ID NO: 388 - DNA, 53 bp, synthetic construct
  misc_feature 1..53: note = Synthetic
  misc_feature <1: note = /5Phos/
  source 1..53: mol_type = other DNA
acaatacaaatagaggcaagccacaaattattacacaacatacactaaagata

SEQ ID NO: 389 - DNA, 57 bp, synthetic construct
  misc_feature <1: note = /5Phos/
  source 1..57: mol_type = other DNA
  misc_feature 1..57: note = Synthetic
agaataacctgaagtcgactcctgtagctaattattacagaacatacactaaagata

SEQ ID NO: 390 - DNA, 55 bp, synthetic construct
  misc_feature 1..55: note = Synthetic
  source 1..55: mol_type = other DNA
  misc_feature <1: note = /5Phos/
agaatatatgaggtcttttgggctcctaattattacagaacatacactaaagata

SEQ ID NO: 391 - DNA, 57 bp, synthetic construct
  misc_feature <1: note = /5Phos/
  misc_feature 1..57: note = Synthetic
  source 1..57: mol_type = other DNA
agaatacattatgtagccagagtaactggaattattacagaacatacactaaagata

SEQ ID NO: 392 - DNA, 55 bp, synthetic construct
  misc_feature 1..55: note = Synthetic
  misc_feature <1: note = /5Phos/
  source 1..55: mol_type = other DNA
agaatagttacaatagctggatgagggaattattacagaacatacactaaagata

SEQ ID NO: 393 - DNA, 53 bp, synthetic construct
  misc_feature 1..53: note = Synthetic
  source 1..53: mol_type = other DNA
  misc_feature <1: note = /5Phos/
agaataacgtgacaaggcaaacacaaattattacagaacatacactaaagata

SEQ ID NO: 394 - DNA, 53 bp, synthetic construct
  source 1..53: mol_type = other DNA
  misc_feature 1..53: note = Synthetic
  misc_feature <1: note = /5Phos/
agaatatgaatccacccgtgatgggaattattacagaacatacactaaagata

SEQ ID NO: 395 - DNA, 54 bp, synthetic construct
  misc_feature <1: note = /5Phos/
  source 1..54: mol_type = other DNA
  misc_feature 1..54: note = Synthetic
ataatacatgtttcaaaacccttgctaattattacataacatacactaaagata

SEQ ID NO: 396 - DNA, 54 bp, synthetic construct
  misc_feature <1: note = /5Phos/
  misc_feature 1..54: note = Synthetic
  source 1..54: mol_type = other DNA
ataatatcggaattaccacatttcctaattattacataacatacactaaagata

SEQ ID NO: 397 - DNA, 53 bp, synthetic construct
  source 1..53: mol_type = other DNA
  misc_feature <1: note = /5Phos/
  misc_feature 1..53: note = Synthetic
ataatagggtgctcttctcgatggtaattattacataacatacactaaagata

SEQ ID NO: 398 - DNA, 55 bp, synthetic construct
  source 1..55: mol_type = other DNA
  misc_feature 1..55: note = Synthetic
  misc_feature <1: note = /5Phos/
ataataagctaattcggatccatgagtaattattacataacatacactaaagata

SEQ ID NO: 399 - DNA, 55 bp, synthetic construct
  misc_feature 1..55: note = Synthetic
  misc_feature <1: note = /5Phos/
  source 1..55: mol_type = other DNA
ataatagatatatactcctgtgtggttaattattacataacatacactaaagata

SEQ ID NO: 400 - DNA, 58 bp, synthetic construct
  misc_feature <1: note = /5Phos/
  source 1..58: mol_type = other DNA
  misc_feature 1..58: note = Synthetic
ataatatttcaagttaatggcgtaggcactaattattacataacatacactaaagata

SEQ ID NO: 401 - DNA, 55 bp, synthetic construct
  misc_feature <1: note = /5Phos/
  source 1..55: mol_type = other DNA
  misc_feature 1..55: note = Synthetic
aacatactcttggtgcatagactgtgtaattattacaacacatacactaaagata

SEQ ID NO: 402 - DNA, 58 bp, synthetic construct
  source 1..58: mol_type = other DNA
  misc_feature 1..58: note = Synthetic
  misc_feature <1: note = /5Phos/
aacataatacactcgtagtgaacactctgtaattattacaacacatacactaaagata

SEQ ID NO: 403 - DNA, 53 bp, synthetic construct
  misc_feature <1: note = /5Phos/
  misc_feature 1..53: note = Synthetic
  source 1..53: mol_type = other DNA
aacataggtgccgttcttgtaggagaattattacaacacatacactaaagata

SEQ ID NO: 404 - DNA, 53 bp, synthetic construct
  misc_feature 1..53: note = Synthetic
  source 1..53: mol_type = other DNA
  misc_feature <1: note = /5Phos/
aacatatctccgtcattgcagttgtaattattacaacacatacactaaagata

SEQ ID NO: 405 - DNA, 59 bp, synthetic construct
  source 1..59: mol_type = other DNA
  misc_feature 1..59: note = Synthetic
  misc_feature <1: note = /5Phos/
aacatacatcttgcagatgctaatagcaggaaattattacaacacatacactaaagata

SEQ ID NO: 406 - DNA, 53 bp, synthetic construct
  misc_feature <1: note = /5Phos/
  source 1..53: mol_type = other DNA
  misc_feature 1..53: note = Synthetic
aacatacaagtccatacctgtgcctaattattacaacacatacactaaagata

SEQ ID NO: 407 - DNA, 53 bp, synthetic construct
  misc_feature <1: note = /5Phos/
  misc_feature 1..53: note = Synthetic
  source 1..53: mol_type = other DNA
accatagaatcgtcaactcttgccgaattattacaccacatacactaaagata

SEQ ID NO: 408 - DNA, 57 bp, synthetic construct
  misc_feature <1: note = /5Phos/
  misc_feature 1..57: note = Synthetic
  source 1..57: mol_type = other DNA
accataaggtataagtgcctccttctgtgaattattacaccacatacactaaagata

SEQ ID NO: 409 - DNA, 58 bp, synthetic construct
  misc_feature 1..58: note = Synthetic
  source 1..58: mol_type = other DNA
  misc_feature <1: note = /5Phos/
accatagtacttcatacatgggtttctgcaaattattacaccacatacactaaagata

SEQ ID NO: 410 - DNA, 55 bp, synthetic construct
  misc_feature <1: note = /5Phos/
  source 1..55: mol_type = other DNA
  misc_feature 1..55: note = Synthetic
accatatcgtacgtcaggatttctggtaattattacaccacatacactaaagata

SEQ ID NO: 411 - DNA, 53 bp, synthetic construct
  misc_feature 1..53: note = Synthetic
  misc_feature <1: note = /5Phos/
  source 1..53: mol_type = other DNA
accataaggtgacttgtttcaggcaaattattacaccacatacactaaagata

SEQ ID NO: 412 - DNA, 53 bp, synthetic construct
  misc_feature 1..53: note = Synthetic
  misc_feature <1: note = /5Phos/
  source 1..53: mol_type = other DNA
accataacgtacacactgaacccttaattattacaccacatacactaaagata

SEQ ID NO: 413 - DNA, 56 bp, synthetic construct
  source 1..56: mol_type = other DNA
  misc_feature <1: note = /5Phos/
  misc_feature 1..56: note = Synthetic
agcatagtttcgttgtcgaaatccccgtaattattacagcacatacactaaagata

SEQ ID NO: 414 - DNA, 59 bp, synthetic construct
  misc_feature <1: note = /5Phos/
  misc_feature 1..59: note = Synthetic
  source 1..59: mol_type = other DNA
agcataaaggttgtaggaatacctgcagctgaattattacagcacatacactaaagata

SEQ ID NO: 415 - DNA, 53 bp, synthetic construct
  misc_feature 1..53: note = Synthetic
  misc_feature <1: note = /5Phos/
  source 1..53: mol_type = other DNA
agcatatgtctaggtaaggcggaggaattattacagcacatacactaaagata

SEQ ID NO: 416 - DNA, 54 bp, synthetic construct
  misc_feature <1: note = /5Phos/
  source 1..54: mol_type = other DNA
  misc_feature 1..54: note = Synthetic
agcatagccatagaagcttctgtcataattattacagcacatacactaaagata

SEQ ID NO: 417 - DNA, 54 bp, synthetic construct
  misc_feature 1..54: note = Synthetic
  misc_feature <1: note = /5Phos/
  source 1..54: mol_type = other DNA
agcataaaaattcccattcccgacacaattattacagcacatacactaaagata

SEQ ID NO: 418 - DNA, 59 bp, synthetic construct
  misc_feature <1: note = /5Phos/
  source 1..59: mol_type = other DNA
  misc_feature 1..59: note = Synthetic
agcatacccaaaacagcttaggagatgactgaattattacagcacatacactaaagata

SEQ ID NO: 419 - DNA, 57 bp, synthetic construct
  source 1..57: mol_type = other DNA
  misc_feature <1: note = /5Phos/
  misc_feature 1..57: note = Synthetic
atcatatcagaagtacagaaccgtctcctaattattacatcacatacactaaagata

SEQ ID NO: 420 - DNA, 54 bp, synthetic construct
  source 1..54: mol_type = other DNA
  misc_feature <1: note = /5Phos/
  misc_feature 1..54: note = Synthetic
atcatacaagagcaaggaagaagatgaattattacatcacatacactaaagata

SEQ ID NO: 421 - DNA, 53 bp, synthetic construct
  source 1..53: mol_type = other DNA
  misc_feature <1: note = /5Phos/
  misc_feature 1..53: note = Synthetic
atcatagcgaactccatcctgtcccaattattacatcacatacactaaagata

SEQ ID NO: 422 - DNA, 55 bp, synthetic construct
  misc_feature <1: note = /5Phos/
  misc_feature 1..55: note = Synthetic
  source 1..55: mol_type = other DNA
atcatatgcagttttcacacattctgtaattattacatcacatacactaaagata

SEQ ID NO: 423 - DNA, 55 bp, synthetic construct
  misc_feature <1: note = /5Phos/
  misc_feature 1..55: note = Synthetic
  source 1..55: mol_type = other DNA
atcataagccggatatggtgcctatgtaattattacatcacatacactaaagata

SEQ ID NO: 424 - DNA, 53 bp, synthetic construct
  misc_feature <1: note = /5Phos/
  misc_feature 1..53: note = Synthetic
  source 1..53: mol_type = other DNA
atcatattcccggtccttgaggggcaattattacatcacatacactaaagata

SEQ ID NO: 425 - DNA, 58 bp, synthetic construct
  source 1..58: mol_type = other DNA
  misc_feature <1: note = /5Phos/
  misc_feature 1..58: note = Synthetic
aagatagtcaactctacactggttcctgagaattattacaagacatacactaaagata

SEQ ID NO: 426 - DNA, 58 bp, synthetic construct
  misc_feature 1..58: note = Synthetic
  misc_feature <1: note = /5Phos/
  source 1..58: mol_type = other DNA
aagataacaatgatgattatggctactgctaattattacaagacatacactaaagata

SEQ ID NO: 427 - DNA, 53 bp, synthetic construct
  source 1..53: mol_type = other DNA
  misc_feature <1: note = /5Phos/
  misc_feature 1..53: note = Synthetic
aagatagccagaatacaggtcccgcaattattacaagacatacactaaagata

SEQ ID NO: 428 - DNA, 56 bp, synthetic construct
  misc_feature <1: note = /5Phos/
  source 1..56: mol_type = other DNA
  misc_feature 1..56: note = Synthetic
aagatataaggggaaatctggagaggcaaattattacaagacatacactaaagata

SEQ ID NO: 429 - DNA, 54 bp, synthetic construct
  misc_feature 1..54: note = Synthetic
  misc_feature <1: note = /5Phos/
  source 1..54: mol_type = other DNA
aagatatgagggccaattaggagaggaattattacaagacatacactaaagata

SEQ ID NO: 430 - DNA, 55 bp, synthetic construct
  misc_feature 1..55: note = Synthetic
  misc_feature <1: note = /5Phos/
  source 1..55: mol_type = other DNA
aagatatctcctatctgtcagactgctaattattacaagacatacactaaagata

SEQ ID NO: 431 - DNA, 55 bp, synthetic construct
  misc_feature <1: note = /5Phos/
  source 1..55: mol_type = other DNA
  misc_feature 1..55: note = Synthetic
acgatacattgtagagctggttggggtaattattacacgacatacactaaagata

SEQ ID NO: 432 - DNA, 57 bp, synthetic construct
  misc_feature 1..57: note = Synthetic
  source 1..57: mol_type = other DNA
  misc_feature <1: note = /5Phos/
acgatagatgaagaggattccatctagcaaattattacacgacatacactaaagata

SEQ ID NO: 433 - DNA, 58 bp, synthetic construct
  source 1..58: mol_type = other DNA
  misc_feature 1..58: note = Synthetic
  misc_feature <1: note = /5Phos/
acgatagcatgaaagaaattggaacatcagaattattacacgacatacactaaagata

SEQ ID NO: 434 - DNA, 57 bp, synthetic construct
  source 1..57: mol_type = other DNA
  misc_feature 1..57: note = Synthetic
  misc_feature <1: note = /5Phos/
acgatatccttgtgagttctgaaccttctaattattacacgacatacactaaagata

SEQ ID NO: 435 - DNA, 54 bp, synthetic construct
  source 1..54: mol_type = other DNA
  misc_feature <1: note = /5Phos/
  misc_feature 1..54: note = Synthetic
acgatacagaccaaagctctgtgcctaattattacacgacatacactaaagata

SEQ ID NO: 436 - DNA, 54 bp, synthetic construct
  source 1..54: mol_type = other DNA
  misc_feature <1: note = /5Phos/
  misc_feature 1..54: note = Synthetic
acgatatgcctctccgcctctcgcctaattattacacgacatacactaaagata

SEQ ID NO: 437 - DNA, 54 bp, synthetic construct
  misc_feature 1..54: note = Synthetic
  source 1..54: mol_type = other DNA
  misc_feature <1: note = /5Phos/
aggatattgggtcataagcttccgggaattattacaggacatacactaaagata

SEQ ID NO: 438 - DNA, 59 bp, synthetic construct
  source 1..59: mol_type = other DNA
  misc_feature 1..59: note = Synthetic
  misc_feature <1: note = /5Phos/
aggatattgaaactgtactgacagagggactaattattacaggacatacactaaagata

SEQ ID NO: 439 - DNA, 56 bp, synthetic construct
  source 1..56: mol_type = other DNA
  misc_feature 1..56: note = Synthetic
  misc_feature <1: note = /5Phos/
aggatattcaataatttcagggacatgtaattattacaggacatacactaaagata

SEQ ID NO: 440 - DNA, 55 bp, synthetic construct
  misc_feature <1: note = /5Phos/
  source 1..55: mol_type = other DNA
  misc_feature 1..55: note = Synthetic
aggatagtctaaacagcttgttacccgaattattacaggacatacactaaagata

SEQ ID NO: 441 - DNA, 55 bp, synthetic construct
  misc_feature <1: note = /5Phos/
  source 1..55: mol_type = other DNA
  misc_feature 1..55: note = Synthetic
aggatatgaaaggtttggcagagacccaattattacaggacatacactaaagata

SEQ ID NO: 442 - DNA, 54 bp, synthetic construct
  source 1..54: mol_type = other DNA
  misc_feature 1..54: note = Synthetic
  misc_feature <1: note = /5Phos/
aggatatagcgggacatgaagagcgcaattattacaggacatacactaaagata

SEQ ID NO: 443 - DNA, 53 bp, synthetic construct
  source 1..53: mol_type = other DNA
  misc_feature <1: note = /5Phos/
  misc_feature 1..53: note = Synthetic
atgatacccctagagagacaaggctaattattacatgacatacactaaagata

SEQ ID NO: 444 - DNA, 55 bp, synthetic construct
  misc_feature 1..55: note = Synthetic
  source 1..55: mol_type = other DNA
  misc_feature <1: note = /5Phos/
atgatatatctgggttcagggacatctaattattacatgacatacactaaagata

SEQ ID NO: 445 - DNA, 53 bp, synthetic construct
  source 1..53: mol_type = other DNA
  misc_feature <1: note = /5Phos/
  misc_feature 1..53: note = Synthetic
atgatacaagactttcccagagcctaattattacatgacatacactaaagata

SEQ ID NO: 446 - DNA, 53 bp, synthetic construct
  misc_feature <1: note = /5Phos/
  misc_feature 1..53: note = Synthetic
  source 1..53: mol_type = other DNA
atgatactcctagctcatggcatctaattattacatgacatacactaaagata

SEQ ID NO: 447 - DNA, 56 bp, synthetic construct
  misc_feature <1: note = /5Phos/
  misc_feature 1..56: note = Synthetic
  source 1..56: mol_type = other DNA
atgatattgaatagaacggaacccaggtaattattacatgacatacactaaagata

SEQ ID NO: 448 - DNA, 54 bp, synthetic construct
  source 1..54: mol_type = other DNA
  misc_feature 1..54: note = Synthetic
  misc_feature <1: note = /5Phos/
atgatagtgcaattcactgctggggtaattattacatgacatacactaaagata

SEQ ID NO: 449 - DNA, 55 bp, synthetic construct
  misc_feature <1: note = /5Phos/
  source 1..55: mol_type = other DNA
  misc_feature 1..55: note = Synthetic
aatatatgtcgtgttgacactggctggaattattacaatacatacactaaagata

SEQ ID NO: 450 - DNA, 54 bp, synthetic construct
  source 1..54: mol_type = other DNA
  misc_feature <1: note = /5Phos/
  misc_feature 1..54: note = Synthetic
aatatatgatgactttctgtgctcagaattattacaatacatacactaaagata

SEQ ID NO: 451 - DNA, 54 bp, synthetic construct
  source 1..54: mol_type = other DNA
  misc_feature 1..54: note = Synthetic
  misc_feature <1: note = /5Phos/
aatatagcactattccaggaccaaggaattattacaatacatacactaaagata

SEQ ID NO: 452 - DNA, 57 bp, synthetic construct
  misc_feature <1: note = /5Phos/
  misc_feature 1..57: note = Synthetic
  source 1..57: mol_type = other DNA
aatatacatccaaaatcagtgttttcagtaattattacaatacatacactaaagata

SEQ ID NO: 453 - DNA, 57 bp, synthetic construct
  misc_feature 1..57: note = Synthetic
  misc_feature <1: note = /5Phos/
  source 1..57: mol_type = other DNA
aatatatgagcgaaggccccgttggggataattattacaatacatacactaaagata

SEQ ID NO: 454 - DNA, 57 bp, synthetic construct
  misc_feature 1..57: note = Synthetic
  source 1..57: mol_type = other DNA
  misc_feature <1: note = /5Phos/
aatatacgaagtgagcgaggagccactggaattattacaatacatacactaaagata

SEQ ID NO: 455 - DNA, 54 bp, synthetic construct
  source 1..54: mol_type = other DNA
  misc_feature 1..54: note = Synthetic
  misc_feature <1: note = /5Phos/
actatactatcatatggctgcagctcaattattacactacatacactaaagata

SEQ ID NO: 456 - DNA, 53 bp, synthetic construct
  misc_feature 1..53: note = Synthetic
  source 1..53: mol_type = other DNA
  misc_feature <1: note = /5Phos/
actataagagtggagaaagctggggaattattacactacatacactaaagata

SEQ ID NO: 457 - DNA, 53 bp, synthetic construct
  misc_feature <1: note = /5Phos/
  source 1..53: mol_type = other DNA
  misc_feature 1..53: note = Synthetic
actataaacgcattgaggggtttctaattattacactacatacactaaagata

SEQ ID NO: 458 - DNA, 54 bp, synthetic construct
  misc_feature 1..54: note = Synthetic
  source 1..54: mol_type = other DNA
  misc_feature <1: note = /5Phos/
actataaaccaggtattcttggtccaaattattacactacatacactaaagata

SEQ ID NO: 459 - DNA, 53 bp, synthetic construct
  misc_feature <1: note = /5Phos/
  misc_feature 1..53: note = Synthetic
  source 1..53: mol_type = other DNA
actatatttgtacatgccactggcaaattattacactacatacactaaagata

SEQ ID NO: 460 - DNA, 54 bp, synthetic construct
  source 1..54: mol_type = other DNA
  misc_feature 1..54: note = Synthetic
  misc_feature <1: note = /5Phos/
actatacaactatattgactagttgtaattattacactacatacactaaagata

SEQ ID NO: 461 - DNA, 55 bp, synthetic construct
  source 1..55: mol_type = other DNA
  misc_feature 1..55: note = Synthetic
  misc_feature <1: note = /5Phos/
agtataggttgccttgaccatcgatgtaattattacagtacatacactaaagata

SEQ ID NO: 462 - DNA, 55 bp, synthetic construct
  source 1..55: mol_type = other DNA
  misc_feature 1..55: note = Synthetic
  misc_feature <1: note = /5Phos/
agtatatggtcagcatgtggctggagtaattattacagtacatacactaaagata

SEQ ID NO: 463 - DNA, 53 bp, synthetic construct
  misc_feature 1..53: note = Synthetic
  misc_feature <1: note = /5Phos/
  source 1..53: mol_type = other DNA
agtatacgcgcaggatgtccctgctaattattacagtacatacactaaagata

SEQ ID NO: 464 - DNA, 55 bp, synthetic construct
  misc_feature <1: note = /5Phos/
  source 1..55: mol_type = other DNA
  misc_feature 1..55: note = Synthetic
agtatagagtagcaaggagcgtagaggaattattacagtacatacactaaagata

SEQ ID NO: 465 - DNA, 53 bp, synthetic construct
  misc_feature 1..53: note = Synthetic
  misc_feature <1: note = /5Phos/
  source 1..53: mol_type = other DNA
agtatacgggcttcatgatactgctaattattacagtacatacactaaagata

SEQ ID NO: 466 - DNA, 55 bp, synthetic construct
  misc_feature <1: note = /5Phos/
  source 1..55: mol_type = other DNA
  misc_feature 1..55: note = Synthetic
agtatatctatgtctcgctctctcagtaattattacagtacatacactaaagata

SEQ ID NO: 467 - DNA, 53 bp, synthetic construct
  misc_feature 1..53: note = Synthetic
  misc_feature <1: note = /5Phos/
  source 1..53: mol_type = other DNA
attatatgatgagttccctctgggaaattattacattacatacactaaagata

SEQ ID NO: 468 - DNA, 53 bp, synthetic construct
  misc_feature 1..53: note = Synthetic
  misc_feature <1: note = /5Phos/
  source 1..53: mol_type = other DNA
attatagtcagcaagtgttcaccgtaattattacattacatacactaaagata

SEQ ID NO: 469 - DNA, 55 bp, synthetic construct
  misc_feature 1..55: note = Synthetic
  misc_feature <1: note = /5Phos/
  source 1..55: mol_type = other DNA
attatattctcagatgggatgagggcgaattattacattacatacactaaagata

SEQ ID NO: 470 - DNA, 55 bp, synthetic construct
  misc_feature <1: note = /5Phos/
  misc_feature 1..55: note = Synthetic
  source 1..55: mol_type = other DNA
attatattgtgcaaatgtagctggaagaattattacattacatacactaaagata

SEQ ID NO: 471 - DNA, 54 bp, synthetic construct
  misc_feature <1: note = /5Phos/
  misc_feature 1..54: note = Synthetic
  source 1..54: mol_type = other DNA
attatatttccataggacttcgcagaaattattacattacatacactaaagata

SEQ ID NO: 472 - DNA, 58 bp, synthetic construct
  misc_feature <1: note = /5Phos/
  source 1..58: mol_type = other DNA
  misc_feature 1..58: note = Synthetic
attatatcattgctcagactactactgggtaattattacattacatacactaaagata

SEQ ID NO: 473 - DNA, 55 bp, synthetic construct
  misc_feature <1: note = /5Phos/
  misc_feature 1..55: note = Synthetic
  source 1..55: mol_type = other DNA
aaactattgggacatcatttctttgctaattattacaaaccatacactaaagata

SEQ ID NO: 474 - DNA, 58 bp, synthetic construct
  source 1..58: mol_type = other DNA
  misc_feature 1..58: note = Synthetic
  misc_feature <1: note = /5Phos/
aaactatgataggatgcagcgtctgataggaattattacaaaccatacactaaagata

SEQ ID NO: 475 - DNA, 59 bp, synthetic construct
  source 1..59: mol_type = other DNA
  misc_feature <1: note = /5Phos/
  misc_feature 1..59: note = Synthetic
aaactaatgaagtaatccgaggtgtaacaggaattattacaaaccatacactaaagata

SEQ ID NO: 476 - DNA, 55 bp, synthetic construct
  source 1..55: mol_type = other DNA
  misc_feature 1..55: note = Synthetic
  misc_feature <1: note = /5Phos/
aaactagtctgtctgcaaggtgtctgtaattattacaaaccatacactaaagata

SEQ ID NO: 477 - DNA, 53 bp, synthetic construct
  misc_feature <1: note = /5Phos/
  misc_feature 1..53: note = Synthetic
  source 1..53: mol_type = other DNA
aaactacagcgggacatctgcacataattattacaaaccatacactaaagata

SEQ ID NO: 478 - DNA, 53 bp, synthetic construct
  source 1..53: mol_type = other DNA
  misc_feature <1: note = /5Phos/
  misc_feature 1..53: note = Synthetic
aaactagctgctacatttccagtgtaattattacaaaccatacactaaagata

SEQ ID NO: 479 - DNA, 54 bp, synthetic construct
  source 1..54: mol_type = other DNA
  misc_feature <1: note = /5Phos/
  misc_feature 1..54: note = Synthetic
acactattctcagtcatggcggaaccaattattacacaccatacactaaagata

SEQ ID NO: 480 - DNA, 53 bp, synthetic construct
  misc_feature <1: note = /5Phos/
  source 1..53: mol_type = other DNA
  misc_feature 1..53: note = Synthetic
acactaaccagttgggcttcatgggaattattacacaccatacactaaagata

SEQ ID NO: 481 - DNA, 56 bp, synthetic construct
  misc_feature <1: note = /5Phos/
  source 1..56: mol_type = other DNA
  misc_feature 1..56: note = Synthetic
acactaaaccctgtagcagtttccacagaattattacacaccatacactaaagata

SEQ ID NO: 482 - DNA, 53 bp, synthetic construct
  source 1..53: mol_type = other DNA
  misc_feature <1: note = /5Phos/
  misc_feature 1..53: note = Synthetic
acactagtcgcggttaacaggatcaaattattacacaccatacactaaagata

SEQ ID NO: 483 - DNA, 54 bp, synthetic construct
  source 1..54: mol_type = other DNA
  misc_feature <1: note = /5Phos/
  misc_feature 1..54: note = Synthetic
acactagggccgaaaggcactgagctaattattacacaccatacactaaagata

SEQ ID NO: 484 - DNA, 53 bp, synthetic construct
  source 1..53: mol_type = other DNA
  misc_feature 1..53: note = Synthetic
  misc_feature <1: note = /5Phos/
acactactgcgcttcagccggctgtaattattacacaccatacactaaagata

SEQ ID NO: 485 - DNA, 53 bp, synthetic construct
  source 1..53: mol_type = other DNA
  misc_feature <1: note = /5Phos/
  misc_feature 1..53: note = Synthetic
agactaacgctttctctcgggtgggaattattacagaccatacactaaagata

SEQ ID NO: 486 - DNA, 56 bp, synthetic construct
  source 1..56: mol_type = other DNA
  misc_feature <1: note = /5Phos/
  misc_feature 1..56: note = Synthetic
agactatgtttgttaattagccagtgcaaattattacagaccatacactaaagata

SEQ ID NO: 487 - DNA, 58 bp, synthetic construct
  misc_feature 1..58: note = Synthetic
  source 1..58: mol_type = other DNA
  misc_feature <1: note = /5Phos/
agactagaatagctacagacttaggaaagtaattattacagaccatacactaaagata

SEQ ID NO: 488 - DNA, 55 bp, synthetic construct
  source 1..55: mol_type = other DNA
  misc_feature <1: note = /5Phos/
  misc_feature 1..55: note = Synthetic
agactatgtaaaagttgggtataccctaattattacagaccatacactaaagata

SEQ ID NO: 489 - DNA, 57 bp, synthetic construct
  misc_feature <1: note = /5Phos/
  misc_feature 1..57: note = Synthetic
  source 1..57: mol_type = other DNA
agactatggagaacctcacttgaccgcctaattattacagaccatacactaaagata

SEQ ID NO: 490 - DNA, 55 bp, synthetic construct
  misc_feature <1: note = /5Phos/
  source 1..55: mol_type = other DNA
  misc_feature 1..55: note = Synthetic
agactagggaacgggtacagagggcctaattattacagaccatacactaaagata

SEQ ID NO: 491 - DNA, 57 bp, synthetic construct
  source 1..57: mol_type = other DNA
  misc_feature <1: note = /5Phos/
  misc_feature 1..57: note = Synthetic
atactacgttgataaacaatcgtcatcctaattattacataccatacactaaagata

SEQ ID NO: 492 - DNA, 54 bp, synthetic construct
  misc_feature <1: note = /5Phos/
  source 1..54: mol_type = other DNA
  misc_feature 1..54: note = Synthetic
atactaatctgctatgtgttcctgttaattattacataccatacactaaagata

SEQ ID NO: 493 - DNA, 55 bp, synthetic construct
  misc_feature <1: note = /5Phos/
  misc_feature 1..55: note = Synthetic
  source 1..55: mol_type = other DNA
atactacgggtaattaggttggctggtaattattacataccatacactaaagata

SEQ ID NO: 494 - DNA, 53 bp, synthetic construct
  misc_feature <1: note = /5Phos/
  source 1..53: mol_type = other DNA
  misc_feature 1..53: note = Synthetic
atactacggagcacgttgacgtcggaattattacataccatacactaaagata

SEQ ID NO: 495 - DNA, 54 bp, synthetic construct
  source 1..54: mol_type = other DNA
  misc_feature 1..54: note = Synthetic
  misc_feature <1: note = /5Phos/
atactacactttctaagtagattcttaattattacataccatacactaaagata

SEQ ID NO: 496 - DNA, 53 bp, synthetic construct
  misc_feature 1..53: note = Synthetic
  source 1..53: mol_type = other DNA
  misc_feature <1: note = /5Phos/
atactacatcttgagctcgcggggtaattattacataccatacactaaagata

SEQ ID NO: 497 - DNA, 53 bp, synthetic construct
  misc_feature 1..53: note = Synthetic
  source 1..53: mol_type = other DNA
  misc_feature <1: note = /5Phos/
aacctagaagtcggggtgttcggccaattattacaacccatacactaaagata

SEQ ID NO: 498 - DNA, 53 bp, synthetic construct
  source 1..53: mol_type = other DNA
  misc_feature 1..53: note = Synthetic
  misc_feature <1: note = /5Phos/
aacctaaagcgtgggacagtcacccaattattacaacccatacactaaagata

SEQ ID NO: 499 - DNA, 53 bp, synthetic construct
  misc_feature <1: note = /5Phos/
  misc_feature 1..53: note = Synthetic
  source 1..53: mol_type = other DNA
aacctaccaagagcagtttcatggtaattattacaacccatacactaaagata

SEQ ID NO: 500 - DNA, 54 bp, synthetic construct
  source 1..54: mol_type = other DNA
  misc_feature <1: note = /5Phos/
  misc_feature 1..54: note = Synthetic
aacctacagattcgtttgcctcgctgaattattacaacccatacactaaagata

SEQ ID NO: 501 - DNA, 53 bp, synthetic construct
  source 1..53: mol_type = other DNA
  misc_feature 1..53: note = Synthetic
  misc_feature <1: note = /5Phos/
aacctaaatgccctcccttgtgtgtaattattacaacccatacactaaagata

SEQ ID NO: 502 - DNA, 54 bp, synthetic construct
  misc_feature 1..54: note = Synthetic
  source 1..54: mol_type = other DNA
  misc_feature <1: note = /5Phos/
aacctacgaatttcctcacttctcctaattattacaacccatacactaaagata

SEQ ID NO: 503 - DNA, 53 bp, synthetic construct
  misc_feature <1: note = /5Phos/
  misc_feature 1..53: note = Synthetic
  source 1..53: mol_type = other DNA
accctatgttatggctggacactggaattattacaccccatacactaaagata

SEQ ID NO: 504 - DNA, 58 bp, synthetic construct
  misc_feature <1: note = /5Phos/
  misc_feature 1..58: note = Synthetic
  source 1..58: mol_type = other DNA
accctatggaggtctatgtgttacaccaggaattattacaccccatacactaaagata

SEQ ID NO: 505 - DNA, 53 bp, synthetic construct
  misc_feature 1..53: note = Synthetic
  source 1..53: mol_type = other DNA
  misc_feature <1: note = /5Phos/
accctactcccattccttgtcctggaattattacaccccatacactaaagata

SEQ ID NO: 506 - DNA, 53 bp, synthetic construct
  misc_feature 1..53: note = Synthetic
  misc_feature <1: note = /5Phos/
  source 1..53: mol_type = other DNA
accctagttgctacggaaaccaccgaattattacaccccatacactaaagata

SEQ ID NO: 507 - DNA, 55 bp, synthetic construct
  source 1..55: mol_type = other DNA
  misc_feature 1..55: note = Synthetic
  misc_feature <1: note = /5Phos/
accctagccatgtttaggcaacagcctaattattacaccccatacactaaagata

SEQ ID NO: 508 - DNA, 53 bp, synthetic construct
  misc_feature <1: note = /5Phos/
  misc_feature 1..53: note = Synthetic
  source 1..53: mol_type = other DNA
accctattcgcgtaaaagccacgataattattacaccccatacactaaagata

SEQ ID NO: 509 - DNA, 56 bp, synthetic construct
  misc_feature 1..56: note = Synthetic
  misc_feature <1: note = /5Phos/
  source 1..56: mol_type = other DNA
agcctatgttcacacactatgtcggagtaattattacagcccatacactaaagata

SEQ ID NO: 510 - DNA, 53 bp, synthetic construct
  misc_feature <1: note = /5Phos/
  misc_feature 1..53: note = Synthetic
  source 1..53: mol_type = other DNA
agcctatcacctacacagaaatgctaattattacagcccatacactaaagata

SEQ ID NO: 511 - DNA, 53 bp, synthetic construct
  source 1..53: mol_type = other DNA
  misc_feature 1..53: note = Synthetic
  misc_feature <1: note = /5Phos/
agcctacatcgtcatctcataggcaaattattacagcccatacactaaagata

SEQ ID NO: 512 - DNA, 56 bp, synthetic construct
  source 1..56: mol_type = other DNA
  misc_feature <1: note = /5Phos/
  misc_feature 1..56: note = Synthetic
agcctaatcgatagttacctggggaagcaattattacagcccatacactaaagata

SEQ ID NO: 513 - DNA, 53 bp, synthetic construct
  source 1..53: mol_type = other DNA
  misc_feature 1..53: note = Synthetic
  misc_feature <1: note = /5Phos/
agcctaactgtgactagaacagggtaattattacagcccatacactaaagata

SEQ ID NO: 514 - DNA, 54 bp, synthetic construct
  source 1..54: mol_type = other DNA
  misc_feature <1: note = /5Phos/
  misc_feature 1..54: note = Synthetic
agcctacctatgttctgctggcagccaattattacagcccatacactaaagata

SEQ ID NO: 515 - DNA, 55 bp, synthetic construct
  misc_feature 1..55: note = Synthetic
  misc_feature <1: note = /5Phos/
  source 1..55: mol_type = other DNA
atcctatgaatgactctgacgagcagaaattattacatcccatacactaaagata

SEQ ID NO: 516 - DNA, 53 bp, synthetic construct
  source 1..53: mol_type = other DNA
  misc_feature <1: note = /5Phos/
  misc_feature 1..53: note = Synthetic
atcctagaccgaggaacccatgctgaattattacatcccatacactaaagata

SEQ ID NO: 517 - DNA, 54 bp, synthetic construct
  source 1..54: mol_type = other DNA
  misc_feature 1..54: note = Synthetic
  misc_feature <1: note = /5Phos/
atcctatatatgcatgccaccttgcaaattattacatcccatacactaaagata

SEQ ID NO: 518 - DNA, 56 bp, synthetic construct
  source 1..56: mol_type = other DNA
  misc_feature <1: note = /5Phos/
  misc_feature 1..56: note = Synthetic
atcctatcctgtgttaagaaccgaggggaattattacatcccatacactaaagata

SEQ ID NO: 519 - DNA, 58 bp, synthetic construct
  misc_feature 1..58: note = Synthetic
  source 1..58: mol_type = other DNA
  misc_feature <1: note = /5Phos/
atcctaaatgaatgatcgtccgttctgtctaattattacatcccatacactaaagata

SEQ ID NO: 520 - DNA, 56 bp, synthetic construct
  source 1..56: mol_type = other DNA
  misc_feature 1..56: note = Synthetic
  misc_feature <1: note = /5Phos/
atcctaggatgccgtgtcatcatggtctaattattacatcccatacactaaagata

SEQ ID NO: 521 - DNA, 55 bp, synthetic construct
  misc_feature 1..55: note = Synthetic
  source 1..55: mol_type = other DNA
  misc_feature <1: note = /5Phos/
aagctaagaactgttctggtcactgctaattattacaagccatacactaaagata

SEQ ID NO: 522 - DNA, 55 bp, synthetic construct
  source 1..55: mol_type = other DNA
  misc_feature 1..55: note = Synthetic
  misc_feature <1: note = /5Phos/
aagctaattattgctggagtgatccggaattattacaagccatacactaaagata

SEQ ID NO: 523 - DNA, 56 bp, synthetic construct
  misc_feature 1..56: note = Synthetic
  source 1..56: mol_type = other DNA
  misc_feature <1: note = /5Phos/
aagctagaatacccaggtcatggcctgtaattattacaagccatacactaaagata

SEQ ID NO: 524 - DNA, 54 bp, synthetic construct
  misc_feature 1..54: note = Synthetic
  misc_feature <1: note = /5Phos/
  source 1..54: mol_type = other DNA
aagctaaaccgagaggaaggaactttaattattacaagccatacactaaagata

SEQ ID NO: 525 - DNA, 53 bp, synthetic construct
  source 1..53: mol_type = other DNA
  misc_feature <1: note = /5Phos/
  misc_feature 1..53: note = Synthetic
aagctagaggaccatgaggacggccaattattacaagccatacactaaagata

SEQ ID NO: 526 - DNA, 55 bp, synthetic construct
  source 1..55: mol_type = other DNA
  misc_feature <1: note = /5Phos/
  misc_feature 1..55: note = Synthetic
aagctaggggccggtgaagctcgtggtaattattacaagccatacactaaagata

SEQ ID NO: 527 - DNA, 54 bp, synthetic construct
  source 1..54: mol_type = other DNA
  misc_feature <1: note = /5Phos/
  misc_feature 1..54: note = Synthetic
acgctattctgccatctcgttgtggtaattattacacgccatacactaaagata

SEQ ID NO: 528 - DNA, 54 bp, synthetic construct
  source 1..54: mol_type = other DNA
  misc_feature <1: note = /5Phos/
  misc_feature 1..54: note = Synthetic
acgctagcctcgatcactcttggagtaattattacacgccatacactaaagata

SEQ ID NO: 529 - DNA, 53 bp, synthetic construct
  source 1..53: mol_type = other DNA
  misc_feature 1..53: note = Synthetic
  misc_feature <1: note = /5Phos/
acgctagagctcttacgtttggcgaaattattacacgccatacactaaagata

SEQ ID NO: 530 - DNA, 53 bp, synthetic construct
  misc_feature 1..53: note = Synthetic
  misc_feature <1: note = /5Phos/
  source 1..53: mol_type = other DNA
acgctatgatccaggaagagcacgcaattattacacgccatacactaaagata

SEQ ID NO: 531 - DNA, 54 bp, synthetic construct
  source 1..54: mol_type = other DNA
  misc_feature <1: note = /5Phos/
  misc_feature 1..54: note = Synthetic
acgctactcatcgacatccattgtctaattattacacgccatacactaaagata

SEQ ID NO: 532 - DNA, 54 bp, synthetic construct
  misc_feature 1..54: note = Synthetic
  source 1..54: mol_type = other DNA
  misc_feature <1: note = /5Phos/
acgctatcagctagaatgttcctggcaattattacacgccatacactaaagata

SEQ ID NO: 533 - DNA, 57 bp, synthetic construct
  source 1..57: mol_type = other DNA
  misc_feature 1..57: note = Synthetic
  misc_feature <1: note = /5Phos/
aggctaccgagtagttttcatcgttgcctaattattacaggccatacactaaagata

SEQ ID NO: 534 - DNA, 58 bp, synthetic construct
  source 1..58: mol_type = other DNA
  misc_feature 1..58: note = Synthetic
  misc_feature <1: note = /5Phos/
aggctatgtaaagacggtgatggtcagagtaattattacaggccatacactaaagata

SEQ ID NO: 535 - DNA, 55 bp, synthetic construct
  source 1..55: mol_type = other DNA
  misc_feature 1..55: note = Synthetic
  misc_feature <1: note = /5Phos/
aggctacgttgaatctcgctacctggtaattattacaggccatacactaaagata

SEQ ID NO: 536 - DNA, 56 bp, synthetic construct
  misc_feature <1: note = /5Phos/
  misc_feature 1..56: note = Synthetic
  source 1..56: mol_type = other DNA
aggctaagactacatgtgcctgatggataattattacaggccatacactaaagata

SEQ ID NO: 537 - DNA, 53 bp, synthetic construct
  source 1..53: mol_type = other DNA
  misc_feature 1..53: note = Synthetic
  misc_feature <1: note = /5Phos/
aggctaatctgaaccgtagccatggaattattacaggccatacactaaagata

SEQ ID NO: 538 - DNA, 54 bp, synthetic construct
  misc_feature 1..54: note = Synthetic
  misc_feature <1: note = /5Phos/
  source 1..54: mol_type = other DNA
aggctaagacacctggtcttaactctaattattacaggccatacactaaagata

SEQ ID NO: 539 - DNA, 55 bp, synthetic construct
  source 1..55: mol_type = other DNA
  misc_feature 1..55: note = Synthetic
  misc_feature <1: note = /5Phos/
atgctagacatgtgggatgtggaggtcaattattacatgccatacactaaagata

SEQ ID NO: 540 - DNA, 53 bp, synthetic construct
  source 1..53: mol_type = other DNA
  misc_feature <1: note = /5Phos/
  misc_feature 1..53: note = Synthetic
atgctaaggtgaaggagatcccactaattattacatgccatacactaaagata

SEQ ID NO: 541 - DNA, 54 bp, synthetic construct
  source 1..54: mol_type = other DNA
  misc_feature <1: note = /5Phos/
  misc_feature 1..54: note = Synthetic
atgctacgaccatcgaagctgatcgaaattattacatgccatacactaaagata

SEQ ID NO: 542 - DNA, 55 bp, synthetic construct
  misc_feature <1: note = /5Phos/
  misc_feature 1..55: note = Synthetic
  source 1..55: mol_type = other DNA
atgctagaaaactactgcttgtgtggtaattattacatgccatacactaaagata

SEQ ID NO: 543 - DNA, 53 bp, synthetic construct
  misc_feature <1: note = /5Phos/
  misc_feature 1..53: note = Synthetic
  source 1..53: mol_type = other DNA
atgctaggaggagtatgtgaggcggaattattacatgccatacactaaagata

SEQ ID NO: 544 - DNA, 56 bp, synthetic construct
  source 1..56: mol_type = other DNA
  misc_feature <1: note = /5Phos/
  misc_feature 1..56: note = Synthetic
atgctaaaaggtcagaagaactgtgggtaattattacatgccatacactaaagata

SEQ ID NO: 545 - DNA, 56 bp, synthetic construct
  misc_feature 1..56: note = Synthetic
  misc_feature <1: note = /5Phos/
  source 1..56: mol_type = other DNA
aatctaccatgcatagggcttgaaaggcaattattacaatccatacactaaagata

SEQ ID NO: 546 - DNA, 54 bp, synthetic construct
  misc_feature 1..54: note = Synthetic
  source 1..54: mol_type = other DNA
  misc_feature <1: note = /5Phos/
aatctatgacctcttgaagctcttgcaattattacaatccatacactaaagata

SEQ ID NO: 547 - DNA, 53 bp, synthetic construct
  misc_feature <1: note = /5Phos/
  misc_feature 1..53: note = Synthetic
  source 1..53: mol_type = other DNA
aatctactgcgttcctgggagtgcaaattattacaatccatacactaaagata

SEQ ID NO: 548 - DNA, 57 bp, synthetic construct
  misc_feature <1: note = /5Phos/
  source 1..57: mol_type = other DNA
  misc_feature 1..57: note = Synthetic
aatctatccttactctgataacctgcggcaattattacaatccatacactaaagata

SEQ ID NO: 549 - DNA, 53 bp, synthetic construct
  source 1..53: mol_type = other DNA
  misc_feature 1..53: note = Synthetic
  misc_feature <1: note = /5Phos/
aatctaaggaacgaggttccttcccaattattacaatccatacactaaagata

SEQ ID NO: 550 - DNA, 57 bp, synthetic construct
  source 1..57: mol_type = other DNA
  misc_feature <1: note = /5Phos/
  misc_feature 1..57: note = Synthetic
aatctacctcggatactctgagttctcgtaattattacaatccatacactaaagata

SEQ ID NO: 551 - DNA, 56 bp, synthetic construct
  misc_feature <1: note = /5Phos/
  misc_feature 1..56: note = Synthetic
  source 1..56: mol_type = other DNA
actctactaggagttgtgacagctgcagaattattacactccatacactaaagata

SEQ ID NO: 552 - DNA, 58 bp, synthetic construct
  misc_feature <1: note = /5Phos/
  misc_feature 1..58: note = Synthetic
  source 1..58: mol_type = other DNA
actctagaccttagagttgctatccaaggtaattattacactccatacactaaagata

SEQ ID NO: 553 - DNA, 55 bp, synthetic construct
  misc_feature 1..55: note = Synthetic
  misc_feature <1: note = /5Phos/
  source 1..55: mol_type = other DNA
actctaatagctgtgctctgaaaacccaattattacactccatacactaaagata

SEQ ID NO: 554 - DNA, 56 bp, synthetic construct
  misc_feature 1..56: note = Synthetic
  source 1..56: mol_type = other DNA
  misc_feature <1: note = /5Phos/
actctagggtatcttgagggtgagtgggaattattacactccatacactaaagata

SEQ ID NO: 555 - DNA, 54 bp, synthetic construct
  misc_feature 1..54: note = Synthetic
  misc_feature <1: note = /5Phos/
  source 1..54: mol_type = other DNA
actctacttttgtacggacacctcttaattattacactccatacactaaagata

SEQ ID NO: 556 - DNA, 53 bp, synthetic construct
  misc_feature 1..53: note = Synthetic
  source 1..53: mol_type = other DNA
  misc_feature <1: note = /5Phos/
actctacactcagtagacactgccaaattattacactccatacactaaagata

SEQ ID NO: 557 - DNA, 55 bp, synthetic construct
  source 1..55: mol_type = other DNA
  misc_feature 1..55: note = Synthetic
  misc_feature <1: note = /5Phos/
agtctatgtggtctgtcctggagaaggaattattacagtccatacactaaagata

SEQ ID NO: 558 - DNA, 57 bp, synthetic construct
  source 1..57: mol_type = other DNA
  misc_feature <1: note = /5Phos/
  misc_feature 1..57: note = Synthetic
agtctacaaattcatctacggtccacactaattattacagtccatacactaaagata

SEQ ID NO: 559 - DNA, 54 bp, synthetic construct
  misc_feature 1..54: note = Synthetic
  source 1..54: mol_type = other DNA
  misc_feature <1: note = /5Phos/
agtctaggtggctacgatgcagcaagaattattacagtccatacactaaagata

SEQ ID NO: 560 - DNA, 54 bp, synthetic construct
  misc_feature <1: note = /5Phos/
  misc_feature 1..54: note = Synthetic
  source 1..54: mol_type = other DNA
agtctaaaaccaatggtagatttcataattattacagtccatacactaaagata

SEQ ID NO: 561 - DNA, 55 bp, synthetic construct
  source 1..55: mol_type = other DNA
  misc_feature 1..55: note = Synthetic
  misc_feature <1: note = /5Phos/
agtctagatcccctttgtcttatcaggaattattacagtccatacactaaagata

SEQ ID NO: 562 - DNA, 56 bp, synthetic construct
  misc_feature 1..56: note = Synthetic
  source 1..56: mol_type = other DNA
  misc_feature <1: note = /5Phos/
agtctattcgagatcttgaggagcaggtaattattacagtccatacactaaagata

SEQ ID NO: 563 - DNA, 54 bp, synthetic construct
  misc_feature 1..54: note = Synthetic
  source 1..54: mol_type = other DNA
  misc_feature <1: note = /5Phos/
attctaatgacgcttatgttgttgctaattattacattccatacactaaagata

SEQ ID NO: 564 - DNA, 56 bp, synthetic construct
  misc_feature <1: note = /5Phos/
  source 1..56: mol_type = other DNA
  misc_feature 1..56: note = Synthetic
attctagctacaatctgagttcagtcagaattattacattccatacactaaagata

SEQ ID NO: 565 - DNA, 55 bp, synthetic construct
  misc_feature <1: note = /5Phos/
  misc_feature 1..55: note = Synthetic
  source 1..55: mol_type = other DNA
attctaggtggaccactcggatgagctaattattacattccatacactaaagata

SEQ ID NO: 566 - DNA, 53 bp, synthetic construct
  source 1..53: mol_type = other DNA
  misc_feature 1..53: note = Synthetic
  misc_feature <1: note = /5Phos/
attctagataatctggctctgcaggaattattacattccatacactaaagata

SEQ ID NO: 567 - DNA, 53 bp, synthetic construct
  misc_feature 1..53: note = Synthetic
  misc_feature <1: note = /5Phos/
  source 1..53: mol_type = other DNA
attctattcaatgactgtgccgtggaattattacattccatacactaaagata

SEQ ID NO: 568 - DNA, 55 bp, synthetic construct
  misc_feature <1: note = /5Phos/
  misc_feature 1..55: note = Synthetic
  source 1..55: mol_type = other DNA
attctaagcgttcattgtctcagagctaattattacattccatacactaaagata

SEQ ID NO: 569 - DNA, 55 bp, synthetic construct
  source 1..55: mol_type = other DNA
  misc_feature <1: note = /5Phos/
  misc_feature 1..55: note = Synthetic
aaagtaggacgtttggcacatccatctaattattacaaagcatacactaaagata

SEQ ID NO: 570 - DNA, 54 bp, synthetic construct
  misc_feature <1: note = /5Phos/
  source 1..54: mol_type = other DNA
  misc_feature 1..54: note = Synthetic
aaagtacctacagacgagctcactctaattattacaaagcatacactaaagata

SEQ ID NO: 571 - DNA, 55 bp, synthetic construct
  misc_feature <1: note = /5Phos/
  source 1..55: mol_type = other DNA
  misc_feature 1..55: note = Synthetic
aaagtacatcgaaaagcccgaaagagtaattattacaaagcatacactaaagata

SEQ ID NO: 572 - DNA, 54 bp, synthetic construct
  misc_feature <1: note = /5Phos/
  source 1..54: mol_type = other DNA
  misc_feature 1..54: note = Synthetic
aaagtagccgtcagtgacgagagacaaattattacaaagcatacactaaagata

SEQ ID NO: 573 - DNA, 56 bp, synthetic construct
  misc_feature <1: note = /5Phos/
  misc_feature 1..56: note = Synthetic
  source 1..56: mol_type = other DNA
aaagtaaatccatttgcatgatgctcttaattattacaaagcatacactaaagata

SEQ ID NO: 574 - DNA, 57 bp, synthetic construct
  misc_feature <1: note = /5Phos/
  source 1..57: mol_type = other DNA
  misc_feature 1..57: note = Synthetic
aaagtagagttcttcttcaagcatggagtaattattacaaagcatacactaaagata

SEQ ID NO: 575 - DNA, 54 bp, synthetic construct
  misc_feature 1..54: note = Synthetic
  source 1..54: mol_type = other DNA
  misc_feature <1: note = /5Phos/
acagtaggtagtctgagggccttctgaattattacacagcatacactaaagata

SEQ ID NO: 576 - DNA, 54 bp, synthetic construct
  misc_feature <1: note = /5Phos/
  misc_feature 1..54: note = Synthetic
  source 1..54: mol_type = other DNA
acagtattgaggttgaccttcacattaattattacacagcatacactaaagata

SEQ ID NO: 577 - DNA, 53 bp, synthetic construct
  misc_feature 1..53: note = Synthetic
  source 1..53: mol_type = other DNA
  misc_feature <1: note = /5Phos/
acagtaagtgaaggggcagctctcaaattattacacagcatacactaaagata

SEQ ID NO: 578 - DNA, 56 bp, synthetic construct
  misc_feature 1..56: note = Synthetic
  misc_feature <1: note = /5Phos/
  source 1..56: mol_type = other DNA
acagtattacaccttcttttcattgtggaattattacacagcatacactaaagata

SEQ ID NO: 579 - DNA, 57 bp, synthetic construct
  misc_feature 1..57: note = Synthetic
  source 1..57: mol_type = other DNA
  misc_feature <1: note = /5Phos/
acagtatctatcagggtcttcattgcggtaattattacacagcatacactaaagata

SEQ ID NO: 580 - DNA, 53 bp, synthetic construct
  source 1..53: mol_type = other DNA
  misc_feature <1: note = /5Phos/
  misc_feature 1..53: note = Synthetic
acagtattcatgtggtggtccagctaattattacacagcatacactaaagata

SEQ ID NO: 581 - DNA, 56 bp, synthetic construct
  misc_feature 1..56: note = Synthetic
  source 1..56: mol_type = other DNA
  misc_feature <1: note = /5Phos/
agagtatacatggactcaaagttctcccaattattacagagcatacactaaagata

SEQ ID NO: 582 - DNA, 54 bp, synthetic construct
  source 1..54: mol_type = other DNA
  misc_feature 1..54: note = Synthetic
  misc_feature <1: note = /5Phos/
agagtaaatgaaacttcctggcgcataattattacagagcatacactaaagata

SEQ ID NO: 583 - DNA, 53 bp, synthetic construct
  misc_feature 1..53: note = Synthetic
  source 1..53: mol_type = other DNA
  misc_feature <1: note = /5Phos/
agagtagtgttagaagcactgcaggaattattacagagcatacactaaagata

SEQ ID NO: 584 - DNA, 53 bp, synthetic construct
  misc_feature 1..53: note = Synthetic
  source 1..53: mol_type = other DNA
  misc_feature <1: note = /5Phos/
agagtacgcctagtcctgagtcgctaattattacagagcatacactaaagata

SEQ ID NO: 585 - DNA, 55 bp, synthetic construct
  misc_feature 1..55: note = Synthetic
  misc_feature <1: note = /5Phos/
  source 1..55: mol_type = other DNA
agagtaataactgtgttcccgaggtgtaattattacagagcatacactaaagata

SEQ ID NO: 586 - DNA, 54 bp, synthetic construct
  misc_feature <1: note = /5Phos/
  source 1..54: mol_type = other DNA
  misc_feature 1..54: note = Synthetic
agagtaacccttcaaacccttcctctaattattacagagcatacactaaagata

SEQ ID NO: 587 - DNA, 54 bp, synthetic construct
  misc_feature <1: note = /5Phos/
  misc_feature 1..54: note = Synthetic
  source 1..54: mol_type = other DNA
atagtaccaaagatccaggacttggcaattattacatagcatacactaaagata

SEQ ID NO: 588 - DNA, 55 bp, synthetic construct
  source 1..55: mol_type = other DNA
  misc_feature 1..55: note = Synthetic
  misc_feature <1: note = /5Phos/
atagtacatcttggcagaagcacacctaattattacatagcatacactaaagata

SEQ ID NO: 589 - DNA, 56 bp, synthetic construct
  misc_feature 1..56: note = Synthetic
  source 1..56: mol_type = other DNA
  misc_feature <1: note = /5Phos/
atagtaggtagcagaaactcatagccagaattattacatagcatacactaaagata

SEQ ID NO: 590 - DNA, 54 bp, synthetic construct
  misc_feature <1: note = /5Phos/
  misc_feature 1..54: note = Synthetic
  source 1..54: mol_type = other DNA
atagtatgatcaaggcctccacttggaattattacatagcatacactaaagata

SEQ ID NO: 591 - DNA, 59 bp, synthetic construct
  misc_feature 1..59: note = Synthetic
  source 1..59: mol_type = other DNA
  misc_feature <1: note = /5Phos/
atagtagaaggcatacaagaaagggttgacgaattattacatagcatacactaaagata

SEQ ID NO: 592 - DNA, 54 bp, synthetic construct
  source 1..54: mol_type = other DNA
  misc_feature 1..54: note = Synthetic
  misc_feature <1: note = /5Phos/
atagtagtggctgacagagaacaaagaattattacatagcatacactaaagata

SEQ ID NO: 593 - DNA, 53 bp, synthetic construct
  misc_feature <1: note = /5Phos/
  source 1..53: mol_type = other DNA
  misc_feature 1..53: note = Synthetic
aacgtaacaggcgtcatcgttccataattattacaacgcatacactaaagata

SEQ ID NO: 594 - DNA, 58 bp, synthetic construct
  misc_feature <1: note = /5Phos/
  misc_feature 1..58: note = Synthetic
  source 1..58: mol_type = other DNA
aacgtaatgacggctacaggaatgaagaggaattattacaacgcatacactaaagata

SEQ ID NO: 595 - DNA, 53 bp, synthetic construct
  source 1..53: mol_type = other DNA
  misc_feature <1: note = /5Phos/
  misc_feature 1..53: note = Synthetic
aacgtattttgttggttcccacccaaattattacaacgcatacactaaagata

SEQ ID NO: 596 - DNA, 54 bp, synthetic construct
  misc_feature 1..54: note = Synthetic
  misc_feature <1: note = /5Phos/
  source 1..54: mol_type = other DNA
aacgtacttgatatagatctccacacaattattacaacgcatacactaaagata

SEQ ID NO: 597 - DNA, 53 bp, synthetic construct
  source 1..53: mol_type = other DNA
  misc_feature <1: note = /5Phos/
  misc_feature 1..53: note = Synthetic
aacgtattgggcaagttaaggagcaaattattacaacgcatacactaaagata

SEQ ID NO: 598 - DNA, 54 bp, synthetic construct
  misc_feature <1: note = /5Phos/
  source 1..54: mol_type = other DNA
  misc_feature 1..54: note = Synthetic
aacgtagttggtcatgaagaaatcctaattattacaacgcatacactaaagata

SEQ ID NO: 599 - DNA, 55 bp, synthetic construct
  misc_feature <1: note = /5Phos/
  source 1..55: mol_type = other DNA
  misc_feature 1..55: note = Synthetic
accgtagcactctgtaaggacactgggaattattacaccgcatacactaaagata

SEQ ID NO: 600 - DNA, 56 bp, synthetic construct
  misc_feature 1..56: note = Synthetic
  source 1..56: mol_type = other DNA
  misc_feature <1: note = /5Phos/
accgtatggaatacttagggcagatcccaattattacaccgcatacactaaagata

SEQ ID NO: 601 - DNA, 54 bp, synthetic construct
  misc_feature <1: note = /5Phos/
  misc_feature 1..54: note = Synthetic
  source 1..54: mol_type = other DNA
accgtactcagacttctgctttggctaattattacaccgcatacactaaagata

SEQ ID NO: 602 - DNA, 54 bp, synthetic construct
  misc_feature 1..54: note = Synthetic
  source 1..54: mol_type = other DNA
  misc_feature <1: note = /5Phos/
accgtatgagcagacagtgaatgaggaattattacaccgcatacactaaagata

SEQ ID NO: 603 - DNA, 53 bp, synthetic construct
  misc_feature <1: note = /5Phos/
  misc_feature 1..53: note = Synthetic
  source 1..53: mol_type = other DNA
accgtacttccgagagtgttgtggcaattattacaccgcatacactaaagata

SEQ ID NO: 604 - DNA, 53 bp, synthetic construct
  source 1..53: mol_type = other DNA
  misc_feature 1..53: note = Synthetic
  misc_feature <1: note = /5Phos/
accgtatcttggaagtggatggcataattattacaccgcatacactaaagata

SEQ ID NO: 605 - DNA, 54 bp, synthetic construct
  source 1..54: mol_type = other DNA
  misc_feature <1: note = /5Phos/
  misc_feature 1..54: note = Synthetic
agcgtatcctagcaagatcagacacaaattattacagcgcatacactaaagata

SEQ ID NO: 606 - DNA, 55 bp, synthetic construct
  misc_feature 1..55: note = Synthetic
  source 1..55: mol_type = other DNA
  misc_feature <1: note = /5Phos/
agcgtaattcggatttgaatttgggctaattattacagcgcatacactaaagata

SEQ ID NO: 607 - DNA, 53 bp, synthetic construct
  misc_feature <1: note = /5Phos/
  source 1..53: mol_type = other DNA
  misc_feature 1..53: note = Synthetic
agcgtattctgcgccatgaatgtccaattattacagcgcatacactaaagata

SEQ ID NO: 608 - DNA, 53 bp, synthetic construct
  misc_feature 1..53: note = Synthetic
  misc_feature <1: note = /5Phos/
  source 1..53: mol_type = other DNA
agcgtaatatgccccaagcctttctaattattacagcgcatacactaaagata

SEQ ID NO: 609 - DNA, 54 bp, synthetic construct
  source 1..54: mol_type = other DNA
  misc_feature <1: note = /5Phos/
  misc_feature 1..54: note = Synthetic
agcgtatccttcctcaatccaatcccaattattacagcgcatacactaaagata

SEQ ID NO: 610 - DNA, 53 bp, synthetic construct
  misc_feature <1: note = /5Phos/
  misc_feature 1..53: note = Synthetic
  source 1..53: mol_type = other DNA
agcgtattgtgggtccggagctggtaattattacagcgcatacactaaagata

SEQ ID NO: 611 - DNA, 58 bp, synthetic construct
  misc_feature 1..58: note = Synthetic
  misc_feature <1: note = /5Phos/
  source 1..58: mol_type = other DNA
atcgtacattgtagggcaactgagtagggtaattattacatcgcatacactaaagata

SEQ ID NO: 612 - DNA, 56 bp, synthetic construct
  misc_feature 1..56: note = Synthetic
  source 1..56: mol_type = other DNA
  misc_feature <1: note = /5Phos/
atcgtatgctgtatgctagggtaggcataattattacatcgcatacactaaagata

SEQ ID NO: 613 - DNA, 54 bp, synthetic construct
  misc_feature <1: note = /5Phos/
  misc_feature 1..54: note = Synthetic
  source 1..54: mol_type = other DNA
atcgtagtacaacggtaggtcccagtaattattacatcgcatacactaaagata

SEQ ID NO: 614 - DNA, 55 bp, synthetic construct
  source 1..55: mol_type = other DNA
  misc_feature 1..55: note = Synthetic
  misc_feature <1: note = /5Phos/
atcgtagtcaaggactttagccttgcgaattattacatcgcatacactaaagata

SEQ ID NO: 615 - DNA, 53 bp, synthetic construct
  misc_feature 1..53: note = Synthetic
  misc_feature <1: note = /5Phos/
  source 1..53: mol_type = other DNA
atcgtattttgcgtaagacctgcctaattattacatcgcatacactaaagata

SEQ ID NO: 616 - DNA, 56 bp, synthetic construct
  source 1..56: mol_type = other DNA
  misc_feature <1: note = /5Phos/
  misc_feature 1..56: note = Synthetic
atcgtataaggacacttaggagaggagtaattattacatcgcatacactaaagata

SEQ ID NO: 617 - DNA, 54 bp, synthetic construct
  source 1..54: mol_type = other DNA
  misc_feature <1: note = /5Phos/
  misc_feature 1..54: note = Synthetic
aaggtaacaacgttcatagaccttggaattattacaaggcatacactaaagata

SEQ ID NO: 618 - DNA, 53 bp, synthetic construct
  misc_feature 1..53: note = Synthetic
  misc_feature <1: note = /5Phos/
  source 1..53: mol_type = other DNA
aaggtaggaaatcgagggaatgtgaaattattacaaggcatacactaaagata

SEQ ID NO: 619 - DNA, 58 bp, synthetic construct
  misc_feature 1..58: note = Synthetic
  source 1..58: mol_type = other DNA
  misc_feature <1: note = /5Phos/
acggtacgcatattttactggaagctccaaaattattacacggcatacactaaagata

SEQ ID NO: 620 - DNA, 55 bp, synthetic construct
  misc_feature 1..55: note = Synthetic
  misc_feature <1: note = /5Phos/
  source 1..55: mol_type = other DNA
acggtacttagctgccttacagaacccaattattacacggcatacactaaagata

SEQ ID NO: 621 - DNA, 58 bp, synthetic construct
  misc_feature <1: note = /5Phos/
  misc_feature 1..58: note = Synthetic
  source 1..58: mol_type = other DNA
acggtacgtccatgtgaaaatgtgtcagctaattattacacggcatacactaaagata

SEQ ID NO: 622 - DNA, 55 bp, synthetic construct
  misc_feature <1: note = /5Phos/
  misc_feature 1..55: note = Synthetic
  source 1..55: mol_type = other DNA
acggtaccacctatttggttagtggctaattattacacggcatacactaaagata

SEQ ID NO: 623 - DNA, 53 bp, synthetic construct
  source 1..53: mol_type = other DNA
  misc_feature 1..53: note = Synthetic
  misc_feature <1: note = /5Phos/
acggtaggcatcgatgcaatggcctaattattacacggcatacactaaagata

SEQ ID NO: 624 - DNA, 55 bp, synthetic construct
  misc_feature <1: note = /5Phos/
  misc_feature 1..55: note = Synthetic
  source 1..55: mol_type = other DNA
acggtaataacgaatgatgcttgcagcaattattacacggcatacactaaagata

SEQ ID NO: 625 - DNA, 56 bp, synthetic construct
  misc_feature <1: note = /5Phos/
  source 1..56: mol_type = other DNA
  misc_feature 1..56: note = Synthetic
agggtatgatcccatacggtcacattgtaattattacagggcatacactaaagata

SEQ ID NO: 626 - DNA, 53 bp, synthetic construct
  source 1..53: mol_type = other DNA
  misc_feature 1..53: note = Synthetic
  misc_feature <1: note = /5Phos/
agggtaacaggatctcagtgctgctaattattacagggcatacactaaagata

SEQ ID NO: 627 - DNA, 53 bp, synthetic construct
  source 1..53: mol_type = other DNA
  misc_feature <1: note = /5Phos/
  misc_feature 1..53: note = Synthetic
agggtaggatatctccttcgcgcagaattattacagggcatacactaaagata

SEQ ID NO: 628 - DNA, 54 bp, synthetic construct
  misc_feature 1..54: note = Synthetic
  misc_feature <1: note = /5Phos/
  source 1..54: mol_type = other DNA
agggtactgagatcgtcttggcagataattattacagggcatacactaaagata

SEQ ID NO: 629 - DNA, 55 bp, synthetic construct
  misc_feature 1..55: note = Synthetic
  source 1..55: mol_type = other DNA
  misc_feature <1: note = /5Phos/
agggtacctcaagatgactgcagaagcaattattacagggcatacactaaagata

SEQ ID NO: 630 - DNA, 54 bp, synthetic construct
  source 1..54: mol_type = other DNA
  misc_feature <1: note = /5Phos/
  misc_feature 1..54: note = Synthetic
agggtagtcaaatgataggttgccctaattattacagggcatacactaaagata

SEQ ID NO: 631 - DNA, 53 bp, synthetic construct
  misc_feature 1..53: note = Synthetic
  source 1..53: mol_type = other DNA
  misc_feature <1: note = /5Phos/
atggtatcggattcgtgggtacgctaattattacatggcatacactaaagata

SEQ ID NO: 632 - DNA, 58 bp, synthetic construct
  misc_feature <1: note = /5Phos/
  source 1..58: mol_type = other DNA
  misc_feature 1..58: note = Synthetic
atggtatgggagatttgtccgaacacttctaattattacatggcatacactaaagata

SEQ ID NO: 633 - DNA, 54 bp, synthetic construct
  source 1..54: mol_type = other DNA
  misc_feature <1: note = /5Phos/
  misc_feature 1..54: note = Synthetic
atggtagttccttcccacggaagacaaattattacatggcatacactaaagata

SEQ ID NO: 634 - DNA, 55 bp, synthetic construct
  misc_feature <1: note = /5Phos/
  misc_feature 1..55: note = Synthetic
  source 1..55: mol_type = other DNA
atggtacagcatttgaccagaagccctaattattacatggcatacactaaagata

SEQ ID NO: 635 - DNA, 53 bp, synthetic construct
  source 1..53: mol_type = other DNA
  misc_feature <1: note = /5Phos/
  misc_feature 1..53: note = Synthetic
atggtatctctctcgggaactcccaaattattacatggcatacactaaagata

SEQ ID NO: 636 - DNA, 53 bp, synthetic construct
  misc_feature 1..53: note = Synthetic
  misc_feature <1: note = /5Phos/
  source 1..53: mol_type = other DNA
atggtagggcgtcatcattttctgcaattattacatggcatacactaaagata

SEQ ID NO: 637 - DNA, 53 bp, synthetic construct
  source 1..53: mol_type = other DNA
  misc_feature <1: note = /5Phos/
  misc_feature 1..53: note = Synthetic
aatgtatacagaaacaggacgcggaaattattacaatgcatacactaaagata

SEQ ID NO: 638 - DNA, 53 bp, synthetic construct
  misc_feature <1: note = /5Phos/
  misc_feature 1..53: note = Synthetic
  source 1..53: mol_type = other DNA
aatgtagaaagatggctcaggcttgaattattacaatgcatacactaaagata

SEQ ID NO: 639 - DNA, 57 bp, synthetic construct
  misc_feature 1..57: note = Synthetic
  source 1..57: mol_type = other DNA
  misc_feature <1: note = /5Phos/
aatgtacgcacctacagaaataggcaggtaattattacaatgcatacactaaagata

SEQ ID NO: 640 - DNA, 56 bp, synthetic construct
  misc_feature <1: note = /5Phos/
  misc_feature 1..56: note = Synthetic
  source 1..56: mol_type = other DNA
aatgtacaacattaggtcagcattgggtaattattacaatgcatacactaaagata

SEQ ID NO: 641 - DNA, 53 bp, synthetic construct
  source 1..53: mol_type = other DNA
  misc_feature <1: note = /5Phos/
  misc_feature 1..53: note = Synthetic
aatgtagctggaatcaggaatccccaattattacaatgcatacactaaagata

SEQ ID NO: 642 - DNA, 53 bp, synthetic construct
  misc_feature <1: note = /5Phos/
  misc_feature 1..53: note = Synthetic
  source 1..53: mol_type = other DNA
aatgtattccataggcttgggagccaattattacaatgcatacactaaagata

SEQ ID NO: 643 - DNA, 54 bp, synthetic construct
  source 1..54: mol_type = other DNA
  misc_feature <1: note = /5Phos/
  misc_feature 1..54: note = Synthetic
actgtacttcatctaaagctgtggctaattattacactgcatacactaaagata

SEQ ID NO: 644 - DNA, 53 bp, synthetic construct
  misc_feature <1: note = /5Phos/
  misc_feature 1..53: note = Synthetic
  source 1..53: mol_type = other DNA
actgtagtggatttccataggacccaattattacactgcatacactaaagata

SEQ ID NO: 645 - DNA, 53 bp, synthetic construct
  misc_feature 1..53: note = Synthetic
  source 1..53: mol_type = other DNA
  misc_feature <1: note = /5Phos/
actgtatcccgtactggttctgggtaattattacactgcatacactaaagata

SEQ ID NO: 646 - DNA, 58 bp, synthetic construct
  misc_feature 1..58: note = Synthetic
  source 1..58: mol_type = other DNA
  misc_feature <1: note = /5Phos/
actgtacgttctcatagaattcagagccctaattattacactgcatacactaaagata

SEQ ID NO: 647 - DNA, 56 bp, synthetic construct
  misc_feature 1..56: note = Synthetic
  misc_feature <1: note = /5Phos/
  source 1..56: mol_type = other DNA
actgtaggatgttaggtggctaaatgccaattattacactgcatacactaaagata

SEQ ID NO: 648 - DNA, 53 bp, synthetic construct
  misc_feature 1..53: note = Synthetic
  misc_feature <1: note = /5Phos/
  source 1..53: mol_type = other DNA
actgtaaccagttctcaacagccagaattattacactgcatacactaaagata

SEQ ID NO: 649 - DNA, 57 bp, synthetic construct
  misc_feature 1..57: note = Synthetic
  source 1..57: mol_type = other DNA
  misc_feature <1: note = /5Phos/
agtgtaagatgatcccttctgctgtgatgaattattacagtgcatacactaaagata

SEQ ID NO: 650 - DNA, 55 bp, synthetic construct
  misc_feature <1: note = /5Phos/
  misc_feature 1..55: note = Synthetic
  source 1..55: mol_type = other DNA
agtgtatgacaagaagaggaggagaggaattattacagtgcatacactaaagata

SEQ ID NO: 651 - DNA, 53 bp, synthetic construct
  misc_feature <1: note = /5Phos/
  misc_feature 1..53: note = Synthetic
  source 1..53: mol_type = other DNA
agtgtacactagagagggagctggaaattattacagtgcatacactaaagata

SEQ ID NO: 652 - DNA, 53 bp, synthetic construct
  source 1..53: mol_type = other DNA
  misc_feature <1: note = /5Phos/
  misc_feature 1..53: note = Synthetic
agtgtaggtgggagaatagggtgggaattattacagtgcatacactaaagata

SEQ ID NO: 653 - DNA, 54 bp, synthetic construct
  misc_feature 1..54: note = Synthetic
  misc_feature <1: note = /5Phos/
  source 1..54: mol_type = other DNA
agtgtatcctcgcccaagttcaccgtaattattacagtgcatacactaaagata

SEQ ID NO: 654 - DNA, 56 bp, synthetic construct
  misc_feature <1: note = /5Phos/
  misc_feature 1..56: note = Synthetic
  source 1..56: mol_type = other DNA
agtgtaacgcggaggtaagtaccacaggaattattacagtgcatacactaaagata

SEQ ID NO: 655 - DNA, 55 bp, synthetic construct
  misc_feature 1..55: note = Synthetic
  misc_feature <1: note = /5Phos/
  source 1..55: mol_type = other DNA
attgtatgaagaagctttgtgtggggcaattattacattgcatacactaaagata

SEQ ID NO: 656 - DNA, 54 bp, synthetic construct
  misc_feature <1: note = /5Phos/
  source 1..54: mol_type = other DNA
  misc_feature 1..54: note = Synthetic
attgtacaaggattcctgctcttgggaattattacattgcatacactaaagata

SEQ ID NO: 657 - DNA, 54 bp, synthetic construct
  source 1..54: mol_type = other DNA
  misc_feature 1..54: note = Synthetic
  misc_feature <1: note = /5Phos/
attgtacgacagaatgcccaagaacaaattattacattgcatacactaaagata

SEQ ID NO: 658 - DNA, 59 bp, synthetic construct
  misc_feature 1..59: note = Synthetic
  source 1..59: mol_type = other DNA
  misc_feature <1: note = /5Phos/
attgtattttgtaggctctgctgggaaaggtaattattacattgcatacactaaagata

SEQ ID NO: 659 - DNA, 53 bp, synthetic construct
  misc_feature <1: note = /5Phos/
  source 1..53: mol_type = other DNA
  misc_feature 1..53: note = Synthetic
attgtagatgggtgcgaagacccctaattattacattgcatacactaaagata

SEQ ID NO: 660 - DNA, 57 bp, synthetic construct
  misc_feature <1: note = /5Phos/
  source 1..57: mol_type = other DNA
  misc_feature 1..57: note = Synthetic
attgtaatgagaattggattcttcagcgtaattattacattgcatacactaaagata

SEQ ID NO: 661 - DNA, 55 bp, synthetic construct
  misc_feature 1..55: note = Synthetic
  misc_feature <1: note = /5Phos/
  source 1..55: mol_type = other DNA
aaattagttgtacctacagaccagggtaattattacaaatcatacactaaagata

SEQ ID NO: 662 - DNA, 57 bp, synthetic construct
  misc_feature 1..57: note = Synthetic
  misc_feature <1: note = /5Phos/
  source 1..57: mol_type = other DNA
aaattagagaaattcagtccacaggtcagaattattacaaatcatacactaaagata

SEQ ID NO: 663 - DNA, 55 bp, synthetic construct
  misc_feature 1..55: note = Synthetic
  misc_feature <1: note = /5Phos/
  source 1..55: mol_type = other DNA
aaattacacactctttccgtggtccgtaattattacaaatcatacactaaagata

SEQ ID NO: 664 - DNA, 53 bp, synthetic construct
  source 1..53: mol_type = other DNA
  misc_feature 1..53: note = Synthetic
  misc_feature <1: note = /5Phos/
aaattagtgtcatccattgccggataattattacaaatcatacactaaagata

SEQ ID NO: 665 - DNA, 58 bp, synthetic construct
  misc_feature 1..58: note = Synthetic
  misc_feature <1: note = /5Phos/
  source 1..58: mol_type = other DNA
aaattacttaccttcagtaccttcacgtctaattattacaaatcatacactaaagata

SEQ ID NO: 666 - DNA, 54 bp, synthetic construct
  source 1..54: mol_type = other DNA
  misc_feature 1..54: note = Synthetic
  misc_feature <1: note = /5Phos/
aaattagagacattgaggtgaatgggaattattacaaatcatacactaaagata

SEQ ID NO: 667 - DNA, 55 bp, synthetic construct
  misc_feature 1..55: note = Synthetic
  source 1..55: mol_type = other DNA
  misc_feature <1: note = /5Phos/
acattatcacgacggtagcgacgagagaattattacacatcatacactaaagata

SEQ ID NO: 668 - DNA, 54 bp, synthetic construct
  source 1..54: mol_type = other DNA
  misc_feature <1: note = /5Phos/
  misc_feature 1..54: note = Synthetic
acattagttgacgctctccacacacaaattattacacatcatacactaaagata

SEQ ID NO: 669 - DNA, 53 bp, synthetic construct
  misc_feature 1..53: note = Synthetic
  source 1..53: mol_type = other DNA
  misc_feature <1: note = /5Phos/
acattacacagggcgatgttgtccaaattattacacatcatacactaaagata

SEQ ID NO: 670 - DNA, 55 bp, synthetic construct
  source 1..55: mol_type = other DNA
  misc_feature 1..55: note = Synthetic
  misc_feature <1: note = /5Phos/
acattacaccgaacacttgattctggtaattattacacatcatacactaaagata

SEQ ID NO: 671 - DNA, 54 bp, synthetic construct
  misc_feature <1: note = /5Phos/
  misc_feature 1..54: note = Synthetic
  source 1..54: mol_type = other DNA
acattaatgtcaatccgtaggaatccaattattacacatcatacactaaagata

SEQ ID NO: 672 - DNA, 53 bp, synthetic construct
  misc_feature 1..53: note = Synthetic
  misc_feature <1: note = /5Phos/
  source 1..53: mol_type = other DNA
acattaaatcaggtgtgttttccttaattattacacatcatacactaaagata

SEQ ID NO: 673 - DNA, 54 bp, synthetic construct
  misc_feature <1: note = /5Phos/
  source 1..54: mol_type = other DNA
  misc_feature 1..54: note = Synthetic
agattagtcttgatacacaggaggccaattattacagatcatacactaaagata

SEQ ID NO: 674 - DNA, 53 bp, synthetic construct
  misc_feature <1: note = /5Phos/
  source 1..53: mol_type = other DNA
  misc_feature 1..53: note = Synthetic
agattaagttgctttcaccccttgcaattattacagatcatacactaaagata

SEQ ID NO: 675 - DNA, 54 bp, synthetic construct
  misc_feature 1..54: note = Synthetic
  misc_feature <1: note = /5Phos/
  source 1..54: mol_type = other DNA
agattactagaggaaaggagtggaggaattattacagatcatacactaaagata

SEQ ID NO: 676 - DNA, 55 bp, synthetic construct
  source 1..55: mol_type = other DNA
  misc_feature 1..55: note = Synthetic
  misc_feature <1: note = /5Phos/
agattaattgaaactcacattcagactaattattacagatcatacactaaagata

SEQ ID NO: 677 - DNA, 53 bp, synthetic construct
  misc_feature <1: note = /5Phos/
  misc_feature 1..53: note = Synthetic
  source 1..53: mol_type = other DNA
agattacgactttcaggtcagagggaattattacagatcatacactaaagata

SEQ ID NO: 678 - DNA, 57 bp, synthetic construct
  misc_feature 1..57: note = Synthetic
  misc_feature <1: note = /5Phos/
  source 1..57: mol_type = other DNA
agattagattcaaaattgtgacttgcagtaattattacagatcatacactaaagata

SEQ ID NO: 679 - DNA, 53 bp, synthetic construct
  misc_feature <1: note = /5Phos/
  source 1..53: mol_type = other DNA
  misc_feature 1..53: note = Synthetic
atattagaagacagctagggcccagaattattacatatcatacactaaagata

SEQ ID NO: 680 - DNA, 53 bp, synthetic construct
  misc_feature <1: note = /5Phos/
  source 1..53: mol_type = other DNA
  misc_feature 1..53: note = Synthetic
atattaccagttggacaagctgcagaattattacatatcatacactaaagata

SEQ ID NO: 681 - DNA, 54 bp, synthetic construct
  misc_feature <1: note = /5Phos/
  misc_feature 1..54: note = Synthetic
  source 1..54: mol_type = other DNA
atattagctaccctgactgtctgaagaattattacatatcatacactaaagata

SEQ ID NO: 682 - DNA, 53 bp, synthetic construct
  source 1..53: mol_type = other DNA
  misc_feature <1: note = /5Phos/
  misc_feature 1..53: note = Synthetic
atattagtgaaggagagccagaaccaattattacatatcatacactaaagata

SEQ ID NO: 683 - DNA, 54 bp, synthetic construct
  source 1..54: mol_type = other DNA
  misc_feature 1..54: note = Synthetic
  misc_feature <1: note = /5Phos/
atattaaaccattacagaaggcggccaattattacatatcatacactaaagata

SEQ ID NO: 684 - DNA, 55 bp, synthetic construct
  source 1..55: mol_type = other DNA
  misc_feature 1..55: note = Synthetic
  misc_feature <1: note = /5Phos/
atattactcagatctctccatttccagaattattacatatcatacactaaagata

SEQ ID NO: 685 - DNA, 55 bp, synthetic construct
  misc_feature <1: note = /5Phos/
  source 1..55: mol_type = other DNA
  misc_feature 1..55: note = Synthetic
aacttaaacacaggcagtagacaggagaattattacaactcatacactaaagata

SEQ ID NO: 686 - DNA, 54 bp, synthetic construct
  source 1..54: mol_type = other DNA
  misc_feature <1: note = /5Phos/
  misc_feature 1..54: note = Synthetic
aacttactctgtctggaccttgtgcgaattattacaactcatacactaaagata

SEQ ID NO: 687 - DNA, 55 bp, synthetic construct
  source 1..55: mol_type = other DNA
  misc_feature 1..55: note = Synthetic
  misc_feature <1: note = /5Phos/
aacttagagtacaggacaatggtagagaattattacaactcatacactaaagata

SEQ ID NO: 688 - DNA, 54 bp, synthetic construct
  misc_feature 1..54: note = Synthetic
  source 1..54: mol_type = other DNA
  misc_feature <1: note = /5Phos/
aacttaatcggtggatggtcatgatgaattattacaactcatacactaaagata

SEQ ID NO: 689 - DNA, 55 bp, synthetic construct
  source 1..55: mol_type = other DNA
  misc_feature 1..55: note = Synthetic
  misc_feature <1: note = /5Phos/
aacttacatatgtaaaggtaccaggggaattattacaactcatacactaaagata

SEQ ID NO: 690 - DNA, 53 bp, synthetic construct
  misc_feature <1: note = /5Phos/
  misc_feature 1..53: note = Synthetic
  source 1..53: mol_type = other DNA
aacttatgtacgctttgtgcttcctaattattacaactcatacactaaagata

SEQ ID NO: 691 - DNA, 56 bp, synthetic construct
  source 1..56: mol_type = other DNA
  misc_feature 1..56: note = Synthetic
  misc_feature <1: note = /5Phos/
accttatatatggctcagaaatgtccagaattattacacctcatacactaaagata

SEQ ID NO: 692 - DNA, 53 bp, synthetic construct
  source 1..53: mol_type = other DNA
  misc_feature <1: note = /5Phos/
  misc_feature 1..53: note = Synthetic
accttaggcatatccggtcaccagtaattattacacctcatacactaaagata

SEQ ID NO: 693 - DNA, 55 bp, synthetic construct
  source 1..55: mol_type = other DNA
  misc_feature 1..55: note = Synthetic
  misc_feature <1: note = /5Phos/
accttattgtgaacaggttgtcctggcaattattacacctcatacactaaagata

SEQ ID NO: 694 - DNA, 56 bp, synthetic construct
  misc_feature 1..56: note = Synthetic
  misc_feature <1: note = /5Phos/
  source 1..56: mol_type = other DNA
accttattgtaaggtatggcctgtagctaattattacacctcatacactaaagata

SEQ ID NO: 695 - DNA, 53 bp, synthetic construct
  misc_feature <1: note = /5Phos/
  misc_feature 1..53: note = Synthetic
  source 1..53: mol_type = other DNA
accttacacaaccaaaattcctgcgaattattacacctcatacactaaagata

SEQ ID NO: 696 - DNA, 53 bp, synthetic construct
  source 1..53: mol_type = other DNA
  misc_feature <1: note = /5Phos/
  misc_feature 1..53: note = Synthetic
accttacaggtaaggctttcctgcaaattattacacctcatacactaaagata

SEQ ID NO: 697 - DNA, 54 bp, synthetic construct
  misc_feature 1..54: note = Synthetic
  source 1..54: mol_type = other DNA
  misc_feature <1: note = /5Phos/
agcttagtagggaaacaggacctggtaattattacagctcatacactaaagata

SEQ ID NO: 698 - DNA, 57 bp, synthetic construct
  misc_feature <1: note = /5Phos/
  source 1..57: mol_type = other DNA
  misc_feature 1..57: note = Synthetic
agcttaacgtgttcaggtaactcgtagccaattattacagctcatacactaaagata

SEQ ID NO: 699 - DNA, 55 bp, synthetic construct
  misc_feature 1..55: note = Synthetic
  source 1..55: mol_type = other DNA
  misc_feature <1: note = /5Phos/
agcttactgtgtgtcaaagagcttgcaaattattacagctcatacactaaagata

SEQ ID NO: 700 - DNA, 54 bp, synthetic construct
  misc_feature 1..54: note = Synthetic
  source 1..54: mol_type = other DNA
  misc_feature <1: note = /5Phos/
agcttactgtgatgagcttcctctgtaattattacagctcatacactaaagata

SEQ ID NO: 701 - DNA, 55 bp, synthetic construct
  misc_feature <1: note = /5Phos/
  source 1..55: mol_type = other DNA
  misc_feature 1..55: note = Synthetic
agcttacattgatatgggatttctgggaattattacagctcatacactaaagata

SEQ ID NO: 702 - DNA, 54 bp, synthetic construct
  source 1..54: mol_type = other DNA
  misc_feature 1..54: note = Synthetic
  misc_feature <1: note = /5Phos/
agcttaagtttcacttacggtgcaggaattattacagctcatacactaaagata

SEQ ID NO: 703 - DNA, 53 bp, synthetic construct
  misc_feature <1: note = /5Phos/
  source 1..53: mol_type = other DNA
  misc_feature 1..53: note = Synthetic
atcttacaagactcccaagttgtgtaattattacatctcatacactaaagata

SEQ ID NO: 704 - DNA, 55 bp, synthetic construct
  source 1..55: mol_type = other DNA
  misc_feature 1..55: note = Synthetic
  misc_feature <1: note = /5Phos/
atcttaagctggtctcataaacaccgtaattattacatctcatacactaaagata

SEQ ID NO: 705 - DNA, 53 bp, synthetic construct
  misc_feature 1..53: note = Synthetic
  source 1..53: mol_type = other DNA
  misc_feature <1: note = /5Phos/
atcttaccataggtgcctacgtggtaattattacatctcatacactaaagata

SEQ ID NO: 706 - DNA, 58 bp, synthetic construct
  misc_feature <1: note = /5Phos/
  source 1..58: mol_type = other DNA
  misc_feature 1..58: note = Synthetic
atcttacttgaatgatgaagatggtgcccaaattattacatctcatacactaaagata

SEQ ID NO: 707 - DNA, 53 bp, synthetic construct
  misc_feature 1..53: note = Synthetic
  misc_feature <1: note = /5Phos/
  source 1..53: mol_type = other DNA
atcttaaaaggccctgggtgtctggaattattacatctcatacactaaagata

SEQ ID NO: 708 - DNA, 54 bp, synthetic construct
  misc_feature <1: note = /5Phos/
  misc_feature 1..54: note = Synthetic
  source 1..54: mol_type = other DNA
aagttatgactgattggagaagaagcaattattacaagtcatacactaaagata

SEQ ID NO: 709 - DNA, 55 bp, synthetic construct
  source 1..55: mol_type = other DNA
  misc_feature <1: note = /5Phos/
  misc_feature 1..55: note = Synthetic
aagttacacactgccctagatggaccgaattattacaagtcatacactaaagata

SEQ ID NO: 710 - DNA, 54 bp, synthetic construct
  misc_feature <1: note = /5Phos/
  source 1..54: mol_type = other DNA
  misc_feature 1..54: note = Synthetic
aagttaacagtgtctgattctgcggtaattattacaagtcatacactaaagata

SEQ ID NO: 711 - DNA, 56 bp, synthetic construct
  misc_feature <1: note = /5Phos/
  source 1..56: mol_type = other DNA
  misc_feature 1..56: note = Synthetic
aagttaccaagttcttatcctcacagctaattattacaagtcatacactaaagata

SEQ ID NO: 712 - DNA, 57 bp, synthetic construct
  misc_feature <1: note = /5Phos/
  source 1..57: mol_type = other DNA
  misc_feature 1..57: note = Synthetic
aagttactgagcacatgcctcgcaatcctaattattacaagtcatacactaaagata

SEQ ID NO: 713 - DNA, 54 bp, synthetic construct
  misc_feature <1: note = /5Phos/
  misc_feature 1..54: note = Synthetic
  source 1..54: mol_type = other DNA
aagttagtcacatgggtggcattgggaattattacaagtcatacactaaagata

SEQ ID NO: 714 - DNA, 55 bp, synthetic construct
  misc_feature 1..55: note = Synthetic
  source 1..55: mol_type = other DNA
  misc_feature <1: note = /5Phos/
acgttattccgtaccaggtttagaaataattattacacgtcatacactaaagata

SEQ ID NO: 715 - DNA, 53 bp, synthetic construct
  misc_feature 1..53: note = Synthetic
  source 1..53: mol_type = other DNA
  misc_feature <1: note = /5Phos/
acgttaactctgtagcttccttgggaattattacacgtcatacactaaagata

SEQ ID NO: 716 - DNA, 53 bp, synthetic construct
  source 1..53: mol_type = other DNA
  misc_feature 1..53: note = Synthetic
  misc_feature <1: note = /5Phos/
acgttagggaataggcccttctcctaattattacacgtcatacactaaagata

SEQ ID NO: 717 - DNA, 54 bp, synthetic construct
  misc_feature 1..54: note = Synthetic
  source 1..54: mol_type = other DNA
  misc_feature <1: note = /5Phos/
acgttactcagtccattgtctgccctaattattacacgtcatacactaaagata

SEQ ID NO: 718 - DNA, 57 bp, synthetic construct
  misc_feature <1: note = /5Phos/
  misc_feature 1..57: note = Synthetic
  source 1..57: mol_type = other DNA
acgttagaccagatagccacatgaaacataattattacacgtcatacactaaagata

SEQ ID NO: 719 - DNA, 55 bp, synthetic construct
  misc_feature <1: note = /5Phos/
  source 1..55: mol_type = other DNA
  misc_feature 1..55: note = Synthetic
acgttatcgtgatcatatgatggtgctaattattacacgtcatacactaaagata

SEQ ID NO: 720 - DNA, 53 bp, synthetic construct
  source 1..53: mol_type = other DNA
  misc_feature <1: note = /5Phos/
  misc_feature 1..53: note = Synthetic
aggttagtctccacggaaacagcataattattacaggtcatacactaaagata

SEQ ID NO: 721 - DNA, 54 bp, synthetic construct
  source 1..54: mol_type = other DNA
  misc_feature 1..54: note = Synthetic
  misc_feature <1: note = /5Phos/
aggttaaaccaacttttgctggtcctaattattacaggtcatacactaaagata

SEQ ID NO: 722 - DNA, 57 bp, synthetic construct
  misc_feature 1..57: note = Synthetic
  misc_feature <1: note = /5Phos/
  source 1..57: mol_type = other DNA
aggttaggctgttggagatactgaacagtaattattacaggtcatacactaaagata

SEQ ID NO: 723 - DNA, 53 bp, synthetic construct
  misc_feature 1..53: note = Synthetic
  misc_feature <1: note = /5Phos/
  source 1..53: mol_type = other DNA
aggttaccagaacacacacaacggtaattattacaggtcatacactaaagata

SEQ ID NO: 724 - DNA, 59 bp, synthetic construct
  misc_feature 1..59: note = Synthetic
  misc_feature <1: note = /5Phos/
  source 1..59: mol_type = other DNA
aggttacttgtgacatacaatgatgagcagcaattattacaggtcatacactaaagata

SEQ ID NO: 725 - DNA, 56 bp, synthetic construct
  source 1..56: mol_type = other DNA
  misc_feature <1: note = /5Phos/
  misc_feature 1..56: note = Synthetic
aggttattcatcaatgtgactgctctctaattattacaggtcatacactaaagata

SEQ ID NO: 726 - DNA, 56 bp, synthetic construct
  source 1..56: mol_type = other DNA
  misc_feature 1..56: note = Synthetic
  misc_feature <1: note = /5Phos/
atgttacggcgattcgcgacttctgagtaattattacatgtcatacactaaagata

SEQ ID NO: 727 - DNA, 53 bp, synthetic construct
  misc_feature 1..53: note = Synthetic
  misc_feature <1: note = /5Phos/
  source 1..53: mol_type = other DNA
atgttaatcagaggtctgggagcctaattattacatgtcatacactaaagata

SEQ ID NO: 728 - DNA, 53 bp, synthetic construct
  source 1..53: mol_type = other DNA
  misc_feature <1: note = /5Phos/
  misc_feature 1..53: note = Synthetic
atgttaaggcgtactgctggtacccaattattacatgtcatacactaaagata

SEQ ID NO: 729 - DNA, 54 bp, synthetic construct
  misc_feature 1..54: note = Synthetic
  misc_feature <1: note = /5Phos/
  source 1..54: mol_type = other DNA
atgttatcagtgtttggctgaaccagaattattacatgtcatacactaaagata

SEQ ID NO: 730 - DNA, 53 bp, synthetic construct
  source 1..53: mol_type = other DNA
  misc_feature <1: note = /5Phos/
  misc_feature 1..53: note = Synthetic
aatttacctgataggaatggcccctaattattacaattcatacactaaagata

SEQ ID NO: 731 - DNA, 55 bp, synthetic construct
  source 1..55: mol_type = other DNA
  misc_feature <1: note = /5Phos/
  misc_feature 1..55: note = Synthetic
aatttatggaacactccactgaggtctaattattacaattcatacactaaagata

SEQ ID NO: 732 - DNA, 55 bp, synthetic construct
  misc_feature <1: note = /5Phos/
  misc_feature 1..55: note = Synthetic
  source 1..55: mol_type = other DNA
aatttatgtttgtctttggggtctgcaaattattacaattcatacactaaagata

SEQ ID NO: 733 - DNA, 54 bp, synthetic construct
  misc_feature 1..54: note = Synthetic
  source 1..54: mol_type = other DNA
  misc_feature <1: note = /5Phos/
aatttaaccaaaagctgaggtgaaggaattattacaattcatacactaaagata

SEQ ID NO: 734 - DNA, 58 bp, synthetic construct
  misc_feature <1: note = /5Phos/
  misc_feature 1..58: note = Synthetic
  source 1..58: mol_type = other DNA
aatttattatgttgaaaccatggacagcagaattattacaattcatacactaaagata

SEQ ID NO: 735 - DNA, 56 bp, synthetic construct
  source 1..56: mol_type = other DNA
  misc_feature 1..56: note = Synthetic
  misc_feature <1: note = /5Phos/
actttaacggttatcaaaacaacgccagaattattacacttcatacactaaagata

SEQ ID NO: 736 - DNA, 53 bp, synthetic construct
  misc_feature <1: note = /5Phos/
  source 1..53: mol_type = other DNA
  misc_feature 1..53: note = Synthetic
actttactaaacgtgatggcagaggaattattacacttcatacactaaagata

SEQ ID NO: 737 - DNA, 55 bp, synthetic construct
  misc_feature <1: note = /5Phos/
  misc_feature 1..55: note = Synthetic
  source 1..55: mol_type = other DNA
actttatatggagtagcttcttcacccaattattacacttcatacactaaagata

SEQ ID NO: 738 - DNA, 53 bp, synthetic construct
  misc_feature 1..53: note = Synthetic
  source 1..53: mol_type = other DNA
  misc_feature <1: note = /5Phos/
actttaagctacacaggcaaggagtaattattacacttcatacactaaagata

SEQ ID NO: 739 - DNA, 54 bp, synthetic construct
  misc_feature <1: note = /5Phos/
  source 1..54: mol_type = other DNA
  misc_feature 1..54: note = Synthetic
actttaggcaggattttgaggtccagaattattacacttcatacactaaagata

SEQ ID NO: 740 - DNA, 54 bp, synthetic construct
  source 1..54: mol_type = other DNA
  misc_feature 1..54: note = Synthetic
  misc_feature <1: note = /5Phos/
actttagcatgtggttgaagaggggtaattattacacttcatacactaaagata

SEQ ID NO: 741 - DNA, 56 bp, synthetic construct
  misc_feature <1: note = /5Phos/
  source 1..56: mol_type = other DNA
  misc_feature 1..56: note = Synthetic
agtttatcgagttgaccgagtactgggtaattattacagttcatacactaaagata

SEQ ID NO: 742 - DNA, 53 bp, synthetic construct
  misc_feature 1..53: note = Synthetic
  source 1..53: mol_type = other DNA
  misc_feature <1: note = /5Phos/
agtttagatcttgcacagacctagtaattattacagttcatacactaaagata

SEQ ID NO: 743 - DNA, 53 bp, synthetic construct
  misc_feature 1..53: note = Synthetic
  misc_feature <1: note = /5Phos/
  source 1..53: mol_type = other DNA
agtttatgatccacagtggactgcgaattattacagttcatacactaaagata

SEQ ID NO: 744 - DNA, 53 bp, synthetic construct
  misc_feature <1: note = /5Phos/
  misc_feature 1..53: note = Synthetic
  source 1..53: mol_type = other DNA
agtttaaaatgacggggttaaggcaaattattacagttcatacactaaagata

SEQ ID NO: 745 - DNA, 57 bp, synthetic construct
  misc_feature <1: note = /5Phos/
  source 1..57: mol_type = other DNA
  misc_feature 1..57: note = Synthetic
agtttaatctctacgcttgtaaccaggctaattattacagttcatacactaaagata

SEQ ID NO: 746 - DNA, 59 bp, synthetic construct
  source 1..59: mol_type = other DNA
  misc_feature <1: note = /5Phos/
  misc_feature 1..59: note = Synthetic
agtttaaactgttctaacttttaatacaactaattattacagttcatacactaaagata

SEQ ID NO: 747 - DNA, 53 bp, synthetic construct
  source 1..53: mol_type = other DNA
  misc_feature 1..53: note = Synthetic
  misc_feature <1: note = /5Phos/
attttacacagcacgtaggttgcagaattattacatttcatacactaaagata

SEQ ID NO: 748 - DNA, 55 bp, synthetic construct
  misc_feature 1..55: note = Synthetic
  misc_feature <1: note = /5Phos/
  source 1..55: mol_type = other DNA
attttaccggagaacattccatttgataattattacatttcatacactaaagata

SEQ ID NO: 749 - DNA, 53 bp, synthetic construct
  source 1..53: mol_type = other DNA
  misc_feature <1: note = /5Phos/
  misc_feature 1..53: note = Synthetic
attttacaatagctccttggtgagtaattattacatttcatacactaaagata

SEQ ID NO: 750 - DNA, 55 bp, synthetic construct
  misc_feature 1..55: note = Synthetic
  source 1..55: mol_type = other DNA
  misc_feature <1: note = /5Phos/
attttatgcctatgggatcagtgaactaattattacatttcatacactaaagata

SEQ ID NO: 751 - DNA, 55 bp, synthetic construct
  misc_feature 1..55: note = Synthetic
  source 1..55: mol_type = other DNA
  misc_feature <1: note = /5Phos/
attttaagttccttcctcatacatgctaattattacatttcatacactaaagata

SEQ ID NO: 752 - DNA, 54 bp, synthetic construct
  misc_feature 1..54: note = Synthetic
  source 1..54: mol_type = other DNA
  misc_feature <1: note = /5Phos/
attttatcatgtcctctggattcccaaattattacatttcatacactaaagata

SEQ ID NO: 753 - protein, 8 aa, synthetic construct
  REGION 1..8: note = Synthetic
SIINFEKL

SEQ ID NO: 754 - protein, 17 aa, synthetic construct
  REGION 1..17: note = Synthetic
ISQAVHAAHAEINEAGR

SEQ ID NO: 755 - protein, 15 aa, synthetic construct
  REGION 1..15: note = Synthetic
GGGGGGGGGGGGGGG

SEQ ID NO: 756 - protein, 15 aa, synthetic construct
  REGION 1..15: note = Synthetic
GGGGSGGGGSGGGGS

SEQ ID NO: 757 - protein, 5 aa, synthetic construct
  REGION 1..5: note = Synthetic
TEFCA

SEQ ID NO: 758 - protein, 9 aa, synthetic construct
  REGION 1..9: note = Synthetic
KYNKANVFL

SEQ ID NO: 759 - DNA, 1493 bp, synthetic construct
  misc_feature 1..1493: note = Synthetic
  source 1..1493: mol_type = other DNA
tggggacggtgattcaattctatgggaagcctttacaaaaaccattctgtctgtcccaaggcccacagtcctctactcttctcctgggtcgcaagcctagatgtgtttagatccagaatgctttcacgtcacctctgcagcctgctaggccaagattgtggagaagaatggggccttaccagagcaggagcctcctacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggccgaggcattcaggcagcgagagcagagcagcgtagagcagcacagctgagctcgtgaggcaggagactcagcccgaggaaatcgcagataagtttttaattaaaaagattgagcagtaaaaagaattagaactctaaacttaagctaatagagtagcttatcgaaatattacttagtcttaataatctaagaagatcttaagagataacatgaaggcttatttaaacagtttgaaaaaggaaatgaggagaaaagtatttgtactgtataatggaggctgaccagagcagtttaggagattgtaaagggaggttttgtgaagttctaaaaggttctagtttgaaggtcggccttgtagattaaaacgaaggttacctaaatagaatctaagtggcatttaaaacagtaaagttgtagagaatagtttgaaaatgaggtgtagttttaaaagattgagaaaagtaggttaagttgacggccgttataaaaatccttcgactggcgcatgtacgtttgaaggcatgagttggaaacagggaagatggaagtgttaggc

SEQ ID NO: 760 - DNA, 985 bp, synthetic construct
  source 1..985: mol_type = other DNA
  misc_feature 1..985: note = Synthetic
tgggggctctctcaccaaagagaccagtatcctgagaggaagcatgtctaacactgccttccctgaccccgcctggaacaccaccctgctatcttgggttgctctctttctcctgggaacaagttcagcaaattctggggttgtccagtctccaagatacataatcaaaggaaagggagaaaggtccattctaaaatgtattcccatctctggacatctctctgtggcctggtatcaacagactcaggggcaggaactaaagttcttcattcagcattatgataaaatggagagagataaaggaaacctgcccagcagattctcagtccaacagtttgatgactatcactctgagatgaacatgagtgccttggagctagaggactctgccgtgtacttctgtgccagctcttccgggacagggagatatgaacagtacttcggtcccggcaccaggctcacggttttagaggatctgagaaatgtgactccacccaaggtctccttgtttgagccatcaaaagcagagattgcaaacaaacaaaaggctaccctcgtgtgcttggccggccaagcacacgagggtagccttttgtttgtttgcaatctctgcttttgatggctcaaacaaggagaccttgggtggagtcacatttctcagatcctctaggacggtgagtcgtgtccctggtccgaagaactgctcagcatagttacacagcagaaaagggctaccaagaattcttcctctttcctcaccttcctcaatacagacccggggttactcacattctccatctacctccagaactggtaaggcaatcttgaccttgcatgaggccgatctcaaattgaggaggagataccacttttggcttgggaacggatatccatctattcactcagggtttgacaaagtgaatttgaaatctgaaggcttcctgttggtgtccaggtagcctccaatgagaaaggacttgtcccc

SEQ ID NO: 761 - DNA, 946 bp, synthetic construct
  misc_feature 1..946: note = Synthetic
  source 1..946: mol_type = other DNA
acagtcctctactcttctcctgggtcgcaagcctagatgtgtttagatccagaatgctttcacgtcacctctgcagcctgctaggccaagattgtggagaagaatggggccttaccagagcaggagcctcctacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaataggattgcggctttcctatgcaagccaccacagctctcttacatctcagtgtaggagtgaatgtggaacatcagaggatctgagaaatgtgactccacccaaggtctccttgtttgagccatcaaaagcagagattgcaaacaaacaaaaggctaccctcgtgtgcttggcc

SEQ ID NO: 762 - DNA, 2877 bp, synthetic construct
  misc_feature 1..2877: note = Synthetic
  source 1..2877: mol_type = other DNA
catttgggtcttttttttttgggggaagatattcagagcaggacagctactcagcatcctcttggactgagcttatctgccagagcagaaggatttctggattggatgctgggaatagagaaacactcatggttggtggggatctgagtcagggttggagcagcaggagcgggaagcactgagtttttgtcctgagcctggaggctgtgagtgcagaaacgctgtattttggctcaggaaccagactgactgttctcgaggatctgagaaatgtgactccacccaaggtctccttgtttgagccatcaaaagcagagattgcaaacaaacaaaaggctaccctcgtgtgcttggccggccaagcacacgagggtagccttttgtttgtttgcaatctctgcttttgatggctcaaacaaggagaccttgggtggagtcacatttctcagatcctctaaaaccgtgagcctggtgccgggaccgaagtactgttcataggagcacagaggctcaaccccacacacaaacccgcggggttcagcgaggtattctcccatttagggaaaatctccctgctactcagcctccctctccagcgcccggcttgggaactcagggtaaagacgccagaagccccagaactccccgctgcagttgccaagaagctcgcataggctgttcaatcggctgccaatcagtcagggcaaggccccaagtcaaagtcggtgaacaggcagagggtgctgtcctgagaccgaggatcttttaactggtacacagcaggttctgggttctggatgtttgatttaacagagagtttagtgcctttcccgaaggttagctttgcattgcttcctccagaattcacagtcatttggggccttacaataaccagcaggcaggaaggaccgagaggcagaccaagaaaagcgacatcatcttcccctctagtggaagccttcctccttgcagccttagtccccagagtctgggaaggcttagagcatcctctcaaaactaacagctggctgtgaagctactctgagcttgatcagaaaccactcccggggtcctaggtaagaaggcagaggccatacaggtgggagagaaggtaggacagtcagccacaaagacccccggagcagaaaaccttagcagagtggatatttaactatccaaggccctgtatcaggaaagaggaggtttgtgccagcatttccaggactgtgcaaacaccgggcagctctactttggtgaaggctcaaagctgacagtgctggaggatctgagaaatgtgactccacccaaggtctccttgtttgagccatcaaaagcagagattgcaaacaaacaaaaggctaccctcgtgtgcttggccgtttctgatgtgcagtcagtcaagctaggagaaactacattcctgccgtgaccctactatggatatctggcttctaggttggataatttttagtttcttggaagcaggacacacaggacccaaagtcttacagatcccaagtcatcaaataatagatatggggcagatggtgaccctcaattgtgacccagtttctaatcacctatatttttattggtataaacagattttaggacagcagatggagtttctggttaatttctacaatggtaaagtcatggagaagtctaaactgtttaaggatcagttttcagttgaaagaccagatggttcatatttcactctgaaaatccaacccacagcactggaggactcagctgtgtacttctgtgccagcagctcgggacaggaaaactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggcctacgataccaggcagtaaagcttcgggtttcttatggggctcattggaggctacctggacaccaacaggaagccttcagatttcaaattcactttgtcaaaccctgagtgaatagatggatatccgttcccaagccaaaagtggtatctcctcctcaatttgagatcggcctcatgcaaggtcaagattgccttaccagttctggaggtagatggagaatgtgagtaaccccgggtctgtattgaggaaggtgaggaaagaggaagaattcttggtagcccttttctgctgtgtaactatgctgagcagttcttcggaccagggacacgactcaccgtcctagaggatctgagaaatgtgactccacccaaggtctccttgtttgagccatcaaaagcagagattgcaaacaaacaaaaggctaccctcgtgtgcttggccggccaagcacacgagggtagccttttgtttgtttgcaatctctgcttttgatggctcaaacaaggagaccttgggtggagtcacatttctcagatcctctacaactgtgagtctggttcctttaccaaagaagacttctgtgtttgcacagtgccataggatgaggagaaaaataatcttaaggggtgaagagaggctcttcccagctagtatctagaggaccataggaggagtaatcgctttgtgtgcatcacaggtattgcacaaaagaacattttgacgatagacttgtggcaatgtcaaacatggtagattccctccagtggaacttacatcccttttgtgaagaatcccccatatatattgttcctctctcaaggtccatcaaagtgaggtagcctgtcagagaacagttttcctggtcctatgatgatattcatcaagagctgagatcttttaaaacaaaacaaacctgtagaactgttcacctctggctccccca

SEQ ID NO: 763 - DNA, 940 bp, synthetic construct
  source 1..940: mol_type = other DNA
  misc_feature 1..940: note = Synthetic
gggggatcatttcaatgacacccagcgccaagaaaaaagaacattcaaaagaagaacagggggcccacagtcctctactcttctcctgggtcgcaagcctagatgtgtttagatccagaatgctttcacgtcacctctgcagcctgctaggccaagattgtggagaagaatggggccttaccagagcaggagcctcctacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggcc

SEQ ID NO: 764 - DNA, 913 bp, synthetic construct
  source 1..913: mol_type = other DNA
  misc_feature 1..913: note = Synthetic
gggggtcacacacagagtcttgattgtgggacaaggcccacagtcctctactcttctcctgggtcgcaagcctagatgtgtttagatccagaatgctttcacgtcacctctgcagcctgctaggccaagattgtggagaagaatggggccttaccagagcaggagcctcctacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggcc

SEQ ID NO: 765 - DNA, 1790 bp, synthetic construct
  misc_feature 1..1790: note = Synthetic
  source 1..1790: mol_type = other DNA
tggggaggcctggagctgtatctcttgcgatctgatcttcatagggaagaatgcacagcctcctggggttgttgttgtggctgcaactgacaagggtgaatagtcaactagcagaagagaattcgtgggccctgagcgtccacgagggtgaaagtgtcacggtgaattgtagttacaagacatccataactgccctacagtggtacagacagaagtcaggcaaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctccttataatgttggtgacaacagtaagctgatttggggcttggggacaagtctggtagtaaatccaaacatccagaacccagaacctgctgtgtaccagttaaaagatcctcggtctcaggacagcaccctctgcctgttcaccgactttgacttggggaaaagatcatttcaatgacacccagcgccaagaaaaaagaacattcaaaagaagaacagggggtaaagaggaaacccctgcattagctcgcatcttaccaccaccttgcacaatgggggtcggggggggatgtcaccttccttatcttcaactcccccccagaggagcagcttatctggtggtttcttccagccctcaaggggtagacctatgggagggtccttttttgtataaagctgtaacattgtggggacagtgcagaaacgctgtattttggctcaggaaccagactgactgttctcgaggatctgagaaatgtgactccacccaaggtctccttgtttgagccatcaaaagcagagattgcaaacaaacaaaaggctaccctcgtgtgcttggcctgggggaagatattcagagcaggacagctactcagcatcctcttggactgagcttatctgccagagcagaaggatttctggattggatgctgggaatagagaaacactcatggttggtggggatctgagtcagggttggagcagcaggagcgggaagcactgagtttttgtcctgagcctggaggctgtgagtgcagaaacgctgtattttggctcaggaaccagactgactgttctcgaggatctgagaaatgtgactccacccaaggtctccttgtttgagccatcaaaagcagagattgcaaacaaacaaaaggctaccctcgtgtgcttggccaatacccgtctggagcctgattccaccatgagctgcaggcttctcctctatgtttccctatgtcttgtggaaacagcactcatgaacactaaaattactcagtcaccaagatatctaatcctgggaagagcaaataagtctttggaatgtgagcaacatctgggacataatgctatgtactggtataaacagagcgctgagaagccgccagagctcatgtttctctacaatcttaaacagttgattcgaaatgagacggtgcccagtcgttttatacctgaatgcccagacagctccaagctacttttacatatatctgccgtggatccagaagactcagctgtctatttttgtgccagcagccaagatagggacagggatgctgagcagttcttcggaccagggacacgactcaccgtcctagaggatctgagaaatgtgactccacccaaggtctccttgtttgagccatcaaaagcagagattgcaaacaaacaaaaggctaccctcgtgtgcttggcc

SEQ ID NO: 766 - DNA, 1802 bp, synthetic construct
  source 1..1802: mol_type = other DNA
  misc_feature 1..1802: note = Synthetic
gtgagtgaggagtgtgaggagatcctgcaggaggattgccctgggagattggtgcactcaaggaccaagtgtcatttcttccatgaacatgcgtcctgacacctgctcagttcttgtgctcctcttaatgctcagaaggaacaatggagactctgtgacccagacagaaggcctggtcactctcaccgaggggttgcctgtgatgctgaactgcacctatcagagtacttactcacctttccttttctggtatgtgcaacatctcaacgaagcccctaagctacttttgaagagcttcacagacaacaagaggcccgagcaccaagggttccacgccactctccataagagcagcagctccttccatctgcagaagtcctcagcgcagctgtcagactctgccctgtactactgtgctttgagtgatcttgttggtgacaacagtaagctgatttggggcttggggacaagtctggtagtaaatccaaacatccagaacccagaacctgctgtgtaccagttaaaagatcctcggtctcaggacagcaccctctgcctgttcaccgactttgacttggggagaacattcaaaagaagaacagggggtaaagaggaaacccctgcattagctcgcatcttaccaccaccttgcacaatgggggtcggggggggatgtcaccttccttatcttcaactcccccccagaggagcagcttatctggtggtttcttccagccctcaaggggtagacctatgggagggtccttttttgtataaagctgtaacattgtggggacaggggggttctggaaatacgctctattttggagaaggaagccggctcattgttgtagaggatctgagaaatgtgactccacccaaggtctccttgtttgagccatcaaaagcagagattgcaaacaaacaaaaggctaccctcgtgtgcttggccaatacccgtctggagcctgattccaccatgagctgcaggcttctcctctatgtttccctatgtcttgtggaaacagcactcatgaacactaaaattactcagtcaccaagatatctaatcctgggaagagcaaataagtctttggaatgtgagcaacatctgggacataatgctatgtactggtataaacagagcgctgagaagccgccagagctcatgtttctctacaatcttaaacagttgattcgaaatgagacggtgcccagtcgttttatacctgaatgcccagacagctccaagctacttttacatatatctgccgtggatccagaagactcagctgtctatttttgtgccagcagccaagacccaaacacagaagtcttctttggtaaaggaaccagactcacagttgtagaggatctgagaaatgtgactccacccaaggtctccttgtttgagccatcaaaagcagagattgcaaacaaacaaaaggctaccctcgtgtgcttggccggggatagatggatatccgttcccaagccaaaagtggtatctcctcctcaatttgagatcggcctcatgcaaggtcaagattgccttaccagttctggaggtagatggagaatgtgagtaaccccgggtctgtattgaggaaggtgaggaaagaggaagaattcttggtagcccttttctgctgtgtaactatgctgagcagttcttcggaccagggacacgactcaccgtcctagaggatctgagaaatgtgactccacccaaggtctccttgtttgagccatcaaaagcagagattgcaaacaaacaaaaggctaccctcgtgtgcttggcc

SEQ ID NO: 767 - DNA, 1077 bp, synthetic construct
  source 1..1077: mol_type = other DNA
  misc_feature 1..1077: note = Synthetic
tatggggcaatgctcagaaaccaaccaccttgaacaggcctggagctgtatctcttgcgatctgatcttcatagggaagaatgcacagcctcctggggttgttgttgtggctgcaactgacaagggtgaatagtcaactagcagaagagaattcgtgggccctgagcgtccacgagggtgaaagtgtcacggtgaattgtagttacaagacatccataactgccctacagtggtacagacagaagtcaggcaaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctccttataatgttggtgacaacagtaagctgatttggggcttggggacaagtctggtagtaaatccaaacatccagaacccagaacctgctgtgtaccagttaaaagatcctcggtctcaggacagcaccctctgcctgttcaccgactttgactaatacccgtctggagcctgattccaccatgagctgcaggcttctcctctatgtttccctatgtcttgtggaaacagcactcatgaacactaaaattactcagtcaccaagatatctaatcctgggaagagcaaataagtctttggaatgtgagcaacatctgggacataatgctatgtactggtataaacagagcgctgagaagccgccagagctcatgtttctctacaatcttaaacagttgattcgaaatgagacggtgcccagtcgttttatacctgaatgcccagacagctccaagctacttttacatatatctgccgtggatccagaagactcagctgtctatttttgtgccagcagccaagatagggacagggatgctgagcagttcttcggaccagggacacgactcaccgtcctagaggatctgagaaatgtgactccacccaaggtctccttgtttgagccatcaaaagcagagattgcaaacaaacaaaaggctaccctcgtgtgcttggcc

SEQ ID NO: 768 - DNA, 907 bp, synthetic construct
  source 1..907: mol_type = other DNA
  misc_feature 1..907: note = Synthetic
ggggagaacattcaaaagaagaacagggggcccacagtcctctactcttctcctgggtcgcaagcctagatgtgtttagatccagaatgctttcacgtcacctctgcagcctgctaggccaagattgtggagaagaatggggccttaccagagcaggagcctcctacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggcc

SEQ ID NO: 769 - DNA, 939 bp, synthetic construct
  misc_feature 1..939: note = Synthetic
  source 1..939: mol_type = other DNA
tggggcacggtgattcaattctatgggaagcctttacaaaaaccattctgtctgtcccaaggcccacagtcctctactcttctcctgggtcgcaagcctagatgtgtttagatccagaatgctttcacgtcacctctgcagcctgctaggccaagattgtggagaagaatggggccttaccagagcaggagcctcctacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggcc

SEQ ID NO: 770 - DNA, 963 bp, synthetic construct
  source 1..963: mol_type = other DNA
  misc_feature 1..963: note = Synthetic
aaagctgtaacattgtggggacagggggccacggtgattcaattctatgggaagcctttacaaaaaccattctgtctgtcccaaggcccacagtcctctactcttctcctgggtcgcaagcctagatgtgtttagatccagaatgctttcacgtcacctctgcagcctgctaggccaagattgtggagaagaatggggccttaccagagcaggagcctcctacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggcc

SEQ ID NO: 771 - DNA, 1340 bp, synthetic construct
  source 1..1340: mol_type = other DNA
  misc_feature 1..1340: note = Synthetic
gaatgacacccagcgccaagaaaaaagaacattcaaaagaagaacagggggcccacagtcctctactcttctcctgggtcgcaagcctagatgtgtttagatccagaatgctttcacgtcacctctgcagcctgctaggccaagattgtggagaagaatggggccttaccagagcaggagcctcctacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggccctcgtgaggcaggagactcagcccgaggagatcgcagataagtttttaattaaaaagattgagcagtaaaaagaattagaactctaaacttaagctaatagagtagcttatcgaaatattacttagtcttaataatctaagaagatcttaagagataacatgaaggcttatttaaacagtttgaaaaaggaaatgaggagaaaagtatttgtactgtataatggaggctgaccagagcagtttaggagattgtaaagggaggttttgtgaagttctaaaaggttctagtttgaaggtcggccttgtagattaaaacgaaggttacctaaatagaatctaagtggcatttaaaacagtaaagttgtagagaatagtttgaaaatgaggtgtagttttaaaagattgagaaaag

SEQ ID NO: 772 - DNA, 1144 bp, synthetic construct
  source 1..1144: mol_type = other DNA
  misc_feature 1..1144: note = Synthetic
ctctggttctcggacgattttgttcatttcttatatgggatccacaaaggtggattcatcataggtccaggaatgtttatggtggagtcaagatgaacctcgggtggaagtcaggaaagagagaggagaaaggctcacagagagggagggggttttgaagtggacccgggaggctgtgttctggaaatacgctctattttggagaaggaagccggctcattgttgtagaggatctgagaaatgtgactccacccaaggtctccttgtttgagccatcaaaagcagagattgcaaacaaacaaaaggctaccctcgtgtgcttggccgggtacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaataggattgcggctttcctatgcaagccaccacagctctcttacatctcagtgtaggagtgaatgtggaacatcagaggatctgagaaatgtgactccacccaaggtctccttgtttgagccatcaaaagcagagattgcaaacaaacaaaaggctaccctcgtgtgcttggcc

SEQ ID NO: 773 - DNA, 1027 bp, synthetic construct
  source 1..1027: mol_type = other DNA
  misc_feature 1..1027: note = Synthetic
tcgtttgatttcttttctggggacggtgattcaattctatgggaagcctttacaaaaaccattctgtctgtcccaaggcccacagtcctctactcttctcctgggtcgcaagcctagatgtgtttagatccagaatgctttcacgtcacctctgcagcctgctaggccaagattgtggagaagaatggggccttaccagagcaggagcctcctacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaataggattgcggctttcctatgcaagccaccacagctctcttacatctcagtgtaggagtgaatgtggaacatcagaggatctgagaaatgtgactccacccaaggtctccttgtttgagccatcaaaagcagagattgcaaacaaacaaaaggctaccctcgtgtgcttggcc

SEQ ID NO: 774 - DNA, 1409 bp, synthetic construct
  misc_feature 1..1409: note = Synthetic
  source 1..1409: mol_type = other DNA
agaaaccaaccaccttgaacaggcctggagctgtatctcttgcgatctgatcttcatagggaagaatgcacagcctcctggggttgttgttgtggctgcaactgacaagggtgaatagtcaactagcagaagagaattcgtgggccctgagcgtccacgagggtgaaagtgtcacggtgaattgtagttacaagacatccataactgccctacagtggtacagacagaagtcaggcaaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctccttataatgttggtgacaacagtaagctgatttggggcttggggacaagtctggtagtaaatccaaacatccagaacccagaacctgctgtgtaccagttaaaagatcctcggtctcaggacagcaccctctgcctgttcaccgactttgactgcatttcttatatggggagatattcagagcaggacagctactcagcatcctcttggactgagcttatctgccagagcagaaggatttctggattggatgctgggaatagagaaacactcatggttggtggggatctgagtcagggttggagcagcaggagcgggaagcactgagtttttgtcctgagcctggaggctgtgagtgcagaaacgctgtattttggctcaggaaccagactgactgttctcgaggatctgagaaatgtgactccacccaaggtctccttgtttgagccatcaaaagcagagattgcaaacaaacaaaaggctaccctcgtgtgcttggccaatacccgtctggagcctgattccaccatgagctgcaggcttctcctctatgtttccctatgtcttgtggaaacagcactcatgaacactaaaattactcagtcaccaagatatctaatcctgggaagagcaaataagtctttggaatgtgagcaacatctgggacataatgctatgtactggtataaacagagcgctgagaagccgccagagctcatgtttctctacaatcttaaacagttgattcgaaatgagacggtgcccagtcgttttatacctgaatgcccagacagctccaagctacttttacatatatctgccgtggatccagaagactcagctgtctatttttgtgccagcagccaagatagggacagggatgctgagcagttcttcggaccagggacacgactcaccgtcctagaggatctgagaaatgtgactccacccaaggtctccttgtttgagccatcaaaagcagagattgcaaacaaacaaaaggctaccctcgtgtgcttggcc

SEQ ID NO: 775 - DNA, 1414 bp, synthetic construct
  misc_feature 1..1414: note = Synthetic
  source 1..1414: mol_type = other DNA
ggtgattcaattctatgggaagcctttacaaaaaccattctgtctgtcccaaggcccacagtcctctactcttctcctgggtcgcaagcctagatgtgtttagatccagaatgctttcacgtcacctctgcagcctgctaggccaagattgtggagaagaatggggccttaccagagcaggagcctcctacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggcccagcgagagcagagcagcgtagagcagcacagctgagctcgtgaggcaggagactcagcccgaggaaatcgcagataagtttttaattaaaaagattgagcagtaaaaagaattagaactctaaacttaagctaatagagtagcttatcgaaatattacttagtcttaataatctaagaagatcttaagagataacatgaaggcttatttaaacagtttgaaaaaggaaatgaggagaaaagtatttgtactgtataatggaggctgaccagagcagtttaggagattgtaaagggaggttttgtgaagttctaaaaggttctagtttgaaggtcggccttgtagattaaaacgaaggttacctaaatagaatctaagtggcatttaaaacagtaaagttgtagagaatagtttgaaaatgaggtgtagttttaaaagattgagaaaagtaggttaagttgacggccgttataaaaatccttc

SEQ ID NO: 776 - DNA, 878 bp, synthetic construct
  misc_feature 1..878: note = Synthetic
  source 1..878: mol_type = other DNA
gcccacagtcctctactcttctcctgggtcgcaagcctagatgtgtttagatccagaatgctttcacgtcacctctgcagcctgctaggccaagattgtggagaagaatggggccttaccagagcaggagcctcctacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggcc

SEQ ID NO: 777 - DNA, 917 bp, synthetic construct
  source 1..917: mol_type = other DNA
  misc_feature 1..917: note = Synthetic
agcgccaagaaaaaagaacattcaaaagaagaacagggggcccacagtcctctactcttctcctgggtcgcaagcctagatgtgtttagatccagaatgctttcacgtcacctctgcagcctgctaggccaagattgtggagaagaatggggccttaccagagcaggagcctcctacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggcc

SEQ ID NO: 778 - DNA, 933 bp, synthetic construct
  misc_feature 1..933: note = Synthetic
  source 1..933: mol_type = other DNA
acggtgattcaattctatgggaagcctttacaaaaaccattctgtctgtcccaaggcccacagtcctctactcttctcctgggtcgcaagcctagatgtgtttagatccagaatgctttcacgtcacctctgcagcctgctaggccaagattgtggagaagaatggggccttaccagagcaggagcctcctacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggcc

SEQ ID NO: 779 - DNA, 1003 bp, synthetic construct
  misc_feature 1..1003: note = Synthetic
  source 1..1003: mol_type = other DNA
ggggaatgacacccagcgccaagaaaaaagaacattcaaaagaagaacagggggcccacagtcctctactcttctcctgggtcgcaagcctagatgtgtttagatccagaatgctttcacgtcacctctgcagcctgctaggccaagattgtggagaagaatggggccttaccagagcaggagcctcctacactgaatgaacataagaagaatggcctagtggccctagcagcaaaggatgcagggaaatcctcttctgccaacgtccataaaacagaccaccatcagtggataggtgagccagaggtgaacagttctacaggtttgttttgttttaaaagatctcagctcttgatgaatatcatcataggaccaggaaaactgttctctgacaggctacctcactttgatggaccttgagagaggaacaatatatatgggggattcttcacaaaagggatgtaagttccactggagggaatctaccatgtttgacattgccacaagtctatcgtcaaaatgttcttttgtgcaatacctgtgatgcacacaaagcgattactcctcctatggtcctctagatactagctgggaagagcctctcttcaccccttaagattatttttctcctcatcctatggcactgtgcaaacacagaagtcttctttggtaaaggaaccagactcacagttgtaggtaagatatctttcaggtaaatttccaggtctctcttcggacaaagcatgtcctccgtgtccatattcgagtatctgtattctgatgtgcaaactccgactacaccttcggctcagggaccaggcttttggtaataggattgcggctttcctatgcaagccaccacagctctcttacatctcagtgtaggagtgaatgtggaacatcagaggatctgagaaatgtgactccacccaaggtctccttgtttgagccatcaaaagcagagattgcaaacaaacaaaaggctaccctcgtgtgcttggcc

SEQ ID NO: 780 - DNA, 644 bp, synthetic construct
  misc_feature 1..644: note = Synthetic
  source 1..644: mol_type = other DNA
acaccaggaagctccttttgaacatctcaagagtcttcctaccattctagtcatcatagttagccaggtggagcccctgcatagcgcgtctccttgtttctcttttaactaatgcccagagccaaagaaagtccctccaaactatgaacaagtgggttttctgctgggtaaccctttgtctccttactgtagagaccacacatggtgatggtggcatcattactcagacacccaaattcctgattggtcaggaagggcaaaaactgaccttgaaatgtcaacagaatttcaatcatgatacaatgtactggtaccgacaggattcagggaaaggattgagactgatctactattcaataactgaaaacgatcttcaaaaaggcgatctatctgaaggctatgatgcgtctcgagagaagaagtcatctttttctctcactgtgacatctgcccagaagaacgagatggccgtttttctctgtgccagcagtatgcacagcaacgaaagattatttttcggtcatggaaccaagctgtctgtcctggaggatctgagaaatgtgactccacccaaggtctccttgtttgagccatcaaaagcagagattgcaaacaaacaaaaggctaccctcgtgtgcttggcc

SEQ ID NO: 781 - protein, 9 aa, synthetic construct
  REGION 1..9: note = Synthetic
KYNKANAFL

SEQ ID NO: 782 - protein, 9 aa, synthetic construct
  REGION 1..9: note = Synthetic
AAHAEINEA